(19) United States
(12) Patent Application Publication
(10) Pub. No.: US 2023/0398217 A1
(43) Pub. Date: Dec. 14, 2023

(54) BINDING PROTEINS RECOGNIZING HA-1 ANTIGEN AND USES THEREOF

(71) Applicant: TScan Therapeutics, Inc., Waltham, MA (US)

(72) Inventors: Ribhu Nayar, Waban, MA (US); Gavin MacBeath, Wakefield, MA (US); Sonal Jangalwe, Framingham, MA (US); Mollie M. Jurewicz, Auburn, MA (US); Andrew S. Basinski, Sudbury, MA (US); Qikai Xu, Chestnut Hill, MA (US); Antoine J. Boudot, Wilmington, MA (US); Tomasz Kula, Brookline, MA (US)

(21) Appl. No.: 18/035,373

(22) PCT Filed: Nov. 8, 2021
(86) PCT No.: PCT/US2021/058382

§ 371 Date: May 4, 2023

Related U.S. Application Data
(60) Provisional application No. 63/175,350, filed on Apr. 15, 2021.
(60) Provisional application No. 63/129,804, filed on Dec. 23, 2020.
(60) Provisional application No. 63/111,462, filed on Nov. 9, 2020.
(60) Provisional application No. 63/110,851, filed on Nov. 6, 2020.

(51) Int. Cl. A61K 39/00; C12N 15/10; G01N 33/50; C07K 14/725; C07K 14/705

(52) U.S. Cl. CPC A61K 39/4632; C12N 15/1055; G01N 33/5011; C07K 14/7051; C07K 14/70517; A61K 39/464401; A61K 2239/48

(57) ABSTRACT

Provided herein are binding proteins recognizing HA-1 antigen and uses thereof.

### CROSS-REFERENCE TO RELATED APPLICATIONS

This application claims the benefit of U.S. Provisional Application No. 63/110,851, filed on 6 Nov. 2020, U.S. Provisional Application No. 63/111,462, filed on 9 Nov. 2020, U.S. Provisional Application No. 63/129,804, filed on 23 Dec. 2020, and U.S. Provisional Application No. 63/175,350, filed on 15 Apr. 2021; the entire contents of each of said applications are incorporated herein in their entirety by this reference.

### BACKGROUND OF THE INVENTION

Approximately 30-40% of AML patients relapse following allogeneic hematopoietic stem cell transplant therapy, leaving them with very few treatment options (Pavletic et al. (2010) Biol Blood Marrow Transplant. 16:871-890; Miller et al. (2010) Biol Blood Marrow Transplant. 16:565-586). Rare patients that naturally develop an HA-1-specific graft-versus-leukemia T cell response, however, show substantially lower relapse rates (Marijt et al. (2003) PNAS. 100:2742-2747; Spierings et al. (2013) Biol Blood Marrow Transplant. 19:1244-1253). HA-1 (VLHDDLLEA, genotype RS_1801284 A/G or A/A) is an HLA-A*02:01—and hematopoietically restricted minor histocompatibility antigen, making it an ideal candidate for TCR immunotherapy for liquid tumors (Bleakley and Riddell (2011) Immunol. Cell Biol. 89:396-407). There is a need for developing HA-1-specific TCR immunotherapy, such as to treat disorders characterized by expression of an HA-1 antigen.

### SUMMARY OF THE INVENTION

The present invention is based, at least in part, on the discovery of binding proteins, including T cell receptors (TCRs), that recognize HA-1 antigen.

In one aspect, a binding protein comprising: a) a T cell receptor (TCR) alpha chain CDR sequence with at least about 80% identity to a TCR alpha chain CDR sequence selected from the group consisting of TCR alpha chain CDR sequences listed in Table 1; and/or b) a TCR beta chain CDR sequence with at least about 80% identity to a TCR beta chain CDR sequence selected from the group consisting of TCR beta chain CDR sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M, is provided.

In another aspect, a binding protein comprising: a) a TCR alpha chain variable (V_{α}) domain sequence with at least about 80% identity to a TCR V_{α} domain sequence selected from the group consisting of TCR V_{α} domain sequences listed in Table 1; and/or b) a TCR beta chain variable (V_{β}) domain sequence with at least about 80% identity to a TCR V_{β} domain sequence selected from the group consisting of TCR V_{β} domain sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M, is provided.

In still another aspect, a binding protein comprising: a) a TCR alpha chain sequence with at least about 80% identity to a TCR alpha chain sequence selected from the group consisting of TCR alpha chain sequences listed in Table 1; and/or b) a TCR beta chain sequence with at least about 80% identity to a TCR beta chain sequence selected from the group consisting of TCR beta chain sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M, is provided.

In yet another aspect, a binding protein comprising: a) a TCR alpha chain CDR sequence selected from the group consisting of TCR alpha chain CDR sequences listed in Table 1; and/or b) a TCR beta chain CDR sequence selected from the group consisting of TCR beta chain CDR sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M, is provided.

In another aspect, a binding protein comprising: a) a TCR alpha chain variable (V_{α}) domain sequence selected from the group consisting of TCR V_{α} domain sequences listed in Table 1; and/or b) a TCR beta chain variable (V_{β}) domain sequence selected from the group consisting of TCR V_{β} domain sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M, is provided.

In still another aspect, a binding protein comprising: a) a TCR alpha chain sequence selected from the group consisting of TCR alpha chain sequences listed in Table 1; and/or b) a TCR beta chain sequence selected from the group consisting of TCR beta chain sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M, is provided.

Numerous embodiments are further provided that may be applied to any aspect encompassed by the present invention and/or combined with any other embodiment described herein. For example, in one embodiment, 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 1, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Table 1, and/or 3) each CDR of the binding protein has up to five amino acid substitutions, insertions, deletions, or a combination thereof as compared to the cognate reference CDR sequence listed in Table 1. In another embodiment, an HA-1 immunogenic peptide comprises the amino acid sequence VLHDDLLEA. In still another embodiment, a binding protein is chimeric, humanized, or human. In yet another embodiment, a binding protein is a TCR, an antigen-binding fragment of a TCR, a single chain TCR (scTCR), a chimeric antigen receptor (CAR), or a fusion protein comprising a TCR and an effector domain, optionally wherein the binding domain comprises a transmembrane domain and an effector domain that is intracellular. In another embodiment, a TCR alpha chain and a TCR beta chain are covalently linked, optionally wherein the TCR alpha chain and the TCR beta chain are covalently linked through a linker peptide. In still another embodiment, a TCR alpha chain and/or a TCR beta chain are covalently linked to a moiety, optionally wherein the covalently linked moiety comprises an affinity tag or a label. In yet another embodiment, an affinity tag is selected from the group consisting of CD34 enrichment tag, Glutathione-S-Transferase (GST), calmodulin binding protein (CBP), protein C tag, Myc tag, HaloTag, HA tag, Flag tag, His tag, biotin tag, and V5 tag, and/or wherein the label is a fluorescent protein. In another embodiment, a covalently linked moiety is selected from the group consisting of an inflammatory agent, cytokine, toxin, cytotoxic molecule, radioactive isotope, or antibody or antigen-binding fragment thereof. In still another embodiment, a binding protein binds to the pMHC complex on a cell surface. In yet another embodiment, an MHC is a MHC multimer, optionally wherein the MHC multimer is a tetramer. In another embodiment, an MHC is a MHC class I molecule. In still another embodiment, an MHC comprises an MHC alpha chain that is an HLA serotype HLA-A*02. In yet another embodiment, an HLA allele is selected from the group consisting of HLA-A*0201, HLA-A*0202, HLA-A*0203, HLA-A*0205, HLA-A*0206, and HLA-A*0207 allele. In another embodiment, binding of a binding protein described herein to an HA-1 peptide-MHC (pMHC) complex elicits an immune response, optionally wherein the immune response is a T cell response. In still another embodiment, a T cell response is selected from the group consisting of T cell expansion, cytokine release, and/or cytotoxic killing. In yet another embodiment, a binding protein is capable of specifically and/or selectively binding to an HA-1 immunogenic peptide-MHC (pMHC) complex with a K_{d} less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, or less than or equal to about 1×10⁻¹² M. In yet another embodiment, a binding protein has a higher binding affinity to a peptide-MHC (pMHC) than does a known T-cell receptor. In another embodiment, a binding protein has at least 1.05 fold higher binding affinity to a peptide-MHC (pMHC) than does a known T-cell receptor. In still another embodiment, a binding protein induces higher T cell expansion, cytokine release, and/or cytotoxic killing than does a known T-cell receptor when contacted with target cells with a heterozygous expression of HA-1. In yet another embodiment, a binding protein induces at least 1.05-fold increase in T cell expansion, cytokine release, and/or cytotoxic killing than does a known T-cell receptor when contacted with target cells with a heterozygous expression of HA-1. As used herein, references to fold changes, in some embodiments, may be in comparison to any reference modality of interest, such as comparison to a different binding protein; comparison to the same binding protein under different context like expression of the same binding protein in a different immune cell, at a different level, in combination with other agents described herein; and the like. In another embodiment, a target cell is a THP-1 or TF1 cell line. In still another embodiment, a target cell is a cancer cell, optionally wherein the cancer cell is a hematopoietic cancer cell, a leukemia cell, a lymphoma cell, a myelodysplastic disorder cell, a myeloproliferative neoplasm cell, or a myeloma cell. In still another embodiment, a binding protein described herein does not bind to HA-1^{R} peptide-MHC (pMHC) complex, wherein the HA-1^{R} peptide comprises the amino acid sequence VLRDDLLEA. In yet another embodiment, a binding protein described herein does not bind to a CCDC114 peptide-MHC (pMHC) complex, optionally wherein the CCDC114 peptide is SMIDDLLGV.

In yet another aspect, a TCR alpha chain and/or beta chain selected from the group consisting of TCR alpha chain and beta chain sequences listed in Table 1, is provided.

In another aspect, an isolated nucleic acid molecule that hybridizes, under stringent conditions, with the complement of a nucleic acid encoding a polypeptide selected from the group consisting of polypeptide sequences listed in Table 1, or a sequence with at least about 80% homology to a nucleic acid encoding a polypeptide selected from the group consisting of the polypeptide sequences listed in Table 1, optionally wherein the isolated nucleic acid molecule comprises 1) a TRAV, TRAJ, and/or TRAC gene or fragment thereof selected from the group of TRAV, TRAJ, and TRAC genes listed in Table 1 and/or 2) a TRBV, TRBJ, and/or TRBC gene or fragment thereof selected from the group of TRBV, TRBJ, and TRBC genes listed in Table 1, is provided.

Numerous embodiments are further provided that may be applied to any aspect encompassed by the present invention and/or combined with any other embodiment described herein. For example, in one embodiment, a nucleic acid is codon optimized for expression in a host cell.

In still another aspect, a vector comprising an isolated nucleic acid described herein, is provided.

Numerous embodiments are further provided that may be applied to any aspect encompassed by the present invention and/or combined with any other embodiment described herein. For example, in one embodiment, a vector is a cloning vector, expression vector, or viral vector. In another embodiment, a vector further comprises a nucleic acid sequence encoding CD8α and/or CD8β. In still another embodiment, a nucleic acid sequence encoding CD8α or CD8β is operably linked to a nucleic acid encoding a tag. In yet another embodiment, a nucleic acid encoding a tag is at the 5′ upstream of the nucleic acid sequence encoding CD8α or CD8β such that the tag is fused to the N-terminal of CD8α or CD8β. In another embodiment, a tag is a CD34 enrichment tag. In still another embodiment, an isolated nucleic acid described herein, and a nucleic acid sequence encoding CD8α and/or CD8β are interconnected with an internal ribosome entry site or a nucleic acid sequence encoding a self-cleaving peptide. In yet another embodiment, a self-cleaving peptide is P2A, E2A, F2A or T2A.

In yet another aspect, a host cell which comprises an isolated nucleic acid described herein, comprises a vector described herein, and/or expresses a binding protein described herein, optionally wherein the cell is genetically engineered, is provided.

Numerous embodiments are further provided that may be applied to any aspect encompassed by the present invention and/or combined with any other embodiment described herein. For example, in one embodiment, a host cell comprises a chromosomal gene knockout of a TCR gene, an HLA gene, or both. In another embodiment, a host cell comprises a knockout of an HLA gene selected from an α1 macroglobulin gene, α2 macroglobulin gene, α3 macroglobulin gene, β1 microglobulin gene, β2 microglobulin gene, and combinations thereof. In still another embodiment, a host cell comprises a knockout of a TCR gene selected from a TCR α variable region gene, TCR f variable region gene, TCR constant region gene, and combinations thereof. In yet another embodiment, a host cell expresses CD8α and/or CD8β. In another embodiment, CD8α and/or CD8β is fused to a CD34 enrichment tag. In still another embodiment, host cells are enriched using a CD34 enrichment tag. In yet another embodiment, a host cell is a hematopoietic progenitor cell, peripheral blood mononuclear cell (PBMC), cord blood cell, or immune cell. In another embodiment, an immune cell is a cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or combination thereof. In another embodiment, a T cell is a naive T cell, central memory T cell, effector memory T cell, or a combination thereof. In still another embodiment, a T cell is a primary T cell or a cell of a T cell line. In yet another embodiment, a T cell does not express or has a lower surface expression of an endogenous TCR. In another embodiment, a host cell is capable of producing a cytokine or a cytotoxic molecule when contacted with a target cell that comprises a peptide-MHC (pMHC) complex comprising the HA-1 peptide epitope in the context of an MHC molecule. In still another embodiment, a host cell is contacted with a target cell in vitro, ex vivo, or in vivo. In yet another embodiment, a cytokine is TNF-α, IL-2, and/or IFN-γ. In another embodiment, a cytotoxic molecule is perforins and/or granzymes, optionally wherein the cytotoxic molecule is granzyme B. In still another embodiment, a host cell is capable of producing a higher level of cytokine or a cytotoxic molecule when contacted with a target cell with a heterozygous expression of HA-1. In yet another embodiment, a host cell is capable of producing an at least 1.05-fold higher level of cytokine or a cytotoxic molecule. In another embodiment, a host cell is capable of killing a target cell that comprises a peptide-MHC (pMHC) complex comprising an HA-1 peptide epitope in the context of an MHC molecule. In still another embodiment, killing is determined by a killing assay. In yet another embodiment, a ratio of a host cell and a target cell in a killing assay is from 20:1 to 0.625:1. In another embodiment, a target cell is a T2 cell pulsed with 1 μg/mL to 50 pg/mL of HA-1 peptide. In still another embodiment, a host cell is capable of killing a higher number of target cells when contacted with target cells with a heterozygous expression of HA-1. In yet another embodiment, a host cell is capable of killing an at least 1.05-fold higher number of target cells. In another embodiment, a target cell is a THP-1 or TF1 cell line. In still another embodiment, an HA-1 immunogenic peptide comprises the amino acid sequence VLHDDLLEA. In yet another embodiment, an MHC molecule is a MHC class I molecule. In another embodiment, an MHC molecule comprises an MHC alpha chain that is an HLA serotype HLA-A*02. In still another embodiment, an HLA allele is selected from the group consisting of HLA-A*02:01, HLA-A*02:02, HLA-A*02:03, HLA-A*02:05, HLA-A*02:06, and HLA-A*02:07 allele. In yet another embodiment, a target cell is a cell line selected from the group consisting of SupM2, THP-1, and TF1 cell line, is a hematopoietic cancer cell expressing HA-1, or is not SU-DHL1. In another embodiment, a cancer cell is of a hematological malignancy selected from the group consisting of a leukemia, a lymphoma, a myelodysplastic disorder, a myeloproliferative neoplasm, or myeloma. In still another embodiment, a) a host cell does not induce T cell expansion, cytokine release, or cytotoxic killing when contacted with a target cell that comprises a peptide-MHC (pMHC) complex comprising a CCDC114 peptide epitope in the context of an MHC molecule and/or b) a host cell does not express HA-1 antigen, is not recognized by a binding protein described herein, is not of serotype HLA-A*02, and/or does not express an HLA-A*02 allele, such as HLA-A*02:01 and/or HLA-A*02:06.

In another aspect, a population of host cells described herein, is provided.

In still another aspect, a composition comprising: a) a binding protein described herein, b) an isolated nucleic acid described herein, c) a vector described herein, d) a host cell described herein, and/or e) a population of host cells described herein, and a carrier, is provided.

In yet another aspect, a device or kit comprising: a) a binding protein described herein, b) an isolated nucleic acid described herein, c) a vector described herein, d) a host cell described herein, and/or e) a population of host cells described herein, said device or kit optionally comprising a reagent to detect binding of a), d) and/or e) to a pMHC complex, is provided.

In another aspect, a method of producing a binding protein described herein, 5 wherein the method comprises the steps of: (i) culturing a transformed host cell which has been transformed by a nucleic acid comprising a sequence encoding a binding protein described herein under conditions suitable to allow expression of said binding protein; and (ii) recovering the expressed binding protein, is provided.

In still another aspect, a method of producing a host cell expressing a binding 10 protein described herein, wherein the method comprises the steps of: (i) introducing a nucleic acid comprising a sequence encoding a binding protein described herein into the host cell; (ii) culturing the transformed host cell under conditions suitable to allow expression of said binding protein, is provided.

In yet another aspect, a method of detecting the presence or absence of an HA-1 antigen and/or a cell expressing HA-1, optionally wherein the cell is a hyperproliferative cell, comprising detecting the presence or absence of said HA-1 antigen in a sample by use of at least one binding protein described herein, or at least one host cell described herein, wherein detection of the HA-1 antigen is indicative of the presence of an HA-1 antigen and/or cell expressing HA-1, is provided.

Numerous embodiments are further provided that may be applied to any aspect encompassed by the present invention and/or combined with any other embodiment described herein. For example, in one embodiment, at least one binding protein, or at least one host cell, forms a complex with an HA-1 peptide in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay. In another embodiment, a method further comprises obtaining a sample from a subject. In still another embodiment, a method further comprises confirming cells expressing HA-1 by bone marrow biopsy.

In another aspect, a method of detecting the level of a non-malignant disorder, a hyperproliferative disorder, or a relapse of a hyperproliferative disorder characterized by expression of an HA-1 antigen in a subject, comprising: a) contacting a sample obtained from the subject with at least one binding protein described herein, at least one host cell described herein, or a population of host cells described herein; and b) detecting the level of reactivity, wherein a higher level of reactivity compared to a control level indicates the level of a non-malignant disorder, a hyperproliferative disorder, or a relapse of a hyperproliferative disorder characterized by expression of an HA-1 antigen in the subject, is provided.

Numerous embodiments are further provided that may be applied to any aspect encompassed by the present invention and/or combined with any other embodiment described herein. For example, in one embodiment, a control level is a reference number. In another embodiment, a control level is a level of a subject without the non-malignant disorder, a hyperproliferative disorder, or a relapse of a hyperproliferative disorder characterized by expression of an HA-1 antigen.

In still another aspect, a method for monitoring the progression of a non-malignant disorder, a hyperproliferative disorder, or a relapse of a hyperproliferative disorder characterized by expression of an HA-1 antigen in a subject, the method comprising: a) detecting in a subject sample at a first point in time the level of an HA-1 antigen or a cell of interest expressing HA-1, as described herein; b) repeating step a) at a subsequent point in time; and c) comparing the level of the HA-1 antigen or the cell of interest expressing HA-1 detected in steps a) and b) to monitor the progression of a non-malignant disorder, a hyperproliferative disorder, or a relapse of a hyperproliferative disorder characterized by expression of an HA-1 antigen in the subject, wherein an absent or reduced level of the HA-1 antigen or the cell of interest expressing HA-1 detected in step b) compared to step a) indicates an inhibited progression of the non-malignant disorder, the hyperproliferative disorder, or the relapse of a hyperproliferative disorder characterized by expression of an HA-1 antigen in the subject.

Numerous embodiments are further provided that may be applied to any aspect encompassed by the present invention and/or combined with any other embodiment described herein. For example, in one embodiment, a subject has undergone treatment to treat the non-malignant disorder, the hyperproliferative disorder, or the relapse of a hyperproliferative disorder characterized by expression of an HA-1 antigen between the first point in time and the subsequent point in time.

In yet another aspect, a method of assessing the efficacy of a therapy for a non-malignant disorder, a hyperproliferative disorder, or a relapse of a hyperproliferative disorder characterized by expression of an HA-1 antigen comprising: a) determining the presence or level of reactivity between a sample obtained from a subject and at least one binding protein described herein, at least one host cell described herein, or a population of host cells described herein, in a first sample obtained from the subject prior to providing at least a portion of the therapy for the non-malignant disorder, the hyperproliferative disorder, or the relapse of a hyperproliferative disorder characterized by expression of an HA-1 antigen to the subject, and b) determining the presence or level of reactivity between a sample obtained from the subject and at least one binding protein described herein, at least one host cell described herein, or a population of host cells described herein, in a second sample obtained from the subject following provision of the portion of the therapy for the non-malignant disorder, the hyperproliferative disorder, or the relapse of a hyperproliferative disorder characterized by expression of an HA-1 antigen, wherein the absence or a reduced level of reactivity in the second sample, relative to the first sample, is an indication that the therapy is efficacious for treating the non-malignant disorder, the hyperproliferative disorder, or the relapse of a hyperproliferative disorder characterized by expression of an HA-1 antigen in the subject, is provided.

Numerous embodiments are further provided that may be applied to any aspect encompassed by the present invention and/or combined with any other embodiment described herein. For example, in one embodiment, a level of reactivity is indicated by a) the presence of binding and/or b) T cell activation and/or effector function. In another embodiment, T cell activation or effector function is T cell proliferation, killing, or cytokine release. In still another embodiment, T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

In another aspect, a method of preventing and/or treating a non-malignant disorder, a hyperproliferative disorder or a relapse of a hyperproliferative disorder characterized by expression of an HA-1 antigen in a subject comprising administering to the subject a therapeutically effective amount of a composition comprising cells expressing at least one binding protein described herein.

Numerous embodiments are further provided that may be applied to any aspect encompassed by the present invention and/or combined with any other embodiment described herein. For example, in one embodiment, a cell is an allogeneic cell, syngeneic cell, or autologous cell. In another embodiment, a cell is genetically modified. In still another embodiment, a cell comprises a chromosomal gene knockout of a TCR gene, an HLA gene, or both a TCR gene and an HLA gene. In yet another embodiment, a cell comprises a knockout of an HLA gene selected from an α1 macroglobulin gene, α2 macroglobulin gene, α3 macroglobulin gene, 31 microglobulin gene, β2 microglobulin gene, and a combination thereof. In another embodiment, a cell comprises a knockout of a TCR gene selected from a TCR a variable region gene, TCR β variable region gene, TCR constant region gene, and combinations thereof. In still another embodiment, a cell expresses CD8α and/or CD8β, optionally wherein the CD8α and/or CD8β is fused to a CD34 enrichment tag. In yet another embodiment, cells are enriched using the CD34 enrichment tag. In another embodiment, a cell is a hematopoietic progenitor cell, peripheral blood mononuclear cell (PBMC), cord blood cell, or immune cell. In still another embodiment, an immune cell is a cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or combination thereof. In yet another embodiment, a T cell is a naive T cell, central memory T cell, effector memory T cell, or combination thereof. In another embodiment, a T cell is a primary T cell or a cell of a T cell line. In still another embodiment, a T cell does not express or has a lower surface expression of an endogenous TCR. In yet another embodiment, a cell is capable of producing a cytokine or a cytotoxic molecule when contacted with a target cell that comprises a peptide-MHC (pMHC) complex comprising an HA-1 peptide epitope in the context of an MHC molecule. In another embodiment, a cytokine is TNF-α, IL-2, and/or IFN-γ. In still another embodiment, a cytotoxic molecule is perforins and/or granzymes, optionally wherein the cytotoxic molecule is granzyme B. In yet another embodiment, a cell is capable of producing a higher level of cytokine or a cytotoxic molecule when contacted with a target cell with a heterozygous expression of HA-1. In another embodiment, a cell is capable of producing an at least 1.05-fold higher level of cytokine or a cytotoxic molecule. In still another embodiment, a host cell is capable of killing a target cell that comprises a peptide-MHC (pMHC) complex comprising an HA-1 peptide epitope in the context of an MHC molecule. In yet another embodiment, a host cell is capable of killing a higher number of target cells when contacted with target cells with a heterozygous expression of HA-1. In another embodiment, a host cell is capable of killing an at least 1.05-fold higher number of target cells. In still another embodiment, an HA-1 immunogenic peptide comprises the amino acid sequence VLHDDLLEA. In yet another embodiment, an MHC molecule is an MHC class I molecule. In another embodiment, an MHC molecule comprises an MHC alpha chain that is an HLA serotype HLA-A*02. In still another embodiment, an HLA allele is selected from the group consisting of HLA-A*02:01, HLA-A*02:02, HLA-A*02:03, HLA-A*02:05, HLA-A*02:06, and HLA-A*02:07 allele. In yet another 5 embodiment, a target cell is a non-malignant cell or a hyperproliferating cell expressing HA-1 antigen in a subject. In another embodiment, a composition further comprises a pharmaceutically acceptable carrier. In still another embodiment, a composition induces an immune response against non-malignant cells or hyperproliferating cells expressing an HA-1 antigen in a subject. In yet another embodiment, a composition induces an antigen-specific T cell immune response against non-malignant cells or hyperproliferating cells expressing HA-1 antigen in a subject. In another embodiment, an antigen-specific T cell immune response comprises at least one of a CD4⁺ helper T lymphocyte (The) response and a CD8+ cytotoxic T lymphocyte (CTL) response. In still another embodiment, a hyperproliferative disorder comprises a hematological malignancy. In yet another embodiment, a hematological malignancy comprises a leukemia, a lymphoma, a myelodysplastic disorder, a myeloproliferative neoplasm, or a myeloma. In another embodiment, a hematological malignancy comprises a leukemia. In still another embodiment, a leukemia is selected from acute myeloid leukemia (AML), acute lymphocytic leukemia (ALL), mixed phenotype acute leukemia (MPAL), chronic myeloid leukemia (CML), B cell prolymphocytic leukemia, hairy cell leukemia, or chronic lymphocytic leukemia (CLL). In yet another embodiment, a hematological disorder comprises a lymphoma. In another embodiment, a lymphoma is selected from Hodgkin's lymphoma (HL), non-Hodgkin's lymphoma (NHL), a central nervous system lymphoma, small lymphocytic lymphoma (SLL), CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, precursor B-lymphoblastic lymphoma, immunoblastic large cell lymphoma, intravascular large B-cell lymphoma, primary effusion lymphoma, or Burkitt's lymphoma. In still another embodiment, a hematological malignancy comprises a myelodysplastic disorder (MDS), optionally wherein the MDS is selected from refractory cytopenia with unilineage dysplasia (refractory anemia, refractory neutropenia, and refractory thrombocytopenia), refractory anemia with ring sideroblasts (RARS), refractory anemia with ring sideroblasts-thrombocytosis (RARS-t), refractory cytopenia with multinieage dysplasia (RCMD), refractory cytopenia with multinieage dysplasia and ring sideroblasts (RCMD-RS), refractory anemia with excess blasts (RAEB), myelodysplasia unclassifiable, refractory cytopenia of childhood, or MDS with isolated del(5q). In yet another embodiment, a non-malignant disorder is an immune deficiency disorder, optionally wherein the immune deficiency disorder is selected from the group consisting of severe combined immunodeficiency (SCID), Wiskott-Aldrich syndrome, Omenn syndrome, X-linked lymphoproliferative syndrome, chronic granulomatous disease, leukocyte adhesion deficiency, DiGeorge syndrome, and indications for hematopoietic stem cell transplantation (HCT). In another embodiment, a non-malignant disorder is a non-malignant hematology, optionally wherein the non-malignant hematology disorder is selected from the group consisting of as sickle cell anemia, thalassemia, aplastic anemia, hemophagocytic lymphohistiocytosis (HLH), severe aplastic anemia, marrow failure syndromes, Fanconi anemia, Diamond-Blackfan anemia, and Shwachman Diamond syndrome. In still another embodiment, a non-malignant disorder is an autoimmune disorder, optionally wherein the autoimmune disorder is systemic sclerosis or multiple sclerosis. In yet another embodiment, a subject is receiving or previously received a hematopoietic cell transplant (HCT), optionally wherein the HCT comprises cells that do not express HA-1 antigen, are not recognized by a binding protein described herein, are not of serotype HLA-A*02, and/or do not express an HLA-A*02:01 allele. In another embodiment, HCT comprises a donor hematopoeitic cell comprising a chromosomal knockout of a gene that encodes an HLA component, a chromosomal knockout of a gene that encodes a TCR component, or both. In still another embodiment, a subject had previously received lymphodepleting chemotherapy. In another embodiment, a lymphodepleting chemotherapy comprised cyclophosphamide, fludarabine, anti-thymocyte globulin, or a combination thereof. In still another embodiment, a method further comprising administering at least one additional treatment for the non-malignant disorder, the hyperproliferative disorder or the relapse of a hyperproliferative disorder to a subject. In yet another embodiment, at least one additional treatment for the non-malignant disorder, the hyperproliferative disorder or the relapse of a hyperproliferative disorder is administered concurrently or sequentially with the composition. In another embodiment, a subject is an animal model of a disorder characterized by HA-1 expression and/or the subject is a mammal, optionally wherein the mammal is a human, a primate, or a rodent. In still another aspect, an expression vector comprising a promoter operably linked to a nucleic acid sequence encoding CD8α and/or CD8β, is provided.

Numerous embodiments are further provided that may be applied to any aspect encompassed by the present invention and/or combined with any other embodiment described herein. For example, in one embodiment, a nucleic acid sequence encoding CD8α or CD8β is operably linked to a nucleic acid encoding a tag such that the tag is fused to the CD8α or CD8β. In another embodiment, a nucleic acid encoding a tag is at the 5′ upstream of the nucleic acid sequence encoding CD8α or CD8β such that the tag is fused to the N-terminal of CD8α or CD8β. In still another embodiment, a tag is a CD34 enrichment tag. In yet another embodiment, a vector further comprises a nucleic sequence encoding a TCRα and/or TCRβ. In another embodiment, a TCRα and/or TCRβ comprises a mutated transmembrane domain and/or a mutated constant domain. In still another embodiment, a mutated transmembrane domain and/or mutated constant domain enhance cellular surface expression of TCRα and/or TCRβ while decreasing expression of endogenous TCRα and/or TCRβ. In yet another embodiment, a nucleic acid sequence encoding CD8α and/or CD8β and the nucleic sequence encoding TCRα and/TCRβ, are interconnected with an internal ribosome entry site or a nucleic acid sequence encoding a self-cleaving peptide. In another embodiment, a self-cleaving peptide is P2A, E2A, F2A or T2A. In still another embodiment, a vector further comprises a nucleic sequence encoding a polypeptide selected from the group consisting of polypeptide sequences listed in Table 1, or a sequence with at least about 80% homology to a nucleic acid encoding a polypeptide selected from the group consisting of the polypeptide sequences listed in Table 1, optionally wherein the isolated nucleic acid molecule comprises 1) a TRAV, TRAJ, and/or TRAC gene or fragment thereof selected from the group of TRAV, TRAJ, and TRAC genes listed in Table 1 and/or 2) a TRBV, TRBJ, and/or TRBC gene or fragment thereof selected from the group of TRBV, TRBJ, and TRBC genes listed in Table 1. In still another embodiment, a vector has a nucleic sequence set forth in Table 3.

### BRIEF DESCRIPTION OF THE DRAWINGS

The patent of application file contains at least one drawing executed in color. Copies of this patent or patent application publication with color drawing(s) will be provided by the Office upon request and payment of the necessary fee.

FIG. 1 shows a schematic diagram of TCR discovery platform. The TCR discovery platform identified 329 novel HA-1 TCRs. CD14⁺ monocytes were isolated from PBMCs of six HA-1 negative healthy donors on day −4 and differentiated to mature DCs. On day −1, naïve CD8 T cells were isolated from autologous PBMCs and rested overnight. Co-culture of CD8 T cells and DCs was performed following 3 h pulsing of DCs with 1 μg/mL HA-1 peptide (VLHDDLLEA), followed by an 11-day cell expansion phase. Dextramer staining was performed with A*02:01-specific HA-1 dextramer to identify clones, which were then rapidly expanded using irradiated unmatched PBMCs in the presence of IL-2. After 7 days, HA-1 dextramer-positive cells were sorted and single cell TCR sequencing was performed.

FIG. 2A and FIG. 2B show that a “validate as you go” (VAYG) screen identified 11 anti-HA-1 TCRs with high expression and cytotoxicity. TSC-100 consistently demonstrates superior function compared to the benchmark “Fred Hutch” TCR and TScan's other 10 TCRs, particularly when HA-1 levels are low, although TScan's other 10 TCRs are also believed to demonstrate favorable binding and response profiles. FIG. 2A shows that pan-T cells were transduced to express 329 HA-1-specific TCRs. Surface expression of the TCRs was assessed by HA-1 (VLHDDLLEA) HLA-A*02:01 dextramer staining. Three of the high-expressing TCRs along with the benchmark “Fred Hutch” TCR are shown as representative data. FIG. 2B shows results of engineered T cells that were co-cultured with NucLight Red labeled T2 cells loaded with 1 ng/mL HA-1 peptide (E:T=5:1) and survival of T2 cells was quantified by time-dependent imaging as a readout of T cell cytotoxicity. Non-transduced cells (NTD) served as a control.

FIG. 3A-FIG. 3D show that an optimal vector can include one or more of (e.g., all of) CD8α and CD8β, a CD34 enrichment tag, and modifications in the constant and transmembrane domains of the HA-1 TCR. FIG. 3A shows that CD4 T cells were isolated and transduced to express an HA-1-specific TCR along with either CD8α alone or both CD8α and CD8β. Engineered T cells were co-cultured with labeled T2 cells loaded with various concentrations of HA-1 (VLHDDLLEA) peptide (E:T=5:1) and the growth of the T2 cells (or lack thereof) was quantified on an IncuCyte as a readout of the cytotoxicity of the T cells. FIG. 3B shows that TSC-100 delivery vector contains a CD34 tag grafted on the N-terminus of CD8a, enabling enrichment of engineered T cells using a GMP-compatible Miltenyi CD34 enrichment kit. T cells were transduced with TSC-100 and evaluated for CD34 expression and TCR expression by HA-1 (VLHDDLLEA) HLA-A*02:01 dextramer staining pre- and post-CD34 enrichment. FIG. 3C shows that the original sequence of TSC-100 was compared to modified sequences carrying optimization of residues in the constant domain (CD) and in the transmembrane domain (TM) designed to ensure surface expression and proper alpha/beta pairing of the TCR. Primary human T cells were transduced with different versions of the TCRs and the surface expression of the properly paired TCR was assessed by HA-1 (VLHDDLLEA) HLA-A*02:01 dextramer staining. FIG. 3D shows a representative vector configuration. Stars indicate CD and TM modifications.

FIG. 4A-FIG. 4F show that TSC-100 is superior to the Fred Hutch TCR, especially when HA-1 and HLA-A*02:01 expression are low. Pan-T cells were transduced to express TSC-100 and the Fred Hutch HA-1-specific TCR and assessed for functional responses to target cells expressing HA-1 and HLA-A*02:01. HA-1-cells express VLRDDLLEA isoform and HA-1⁺ express VLHDDLLEA isoform of the HA-1 protein. FIG. 4A provides dot plots showing expression of TSC-100 and the Fred Hutch TCR as assessed by HA-1 (VLHDDLLEA) HLA-A*02:01 dextramer staining. FIGS. 4B-4F show functional responses of TSC-100 and Fred Hutch TCR to target cell lines. SUP-M2 (HLA-A*02:01⁺HA-1⁺) (FIG. 4B), THP-1 (HLA-A*02:01⁺HA-1⁺) (FIG. 4C), TF-1 (HLA-A*02:01^{lo} HA-1^{lo}) (FIG. 4D), SU-DHL1 (HLA-A*02:01⁺HA-1⁻) (FIG. 4E) and T cells only (FIG. 4F) conditions. The left panel shows that engineered T cells were co-cultured with Nuclight Red labeled HLA-A*02:01⁺ cell lines endogenously expressing varying levels of HA-1 at indicated E:T ratios, and their survival was quantified on an IncuCyte as a readout of cytotoxicity of the T cells. The middle panel shows production of IFN-γ, IL-2, TNF-α and granzyme B in co-culture supernatants at 24 hours (E:T 1:1). SUP-M2 and SU-DHL1 cells endogenously produce granzyme B. A T cell-only condition was used to determine background level of cytokine production. The right panel shows proliferation of TSC-100—and Fred Hutch TCR-expressing T cells. Engineered T cells were labeled with a proliferation dye and co-cultured with target cell lines for 96 hours (E:T 1:1). Dye dilution was used to assess proliferation of CD8 and CD4 T cells. Counting beads were added to the samples prior to analysis by flow cytometry and absolute numbers of divided CD8 and CD4 T cells were determined. TCRs were compared by one-way ANOVA followed by Dunnett's multiple comparison test. *P<0.05, **P<0.005, ***P<0.0005, ****P<0.0001. These experiments were performed in 2-3 donors and representative data from one donor are shown. Differences in proliferation were observed in 1 of 2 donors tested for TF-1 cells.

FIG. 5 shows that TSC-100 shows no alloreactivity to 104 HLA types. TSC-100 and Fred Hutch TCR were tested in an alloreactivity assay. Endogenous MHCs were knocked out of HEK293T cells using CRISPR/Cas9-gRNA ribonucleoprotein delivery and a cell line was prepared that stably expresses a 90-residue protein fragment that includes the HA-1 sequence. Individual MHCs representing the 104 most prevalent HLA types were then introduced into both the HA-1⁺ and HA-1⁻ cell lines by lentiviral transduction. Cells in 96-well format were incubated overnight with human primary CD8⁺ T cells expressing TSC-100 and the Fred Hutch TCR, and IFN-γ concentration in the supernatant was measured by ELISA. Concentrations above 20,000 μg/mL exceeded the linear detection range of the assay.

FIG. 6A and FIG. 6B show that TScan's genome-wide screen determined TSC-100 as having no detectable off-targets, whereas CCDC114 (such as CCDC114 peptide antigen SMIDDLLGV) was identified as an off-target of the Fred Hutch TCR. TSC-100 (FIG. 6A) and Fred Hutch (FIG. 6B) TCRs were evaluated for off-target reactivity using a T-Scan platform (Kula et al. (2019) Cell 178:1016-1028). In this genome-wide safety screen, T cells expressing TSC-100 or the Fred Hutch TCR were co-cultured with a library of HLA-A*02:01 Vtarget cells, each expressing a different 90-amino acid protein fragment. Collectively, the library includes fragments that tile across every protein in 22-amino acid steps. Fragments were processed naturally by the target cells and the resulting peptides are displayed on cell-surface MHCs. If a T cell recognizes its target, it attempts to kill the target cell, thereby activating a fluorescent reporter. By isolating fluorescent cells and sequencing their expression cassettes, the natural target(s) of the TCR are revealed. Out of ˜600,000 clones, only the three clones in the library that contain the HA-1 epitope were significantly enriched in the screen with TSC-100. Similar screen with Fred Hutch TCR identified CCDC114 as an off-target. The x-axis represents protein fragments in the TScan peptidome library and the y-axis represents fold enrichment of individual tiles over input library.

FIG. 7A-FIG. 7Q show that 10 other TScan HA-1-specific TCRs are functionally active similar to TSC-100. As described above for TSC-100, 10 additional HA-1-specific TCRs were analyzed using the same methods and in the same experiments as the data provided for TSC-100 in the description above (e.g., data shown in FIG. 4 are a subgroup of data provided in FIG. 7A-FIG. 7Q). For example, pan-T cells were transduced to express 11 HA-1-specific TCRs, including TSC-100 and Fred Hutch, as well as a non-functional control TCR (TK-1) and assessed for functional responses to target cells expressing HA-1 and HLA-A*02:01. HA-1⁻ cells express VLRDDLLEA isoform and HA-1⁺ express VLHDDLLEA isoform of the HA-1 protein. FIG. 7A-FIG. 7C provide dot plots showing expression of HA-1 TCRs as assessed by HA-1 (VLHDDLLEA) HLA-A*02:01 dextramer staining on three unique donors. FIG. 7D-FIG. 7Q shows functional responses of the TCRs to target cell lines. Results of conditions using SUP-M2 (HLA-A*02:01⁺HA-1⁺) cells (FIG. 7D-FIG. 7F), THP-1 (HLA-A*02:01⁺HA-1⁺) cells (FIG. 7G-FIG. 7I), TF-1 (HLA-A*02:01^{lo}HA-1^{lo}) cells (FIG. 7J-FIG. 7L), SU-DHL1 (HLA-A*02:01⁺HA-1⁻) cells (FIG. 7M-FIG. 7O), and T cells only (FIG. 7P and FIG. 7Q) are shown. Engineered T cells were co-cultured with Incucyte® NucLight Red-labeled HLA-A*02:01⁺ cell lines endogenously expressing varying levels of HA-1 at indicated E:T ratios, and their survival was quantified on an IncuCyte® as a readout of cytotoxicity of the T cells. Production of IFN-γ, IL-2, TNF-α and granzyme B in co-culture supernatants at 24 h (E:T 1:1) was assessed. To quantify proliferation of HA-1 specific TCR-expressing T cells, engineered T cells were labeled with a proliferation dye and co-cultured with target cell lines for 96 h (E:T 1:1). Dye dilution was used to assess proliferation of CD8 and CD4 T cells. Counting beads were added to the samples prior to analysis by flow cytometry and absolute numbers of divided CD8 and CD4 T cells were determined. A ‘T cell only’ condition was used to determine background levels of cytokine production and proliferation. SUP-M2 and SU-DHL1 produce granzyme B endogenously (data not shown). IL-2 production by TF-1 cells was similar to background levels for T cell only condition for all donors tested. Cytotoxicity and cytokine production in response to TF-1 cells were observed in 2 out of 3 donors tested. Proliferation for donor 1 in response to TF-1 cells was similar to T cell only condition (data not shown). ND indicates experiment not performed. TCRs were compared by one-way ANOVA followed by Dunnett's multiple comparison test using Fred Hutch as the control TCR. *P<0.05.

FIG. 8 shows that TSC-100 displays specific cytotoxic activity towards HA-1-positive cells. A flow cytometry-based cytotoxicity assay was performed. TSC-100 engineered T cells or non-transduced control T cells (labeled with CellTrace™ Violet) were co-cultured with the indicated primary target cells (labeled with CellTrace™ FarRed) at a 1:1 ratio for 24 hours. The relative viability of the target cells (i.e., CellTrace™ FarRed event) was measured by flow cytometry using a fixable viability dye. Data are expressed as viability of target cells relative to the non-transduced control T cell co-culture condition.

For any figure showing a bar histogram, curve, or other data associated with a legend, the bars, curve, or other data presented from left to right for each indication correspond directly and in order to the boxes from top to bottom, or from left to right, of the legend.

### DETAILED DESCRIPTION OF THE INVENTION

The present invention is based, at least in part, on the discovery of binding proteins, including T cell receptors (TCRs), that recognize HA-1 antigen (e.g., immunogenic peptide comprising the amino acid sequence, VLHDDLLEA).

Accordingly, the present invention relates, in part, to the identified binding proteins (e.g., TCRs), host cells expressing binding proteins (e.g., TCRs), compositions comprising binding proteins (e.g., TCRs) and host cells expressing binding proteins (e.g., TCRs), methods of diagnosing, prognosing, and monitoring T cell response to cells expressing the HA-1 antigen, and methods for preventing and/or treating a non-malignant disorder, a hyperproliferative disorder, or a relapse of a hyperproliferative disorder characterized by expression of the HA-1 antigen by administering host cells expressing binding proteins (e.g., TCRs).

### I. Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The term “administering” means providing a pharmaceutical agent or composition to a subject, and includes, but is not limited to, administering by a medical professional and self-administering. This involves the physical introduction of a composition comprising a therapeutic agent to a subject, using any of the various methods and delivery systems known to those skilled in the art. In some embodiments, routes of administration for binding proteins described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a binding protein described herein may be administered via a non-parenteral route, such as a topical, epidermal or mucosal route of administration, for example, intranasally, orally, vaginally, rectally, sublingually or topically. Administering may also be performed, for example, once, a plurality of times, and/or over one or more extended periods.

As used herein, the term “antigen” refers to any natural or synthetic immunogenic substance, such as a protein, peptide, or hapten. An antigen may be an HA-lantigen, or a fragment thereof, against which protective or therapeutic immune responses are desired.

The term “adjuvant” as used herein refers to substances, which when administered prior, together or after administration of an antigen accelerates, prolong and/or enhances the quality and/or strength of an immune response to the antigen in comparison to the administration of the antigen alone. Adjuvants can increase the magnitude and duration of the immune response induced by vaccination.

The term “antibody” as used to herein includes whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions may be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen presenting cell” or “APC” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “antigen-binding portion” of a binding protein, such as a TCR, as used herein, refers to one or more portions of a TCR that retain the ability to bind (e.g., specifically and/or selectively) to an antigen (e.g., an HA-1 antigen and cognate MHC/HLA. Such portions are, for example, between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of a TCR can be performed by fragments of a full-length TCR Examples of binding portions encompassed within the term “antigen-binding portion” of a TCR, include (i) a Fv fragment consisting of the V_{α} and V_{β} domains of a TCR, (ii) an isolated complementarity determining region (CDR) or (iii) a combination of two or more isolated CDRs which may optionally be joined by a synthetic linker. Furthermore, although V_{α} and V_{β}, are coded by separate genes, they may be joined, using recombinant methods, by a synthetic linker that enables them to be made as a single protein chain in which the V_{α} and V_{β} regions pair to form monovalent molecules (known as single chain TCR (scTCR)). Such single chain TCRs are also intended to be encompassed within the term “antigen-binding portion” of a TCR These TCR fragments can be obtained using conventional techniques known to those with skill in the art, and the fragments are screened for utility in the same manner as are complete binding proteins. Antigen-binding portions may be produced by recombinant DNA techniques, or by enzymatic or chemical cleavage of intact immunoglobulins.

The terms “complementarity determining region” and “CDR” are synonymous with “hypervariable region” or “HVR” and are known in the art to refer to non-contiguous sequences of amino acids within certain binding proteins, such as TCR variable regions, which confer antigen specificity and/or binding affinity. For TCRs, in general, there are three CDRs in each α-chain variable region (αCDR1, αCDR2, and αCDR3) and three CDRs in each p-chain variable region (βCDR1, βCDR2, and βCDR3). CDR3 is believed to be the main CDR responsible for recognizing processed antigen. CDR1 and CDR2 mainly interact with the MHC.

The term “body fluid” refers to fluids that are excreted or secreted from the body as well as fluids that are normally not excreted or secreted from the body (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In some embodiments, the body fluid comprises immune cells, optionally wherein the immune cells are cytotoxic lymphocytes such as cytotoxic T cells and/or NK cells, CD4+ T cells, and the like.

The term “coding region” refers to regions of a nucleotide sequence comprising codons that are translated into amino acid residues, whereas the term “non-coding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand which is anti-parallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and, in other embodiments, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, of the nucleotide residues of the first portion are capable of base pairing with nucleotide residues in the second portion. In some embodiments, all nucleotide residues of the first portion are capable of base pairing with nucleotide residues in the second portion.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal may result in cytokine secretion, e.g., in a T cell that has received a T cell-receptor-mediated signal. Immune cells that have received a cell-receptor mediated signal, e.g., via an activating receptor are referred to herein as “activated immune cells.”

“CD3” is known in the art as a multi-protein complex of six chains (see, Abbas and Lichtman, Cellular and Molecular Immunology (9^{th} Edition) (2018); Janeway et al. (Immunobiology) (9^{th} Edition) (2016)). In mammals, the complex comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is a characteristic that is believed to allow these chains to associate with positively charged regions or residues of T cell receptor chains. The intracellular tails of the CD3γ, CD3δ, and CD3ε chains each contain a single conserved motif known as an immunoreceptor tyrosine-based activation motif or IT AM, whereas each CD3ζ chain has three ITAMs. Without wishing to be bound by theory, it is believed that the IT AMs are important for the signaling capacity of a TCR complex. CD3 used in accordance with the present invention may be from various animal species, including human, mouse, rat, or other mammals.

A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ, or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Chimeric antigen receptor” or “CAR” refers to a fusion protein that is engineered to contain two or more amino acid sequences linked together in a way that does not occur naturally or does not occur naturally in a host cell, which fusion protein can function as a receptor when present on a surface of a cell. CARs encompassed by the present invention include an extracellular portion comprising an antigen-binding domain (i.e., obtained or derived from an immunoglobulin or immunoglobulin-like molecule, such as a TCR specific for an HA-1 antigen, a single chain TCR-derived binding protein, an scFv derived from an antibody, an antigen binding domain derived or obtained from a killer immunoreceptor from an NK cell, and the like) linked to a transmembrane domain and one or more intracellular signaling domains (such as an effector domain, optionally containing co-stimulatory domain(s)) (see, e.g., Sadelain et al. (2013) Cancer Discov. 3:388; see also Harris and Kranz (2016) Trends Pharmacol. Sci. 37: 220; Stone et al. (2014) Cancer Immunol. Immunother. 63:1163).

As used herein, the term “cytotoxic T lymphocyte (CTL) response” refers to an immune response induced by cytotoxic T cells. CTL responses are mediated primarily by CD8⁺ T cells.

The term “consisting essentially of is not equivalent to “comprising” and refers to the specified materials or steps of a claim, or to those that do not materially affect the basic characteristics of a claimed subject matter. For example, a protein domain, region, or module (e.g., a binding domain, hinge region, linker module) or a protein (which may have one or more domains, regions, or modules) “consists essentially of a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not

substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

The term “CCDC114” refers to Coiled-Coil Domain Containing 114, a coiled-coil domain-containing protein that is a component of the outer dynein arm docking complex in cilia cells. Mutations in this gene may cause primary ciliary dyskinesia 20. In one embodiment, CCDC114 has 670 amino acids and a molecular weight of 75046 Da. Representative mRNA and protein sequences of CCDC114 are shown in Table 4 below.

The term “determining a suitable treatment regimen for the subject” is taken to mean the determination of a treatment regimen (i.e., a single therapy or a combination of different therapies that are used for the prevention and/or treatment of the viral infection in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

“Fred Hutch T-cell receptor” refers to at least one benchmark T-cell receptor that has been reported in PCT Publ. No. WO 2018/058002, U.S. Pat. No. 10,538,574, and U.S. Pat. Publ. 2020/0231649. In some embodiments, the Fred Hutch T-cell receptor has sequences set forth in Table 2.

As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin-CD117⁺ phenotype or those found in the thymus (referred to as progenitor thymocytes).

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. In some embodiments, the first region comprises a first portion and the second region comprises a second portion, whereby, at least about 50%, and, in other embodiments, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, of the nucleotide residue positions of each of the portions are occupied by the same nucleotide residue. In some embodiments, all nucleotide residue positions of each of the portions are occupied by the same nucleotide residue.

As used herein, the term “HA-1 antigen” or “HA-1 peptide antigen” or “HA-1-containing peptide antigen” or “HA-1 epitope” or “HA-1 peptide epitope” or “HA-1 peptide” refers to a naturally or synthetically produced peptide portion of a HMHA1 protein, which is also known as ARHGAP45, and is an intracellular protein expressed at high levels in all blood cells, but not in any other tissue. ARHGAP45 comes in two forms. In HA-1-positive individuals, the peptide has the sequence VLHDDLLEA and, if the individual has an HLA A*02 serotype such as HLA type A*02:01 or HLA type A*02:06 (Torikai et al. (2007) Bone Marrow Transplant. 40:165-174), then the antigen is efficiently displayed on the surface of blood cells. In HA-1-negative individuals, the peptide has the sequence VLRDDLLEA, and the HA-1 antigen is not displayed. Approximately 60% of people have the VLHDDLLEA sequence and approximately 42% of people in the United States have the HLA type A*02:01, which means that approximately 25% of individuals in the United States are HA-1-positive and HLA type A*02:01 positive. Studies of patients receiving HCT have shown that in cases where the T cells of an HA-1-negative donor naturally develop a response to HA-1 in an HA-1-positive patient, the T cells mediate a specific graft-versus-leukemia (GvL) effect and the patient often experiences a long-term remission. In some embodiments, compositions and therapeutic methods described herein are based on this clinical observation and is designed to specifically cause this GvL effect in patients receiving HCT. HA-1 antigen protein can range in length from about 7 amino acids, about 8 amino acids, about 9 amino acids, about 10 amino acids, up to about 20 amino acids, and comprising the R139H substitution polymorphism), which can form a complex with a MIC (e.g., HLA) molecule, and a binding protein of this disclosure specific for an HA-1 peptide:MHC (e.g., HLA) complex can bind (e.g., specifically bind) to such as complex. An exemplary HA-1 peptide antigen comprises a peptide having the amino acid VLHDDLLEA, wherein the bolded histidine in the sequence represents the R139H polymorphism. The term “HA-1^{R}” or “HA-1⁻ H” refers to HA-1 antigen with R139H.

The term “hyperproliferative disorder characterized by expression of an HA-1 antigen” can be any hyperproliferative disorder where the HA-1 antigen is present in a MIC (e.g., HLA) complex expressed by at least some hyperproliferating cells in the subject. Examples of hyperproliferative disorders characterized by HA-1:HLA complexes include hematological malignancies. In certain embodiments, the hematological malignancy comprises a leukemia (e.g., an acute leukemia or a chronic leukemia). In specific embodiments, the leukemia comprises acute myeloid leukemia (AML), acute lymphocytic leukemia (ALL), mixed phenotype acute leukemia (MPAL), chronic myeloid leukemia (CML), B cell prolymphocytic leukemia, hairy cell leukemia, or chronic lymphocytic leukemia (CLL). In certain embodiments, the hematological malignancy comprises a lymphoma. In certain embodiments, the lymphoma comprises Hodgkin's lymphoma (HL), non-Hodgkin's lymphoma (NHL), a central nervous system lymphoma, small lymphocytic lymphoma (SLL), CD37⁺ dendritic cell lymphoma, lymphoplasmacytic lymphoma, splenic marginal zone lymphoma, plasma cell myeloma, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, precursor B-lymphoblastic lymphoma, immunoblastic large cell lymphoma, intravascular large B-cell lymphoma, primary effusion lymphoma Burkitt's lymphoma/leukemia, B-cell proliferations of uncertain malignant potential, lymphomatoid granulomatosis, and post-transplant lymphoproliferative disorder. In certain embodiments, the hematological malignancy comprises a myelodysplastic disorder (MDS), such as, for example, refractory cytopenia with unilineage dysplasia (refractory anemia, refractory neutropenia, and refractory thrombocytopenia), refractory anemia with ring sideroblasts (RARS), refractory anemia with ring sideroblasts-thrombocytosis (RARS-t), refractory cytopenia with multinieage dysplasia (RCMD), refractory cytopenia with multinieage dysplasia and ring sideroblasts (RCMD-RS), refractory anemia with excess blasts (RAEB), myelodysplasia unclassifiable, and refractory cytopenia of childhood, MDS with isolated del(5q), and other well-known MDS subtypes (see, for example, 2016 World Health Organization MDS classification available on the World Wide Web at nbei.nlm.nih.gov/pmc/articles/PMC655460/). In certain embodiments, the hematological malignancy comprises a myeloproliferative neoplasms (MPN), such as for example, myelofibrosis, polycythemia vera, essential thrombocythemia, chronic myelomonocytic leukemia, chronic myeloproliferative disease-unclassifiable, chronic neutrophilic leukemia, chronic eosinophilic leukemia and MPN—not otherwise specified. In further embodiments, the hematological malignancy comprises a myeloma.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system may be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity may be enhanced by at least 10%, 20%, 30%, 40%, 50%, 60%, 70%, 80%, 90%, 100%, 110%, 120%, 130%, 140%, 150%, 160%, 170%, 180%, 190%, 200%, 250%, 300%, 350%, 400%, 500%, or more.

The term “immunotherapeutic agent” may include any molecule, peptide, antibody or other agent which can stimulate a host immune system to generate an immune response to a viral infection in the subject Various immunotherapeutic agents are useful in the compositions and methods described herein.

The term “immune cell” refers to any cell of the immune system that originates from a hematopoietic stem cell in the bone marrow, which gives rise to two major lineages: a myeloid progenitor cell (which give rise to myeloid cells such as monocytes, macrophages, dendritic cells, megakaryocytes and granulocytes); and a lymphoid progenitor cell (which give rise to lymphoid cells such as T cells, B cells and natural killer (NK) cells). Exemplary immune system cells include a CD4⁺ T cell, a CD8⁺ T cell, a CD4 CD8 double negative T cell, a gd T cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the binding protein, antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), or, in some embodiments, less than about 25%, 20%, 15%, 10%, 5%, 1%, or less, or any range in between inclusive, such as less than about 1% to 5%, of non-biomarker protein. When binding protein, antibody, polypeptide, peptide or fusion protein or fragment thereof, e.g., a biologically active fragment thereof, is recombinantly produced, it may be substantially free of culture medium, i.e., culture medium represents less than about 20%, 15%, 10%, 5%, 1%, or less, or any range in between inclusive, such as less than about 10% to 5%, of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular binding protein-antigen interaction. The binding affinity of binding proteins encompassed by the present invention may be measured or determined by standard binding protein-target binding assays, for example, competitive assays, saturation assays, or standard immunoassays, such as ELISA or RIA. A relatively lower K_{d} value indicates a relatively higher binding affinity (e.g., K_{d} values of less than or equal to about 5×10⁻⁴ M (500 uM) include a K_{d} value of 1×10⁻⁴ M (100 uM) and a 100 uM Kd indicates a relatively higher binding affinity as compared to a 500 uM Kd).

A “kit” is any manufacture (e.g., a package or container) comprising at least one reagent, e.g., a probe or small molecule, for specifically detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In some embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit may be included.

As used herein, the term “linked” refers to the association of two or more molecules. The linkage may be covalent or non-covalent. The linkage also may be genetic (i.e., recombinantly fused). Such linkages may be achieved using a wide variety of art recognized techniques, such as chemical conjugation and recombinant protein production.

A “linker,” in some embodiments, may refer to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In some embodiments, a linker is comprised of about two to about 35 amino acids, for instance, or about four to about 20 amino acids or about eight to about 15 amino acids or about 15 to about 25 amino acids.

“Major histocompatibility complex” (MHC) refers to glycoproteins that deliver peptide antigens to a cell surface. MHC class I molecules are heterodimers having a membrane spanning a chain (with three a domains) and a non-covalently associated b2 microglobulin. MHC class II molecules are composed of two transmembrane glycoproteins, a and b, both of which span the membrane. Each chain has two domains. MHC class I molecules deliver peptides originating in the cytosol to the cell surface, where a peptide antigen-MHC (pMHC) complex is recognized by CD8⁺ T cells. MHC class II molecules deliver peptides originating in the vesicular system to the cell surface, where they are recognized by CD4⁺ T cells. Human MHC is referred to as human leukocyte antigen (HLA).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of a viral infection or the likelihood of recovery from the disease. In some embodiments, the use of statistical algorithms provides a prognosis of a viral infection in an individual. For example, the prognosis may be surgery, development of a clinical subtype of a viral infection, development of one or more clinical factors, or recovery from the disease.

As used herein, “percent identity” between amino acid sequences is synonymous with “percent homology,” which can be determined using the algorithm of Karlin and Altschul (1990) Proc. Natl. Acad. Sci. USA 87:2264-2268, modified by Karlin and Altschul (1993) Proc. Natl. Acad. Sci. USA 90:5873-5877. The noted algorithm is incorporated into the NBLAST and XBLAST programs of Altschul et al. (1990) J. Mol. Biol. 215:403-410. BLAST nucleotide searches are performed with the NBLAST program, score=100, wordlength=12, to obtain nucleotide sequences homologous to a polynucleotide described herein. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (1997) Nuc. Acids Res. 25:3389-3402. When utilizing BLAST and Gapped BLAST programs, the default parameters of the respective programs (e.g., XBLAST and NBLAST) may be used.

The phrase “pharmaceutically-acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

The term “recombinant host cell” (or simply “host cell”) refers to a cell that comprises a nucleic acid that is not naturally present in the cell, such as a cell into which a recombinant expression vector has been introduced. It should be understood that cells according to the present invention is intended to refer not only to the particular subject cell, but also encompasses progeny of such a cell. Because certain modifications may occur in succeeding generations due to either mutation or environmental influences, such progeny may not, in fact, be identical to the parent cell, but are still included within the scope of the term cell according to the present invention.

The term “cancer response,” “response to immunotherapy,” or “response to modulators of T-cell mediated cytotoxicity/immunotherapy combination therapy” relates to any response of the hyperproliferative disorder (e.g., cancer) to a cancer agent, such as a modulator of T-cell mediated cytotoxicity, and an immunotherapy, preferably to a change in tumor mass and/or volume after initiation of neoadjuvant or adjuvant therapy. The term “neoadjuvant therapy” refers to a treatment given before the primary treatment. Examples of neoadjuvant therapy may include chemotherapy, radiation therapy, and hormone therapy. Hyperproliferative disorder response may be assessed, for example for efficacy or in a neoadjuvant or adjuvant situation, where the size of a tumor after systemic intervention may be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment may be expanded to include response to chemotherapy, probability of survival, probability of metastasis within a given time period, and probability of tumor recurrence. For example, in order to determine appropriate threshold values, a particular cancer therapeutic regimen may be administered to a population of subjects and the outcome may be correlated to biomarker measurements that were determined prior to administration of any cancer therapy. The outcome measurement may be pathologic response to therapy given in the neoadjuvant setting. Alternatively, outcome measures, such as overall survival and disease-free survival may be monitored over a period of time for subjects following cancer therapy for which biomarker measurement values are known. In certain embodiments, the doses administered are standard doses known in the art for cancer therapeutic agents. The period of time for which subjects are monitored may vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy may be determined using well-known methods in the art, such as those described in the Examples section.

As indicated, the terms may also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive. The reduction in response may be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance may be mediated by P-glycoprotein or may be mediated by other mechanisms, or it may occur when a mammal is infected with a multi-drug-resistant microorganism or a combination of microorganisms. The determination of resistance to a therapeutic treatment is routine in the art and within the skill of an ordinarily skilled clinician, for example, may be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing logarithmically.

The term “sample” used for detecting or determining the absence, presence, or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In some embodiments, methods encompassed by the present invention further comprises obtaining the sample from the individual prior to detecting or determining the absence, presence, or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa et al. (1982) Cancer Res. 42:2159-2164) and cell death assays (Weisenthal et al. (1984) Cancer Res. 94:161-173; Weisenthal et al. (1985) Cancer Treat Rep. 69:615-632; Weisenthal et al., In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993:415-432; Weisenthal (1994) Contrib. Gynecol. Obstet. 19:82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy may be equally applied to methods for sensitizing hyperproliferative or otherwise cancerous cells (e.g., resistant cells) to the cancer therapy.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which may be screened for activity include, but are not limited to, peptides, peptidomimetics, nucleic acids, carbohydrates, small organic molecules (e.g., polyketides) (Cane et al. (1998) Science 282:63-68), and natural product extract libraries. In another embodiment, the compounds are small, organic non-peptidic compounds. In a further embodiment, a small molecule is not biosynthetic.

The term “specific binding” refers to binding protein binding to a predetermined antigen. Typically, the binding protein binds with an affinity (K_{D}) of approximately less than or equal to about 5×10⁻⁴ M, less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, less than or equal to about 1×10⁻¹² M, or even lower, or any range in between, inclusive, such as between about 1-50 micromolar, 1-100 micromolar, 0.1-500 micromolar, and the like, when determined by a binding assay, such as surface plasmon resonance (SPR) technology in a BIAcore™ assay instrument using an antigen of interest as the analyte and the binding protein as the ligand. In some embodiments, the binding protein binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “a binding protein recognizing an antigen” and “a binding protein specific for an antigen” are used interchangeably herein with the term “a binding protein which binds specifically to an antigen.” Selective binding is a relative term referring to the ability of a binding protein to discriminate the binding of one antigen over another, such as a particular family member or antigen target over a related family member or antigen target. For example, analytical data provided in the Examples section demonstrate that binding proteins described herein specifically bind HA-1 immunogenic epitopes and/or selectively bind a number of related epitopes (e.g., HA-1 immunogenic epitopes and closely related sequences) discriminating such targets from the vast majority of other possible epitopes available in the human genome.

The term “subject” refers to any healthy animal, mammal or human, or any animal, mammal or human afflicted with a non-malignant disorder, a hyperproliferative disorder, or a relapse of a hyperproliferative disorder characterized by expression of an HA-1 antigen. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment may be expanded to include response to chemotherapy, probability of survival, probability of metastasis within a given time period, and probability of tumor recurrence.

The term “synergistic effect” refers to the combined effect of two or more agents (e.g., an HA-1-related agent described herein and another therapy for treating a disorder characterized by HA-1 expression) that is greater than the sum of the separate effects of the cancer agents/therapies alone.

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

A “T cell” is an immune system cell that matures in the thymus and produces T cell receptors (TCRs). T cells may be naive (not exposed to antigen; increased expression of CD62L, CCR7, CD28, CD3, CD 127, and CD45RA, and decreased expression of CD45RO as compared to T_{CM}), memory T cells (T_{M}) (antigen-experienced and long-lived), and effector cells (antigen-experienced, cytotoxic). T_{M} may be further divided into subsets of central memory T cells (T_{CM}, increased expression of CD62L, CCR7, CD28, CD127, CD45RO, and CD95, and decreased expression of CD54RA as compared to naive T cells) and effector memory T cells (T_{EM}, decreased expression of CD62L, CCR7, CD28, CD45RA, and increased expression of CD127 as compared to naive T cells or T_{CM}). Effector T cells (T_{E}) refers to antigen-experienced CD8⁺ cytotoxic T lymphocytes that have decreased expression of CD62L,CCR7, CD28, and are positive for granzyme and perforin as compared to T_{CM}. Other exemplary T cells include regulatory T cells, such as CD4⁺ CD25⁺ (Foxp3⁺) regulatory T cells and Tregl7 cells, as well as Trl, Th3, CD8⁺CD28, and Qa-1 restricted T cells.

Conventional T cells, also known as Tconv or Teffs, have effector functions (e.g., cytokine secretion, cytotoxic activity, anti-self-recognition, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+T lymphocytes. “Naïve Tcons” are CD4⁺ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637).

“T effector” (“T_{eff}” or “T_{E}”) cells refers to T cells (e.g., CD4+ and CD8+ T cells) with cytolytic activities as well as T helper (Th) cells, which secrete cytokines and activate and direct other immune cells, but does not include regulatory T cells (Treg cells).

“T cell receptor” or “TCR” refers to an immunoglobulin superfamily member (having a variable binding domain, a constant domain, a transmembrane region, and a short cytoplasmic tail; see, e.g., Janeway et al. (1997) Curr. Biol. Publ. 4:33) that is capable of binding (e.g., specifically and/or selectively) to an antigen peptide bound to a MHC receptor. A TCR can be found on the surface of a cell or in soluble form and generally is comprised of a heterodimer having alpha and beta chains (also known as TCRα and TCRβ, respectively), or γ and δ chains (also known as TCRγ and TCRδ, respectively). Like immunoglobulins (e.g., antibodies), the extracellular portion of TCR chains (e.g., α-chain and β-chain) contain two immunoglobulin domains: a variable domain (e.g., α-chain variable domain or V_{α} and p-chain variable domain or V_{β}; typically amino acids 1 to 116 based on Kabat numbering (Kabat et al. (1991) “Sequences of Proteins of Immunological Interest, US Dept Health and Human Services, Public Health Service National Institutes of Health, 5^{th} ed.) at the N-terminal end, and one constant domain (e.g., α-chain constant domain or C_{α}, typically amino acids 117 to 259 based on Kabat, β-chain constant domain or C_{β}, typically amino acids 117 to 295 based on Kabat) at the C-terminal end and adjacent to the cell membrane. Also like immunoglobulins, the variable domains contain complementary determining regions (“CDRs”, also called hypervariable regions or “HVRs”) separated by framework regions (“FRs”) (see, e.g., Fores et al. (1990) Proc. Natl. Acad Sci. US.A. 87:9138; Chothia et al. (1988) EMBO J. 7:3745; Lefranc et al. (2003) Dev. Comp. Immunol. 27:55). In some embodiments, a TCR is found on the surface of a T cell (or T lymphocyte) and associates with the CD3 complex. The source of a TCR encompassed by the present invention may be from various animal species, such as a human, mouse, rat, rabbit or other mammal.

The term “T cell receptor” or “TCR” should be understood to encompass full TCRs as well as antigen-binding portions or antigen-binding fragments thereof. In some embodiments, the TCR is an intact or full-length TCR, including TCRs in the as form or γ6 form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR may contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a chain and variable p chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

Nomenclature established by the International Immunogenetics Information System (IMGT) (see also Scaviner and Lefranc (2000) Exp. Clin. Immunogenet. 17:83-96 and 97-106; Folch and Lefranc (2000) Exp. Clin. Immunogenet, 17:107-114; T Cell Receptor Factsbook”, (2001) LeFranc and LeFranc, Academic Press, ISBN 0-12-441352-8). The IMGT provides unique sequences used to describe a TCR, and sequences described herein may be identified by reference to such unique sequences provided herein. TCR sequences are publicly available at the IMGT database at imgt.org.

As described above, native alpha/beta heterodimeric TCRs have an alpha chain and a beta chain. Broadly, each chain comprises variable, joining and constant regions, and the beta chain also usually contains a short diversity region between the variable and joining regions, but this diversity region is often considered as part of the joining region. Each variable region comprises three hypervariable CDRs (Complementarity Determining Regions) embedded in a framework sequence. CDR3 is well-known to be the main mediator of antigen recognition. There are several types of alpha chain variable (Vα) regions and several types of beta chain variable (VO) regions distinguished by their framework, CDR1 and CDR2 sequences, and by a partly defined CDR3 sequence. The Vα types are referred to in IMGT nomenclature by a unique TRAV number. For example, “TRAV4” defines a TCR V_{α} region having unique framework and CDR1 and CDR2 sequences, and a CDR3 sequence which is partly defined by an amino acid sequence which is preserved from TCR to TCR but which also includes an amino acid sequence which varies from TCR to TCR. Similarly, “TRBV2” defines a TCR V region having unique framework and CDR1 and CDR2 sequences, but with only a partly defined CDR3 sequence. It is known that there are 54 alpha variable genes, of which 44 are functional, and 67 beta variable genes, of which 42 are functional, within the alpha and beta loci, respectively.

The joining regions of the TCR are similarly defined by the unique IMGT TRAJ and TRBJ nomenclature, and the constant regions by the IMGT TRAC and TRBC nomenclature. The beta chain diversity region is referred to in IMGT nomenclature by the abbreviation TRBD, and, as mentioned, the concatenated TRBD/TRBJ regions are often considered together as the joining region.

The gene pools that encode the TCR alpha and beta chains are located on different chromosomes and contain separate V, (D), J and C gene segments, which are brought together by rearrangement during T cell development. This leads to a very high diversity of T cell alpha and beta chains due to the large number of potential recombination events that occur between the 54 TCR alpha variable genes and 61 alpha J genes or between the 67 beta variable genes, two beta D genes and 13 beta J genes. The recombination process is not precise and introduces further diversity within the CDR3 region. Each alpha and beta variable gene may also comprise allelic variants, designated in IMGT nomenclature as TRAVxx*01 and *02, or TRBVx-x*01 and *02 respectively, thus further increasing the amount of variation. In the same way, some of the TRBJ sequences have two known variations. (Note that the absence of a “*” qualifier means that only one allele is known for the relevant sequence). The natural repertoire of human TCRs resulting from recombination and thymic selection has been estimated to comprise approximately 10⁶ unique beta chain sequences, determined from CDR3 diversity (Arstila et al. (1999) Science 286:958-961) and could be even higher (Robins et al. (2009) Blood 114:4099-4107). Each beta chain is estimated to pair with at least 25 different alpha chains, thus generating further diversity (Arstila et al. (1999) Science 286:958-961).

The term “TCR alpha variable domain” therefore refers to the concatenation of TRAV and TRAJ regions; a TRAV region only; or TRAV and a partial TRAJ region, and the term TCR alpha constant domain refers to the extracellular TRAC region, or to a C-terminal truncated or full length TRAC sequence. Likewise the term “TCR beta variable domain” refers to the concatenation of TRBV and TRBD/TRBJ regions; to the TRBV and TRBD regions only; to the TRBV and TRBJ regions only; or to the TRBV and partial TRBD and/or TRBJ regions, and the term TCR beta constant domain refers to the extracellular TRBC region, or to a C-terminal truncated or full length TRBC sequence. These TCR alpha variable domain and TCR beta variable domain nomenclature similarly applies to the variable domains of TCR gamma and TCR delta chains, respectively, for gamma/delta TCRs. An ordinarily skilled artisan can obtain TRAV, TRAJ, TRAC, TRBV, TRBJ, and TRBC gene sequences, such as through the publicly available IMGT database.

The term “TCR complex” refers to a complex formed by the association of CD3 with TCR For example, a TCR complex may be composed of a CD3γ chain, a CD3δ chain, two CD3ε chains, a homodimer of CD3ζ chains, a TCRα chain, and a TCRβ chain. Alternatively, a TCR complex may be composed of a CD3γ chain, a CD3δ chain, two CD3ε chains, a homodimer of CD3ζ chains, a TCRγ chain, and a TCRδ chain.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically effective amount” and “effective amount” means that amount of a substance that produces some desired effect, such as a desired local or systemic therapeutic effect, in at least a sub-population of cells in an animal at a reasonable benefit/risk ratio applicable to any treatment. In some embodiments, a therapeutically effective amount of a substance will depend on the substance's therapeutic index, solubility, pharmacokinetics, half-life, and the like. Toxicity and therapeutic efficacy of subject compounds may be determined by standard pharmaceutical procedures in cell cultures or experimental animals, e.g., for determining the LD₅₀ and the ED₅₀. In some embodiments, compositions that exhibit large therapeutic indices are used. In some embodiments, the LD₅₀ (lethal dosage) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, T cell immune response in an assay may be increased by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a viral load may be achieved.

The term “treat” refers to the therapeutic management or improvement of a condition (e.g., a disease or disorder) of interest. Treatment may include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. A therapeutic agent may be administered to a subject who has a disease or is at increased risk of developing a disease relative to a member of the general population. In some embodiments, a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease.

The term “unresponsiveness” includes refractivity of cancer cells to therapy or refractivity of therapeutic cells, such as immune cells, to stimulation, e.g., stimulation via an activating receptor or a cytokine. Unresponsiveness may occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells may, however, proliferate if cultured with cytokines (e.g., IL-2). For example, T cell anergy may also be observed by the lack of IL-2 production by T lymphocytes as measured by ELISA or by a proliferation assay using an indicator cell line. Alternatively, a reporter gene construct may be used. For example, anergic T cells fail to initiate IL-2 gene transcription induced by a heterologous promoter under the control of the 5′ IL-2 gene enhancer or by a multimer of the AP1 sequence that may be found within the enhancer (Kang et al. (1992) Science 257:1134).

The term “variable region” or “variable domain” refers to the domain of an immunoglobulin superfamily binding protein (e.g., a TCR α-chain or β-chain (or γ chain and 6 chain for γ6 TCRs)) that is involved in binding of the immunoglobulin superfamily binding protein (e.g., TCR) to antigen. The variable domains of the α-chain and β-chain (V_{α} and V_{β}, respectively) of a native TCR generally have similar structures, with each domain comprising four conserved framework regions (FRs) and three CDRs. The V_{α} domain is encoded by two separate DNA segments, the variable gene segment and the joining gene segment (V-J); the V_{β} domain is encoded by three separate DNA segments, the variable gene segment, the diversity gene segment, and the joining gene segment (V-D-J). A single V_{α} or V_{β} domain may be sufficient to confer antigen-binding specificity. Furthermore, TCRs that bind a particular antigen may be isolated using a V_{α} or V_{β} domain from a TCR that binds the antigen to screen a library of complementary V_{α} or V_{β} domains, respectively.

The term “vector” refers to a nucleic acid molecule capable of transporting another nucleic acid to which it has been linked. In some embodiments, a vector is an episome, i.e., a nucleic acid capable of extra-chromosomal replication. In some embodiments, vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the present invention is intended to include such other forms of expression vectors that serve equivalent functions and which become subsequently known in the art.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) may be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Binding Proteins

In an aspect encompassed by the present invention, provided herein are binding proteins that bind (e.g., specifically and/or selectively) to a peptide-MHC (pMHC) complex comprising an HA-1 immunogenic peptide in the context of an MHC molecule (e.g., a MHC class I molecule). In some embodiments, the binding protein is capable of binding (e.g., specifically and/or selectively) to an HA-1 peptide-MHC (pMHC) complex with a K_{d} less than or equal to about 5×10⁻⁴ M, less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, less than or equal to about 1×10⁻¹² M, or any range in between, inclusive, such as between about 1-50 micromolar, 1-100 micromolar, 0.1-500 micromolar, and the like. In some embodiments, the MHC molecule comprises an MHC alpha chain that is an HLA serotype HLA-A*02. In some embodiments, the HLA allele is selected from the group consisting of HLA-A*0201, HLA-A*0202, HLA-A*0203, HLA-A*0205, HLA-A*0206, and HLA-A*0207 allele. In a specific embodiment, the HLA allele is HLA-A*0201. In some embodiments, the binding proteins provided herein are genetically engineered, isolated, and/or purified.

In some embodiments, the binding proteins have a higher binding affinity to the HA-1 peptide-MHC (pMHC) than does a known T-cell receptor (e.g., a Fred Hutchinson TCR described herein). For example, the binding proteins may have at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, fold, 40 fold, 45 fold, 50 fold, 60 fold, 70 fold, 80 fold, 90 fold, 100 fold, 1000 fold, 5000 fold, 10000 fold, 50000 fold, 100000 fold, 500000 fold, 1000000 fold, or more, or any range in between, inclusive, such as 1.2 fold to 2 fold, higher binding affinity to the HA-1 peptide-MHC (pMHC) than does a known T-cell receptor (e.g., a Fred Hutchinson TCR described herein).

In some embodiments, the binding protein induces higher T cell expansion, cytokine release, and/or cytotoxic killing than does a known T-cell receptor (e.g., a Fred Hutchinson TCR described herein) when contacted with target cells with expression of HA-1 at a certain level or below. For example, in some embodiments of any aspect described herein, HA-1 level can be expressed in terms of transcripts per million and may be, for example, less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 35 TPM). In some embodiments, the low HA-1 expression level is termed “heterozygous expression” meaning between about 1 TPM and about 35 TPM, or any range in between, inclusive, such as 32 TPM or 1-32 TPM. A higher expression is 36 TPM and higher. As described further herein, TPM is measured according to well-known techniques, such as RNA-Seq, and gene expression TPM data are well known in the art for a variety of cell lines, tissue types, and the like (see, for example, the Broad Institute Cancer Cell Line Encyclopedia (CCLE) on the World Wide Web at portals.broadinstitute.org). In some embodiment, the binding protein induces at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, increase in T cell expansion, cytokine release, and/or cytotoxic killing than does a known T-cell receptor (e.g., a Fred Hutchinson TCR described herein) when contacted with target cells with heterozygous expression of HA-1.

In some embodiments, the expression of HA-1 is detected using RNA-sequencing (RNA-seq). RNA-seq generally comprises the following steps: obtaining a sample containing genetic material, isolating total RNA from the sample obtained, preparing an amplified cDNA library from the total RNA, sequencing the amplified cDNA library, and analyzing and profiling the amplified cDNA to assess the expression level of different transcripts. The sample can be a population of cells, a tissue sample, a biopsy sample, a cell culture, or a single cell. Total RNA can be isolated from the biological sample using any method known in the art. In certain embodiments, total RNA is extracted from plasma. Plasma RNA extraction is described in Enders et al., “The Concentration of Circulating Corticotropin-Releasing Homer mRNA in Material Plasma Is Inclined in Preeclampsia,” Clinr. As described therein, the plasma collected after the centrifugation step is mixed with Trizol LS reagent (Invitrogen) and chloroform. The mixture is centrifuged and the aqueous layer is transferred to a new tube. Ethanol is added to this aqueous layer. The mixture is then placed in an RNeasy mini column (Qiagen) and processed according to the manufacturer's recommendations.

In some embodiments, RNA-seq described herein includes the step of preparing amplified cDNA from total RNA. For example, cDNA is prepared and the isolated RNA sample is randomly amplified without dilution, or the mixture of genetic material in the isolated RNA is dispersed into individual reaction samples. In certain embodiments, amplification is initiated randomly at the 3′end and throughout the entire transcriptome in the sample to amplify both mRNA and non-polyadenylated transcripts. In this way, double-stranded cDNA amplification products are optimized for the generation of sequencing libraries for next generation sequencing platforms. A kit suitable for amplification of cDNA by the method encompassed by the present invention includes, for example, Ovation® RNA-Seq System.

In some embodiments, RNA-seq described herein includes the step of sequencing the amplified cDNA. Any known sequencing method can be used to sequence the amplified cDNA mixture including the single molecule sequencing method. In certain embodiments, the amplified cDNA is sequenced by whole transcriptome shotgun sequencing. Whole transcriptome shotgun sequencing can be performed using various next generation sequencing platforms such as Illumina® Genome Analyzer platform, ABI SOLiD™ Sequencing platform, or Life Science's 454 Sequencing platform.

In some embodiments, RNA-seq described herein further comprises performing digital counting and analysis on the cDNA. The number of amplified sequences for each transcript in the amplified sample can be quantified by sequence reading (one reading per amplified strand). In some embodiments, transcript per million (TPM) is used to quantify the expression level of a particular transcript. TPM may be calculated as shown in Wagner et al. (2012) Theory in Biosciences 131:281-285, the content of which is incorporated by reference herein in its entirety.

In some embodiments, the binding proteins do not bind to HA-1^{R} peptide-MHC (pMHC) complex, wherein the HA-1^{R} peptide comprises the amino acid sequence VLRDDLLEA.

In some embodiments, the binding protein does not bind to a CCDC114 peptide-MHC (pMHC) complex.

In some embodiments, the binding proteins provided herein include (e.g., comprise, consist essentially of, or consist of): a) a TCR alpha chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain sequence selected from the group consisting of the TCR alpha sequences listed in Table 1; and/or b) a TCR beta chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain sequence selected from the group consisting of the TCR beta chain sequences listed in Table 1.

In some embodiments, the binding proteins provided herein include (e.g., comprise, consist essentially of, or consist of): a) a TCR alpha chain sequence selected from the group consisting of the TCR alpha chain sequences listed in Table 1; and/or b) a TCR beta chain sequence selected from the group consisting of the TCR beta chain sequences listed in Table 1.

In some embodiments, the binding proteins provided herein include (e.g., comprise, consist essentially of, or consist of): a) a TCR alpha chain variable (V_{α}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain variable (V_{α}) domain sequence selected from the group consisting of the TCR V_{α} domain sequences listed in Table 1; and/or b) a TCR beta chain variable (V_{β}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain variable (V_{β}) domain sequence selected from the group consisting of the TCR V_{β} domain sequences listed in Table 1.

In some embodiments, the binding proteins provided herein include (e.g., comprise, consist essentially of, or consist of): a) a TCR alpha chain variable (V_{α}) domain sequence selected from the group consisting of the TCR V_{α} domain sequences listed in Table 1; and/or b) a TCR beta chain variable (V_{β}) domain sequence selected from the group consisting of the TCR V_{β} domain sequences listed in Table 1.

In some embodiments, the binding proteins provided herein include (e.g., comprise, consist essentially of, or consist of at least one (e.g., one, two or three, such as CDR3 alone or in combination with a CDR1 and CDR2)) TCR alpha chain complementarity determining region (CDR) sequence with at least about 80%, 81%, 82%, 83%, 84%, 85%, 86%, 87%, 88%, 89%, 90%, 91%, 92%, 93%, 94%, 95% 96%, 97%, 98%, 99%, or more identity to a TCR alpha chain CDR sequence selected from the group consisting of the TCR alpha chain CDR sequences listed in Table 1. CDR3 is believed to be the main CDR responsible for recognizing processed antigen and CDR1 and CDR2 mainly interact with the MHC, so, in some embodiments, binding protein comprising a CDR3 alone from a TCR alpha chain and/or a CDR3 alone from a TCR beta chain listed in Table 1, each CDR3 having a sequence homology as recited in this paragraph, are provided.

In some embodiments, the binding proteins provided herein may also include (e.g., comprise, consist essentially of, or consist of at least one (e.g., one, two or three, such as CDR3 alone or in combination with a CDR1 and CDR2)) TCR beta chain complementarity determining region (CDR) sequence with at least about 80%, 81%, 82%, 83%, 84%, 85%, 86%, 87%, 88%, 89%, 90%, 91%, 92%, 93%, 94%, 95% 96%, 97%, 98%, 99%, or more identity to a TCR beta chain CDR sequence selected from the group consisting of the TCR beta chain CDR sequences listed in Table 1. As described above, CDR3 is believed to be the main CDR responsible for recognizing processed antigen and CDR1 and CDR2 mainly interact with the MHC, so, in some embodiments, binding protein comprising a CDR3 alone from a TCR beta chain and/or a CDR3 alone from a TCR alpha chain listed in Table 1, each CDR3 having a sequence homology as recited in this paragraph, are provided.

In some embodiments, the binding proteins provided herein include (e.g., comprise, consist essentially of, or consist of at least one (e.g., one, two or three)) TCR alpha chain complementarity determining region (CDR) listed in Table 1.

In some embodiments, the binding proteins provided herein may also include (e.g., comprise, consist essentially of, or consist of at least one (e.g., one, two or three)) TCR beta chain complementarity determining region (CDR) listed in Table 1.

In some embodiments, the binding proteins provided herein include (e.g., comprise, consist essentially of, or consist of) a TCR alpha chain constant region (C_{α}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR Cα sequence listed in Table 1.

In some embodiments, the binding proteins provided herein may also include (e.g., comprise, consist essentially of, or consist of) a TCR beta chain constant region (C_{β}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR C_{β} sequence listed in Table 1.

In some embodiments, the binding proteins provided herein include (e.g., comprise, consist essentially of, or consist of) a TCR alpha chain constant region (C_{α}) sequence selected from the group consisting of the TCR Cₐ sequences listed in Table 1.

In some embodiments, the binding proteins provided herein may also include (e.g., comprise, consist essentially of, or consist of) a TCR beta chain constant region (C_{β}) sequence selected from the group consisting of the TCR C_{β} sequences listed in Table 1.

In some embodiments, the binding proteins provided herein comprise a constant region that is chimeric, humanized, human, primate, or rodent (e.g., rat or mouse). For example, a human variable region may be chimerized with a murine constant region or a murine variable region may be humanized with a human constant region and/or human framework regions. In some embodiments, the constant regions may be mutated to modify functionality (e.g., introduction of non-naturally occurring cysteine substitutions in opposing residue locations in TCR alpha and beta chains to provide disulfide bonds useful for increasing affinity between the TCR alpha and beta chains). Similarly, mutations may be made in the transmembrane domain of the constant region to modify functionality (e.g., increase hydrophobicity by introducing a non-naturally occurring substitution of a residue with a hydrophobic amino acid). In some embodiments, mutations may be made to the constant region to increase cell surface expression.

TABLE 1
HA1-TSC-100 wild type sequence (same CDRs as “TCR-100a”):
Alpha chain:
TRAV21/TRAJ40/TRAC
METLLGLLILWLQLQWVSSKQEVTQIPAALSVPEGENLVLNCSFTDSAIYNLQWFR
QDPGKGLTSLLLIQSSQREQTSGRLNASLDKSSGRSTLYIAASQPGDSATYLCAVR
GGTSGTYKYIFGTGTRLKVLANiqnpdpavyqlrdskssdksvclftdfdsqtnvsqskdsdvyitdktvld
mrsmdfksnsavawsnksdfacanafnnsiipedtffpspesscdvklveksfetdtnlnfqnlsvigfrilllkvagfnllmtlrl
ws (SEQ ID NO: 1)
Beta chain:
TRBV7-9/TRBJ2-3/TRBC1
MGTSLLCWMALCLLGADHADTGVSQDPRHKITKRGQNVTFRCDPISEHNRLYWY
RQTLGQGPEFLTYFQNEAQLEKSRLLSDRFSAERPKGSFSTLEIQRTEQGDSAMYL
CASSRTAGDTQYFGPGTRLTVLedlnkvfppevavfepseaeishtqkatlvclatgffpdhvelswwvngk
evhsgvstdpqplkeqpalndsryclssrlrvsatfwqnprnhfrcqvqfyglsendewtqdrakpvtqivsaeawgradcgft
svsyqqgvlsatilyeillgkatlyavlvsalvlmamvkrkdf (SEQ ID NO: 2)
HA1-TSC-100 CDTM sequence (also known as “TCR-100a”):
Alpha chain:
TRAV21/TRAJ40
METLLGLLILWLQLQWVSSKQEVTQIPAALSVPEGENLVLNCSFTDSAIYNLQWFR
QDPGKGLTSLLLIQSSQREQTSGRLNASLDKSSGRSTLYIAASQPGDSATYLCAVR
GGTSGTYKYIFGTGTRLKVLANiqnpdpavyqlrdskssdksvclftdfdsqtnvsqskdsdvyitdktvld
mrsmdfksnsavawsnksdfacanafnnsiipedtffpssdvpcdvklveksfetdtnlnfqnllvivlrilllkvagfnllmtlrl
ws (SEQ ID NO: 3)
Beta chain:
TRBV7-9/TRBJ2-3
MGTSLLCWMALCLLGADHADTGVSQDPRHKITKRGQNVTFRCDPISEHNRLYWY
RQTLGQGPEFLTYFQNEAQLEKSRLLSDRFSAERPKGSFSTLEIQRTEQGDSAMYL
CASSRTAGDTQYFGPGTRLTVLedlnkvfppevavfepskaeiahtqkatlvclatgffpdhvelswwvng
kevhsgvstdpqplkeqpalndsryclssrlrvsatfwqnprnhfreqvqfyglsendewtqdrakpvtqivsaeawgradcgi
tsasyhqgvlsatilyeillgkatlyavlvsalvlmamvkrkdf (SEQ ID NO: 4)
HA1-1-12 CDTM sequence:
Alpha chain:
TRAV20/TRAJ52
MEKMLECAFIVLWLQLGWLSGEDQVTQSPEALRLQEGESSSLNCSYTVSGLRGLF
WYRQDPGKGPEFLFTLYSAGEEKEKERLKATLTKKESFLHITAPKPEDSATYLCAV
HLNAGGTSYGKLTFGQGTILTVHPNiqnpdpavyqlrdskssdksvclftdfdsqtnvsqskdsdvyitdk
tvldmrsmdfksnsavawsnksdfacanafnnsiipedtffpssdvpcdvklveksfetdtnlnfqnllvivlrilllkvagfnll
mtlrlws (SEQ ID NO: 5)
Beta chain:
TRBV5-5/TRBJ1-2
MGPGLLCWVLLCLLGAGPVDAGVTQSPTHLIKTRGQHVTLRCSPISGHKSVSWYQ
QVLGQGPQFIFQYYEKEERGRGNFPDRFSARQFPNYSSELNVNALLLGDSALYLCA
SSFDRGTNGYTFGSGTRLTVVedlnkvfppevavfepskaeiahtqkatlvclatgffpdhvelswwvngke
vhsgvstdpqplkeqpalndsryclssrlrvsatfwqnprnhfrcqvqfyglsendewtqdrakpvtqivsaeawgradcgits
asyhqgvlsatilyeillgkatlyavlvsalvlmamvkrkdf (SEQ ID NO: 6)
HA1-9-46 CDTM sequence:
Alpha chain:
TRAV13-1/TRAJ43
MTSIRAVFIFLWLQLDLVNGENVEQHPSTLSVQEGDSAVIKCTYSDSASNYFPWYK
QELGKRPQLIIDIRSNVGEKKDQRIAVTLNKTAKHFSLHITETQPEDSAVYFCAASI
MRFGAGTRLTVKPNiqnpdpavyqlrdskssdksvclftdfdsqtnvsqskdsdvyitdktvldmrsmdfksnsa
vawsnksdfacanafnnsiipedtffpssdvpcdvklveksfetdtnlnfqnllvivlrilllkvagfnllmtlrlws (SEQ ID
NO: 7)
Beta chain:
TRBV7-9/TRBJ2-1
MGTSLLCWMALCLLGADHADTGVSQNPRHKITKRGQNVTFRCDPISEHNRLYWY
RQTLGQGPEFLTYFQNEAQLEKSRLLSDRFSAERPKGSFSTLEIQRTEQGDSAMYL
CASSLSVVDEQFFGPGTRLTVLedlnkvfppevavfepskaeiahtqkatlvclatgffpdhvelswwvngk
evhsgvstdpqplkeqpalndsryclssrlrvsatfwqnprnhfrcqvqfyglsendewtqdrakpvtqivsaeawgradcgit
sasyhqgvlsatilyeillgkatlyavlvsalvlmamvkrkdf (SEQ ID NO: 8)
HA1-9-105 CDTM sequence:
Alpha chain:
TRAV13-1/TRAJ26
MTSIRAVFIFLWLQLDLVNGENVEQHPSTLSVQEGDSAVIKCTYSDSASNYFPWYK
QELGKRPQLIIDIRSNVGEKKDQRIAVTLNKTAKHFSLHITETQPEDSAVYFCAVGQ
NFVFGPGTRLSVLPYiqnpdpavyqlrdskssdksvclftdfdsqtnvsqskdsdvyitdktvldmrsmdfksnsa
vawsnksdfacanafnnsiipedtffpssdvpcdvklveksfetdtnlnfqnllvivlrilllkvagfnllmtlrlws (SEQ ID
NO: 9)
Beta chain:
TRBV7-9/TRBJ1-1
MGTSLLCWMALCLLGADHADTGVSQDPRHKITKRGQNVTFRCDPISEHNRLYWY
RQTLGQGPEFLTYFQNEAQLEKSRLLSDRFSAERPKGSFSTLEIQRTEQGDSAMYL
CASSPTTINEAFFGQGTRLTVVedlnkvfppevavfepskaeiahtqkatlvclatgffpdhvelswwvngk
evhsgvstdpqplkeqpalndsryclssrlrvsatfwqnprnhfreqvqfyglsendewtqdrakpvtqivsaeawgradcgit
sasyhqgvlsatilyeillgkatlyavlvsalvlmamvkrkdf (SEQ ID NO: 10)
HA1-10-104 CDTM sequence:
Alpha chain:
TRAV8-3/TRAJ52
MLLELIPLLGIHFVLRTARAQSVTQPDIHITVSEGASLELRCNYSYGATPYLFWYVQ
SPGQGLQLLLKYFSGDTLVQGIKGFEAEFKRSQSSFNLRKPSVHWSDAAEYFCAV
ANAGGTSYGKLTFGQGTILTVHPNiqnpdpavyqlrdskssdksvclftdfdsqtnvsqskdsdvyitdkt
vldmrsmdfksnsavawsnksdfacanafnnsiipedtffpssdvpcdvklveksfetdtnlnfqnllvivlrilllkvagfnllm
tlrlws (SEQ ID NO: 11)
Beta chain:
TRBV6-2/TRBJ2-1
MSLGLLCCGAFSLLWAGPVNAGVTQTPKFRVLKTGQSMTLLCAQDMNHEYMYW
YRQDPGMGLRLIHYSVGEGTTAKGEVPDGYNVSRLKKQNFLLGLESAAPSQTSVY
FCASGRSGTKFFGPGTRLTVLedlnkvfppevavfepskaeiahtqkatlvclatgffpdhvelswwvngke
vhsgvstdpqplkeqpalndsryclssrlrvsatfwqnprnhfrcqvqfyglsendewtqdrakpvtqivsaeawgradcgits
asyhqgvlsatilyeillgkatlyavlvsalvlmamvkrkdf (SEQ ID NO: 12)
HA1-10-93 CDTM sequence:
Alpha chain:
TRAV13-1/TRAJ49
MTSIRAVFIFLWLQLDLVNGENVEQHPSTLSVQEGDSAVIKCTYSDSASNYFPWYK
QELGKRPQLIIDIRSNVGEKKDQRIAVTLNKTAKHFSLHITETQPEDSAVYFCPIRQ
FYFGTGTSLTVIPNiqnpdpavyqlrdskssdksvclftdfdsqtnvsqskdsdvyitdktvldmrsmdfksnsava
wsnksdfacanafnnsiipedtffpssdvpcdvklveksfetdtnlnfqnllvivlrilllkvagfnllmtlrlws (SEQ ID
NO: 13)
Beta chain:
TRBV7-9/TRBJ2-3
MGTSLLCWMALCLLGADHADTGVSQDPRHKITKRGQNVTFRCDPISEHNRLYWY
RQTLGQGPEFLTYFQNEAQLEKSRLLSDRFSAERPKGSFSTLEIQRTEQGDSAMYL
CASSLVVVDTQYFGPGTRLTVLedlnkvfppevavfepskaeiahtqkatlvclatgffpdhvelswwvngk
evhsgvstdpqplkeqpalndsryclssrlrvsatfwqnprnhfrcqvqfyglsendewtqdrakpvtqivsaeawgradcgit
sasyhqgvlsatilyeillgkatlyavlvsalvlmamvkrkdf (SEQ ID NO: 14)
HA1-2-23 CDTM sequence:
Alpha chain:
TRAV21/TRAJ40
METLLGLLILWLQLQWVSSKQEVTQIPAALSVPEGENLVLNCSFTDSAIYNLQWFR
QDPGKGLTSLLLIQSSQREQTSGRLNASLDKSSGRSTLYIAASQPGDSATYLCAVR
PTTSGTYKYIFGTGTRLKVLANiqnpdpavyqlrdskssdksvclftdfdsqtnvsqskdsdvyitdktvldm
rsmdfksnsavawsnksdfacanafnnsiipedtffpssdvpcdvklveksfetdtnlnfqnllvivlrilllkvagfnllmtlrlw
s (SEQ ID NO: 15)
Beta chain:
TRBV7-9/TRBJ1-1
MGTSLLCWMALCLLGADHADTGVSQDPRHKITKRGQNVTFRCDPISEHNRLYWY
RQTLGQGPEFLTYFQNEAQLEKSRLLSDRFSAERPKGSFSTLEIQRTEQGDSAMYL
CASILVGGTEAFFGQGTRLTVVedlnkvfppevavfepskaeiahtqkatlvclatgffpdhvelswwvngk
evhsgvstdpqplkeqpalndsryclssrlrvsatfwqnprnhfrcqvqfyglsendewtqdrakpvtqivsaeawgradcgit
sasyhqgvlsatilyeillgkatlyavlvsalvlmamvkrkdf (SEQ ID NO: 16)
HA1-10-34 wild type sequence (same CDRs as “TCR-100b”):
Alpha chain:
TRAV13-1/TRAJ40
MTSIRAVFIFLWLQLDLVNGENVEQHPSTLSVQEGDSAVIKCTYSDSASNYFPWYK
QELGKRPQLIIDIRSNVGEKKDQRIAVTLNKTAKHFSLHITETQPEDSAVYFCAARG
PGTYKYIFGTGTRLKVLANiqnpdpavyqlrdskssdksvclftdfdsqtnvsqskdsdvyitdktvldmrsm
dfksnsavawsnksdfacanafnnsiipedtffpspesscdvklveksfetdtnlnfqnlsvigfrilllkvagfnllmtlrlws
(SEQ ID NO: 17)
Beta chain:
TRBV7-9/TRBJ2-1
MGTSLLCWMALCLLGADHADTGVSQDPRHKITKRGQNVTFRCDPISEHNRLYWY
RQTLGQGPEFLTYFQNEAQLEKSRLSDRFSAERPKGSFSTLEIQRTEQGDSAMYLC
ASVAGTVYNEQFFGPGTRLTVLedlnkvfppevavfepseaeishtqkatlvclatgffpdhvelswwvngk
evhsgvstdpqplkeqpalndsryclssrlrvsatfwqnprnhfrcqvqfyglsendewtqdrakpvtqivsaeawgradcgft
svsyqqgvlsatilyeillgkatlyavlvsalvlmamvkrkdf (SEQ ID NO: 18)
HA1-10-34 CDTM sequence (also known as “TCR-100b”): TCR-100b
Alpha chain:
TRAV13-1/TRAJ40
MTSIRAVFIFLWLQLDLVNGENVEQHPSTLSVQEGDSAVIKCTYSDSASNYFPWYK
QELGKRPQLIIDIRSNVGEKKDQRIAVTLNKTAKHFSLHITETQPEDSAVYFCAARG
PGTYKYIFGTGTRLKVLANiqnpdpavyqlrdskssdksvclftdfdsqtnvsqskdsdvyitdktvldmrsm
dfksnsavawsnksdfacanafnnsiipedtffpssdvpcdvklveksfetdtnlnfqnllvivlrilllkvagfnllmtlrlws
(SEQ ID NO: 19)
Beta chain:
TRBV7-9/TRBJ2-1
MGTSLLCWMALCLLGADHADTGVSQDPRHKITKRGQNVTFRCDPISEHNRLYWY
RQTLGQGPEFLTYFQNEAQLEKSRLSDRFSAERPKGSFSTLEIQRTEQGDSAMYLC
ASVAGTVYNEQFFGPGTRLTVLedlnkvfppevavfepskaeiahtqkatlvclatgffpdhvelswwvng
kevhsgvstdpqplkeqpalndsryclssrlrvsatfwqnprnhfreqvqfyglsendewtqdrakpvtqivsaeawgradcgi
tsasyhqgvlsatilyeillgkatlyavlvsalvlmamvkrkdf (SEQ ID NO: 20)
HA1-10-26 wild type sequence (same CDRs as “TCR-100c”):
Alpha chain:
TRAV13-1/TRAJ28
MTSIRAVFIFLWLQLDLVNGENVEQHPSTLSVQEGDSAVIKCTYSDSASNYFPWYK
QELGKRPQLIIDIRSNVGEKKDQRIAVTLNKTAKHFSLHITETQPEDSAVYFCAASG
LGSGAGSYQLTFGKGTKLSVIPNiqnpdpavyqlrdskssdksvclftdfdsqtnvsqskdsdvyitdktvld
mrsmdfksnsavawsnksdfacanafnnsiipedtffpspesscdvklveksfetdtnlnfqnlsvigfrilllkvagfnllmtlrl
ws (SEQ ID NO: 21)
Beta chain:
TRBV7-9/TRBJ1-4
MGTSLLCWMALCLLGADHADTGVSQDPRHKITKRGQNVTFRCDPISEHNRLYWY
RQTLGQGPEFLTYFQNEAQLEKSRLLSDRFSAERPKGSFSTLEIQRTEQGDSAMYL
CASSLLAGEKLFFGSGTQLSVLedlnkvfppevavfepseaeishtqkatlvclatgffpdhvelswwvngk
evhsgvstdpqplkeqpalndsryclssrlrvsatfwqnprnhfrcqvqfyglsendewtqdrakpvtqivsaeawgradcgft
svsyqqgvlsatilyeillgkatlyavlvsalvlmamvkrkdf (SEQ ID NO: 22)
HA1-10-26 CDTM sequence (also known as “TCR-100c”):
Alpha chain:
TRAV13-1/TRAJ28
MTSIRAVFIFLWLQLDLVNGENVEQHPSTLSVQEGDSAVIKCTYSDSASNYFPWYK
QELGKRPQLIIDIRSNVGEKKDQRIAVTLNKTAKHFSLHITETQPEDSAVYFCAASG
LGSGAGSYQLTFGKGTKLSVIPNiqnpdpavyqlrdskssdksvclftdfdsqtnvsqskdsdvyitdktvld
mrsmdfksnsavawsnksdfacanafnnsiipedtffpssdvpcdvklveksfetdtnlnfqnllvivlrilllkvagfnllmtlrl
ws (SEQ ID NO: 23)
Beta chain:
TRBV7-9/TRBJ1-4
MGTSLLCWMALCLLGADHADTGVSQDPRHKITKRGQNVTFRCDPISEHNRLYWY
RQTLGQGPEFLTYFQNEAQLEKSRLLSDRFSAERPKGSFSTLEIQRTEQGDSAMYL
CASSLLAGEKLFFGSGTQLSVLedlnkvfppevavfepskaeiahtqkatlvclatgffpdhvelswwvngk
evhsgvstdpqplkeqpalndsryclssrlrvsatfwqnprnhfrcqvqfyglsendewtqdrakpvtqivsaeawgradcgit
sasyhqgvlsatilyeillgkatlyavlvsalvlmamvkrkdf (SEQ ID NO: 24)
HA1-2-21 CDTM sequence:
Alpha chain:
TRAV25/TRAJ29
MLLITSMLVLWMQLSQVNGQQVMQIPQYQHVQEGEDFTTYCNSSTTLSNIQWYK
QRPGGHPVFLIQLVKSGEVKKQKRLTFQFGEAKKNSSLHITATQTTDVGTYFCAGP
SGNTPLVFGKGTRLSVIANiqnpdpavyqlrdskssdksvclftdfdsqtnvsqskdsdvyitdktvldmrsmd
fksnsavawsnksdfacanafnnsiipedtffpssdvpcdvklveksfetdtnlnfqnllvivlrilllkvagfnllmtlrlws
(SEQ ID NO: 25)
Beta chain:
TRBV7-9/TRBJ2-1
MGTSLLCWMALCLLGADHADTGVSQDPRHKITKRGQNVTFRCDPISEHNRLYWY
RQTLGQGPEFLTYFQNEAQLEKSRLLSDRFSAERPKGSFSTLEIQRTEQGDSAMYL
CASSLLTTNEQFFGPGTRLTVLedlnkvfppevavfepskaeiahtqkatlvclatgffpdhvelswwvngk
evhsgvstdpqplkeqpalndsryclssrlrvsatfwqnprnhfrcqvqfyglsendewtqdrakpvtqivsaeawgradcgit
sasyhqgvlsatilyeillgkatlyavlvsalvlmamvkrkdf (SEQ ID NO: 26)
HA1-6-2 CDTM sequence:
Alpha chain:
TRAV38-1/TRAJ28
MTRVSLLWAVVVSTCLESGMAQTVTQSQPEMSVQEAETVTLSCTYDTSESNYYLF
WYKQPPSRQMILVIRQEAYKQQNATENRFSVNFQKAAKSFSLKISDSQLGDTAMY
FCALYSGAGSYQLTFGKGTKLSVIPNiqnpdpavyqlrdskssdksvclftdfdsqtnvsqskdsdvyitd
ktvldmrsmdfksnsavawsnksdfacanafnnsiipedtffpssdvpcdvklveksfetdtnlnfqnllvivlrilllkvagfill
mtlrlws (SEQ ID NO: 27)
Beta chain:
TRBV7-9/TRBJ2-1
MGTSLLCWMALCLLGADHADTGVSQDPRHKITKRGQNVTFRCDPISEHNRLYWY
RQTLGQGPEFLTYFQNEAQLEKSRLLSDRFSAERPKGSFSTLEIQRTEQGDSAMYL
CASFELAGGYEQFFGPGTRLTVLedlnkvfppevavfepskaeiahtqkatlvclatgffpdhvelswwvn
gkevhsgvstdpqplkeqpalndsryclssrlrvsatfwqnprnhfrcqvqfyglsendewtqdrakpvtqivsaeawgradc
gitsasyhqgvlsatilyeillgkatlyavlvsalvlmamvkrkdf (SEQ ID NO: 28)

TABLE 2
HA1-Fred Hutch sequence:
Alpha chain (with T48C modification)
TRAV21/TRAJ40/TRAC
METLLGLLILWLQLQWVSSKQEVTQIPAALSVPEGENLVLNCSFTDSAIYNLQWFR
QDPGKGLTSLLLIQSSQREQTSGRLNASLDKSSGRSTLYIAASQPGDSATYLCAVIG
LGGTYKYIFGTGTRLKVLANiqnpdpavyqlrdskssdksvclftdfdsqtnvsqskdsdvyitdkcvldmrs
mdfksnsavawsnksdfacanafnnsiipedtffpspesscdvklveksfetdtnlnfqnlsvigfrilllkvagfnllmtlrlws
(SEQ ID NO: 29)
Beta chain (with S57C modification)
TRBV7-9/TRBJ2-3/TRBC1
MGTSLLCWMALCLLGADHADTGVSQDPRHKITKRGQNVTFRCDPISEHNRLYWY
RQTLGQGPEFLTYFQNEAQLEKSRLLSDRFSAERPKGSFSTLEIQRTEQGDSAMYL
CASSLVKGEKLFFGSGTQLSVLedInkvfppevavfepseaeishtqkatlvclatgffpdhvelswwvngk
evhsgvctdpqplkeqpalndsryclssrlrvsatfwqnprnhfrcqvqfyglsendewtqdrakpvtqivsaeawgradcgft
svsyqqgvlsatilyeillgkatlyavlvsalvlmamvkrkdf (SEQ ID NO: 30)

Table 1 providing representative TCR sequences are grouped according to MHC serotype presentation and sub-grouped according to different peptides presented by the MHC serotype and bound by the sub-grouped TCRs. Individual TCRs, such as those representatively exemplified in the tables, are described and claimed, as well as the genus of binding proteins that bind a peptide epitope sequence described herein either alone or in a complex with an MHC, such as those grouped in the tables provided herein. In addition, TRAV, TRAJ, and TRAC genes for each TCR alpha chain described herein, and TRBV, TRBJ, and TRBC genes for each TCR beta chain described herein, are provided. Sequences for each TCR described herein are provided as pairs of cognate alpha chain and beta chains for each named TCR. TCR sequences described herein are annotated. Variable domain sequences are capitalized. Constant domain sequences are in lower case. CDR1, CDR2, and CDR3 sequences are annotated using bold and underlined text. CDR1, CDR2, and CDR3 are shown in standard order of appearance from left (N-terminus) to right (C-terminus). TRAV, TRAJ, and TRAC genes for each TCR alpha chain described herein, and TRBV, TRBJ, and TRBC genes for each TCR beta chain described herein, are annotated according to well-known IMGT nomenclature described herein.

TABLE 3
Vector: pTSLV102-MSCV-HA1-10-30-MGTM-Q-CD8:
(SEQ ID NO: 31)
tggaagggctaattcactcccaaagaagacaagatatccttgatctgtggatctaccacacacaaggctacttccctgattagcagaa
ctacacaccagggccaggggtcagatatccactgacctttggatggtgctacaagctagtaccagttgagccagataaggtagaag
aggccaataaaggagagaacaccagcttgttacaccctgtgagcctgcatgggatggatgacccggagagagaagtgttagagtg
gaggtttgacagccgcctagcatttcatcacgtggcccgagagctgcatccggagtacttcaagaactgctgatatcgagcttgctac
aagggactttccgctggggactttccagggaggcgtggcctggggggactggggagtggcgagccctcagatcctgcatataa
gcagctgctttttgcctgtactgggtctctctggttagaccagatctgagcctgggagctctctggctaactagggaacccactgctta
agcctcaataaagcttgccttgagtgcttcaagtagtgtgtgcccgtctgttgtgtgactctggtaactagagatccctcagaccctttta
gtcagtgtggaaaatctctagcagtggcgcccgaacagggacttgaaagcgaaagggaaaccagaggagctctctcgacgcagg
actcggcttgctgaagcgcgcacggcaagaggcgaggggcggcgactggtgagtacgccaaaaattttgactagcggaggcta
gaaggagagagatgggtgcgagagcgtcagtattaagcgggggagaattagatcgcgatgggaaaaaattcggttaaggccagg
gggaaagaaaaaatataaattaaaacatatagtatgggcaagcagggagctagaacgattcgcagttaatcctggcctgttagaaac
atcagaaggctgtagacaaatactgggacagctacaaccatcccttcagacaggatcagaagaacttagatcattatataatacagta
gcaaccctctattgtgtgcatcaaaggatagagataaaagacaccaaggaagctttagacaagatagaggaagagcaaaacaaaa
gtaagaccaccgcacagcaagcggccggccgctgatcttcagacctggaggaggagatatgagggacaattggagaagtgaatt
atataaatataaagtagtaaaaattgaaccattaggagtagcacccaccaaggcaaagagaagagtggtgcagagagaaaaaaga
gcagtgggaataggagctttgttccttgggttcttgggagcagcaggaagcactatgggcgcagcgtcaatgacgctgacggtaca
ggccagacaattattgtctggtatagtgcagcagcagaacaatttgctgagggctattgaggcgcaacagcatctgttgcaactcaca
gtctggggcatcaagcagctccaggcaagaatcctggctgtggaaagatacctaaaggatcaacagctcctggggatttggggttg
ctctggaaaactcatttgcaccactgctgtgccttggaatgctagttggagtaataaatctctggaacagatttggaatcacacgacct
ggatggagtgggacagagaaattaacaattacacaagcttaatacactccttaattgaagaatcgcaaaaccagcaagaaaagaat
gaacaagaattattggaattagataaatgggcaagtttgtggaattggtttaacataacaaattggctgtggtatataaaattattcataat
gatagtaggaggcttggtaggtttaagaatagtttttgctgtactttctatagtgaatagagttaggcagggatattcaccattatcgtttc
agacccacctcccaaccccgaggggacccgacaggcccgaaggaatagaagaagaaggtggagagagagacagagacagat
ccattcgattagtgaacggatctcgacggtatcgccgaattaattcacaaatggcagtattcatccacaattttaaaagaaaaggggg
gattggggggtacagtgcaggggaaagaatagtagacataatagcaacagacatacaaactaaagaattacaaaaacaaattaca
aaaattcaaaattttcgggtttattacaggCGcGCcagagatccagtttggacCTgcAGGTGAAAGACCCCAC
CTGTAGGTTTGGCAAGtTAGCTTAAGTAACGCCATTTTGCAAGGCATGGAA
AATACATAACTGAGAATAGAGAAGTTCAGATCAAGGTTAGGAACAGAGAG
ACAGCAGAATATGGGCCAAACAGGATATCTGTGGTAAGCAGTTCCTGCCC
CGGCTCAGGGCCAAGAACAGATGGTCCCCAGATGCGGTCCCGCCCTCAGC
AGTTTCTAGCGAACCATCAGATGTTTCCAGGGTGCCCCAAGGACCTGAAAT
GACCCTGTGCCTTATTTGAACTAACCAATCAGTTtGCTTCTtGCTTCTGTTtGt
GtGCTTCTGCTCCCtGAGCTCAATAAAAGAGCCCACAACCCCTCACTtGGtGg
GCCAGTCCTCtGATAGACTGtGTCcCCtGGaTACCCGTAcggtaccgctagcgccacc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

CGGCAGAGCCAAAAGGTCCGGGAGCGGTGCGACAAACTTTAGCCTGTTGAAAC
AAGCCGGCGACGTTGAAGAGAACCCCGGACCTATGGAAACCCTcTTGGGCCT
GCTTATCCTTTGGCTGCAGCTGCAATGGGTGAGCAGCAAACAGGAGGTGA
CTCAGATTCCTGCAGCTCTGAGTGTCCCAGAAGGAGAAAACTTGGTTCTCA
ACTGCAGTTTCACTGATAGCGCTATTTACAACCTCCAGTGGTTTAGGCAGG
ACCCTGGGAAAGGCCTCACATCTCTGTTGCTTATTCAGTCAAGTCAGAGAG
AGCAAACAAGTGGACGCCTTAATGCCTCTCTGGATAAATCATCAGGACGCA
GTACTCTTTACATTGCAGCTTCTCAGCCTGGTGATTCAGCCACCTACCTGT
GCGCTGTGAGGGGTGGTACCTCAGGAACCTACAAATACATCTTTGGAACA
GGCACCAGGCTGAAGGTTCTTGCAAACATCCAGAACCCCGACCCCGCCGT
GTACCAGCTGAGGGACTCCAAGTCCAGCGACAAGAGCGTGTGTCTGTTTA
CGGACTTCGACAGCCAGACCAACGTGAGTCAAAGCAAGGACAGCGACGTC
TACATAACGGATAAGACCGTGCTGGACATGCGGAGCATGGACTTCAAGAG
CAACAGCGCCGTGGCCTGGTCCAACAAGAGCGACTTCGCCTGCGCCAACG
CCTTCAACAACAGCATCATCCCCGAGGACACCTTCTTCCCCAGCAGCGACG
TGCCCTGCGACGTGAAACTGGTGGAGAAGTCCTTCGAGACAGACACCAAT
CTGAACTTTCAGAACCTGCTGGTGATCGTGCTGCGGATTCTGCTGCTGAAA
GTGGCCGGCTTCAATCTGCTGATGACCCTGCGGCTGTGGAGCAGCAGGGC
TAAGAGGTCCGGCAGCGGAGCCACCAATTTTTCCCTGCTGAAACAGGCTGGTG
ACGTGGAAGAAAACCCTGGCCCCATGGCGCTGCCCGTCACCGCGCTGCTGCTG
CCCCTGGCGCTGCTGTTACACGCCGCTCGGCCAGAGCTTCCCACCCAGGGCACAT
TCTCCAACGTGTCCACCAATGTGTCGGGAGGCGGCGGATCGTCCCAGTTCAGAGTG
TCCCCTCTGGACCGCACCTGGAACCTGGGCGAGACCGTGGAGCTGAAATGTCAGGT
CCTGCTGAGCAACCCGACCTCCGGGTGCAGTTGGCTGTTCCAGCCGCGTGGTGCTG
CCGCAAGCCCTACGTTCCTGCTTTACCTGAGCCAGAACAAGCCCAAGGCGGCCGAG
GGCCTGGACACCCAGAGATTCTCCGGCAAGCGCCTGGGGGACACATTCGTGCTTAC
TTTGAGCGATTTCCGCAGAGAGAACGAGGGCTACTATTTCTGTTCGGCGCTGAGCAA
TTCCATCATGTATTTCAGCCACTTTGTGCCAGTGTTCCTGCCTGCCAAGCCTACCACA
ACACCAGCTCCCCGTCCCCCGACTCCGGCGCCTACCATCGCGAGTCAACCGTTGAG
CCTGAGGCCTGAGGCTTGTCGGCCCGCTGCGGGGGGTGCCGTCCACACCAGGGGC
CTCGACTTTGCGTGCGACATCTATATTTGGGCGCCTCTGGCGGGTACCTGCGGGGT
GCTGCTGCTGTCATTGGTGATTACCCTGTACTGCAATCACCGCAACCGCCGGCGGGT
CTGTAAGTGCCCACGGCCTGTGGTCAAGTCCGGTGACAAACCGTCGCTCTCGGCTC
GCTACGTGCGCGCTAAGCGCAGCGGTTCCGGGGCCACCAACTTTTCATTGCTGA
AGCAGGCCGGTGATGTGGAGGAGAATCCAGGGCCCATGCGCCCCAGGCTTTGG
CTCCTTCTTGCTGCTCAGCTCACTGTCTTGCATGGCAACTCCGTTCTGCAGCAGA
CTCCCGCCTACATCAAGGTGCAGACGAACAAGATGGTGATGCTGTCATGCGAG
GCCAAGATCTCTCTTTCAAATATGAGAATTTATTGGCTACGACAGCGCCAGGCC
CCCTCCAGCGACAGCCACCACGAGTTCCTGGCGCTTTGGGATTCTGCTAAAGGC
ACCATCCATGGAGAGGAGGTGGAACAGGAGAAGATAGCTGTCTTCCGCGACGC
ATCCCGCTTCATCCTGAACCTGACCAGCGTGAAGCCGGAGGACAGCGGCATCT
ACTTCTGTATGATCGTTGGCTCCCCCGAGCTGACCTTCGGCAAAGGCACCCAGC
TGTCCGTGGTGGACTTCCTGCCCACCACAGCCCAGCCAACCAAGAAATCCACCC
TCAAGAAGCGCGTGTGCCGACTGCCCCGCCCTGAAACCCAGAAGGGCCCTCTG
TGCTCCCCCATCACCCTTGGACTGCTGGTGGCGGGAGTCCTGGTGCTGCTCGTA
TCTCTGGGTGTCGCCATCCACCTGTGCTGCCGCCGCCGCCGCGCCCGCCTGAGG
TTTATGAAACAGTTTTACAAGTGATAAatcgatagatcctaatcaacctctggattacaaaatttgtgaaag
attgactggtattcttaactatgttgctccttttacgctatgtggatacgctgctttaatgcctttgtatcatgctattgcttcccgtatggc
tttcattttctcctccttgtataaatcctggttgctgtctctttatgaggagttgtggcccgttgtcaggcaacgtggcgtggtgtgcactgt
gtttgctgacgcaacccccactggttggggcattgccaccacctgtcagctcctttccgggactttcgctttccccctccctattgccacg
gcggaactcatcgccgcctgccttgcccgctgctggacaggggctcggctgttgggcactgacaattccgtggtgttgtcggggaa
atcatcgtcctttccttggctgctcgcctgtgttgccacctggattctgcgcgggacgtccttctgctacgtcccttcggccctcaatcca
gcggaccttccttcccgcggcctgctgccggctctgcggcctcttccgcgtcttcgccttcgccctcagacgagtcggatctcccttt
gggccgcctccccgcctgagatcctttaagaccaatgacttacaaggcagctgtagatcttagccactttttaaaagaaaagggggg
actggaagggctaattcactcccaacgaagacaagatctgctttttgcttgtactgggtctctctggttagaccagatctgagcctggg
agctctctggctaactagggaacccactgcttaagcctcaataaagcttgccttgagtgcttcaagtagtgtgtgcccgtctgttgtgtg
actctggtaactagagatccctcagacccttttagtcagtgtggaaaatctctagcagtagtagttcatgtcatcttattattcagtatttat
aacttgcaaagaaatgaatatcagagagtgagaggcccgggttaattaaggaaagggctagatcattcttgaagacgaaagggcct
cgtgatacgcctatttttataggttaatgtcatgataataatggtttcttagacgtcaggtggcacttttcggggaaatgtgcgcggaacc
cctatttgtttatttttctaaatacattcaaatatgtatccgctcatgagacaataaccctgataaatgcttcaataatattgaaaaaggaag
agtatgagtattcaacatttccgtgtcgcccttattcccttttttgcggcattttgccttcctgtttttgctcacccagaaacgctggtgaaa
gtaaaagatgctgaagatcagttgggtgcacgagtgggttacatcgaactggatctcaacagcggtaagatccttgagagttttcgcc
ccgaagaacgttttccaatgatgagcacttttaaagttctgctatgtggcgcggtattatcccgtgttgacgccgggcaagagcaactc
ggtcgccgcatacactattctcagaatgacttggttgagtactcaccagtcacagaaaagcatcttacggatggcatgacagtaaga
gaattatgcagtgctgccataaccatgagtgataacactgcggccaacttacttctgacaacgatcggaggaccgaaggagctaac
cgcttttttgcacaacatgggggatcatgtaactcgccttgatcgttgggaaccggagctgaatgaagccataccaaacgacgagcg
tgacaccacgatgcctgtagcaatggcaacaacgttgcgcaaactattaactggcgaactacttactctagcttcccggcaacaatta
atagactggatggaggcggataaagttgcaggaccacttctgcgctcggcccttccggctggctggtttattgctgataaatctggag
ccggtgagcgtgggtctcgcggtatcattgcagcactggggccagatggtaagccctcccgtatcgtagttatctacacgacgggg
agtcaggcaactatggatgaacgaaatagacagatcgctgagataggtgcctcactgattaagcattggtaactgtcagaccaagttt
actcatatatactttagattgatttaaaacttcatttttaatttaaaaggatctaggtgaagatcctttttgataatctcatgaccaaaatcc
cttaacgtgagttttcgttccactgagcgtcagaccccgtagaaaagatcaaaggatcttcttgagatcctttttttctgcgcgtaatctgct
gcttgcaaacaaaaaaaccaccgctaccagcggtggtttgtttgccggatcaagagctaccaactctttttccgaaggtaactggcttca
gcagagcgcagataccaaatactgttcttctagtgtagccgtagttaggccaccacttcaagaactctgtagcaccgcctacatacct
cgctctgctaatcctgttaccagtggctgctgccagtggcgataagtcgtgtcttaccgggttggactcaagacgatagttaccggat
aaggcgcagcggtcgggctgaacggggggttcgtgcacacagcccagcttggagcgaacgacctacaccgaactgagatacct
acagcgtgagctatgagaaagcgccacgcttcccgaagggagaaaggcggacaggtatccggtaagcggcagggtcggaaca
ggagagcgcacgagggagcttccagggggaaacgcctggtatctttatagtcctgtcgggtttcgccacctctgacttgagcgtcg
atttttgtgatgctcgtcaggggggcggagcctatggaaaaacgccagcaacgcggcctttttacggttcctggccttttgctggcctt
ttgctcacatgttctttcctgcgttatccCCTGATTCTGTGGATAACCGTATTACCGCCTTTGAGTG
AGCTGATACCGCTCGCCGCAGCCGAACGACCGAGCGCAGCGAGTCAGTGAGCG
AGGAAGCGGAAGAGCGCCCAATACGCAAACCGCCTCTCCCCGCGCGTTGGCCG
ATTCATTAATGCAGCAAGCTCATGGCTGACTAATTTTTTTTATTTATGCAGAGGC
CGAGGCCGCCTCGGCCTCTGAGCTATTCCAGAAGTAGTGAGGAGGCTTTTTTGG
AGGCCTAGGCTTTTGCAAAAAGCTCCCCGTGGCACGACAGGTTTCCCGACTGGA
AAGCGGGCAGTGAGCGCAACGCAATTAATGTGAGTTAGCTCACTCATTAGGCA
CCCCAGGCTTTACACTTTATGCTTCCGGCTCGTATGTTGTGTGGAATTGTGAGCG
GATAACAATTTCACACAGGAAACAGCTATGACATGATTACGAATTTCACAAAT
AAAGCATTTTTTTCACTGCATTCTAGTTGTGGTTTGTCCAAACTCATCAATGTAT
CTTATCATGTCTGGATCAACTGGATAACTCAAGCTAACCAAAATCATCCCAAAC
TTCCCACCCCATACCCTATTACCACTGCCAATTACCTGTGGTTTCATTTACTCTA
AACCTGTGATTCCTCTGAATTATTTTCATTTTAAAGAAATTGTATTTGTTAAATA
TGTACTACAAACTtagtagt
*MSCV promoter is in bold. Beta chain is annotated using bold and italic text. Alpha
chain is annotated using bold and underlined text. Q tag is annotated using italic and
underlined text. CD8-alpha is in italic. CD8-beta is underlined.
Vector: TSC-100 npDNA Transposon:
(SEQ ID NO: 32)
AATTAATTGCTTGCAATTAACCCTTTAACGGTTATAAGGATCTAGATGAGATAGAAAGATTTGGTTTTCGGA
TTTGTGTTACATAAGATGCCTAAAATAAAAATTGAGATTCAATTTTTTTTAAACTTTTTTTTAATTGGTGGTA
AGAATATTCCCTCTACCTGTTTGAGAGTAATGAAATTGTAGTATGATTTTTCAACAAACTAAAAAAACAACA
TAAATCTCACATAATAACTTTATTTCAATCACACAATTGAATACCAATATTAATTAATGGTTGACAGTACTTA
CCAGCCTGCAGGTGAAAGACCCCACCTGTAGGTTTGGCAAGTTAGCTTAAGTAACGCCATTTTGCAAGGCAT
GGAAAATACATAACTGAGAATAGAGAAGTTCAGATCAAGGTTAGGAACAGAGAGACAGCAGAATATGGGC
CAAACAGGATATCTGTGGTAAGCAGTTCCTGCCCCGGCTCAGGGCCAAGAACAGATGGTCCCCAGATGCGGT
CCCGCCCTCAGCAGTTTCTAGCGAACCATCAGATGTTTCCAGGGTGCCCCAAGGACCTGAAATGACCCTGTG
CCTTATTTGAACTAACCAATCAGTTTGCTTCTTGCTTCTGTTTGTGTGCTTCTGCTCCCTGAGCTCAATAAAA
GAGCCCACAACCCCTCACTTGGTGGGCCAGTCCTCTGATAGACTGTGTCCCCTGGATACCCGTACGGTACCG
CTAGCGCCACCATGGGCACCAGCCTCCTCTGCTGGATGGCCCTGTGTCTCCTGGGGGCAGATCACGCAGATA
CTGGAGTCTCCCAGGACCCCAGACACAAGATCACAAAGAGGGGACAGAATGTTACTTTCAGGTGTGATCCA
ATTTCTGAACACAACCGCCTTTATTGGTACCGCCAGACCCTGGGGCAGGGCCCAGAGTTTCTGACTTACTTC
CAGAATGAAGCTCAACTTGAAAAATCAAGGCTGCTCAGTGATCGGTTCTCTGCAGAGAGGCCTAAGGGATC
TTTCTCCACCTTGGAGATCCAGCGCACAGAGCAGGGGGACTCTGCCATGTATCTCTGTGCCAGCAGCCGCAC
TGCTGGAGATACTCAGTATTTTGGCCCAGGCACCCGGCTGACAGTGCTCGAAGATCTGAACAAGGTGTTCCC
TCCAGAGGTGGCCGTGTTCGAGCCTTCTAAGGCCGAGATCGCCCACACACAAAAAGCCACCCTCGTGTGCCT
GGCCACCGGCTTTTTCCCCGACCACGTGGAACTGTCTTGGTGGGTCAACGGCAAAGAGGTGCACTCCGGCGT
GTCAACGGATCCCCAGCCTCTGAAAGAACAGCCTGCCCTGAACGACAGCCGGTACTGCCTGAGCTCCAGACT
GAGAGTGTCCGCCACCTTCTGGCAGAACCCCCGGAACCACTTCAGATGCCAGGTGCAGTTTTACGGCCTGAG
CGAGAACGACGAGTGGACCCAGGACAGAGCCAAGCCCGTGACACAAATCGTGTCTGCCGAAGCCTGGGGA
AGAGCCGATTGCGGCATCACCAGCGCCTCCTATCACCAGGGCGTGCTGAGCGCCACAATCCTGTACGAAAT
CCTGCTGGGCAAGGCCACCCTGTACGCCGTGCTGGTGTCTGCTCTGGTGCTGATGGCCATGGTCAAGCGGAA
GGACTTTGGCAGCGGCAGAGCCAAAAGGTCCGGGAGCGGTGCGACAAACTTTAGCCTGTTGAAACAAGCCG
GCGACGTTGAAGAGAACCCCGGACCTATGGAAACCCTCTTGGGCCTGCTTATCCTTTGGCTGCAGCTGCAATGG
GTGAGCAGCAAACAGGAGGTGACTCAGATTCCTGCAGCTCTGAGTGTCCCAGAAGGAGAAAACTTGGTTCTC
AACTGCAGTTTCACTGATAGCGCTATTTACAACCTCCAGTGGTTTAGGCAGGACCCTGGGAAAGGCCTCACA
TCTCTGTTGCTTATTCAGTCAAGTCAGAGAGAGCAAACAAGTGGACGCCTTAATGCCTCTCTGGATAAATCA
TCAGGACGCAGTACTCTTTACATTGCAGCTTCTCAGCCTGGTGATTCAGCCACCTACCTGTGCGCTGTGAGGGGT
GGTACCTCAGGAACCTACAAATACATCTTTGGAACAGGCACCAGGCTGAAGGTTCTTGCAAACATCCAGAAC
CCCGACCCCGCCGTGTACCAGCTGAGGGACTCCAAGTCCAGCGACAAGAGCGTGTGTCTGTTTACGGACTTC
GACAGCCAGACCAACGTGAGTCAAAGCAAGGACAGCGACGTCTACATAACGGATAAGACCGTGCTGGACA
TGCGGAGCATGGACTTCAAGAGCAACAGCGCCGTGGCCTGGTCCAACAAGAGCGACTTCGCCTGCGCCAAC
GCCTTCAACAACAGCATCATCCCCGAGGACACCTTCTTCCCCAGCAGCGACGTGCCCTGCGACGTGAAACTG
GTGGAGAAGTCCTTCGAGACAGACACCAATCTGAACTTTCAGAACCTGCTGGTGATCGTGCTGCGGATTCTGCT
GCTGAAAGTGGCCGGCTTCAATCTGCTGATGACCCTGCGGCTGTGGAGCAGCAGGGCTAAGAGGTCCGGCAG
CGGAGCCACCAATTTTTCCCTGCTGAAACAGGCTGGTGACGTGGAAGAAAACCCTGGCCCCATGGCGCTGC
CCGTCACCGCGCTGCTGCTGCCCCTGGCGCTGCTGTTACACGCCGCTCGGCCAGAGCTTCCCACCCAGGGCA
CATTCTCCAACGTGTCCACCAATGTGTCGGGAGGCGGCGGATCGTCCCAGTTCAGAGTGTCCCCTCTGGACC
GCACCTGGAACCTGGGCGAGACCGTGGAGCTGAAATGTCAGGTCCTGCTGAGCAACCCGACCTCCGGGTGC
AGTTGGCTGTTCCAGCCGCGTGGTGCTGCCGCAAGCCCTACGTTCCTGCTTTACCTGAGCCAGAACAAGCCC
AAGGCGGCCGAGGGCCTGGACACCCAGAGATTCTCCGGCAAGCGCCTGGGGGACACATTCGTGCTTACTTT
GAGCGATTTCCGCAGAGAGAACGAGGGCTACTATTTCTGTTCGGCGCTGAGCAATTCCATCATGTATTTCAG
CCACTTTGTGCCAGTGTTCCTGCCTGCCAAGCCTACCACAACACCAGCTCCCCGTCCCCCGACTCCGGCGCC
TACCATCGCGAGTCAACCGTTGAGCCTGAGGCCTGAGGCTTGTCGGCCCGCTGCGGGGGGTGCCGTCCACA
CCAGGGGCCTCGACTTTGCGTGCGACATCTATATTTGGGCGCCTCTGGCGGGTACCTGCGGGGTGCTGCTGC
TGTCATTGGTGATTACCCTGTACTGCAATCACCGCAACCGCCGGCGGGTCTGTAAGTGCCCACGGCCTGTGG
TCAAGTCCGGTGACAAACCGTCGCTCTCGGCTCGCTACGTGCGCGCTAAGCGCAGCGGTTCCGGGGCCACC
AACTTTTCATTGCTGAAGCAGGCCGGTGATGTGGAGGAGAATCCAGGGCCCATGCGCCCCAGGCTTTGGCTC
CTTCTTGCTGCTCAGCTCACTGTCTTGCATGGCAACTCCGTTCTGCAGCAGACTCCCGCCTACATCAAGGTGC
AGACGAACAAGATGGTGATGCTGTCATGCGAGGCCAAGATCTCTCTTTCAAATATGAGAATTTATTGGCTAC
GACAGCGCCAGGCCCCCTCCAGCGACAGCCACCACGAGTTCCTGGCGCTTTGGGATTCTGCTAAAGGCACCA
TCCATGGAGAGGAGGTGGAACAGGAGAAGATAGCTGTCTTCCGCGACGCATCCCGCTTCATCCTGAACCTG
ACCAGCGTGAAGCCGGAGGACAGCGGCATCTACTTCTGTATGATCGTTGGCTCCCCCGAGCTGACCTTCGGC
AAAGGCACCCAGCTGTCCGTGGTGGACTTCCTGCCCACCACAGCCCAGCCAACCAAGAAATCCACCCTCAA
GAAGCGCGTGTGCCGACTGCCCCGCCCTGAAACCCAGAAGGGCCCTCTGTGCTCCCCCATCACCCTTGGACT
GCTGGTGGCGGGAGTCCTGGTGCTGCTCGTATCTCTGGGTGTCGCCATCCACCTGTGCTGCCGCCGCCGCCG
CGCCCGCCTGAGGTTTATGAAACAGTTTTACAAGTGATAAATCGATAGATCCTAATCAACCTCTGGATTACA
AAATTTGTGAAAGATTGACTGGTATTCTTAACTATGTTGCTCCTTTTACGCTATGTGGATACGCTGCTTTAATG
CCTTTGTATCATGCTATTGCTTCCCGTATGGCTTTCATTTTCTCCTCCTTGTATAAATCCTGGTTGCTGTCTCTT
TATGAGGAGTTGTGGCCCGTTGTCAGGCAACGTGGCGTGGTGTGCACTGTGTTTGCTGACGCAACCCCCACT
GGTTGGGGCATTGCCACCACCTGTCAGCTCCTTTCCGGGACTTTCGCTTTCCCCCTCCCTATTGCCACGGCGG
AACTCATCGCCGCCTGCCTTGCCCGCTGCTGGACAGGGGCTCGGCTGTTGGGCACTGACAATTCCGTGGTGTT
GTCGGGGAAATCATCGTCCTTTCCTTGGCTGCTCGCCTGTGTTGCCACCTGGATTCTGCGCGGGACGTCCTTC
TGCTACGTCCCTTCGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCCTGAGATCCTTTAAGA
CCAATGCTGGAGTTCTTCGCCCACCCCAACTTGTTTATTGCAGCTTATAATGGTTACAAATAAAGCAATAGC
ATCACAAATTTCACAAATAAAGCATTTTTTTCACTGCATTCTAGTTGTGGTTTGTCCAAACTCATCAATGTATC
TTATCATGTCTGTATACAGGTTACCTCAGTCTCCTAGGTACGTCTTATATCTATGAAAAAACATTCAAAAGCA
CAACATCTAGAAGAACTTACCTTTTTTCACCACTCTATTGCAAAGATATGTACCGATTTCTCTCGAAGTACAA
AAAACCGCTAGTTTTCAAATTCACCTCAAGACTTTGAAAAAAAATTGAATCTGTCAATGTCAAATAAAATCA
GAAACAAATGTCATAATGTTACGTTAATGTTGTCAGGTCGAAAAATAAAATTGCAAATAGAAATTTTGTTCCTT
TTTTATTGGTTTTTATTGGTGGGAAAAATATTCCCTCTAACTGCAAAAGGGTTAATTATGTTAGAGGTAGAGTC
GACGCTAGCTGGCTTGTTGTCCACAACCATTAAACCTTAAAAGCTTTAAAAGCCTTATATATTCTTTTTTTTC
TTATAAAACTTAAAACCTTAGAGGCTATTTAAGTTGCTGATTTATATTAATTTTATTGTTCAAACATGAGAGC
TTAGTACGTGAAACATGAGAGCTTAGTACATTAGCCATGAGAGCTTAGTACATTAGCCATGAGGGTTTAGTT
CATTAAACATGAGAGCTTAGTACATTAAACATGAGAGCTTAGTACATACTATCAACAGGTTGAACTGCTGAT
CTGTACAGTAGAATTGGTAAAGAGAGTTGTGTAAAATATTGAGTTCGCACATCTTGTTGTCTGATTATTGATT
TTTGGCGAAACCATTTGATCATATGACAAGATGTGTATCTACCTTAACTTAATGATTTTGATAAAAATCATTA
GGTACC

The TSC-100 npDNA Transposon is a 5,709-bp supercoiled, Nanoplasmid™ DNA (npDNA) carrying the minimal R6K bacterial replication origin and anti-sense RNA-based antibiotic-free selection marker (RNA-OUT). The terminal inverted repeats (TIRs) of the armyworm Spodoptera frugiperda transposon (required for transposase-mediated integration) are connected to the two ends of the exogenous DNA sequence. The TSC-100 HA-1-specific TCR α- and β-chain genes and the codon-diversified CD8 α- and β-chain genes are placed under the control of the murine stem cell virus (MSCV) promoter. The constant regions of the TCR α- and β-chains contain amino-acid substitutions that stabilize their expression on the surface of engineered T cells. These four open reading frames are encoded on a single mRNA molecule along with woodchuck post-transcriptional regulatory element (WPRE), which is known to enhance gene expression (Loeb, 1999). The ribosomal skipping that takes places on the self-cleaving peptide P2A leads to production of the four separate polypeptides. The CD34 epitope recognized by the mouse monoclonal antibody QBend/10 is fused to the N-terminus of the exogenous CD8 α-chain, allowing for purification of transgene-expressing cells to increase the safety and potency of the drug substance. The Nanoplasmid vector backbone (<500 base pairs) is substantially smaller than conventional plasmids (>1500 base pairs). It contains no antibiotic-resistant gene, and its replication origin (R6K) necessary for plasmid preparation can support propagation in a narrower range of bacterial species than a commonly used origin (pUC).

TABLE 4
SEQ ID NO: 33 Human CCDC114 cDNA Sequence variant 1 (NM_001364171.2;
CDS: 358-2481)

| | |
|---|---|
| 1 | ccgccctcca gcgcgcggac gtggtaggcg gccaagcgtt tttggctgct tcccgggaag |
| 61 | ccgttggtcg cgcttgcgta gtgaaccgct gaggctgtgt tgagcgctcc ttctccgcgc |
| 121 | aactcccggc ttcgacaccg ccttccttcc tatgaatcct taatttaacg tggtcacagt |
| 181 | cagcttaaga ccggcacctc tegggctccg tcttgtagga cctcggcctg gcttcgaggg |
| 241 | cttccataca ggtcaccgca ggctctttca actcccgccg cttccaggcc cgcgtaacca |
| 301 | gaccacaaca ctgaacttgg agcaccaaga acaggagccc caccctgttt ggccaggatg |
| 361 | cctttgggac gcttggcagg gagcgcccgc tccgaggagg gaagcgaggc atttctggag |
| 421 | ggaatggtgg attgggagct gagtaggctg cagcgacaat gcaaagtgat ggaaggggag |
| 481 | aggcgggcct acagcaagga agtccaccag cgcatcaaca agcaacttga ggagatccgg |
| 541 | cgcttggagg aggtacgggg cgatctccag gtgcagatca gcgcagccca gaaccaggtc |
| 601 | aagcggcttc gggacagtca gcggctggag aacatggacc gcctgctgaa gggccgggcc |
| 661 | caggtgcagg cggagatcga ggagctgcag gagcagacca gggccctgga caagcagatc |
| 721 | caggagtggg agacgcggat ctttacccac agtaagaatg tcaggtcccc gggattcatc |
| 781 | ctggatcaga aggtcaagat caggcgaagg atcaggatcc tagaaaacca gttggacagg |
| 841 | gtcacctgtc actttgacaa ccagctggta cggaatgcgg ccctgcggga ggagctggat |
| 901 | ctgctgcgga tcgacaggaa ccgctatctg aacgtggacc gcaagctgaa gaaggagatc |
| 961 | caccacctgc atcacctggt cagcaccctt atcctctcct ccacctctgc ctacgccgtc |
| 1021 | agggaggagg cgaaggccaa gatgggcttg ctgcgggagc gcgcggagaa agaggaggcc |
| 1081 | cagagcgaga tggaggcgca ggtcctgcag cggcagatct tgcacctgga gcagctgcac |
| 1141 | cacttcctca agctcaagaa caacgaccgg cagccggatc ccgatgtcct ggagaagcgt |
| 1201 | gaaaagcagg ccggggaggt ggccgagggc gtctggaaga cctcccagga gaggctggtg |
| 1261 | ctttgctacg aggacgccct gaataaactg toccagctga tgggggagag tgaccctgac |
| 1321 | ctgttggtgc agaagtatct ggagatcgag gagcgcaact ttgctgagtt caacttcatc |
| 1381 | aacgagcaga acttggagct ggagcatgtg caggaagaga tcaaggagat gcaggaggct |
| 1441 | ttggtgagcg cacgtgccag caaggatgac cagcatttgc tgcaggagca gcagcagaag |
| 1501 | gtgttgcagc agcgcatgga caaggtgcac tcggaggctg agcgccttga ggcccgcttc |
| 1561 | caggatgtgc ggggacagct ggagaagctc aaggctgata tccagctcct cttcaccaag |
| 1621 | gcccattgcg acagcagcat gatcgatgac ctccttgggg tcaagaccag catgggagac |
| 1681 | cgggacatgg gcctcttcct gagcctcatt gagaagcggc tggtggagct cctgacagtg |
| 1741 | caggccttcc tacatgccca gagcttcacc tccctggccg acgctgccct cctagtgctg |
| 1801 | ggccagagcc tggaggacct tccgaagaag atggccccac ttcagccccc tgacactcta |
| 1861 | gaagaccccc cgggttttga ggccagcgat gactacccca tgagcaggga ggagctgctg |
| 1921 | agccaagtgg agaagctggt ggagctccag gagcaggcgg aggcgcagcg ccagaaggac |
| 1981 | ctggccgccg ccgccgcgaa gctggacggc accctgagcg tggacctggc cagcacccag |
| 2041 | agggccggct ccagtaccgt cctggtgccc accaggcacc cccatgccat ccccgggtcc |
| 2101 | attttgagcc acaagactag cagagaccgt ggctctcttg gccacgtcac ttttggcggc |
| 2161 | ctcagctcca gcactgggca tttgcccage cacatcacgc acggtgaccc caacactggc |
| 2221 | cacgtgacct tcggctccac cagtgcctcg agtgggggcc acgtgacctt cagacccgtc |
| 2281 | agcgccagca gctacctggg ctccactgga tacgtggggt ccagcagggg cggagaaaac |
| 2341 | acagagggtg gtgtggagag cggaggcaca gcgtctgatt cgagcggagg cctcgggtcc |
| 2401 | agcagagacc acgtctccag caccggccct gcctccagca ctggcccggg ctcctccacc |
| 2461 | agcaaagact cccggggcta acacgagggg cacgcagccc ccaccctgcc ctggctctct |
| 2521 | gtggggtctt tttttgtgtc tctacttccc ctgtttgcct tgtgggtctc tgtctttttc |
| 2581 | tcccagtggc aggcctctgt ctttttgccc cttcaactgt tttcatctgc ccttcatcac |
| 2641 | cgcctcacct gtgtctcccg gttcctttct ctctgctcct gacactgctt ttccctctga |
| 2701 | ctcctgatct cctgttctct gggcttcctg gtcagtgtct gtccgtcttg tctccatatc |
| 2761 | tgccccttcc ctgtctgctg agccctggcc ccctgtggat cccccacctc ctgtttccgg |
| 2821 | ctctctctcc tcatctccat ctcccaggct ctccacgcat cctgtgtccc tgtctctgta |
| 2881 | gctccttact ccccaagagc cccccagtgc tgacaggtgt gggaaaagtc agggcgaaga |
| 2941 | cgccaggttc agcatgggaa aatgggactg tggaaataaa gtatatgtgt ggaga |

SEQ ID NO: 34 Human CCDC114 cDNA Sequence variant 2 (NM_144577.4; CDS:
108-2120)

| | |
|---|---|
| 1 | aatcacccgc tgattgaaaa ctactgggcc tttggagatg gggagcatac gggcaagact |
| 61 | ggagagtgga ttgggagctg agtaggctgc agcgacaatg caaagtgatg gaaggggaga |
| 121 | ggcgggccta cagcaaggaa gtccaccagc gcatcaacaa gcaacttgag gagatccggc |
| 181 | gcttggagga ggtacggggc gatctccagg tgcagatcag cgcagcccag aaccaggtca |
| 241 | agcggcttcg ggacagtcag cggctggaga acatggaccg cctgctgaag ggccgggccc |
| 301 | aggtgcaggc ggagatcgag gagctgcagg agcagaccag ggccctggac aagcagatcc |
| 361 | aggagtggga gacgcggatc tttacccaca gtaagaatgt caggtccccg ggattcatcc |
| 421 | tggatcagaa ggtcaagatc aggcgaagga tcaggatcct agaaaaccag ttggacaggg |
| 481 | tcacctgtca ctttgacaac cagctggtac ggaatgcggc cctgcgggag gagctggatc |
| 541 | tgctgcggat cgacaggaac cgctatctga acgtggaccg caagctgaag aaggagatcc |
| 601 | accacctgca tcacctggtc agcaccetta tectctcctc cacctctgcc tacgccgtca |
| 661 | gggaggaggc gaaggccaag atgggcttgc tgcgggagcg cgcggagaaa gaggaggccc |
| 721 | agagcgagat ggaggcgcag gtcctgcagc ggcagatctt gcacctggag cagctgcacc |
| 781 | acttcctcaa gctcaagaac aacgaccggc agccggatcc cgatgtcctg gagaagcgtg |
| 841 | aaaagcaggc cggggaggtg gccgagggcg tctggaagac ctcccaggag aggctggtgc |
| 901 | tttgctacga ggacgccctg aataaactgt cccagctgat gggggagagt gaccctgacc |
| 961 | tgttggtgca gaagtatctg gagatcgagg agcgcaactt tgctgagttc aacttcatca |
| 1021 | acgagcagaa cttggagctg gagcatgtgc aggaagagat caaggagatg caggaggctt |
| 1081 | tggtgagcgc acgtgccagc aaggatgacc agcatttgct gcaggagcag cagcagaagg |
| 1141 | tgttgcagca gcgcatggac aaggtgcact cggaggctga gcgccttgag gcccgcttcc |
| 1201 | aggatgtgcg gggacagctg gagaagctca aggctgatat ccagctcctc ttcaccaagg |
| 1261 | cccattgcga cagcagcatg atcgatgacc tccttggggt caagaccage atgggagacc |
| 1321 | gggacatggg cctcttcctg agcctcattg agaagcggct ggtggagctc ctgacagtgc |
| 1381 | aggccttcct acatgcccag agcttcacct ccctggccga cgctgccctc ctagtgctgg |
| 1441 | gccagagcct ggaggacctt ccgaagaaga tggccccact tcagccccct gacactctag |
| 1501 | aagacccccc gggttttgag gccagcgatg actaccccat gagcagggag gagctgctga |
| 1561 | gccaagtgga gaagctggtg gagctccagg agcaggcgga ggcgcagcgc cagaaggacc |
| 1621 | tggccgccgc cgccgcgaag ctggacggca ccctgagcgt ggacctggcc agcacccaga |
| 1681 | gggccggctc cagtaccgtc ctggtgccca ccaggcaccc ccatgccatc cccgggtcca |
| 1741 | ttttgagcca caagactagc agagaccgtg gctctcttgg ccacgtcact tttggcggcc |
| 1801 | tcagctccag cactgggcat ttgcccagcc acatcacgca cggtgacccc aacactggcc |
| 1861 | acgtgacctt cggctccacc agtgcctcga gtgggggcca cgtgaccttc agacccgtca |
| 1921 | gcgccagcag ctacctgggc tccactggat acgtggggtc cagcaggggc ggagaaaaca |
| 1981 | cagagggtgg tgtggagagc ggaggcacag cgtctgattc gagcggaggc ctcgggtcca |
| 2041 | gcagagacca cgtctccage accggccctg cctccagcac tggcccgggc tcctccacca |
| 2101 | gcaaagactc ccggggctaa cacgaggggc acgcagcccc caccctgccc tggctctctg |
| 2161 | tggggtcttt ttttgtgtct ctacttcccc tgtttgcctt gtgggtctct gtctttttct |
| 2221 | cccagtggca ggcctctgtc tttttgcccc ttcaactgtt ttcatctgcc cttcatcacc |
| 2281 | gcctcacctg tgtctcccgg ttcctttctc tctgctcctg acactgcttt tccctctgac |
| 2341 | tcctgatctc ctgttctctg ggcttcctgg tcagtgtctg tccgtcttgt ctccatatct |
| 2401 | gccccttccc tgtctgctga gccctggccc cctgtggatc ccccacctcc tgtttccggc |
| 2461 | tctctctcct catctccatc toccaggctc tccacgcatc ctgtgtccct gtctctgtag |
| 2521 | ctccttactc cccaagagcc ccccagtgct gacaggtgtg ggaaaagtca gggcgaagac |
| 2581 | gccaggttca gcatgggaaa atgggactgt ggaaataaag tatatgtgtg gagagctggc |
| 2641 | ttcaaaaaaa aaaaaaaa |

SEQ ID NO: 35 Human CCDC114 Amino Acid Sequence isoform 1
(NP_001351100.1)

| | |
|---|---|
| 1 | mplgrlagsa rseegseafl egmvdwelsr lqrqckvmeg errayskevh qrinkqleei |
| 61 | rrleevrgdl qvqisaaqnq vkrlrdsqrl enmdrllkgr aqvqaeieel qeqtraldkq |
| 121 | iqewetrift hsknvrspgf ildqkvkirr ririlenqld rvtchfdnql vrnaalreel |
| 181 | dllridrnry lnvdrklkke ihhlhhlvst lilsstsaya vreeakakmg llreraekee |
| 241 | aqsemeaqvl qrqilhleql hhflklknnd rqpdpdvlek rekqagevae gvwktsqerl |
| 301 | vlcyedalnk lsqlmgesdp dllvqkylei eernfaefnf ineqnleleh vqeeikemqe |
| 361 | alvsaraskd dqhllqeqqq kvlqqrmdkv hseaerlear fqdvrgqlek lkadiqllft |
| 421 | kahcdssmid dllgvktsmg drdmglflsl iekrlvellt vqaflhaqsf tsladaallv |
| 481 | lgqsledlpk kmaplqppdt ledppgfeas ddypmsreel lsqveklvel qeqaeaqrqk |
| 541 | dlaaaaakld gtlsvdlast qragsstvlv ptrhphaipg silshktsrd rgslghvtfg |
| 601 | glssstghlp shithgdpnt ghvtfgstsa ssgghvtfrp vsassylgst gyvgssrgge |
| 661 | nteggvesgg tasdssgglg ssrdhvsstg passtgpgss tskdsrg |

SEQ ID NO: 36 Human CCDC114 Amino Acid Sequence isoform 2 (NP 653178.3)

| | |
|---|---|
| 1 | megerraysk evhqrinkql eeirrleevr gdlqvqisaa qnqvkrlrds qrlenmdrll |
| 61 | kgraqvqaei eelqeqtral dkqiqewetr ifthsknvrs pgfildqkvk irrririlen |
| 121 | qldrvtchfd nqlvrnaalr eeldllridr nrylnvdrkl kkeihhlhhl vstlilssts |
| 181 | ayavreeaka kmgllrerae keeaqsemea qvlqrqilhl eqlhhflklk nndrqpdpdv |
| 241 | lekrekqage vaegvwktsq erlvlcyeda lnklsqlmge sdpdllvqky leieernfae |
| 301 | infineqnle lehvqeeike mqealvsara skddqhllqe qqqkvlqqrm dkvhseaerl |
| 361 | earfqdvrgq leklkadiql lftkahcdss middllgvkt smgdrdmglf lsliekrlve |
| 421 | lltvqaflha qsftsladaa llvlgqsled lpkkmaplqp pdtledppgf easddypmsr |
| 481 | eellsqvekl velgeqaeaq rqkdlaaaaa kldgtlsvdl astqragsst vlvptrhpha |
| 541 | ipgsilshkt srdrgslghv tfgglssstg hlpshithgd pntghvtfgs tsassgghvt |
| 601 | frpvsassyl gstgyvgssr ggenteggve sggtasdssg glgssrdhvs stgpasstgp |
| 661 | gsstskdsrg |

Included in Tables 1, 2 and 4 herein are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Tables 1, 2 and 4, or a portion thereof. Such polypeptides may have a function of the full-length peptide or polypeptide as described further herein.
Included in Tables 3 and 4 are RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any sequence listed in Tables 3 and 4, or a portion thereof. Such nucleic acid molecules can have a function of the full-length nucleic acid as described further herein.

In some embodiments, the binding proteins disclosed herein may comprise a T cell receptor (TCR), an antigen-binding fragment of a TCR, or a chimeric antigen receptor (CAR). In some embodiments, the binding protein disclosed herein may comprise two polypeptide chains, each of which comprises a variable region comprising a CDR3 of a TCR alpha chain and a CDR3 of a TCR beta chain, or a CDR1, CDR2, and CDR3 of both a TCR alpha chain and a TCR beta chain. In some embodiments, a binding protein comprises a single chain TCR (scTCR), which comprises both the TCR V_{α} and TCR V_{β} domains, but only a single TCR constant domain (C_{α} or C_{β}). The term “chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs encompassed by the present invention may include an extracellular portion comprising an antigen-binding domain (i.e., obtained or derived from an immunoglobulin or immunoglobulin-like molecule, such as an antibody or TCR, or an antigen binding domain derived or obtained from a killer immunoreceptor from an NK cell) linked to a transmembrane domain and one or more intracellular signaling domains (optionally containing co-stimulatory domain(s)) (see, e.g., Sadelain et al. (2013) Cancer Discov. 3:388, Harris and Kranz (2016) Trends Pharmacol. Sci. 37:220, and Stone et al. (2014) Cancer Immunol. Immunother. 63:1163).

In some embodiments, 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 1, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Table 1, and/or 3) each CDR of the binding protein has up to five amino acid substitutions, insertions, deletions, or a combination thereof as compared to the cognate reference CDR sequence listed in Table 1.

In some embodiments, the binding proteins (e.g., the TCR, antigen-binding fragment of a TCR, or chimeric antigen receptor (CAR)) disclosed herein is chimeric (e.g., comprises amino acid residues or motifs from more than one donor or species), humanized (e.g., comprises residues from a non-human organism that are altered or substituted so as to reduce the risk of immunogenicity in a human), or human.

Methods for producing engineered binding proteins, such as TCRs, CARs, and antigen-binding fragments thereof, are well-known in the art (e.g., Bowerman et al. (2009) Mol. Immunol. 5:3000; U.S. Pat. Nos. 6,410,319; 7,446,191; U.S. Pat. Publ. No. 2010/065818; U.S. Pat. No. 8,822,647; PCT Publ. No. WO 2014/031687; U.S. Pat. No. 7,514,537; and Brentjens et al. (2007) Clin. Cancer Res. 73:5426).

In some embodiments, the binding protein described herein is a TCR, or antigen-binding fragment thereof, expressed on a cell surface, wherein the cell surface-expressed TCR is capable of more efficiently associating with a CD3 protein as compared to endogenous TCR A binding protein encompassed by the present invention, such as a TCR, when expressed on the surface of a cell like a T cell, may also have higher surface expression on the cell as compared to an endogenous binding protein, such as an endogenous TCR. In some embodiments, provided herein is a CAR, wherein the binding domain of the CAR comprises an antigen-specific TCR binding domain (see, e.g., Walseng et al. (2017) Scientific Reports 7: 10713).

Also provided are modified binding proteins (e.g., TCRs, antigen-binding fragments of TCRs, or CARs) that may be prepared according to well-known methods using a binding protein having one or more of the V_{α} and/or V_{β} sequences disclosed herein as starting material to engineer a modified binding protein that may have altered properties from the starting binding protein. A binding protein may be engineered by modifying one or more residues within one or both variable regions (i.e., V_{α} and/or V_{β}), for example within one or more CDR regions and/or within one or more framework regions. Additionally or alternatively, a binding protein may be engineered by modifying residues within the constant region(s).

Another type of variable region modification is to mutate amino acid residues within the V_{α} and/or V_{β} CDR1, CDR2 and/or CDR3 regions to thereby improve one or more binding properties (e.g., affinity) of the binding protein of interest. Site-directed mutagenesis or PCR-mediated mutagenesis may be performed to introduce the mutation(s) and the effect on protein binding, or other functional property of interest, may be evaluated in in vitro, ex vivo, or in vivo assays as described herein and provided in the Examples. In some embodiments, conservative modifications (as discussed above) may be introduced. The mutations may be amino acid substitutions, additions or deletions. In some embodiments, the mutations are substitutions. Moreover, typically no more than one, two, three, four or five residues within a CDR region are modified.

In some embodiments, binding proteins (e.g., TCRs, antigen-binding fragments of TCRs, or CARs) described herein may possess one or more amino acid substitutions, deletions, or additions relative to a naturally occurring TCR. In some embodiments, each CDR of the binding protein has up to five amino acid substitutions, insertions, deletions, or a combination thereof as compared to the cognate reference CDR sequence listed in Table 1. Conservative substitutions of amino acids are well-known and may occur naturally or may be introduced when the binding protein is recombinantly produced. Amino acid substitutions, deletions, and additions may be introduced into a protein using mutagenesis methods known in the art (see, e.g., Sambrook et al. (2001) Molecular Cloning: A Laboratory Manual, 3d ed., Cold Spring Harbor Laboratory Press, NY). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al. supra).

A variety of criteria known to the ordinarily skilled artisan indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In some embodiments, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS™, Align, the BLAST algorithm, or other algorithms described herein and practiced in the art).

In some embodiments, an encoded binding protein (e.g., TCR, antigen-binding fragment of a TCR, or CAR) may comprise a “signal peptide” (also known as a leader sequence, leader peptide, or transit peptide). Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. In some embodiments, a binding protein (e.g., TCR, antigen-binding fragment of a TCR, or CAR) described herein comprises a mature V domain, a mature V_{β} domain, or both. In some embodiments, a binding protein (e.g., TCR, antigen-binding fragment of a TCR, or CAR) described herein comprises a mature TCR β-chain, a mature TCR α-chain, or both.

In some embodiments, the binding proteins are fusion proteins comprising: (a) an extracellular component comprising a TCR or antigen-binding fragment thereof, (b) an intracellular component comprising an effector domain or a functional portion thereof, and (c) a transmembrane domain connecting the extracellular and intracellular components. In some embodiments, the fusion protein is capable of binding (e.g., specifically and/or selectively) to a peptide-MHC (pMHC) complex comprising an HA-1 immunogenic peptide in the context of an MHC molecule (e.g., a MHC class I molecule). In some embodiments, the MHC molecule comprises an MHC alpha chain that is an HLA serotype HLA-A*02. In some embodiments, the HLA allele is selected from the group consisting of HLA-A*0201, HLA-A*0202, HLA-A*0203, HLA-A*0205, HLA-A*0206, and HLA-A*0207 allele. In specific embodiments, the HLA allele is HLA-A*0201.

As used herein, an “effector domain” or “immune effector domain” is an intracellular portion or domain of a fusion protein or receptor that can directly or indirectly promote an immune response in a cell when receiving an appropriate signal. In some embodiments, an effector domain is from an immune cell protein or portion thereof or immune cell protein complex that receives a signal when bound (e.g., CD3ζ, or when the immune cell protein or portion thereof or immune cell protein complex binds directly to a target molecule and triggers signal transduction from the effector domain in an immune cell.

An effector domain may directly promote a cellular response when it contains one or more signaling domains or motifs, such as an intracellular tyrosine-based activation motif (ITAM), such as those found in costimulatory molecules. Without wishing to be bound by theory, it is believed that ITAMs are useful for T cell activation following ligand engagement by a T cell receptor or by a fusion protein comprising a T cell effector domain. In some embodiments, the intracellular component or functional portion thereof comprises an ITAM. Exemplary immune effector domains include but are not limited to those from, CD3ε, CD3δ, CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In some embodiments, an effector domain comprises a lymphocyte receptor signaling domain (e.g., CD3ζ or a functional portion or variant thereof).

In further embodiments, the intracellular component of the fusion protein comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that binds (e.g., specifically and/or selectively) with CD83, or a functional variant thereof, or any combination thereof. In some embodiments, the intracellular component comprises a CD28 costimulatory domain or a functional portion or variant thereof (which may optionally include a LL-GG mutation at positions 186-187 of the native CD28 protein (e.g., Nguyen et al. (2003) Blood 702:4320), a 4-1BB costimulatory domain or a functional portion or variant thereof, or both.

In some embodiments, an effector domain comprises a CD3ε endodomain or a functional (e.g., signaling) portion thereof, or a functional variant thereof. In further embodiments, an effector domain comprises a CD27 endodomain or a functional (e.g., signaling) portion thereof, or a functional variant thereof. In further embodiments, an effector domain comprises a CD28 endodomain or a functional (e.g., signaling) portion thereof, or a functional variant thereof. In still further embodiments, an effector domain comprises a 4-1BB endodomain or a functional (e.g., signaling) portion thereof, or a functional variant thereof. In further embodiments, an effector domain comprises an OX40 endodomain or a functional (e.g., signaling) portion thereof, or a functional variant thereof. In further embodiments, an effector domain comprises a CD2 endodomain or a functional (e.g., signaling) portion thereof, or a functional variant thereof. In further embodiments, an effector domain comprises a CD5 endodomain or a functional (e.g., signaling) portion thereof, or a functional variant thereof. In further embodiments, an effector domain comprises an ICAM-1 endodomain or a functional (e.g., signaling) portion thereof, or a functional variant thereof. In further embodiments, an effector domain comprises a LFA-1 endodomain or a functional (e.g., signaling) portion thereof, or a functional variant thereof. In further embodiments, an effector domain comprises an ICOS endodomain or a functional (e.g., signaling) portion thereof, or a functional variant thereof.

An extracellular component and an intracellular component encompassed by the present invention are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In some embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof).

In some embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker encompassed by the present invention can position the binding domain away from the surface of a host cell expressing the fusion protein to enable proper contact between the host cell and a target cell, antigen binding, and activation (Patel et al. (1999) Gene Therapy 6:412-419). Linker length may be varied to maximize antigen recognition based on the selected target molecule, selected binding epitope, or antigen binding domain seize and affinity (see, e.g., Guest et al. (2005) Immunother. 28:203-11 and PCT Publ. No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂, (Gly₃Ser)₂, Gly₂Ser, or a combination thereof, such as ((Gly₃Ser)₂Gly₂Ser)).

Binding proteins encompassed by the present invention may, in some embodiments, be covalently linked to a moiety. In some embodiments, the covalently linked moiety comprises an affinity tag or a label. The affinity tag may be selected from the group consisting of Glutathione-S-Transferase (GST), calmodulin binding protein (CBP), protein C tag, Myc tag, HaloTag, HA tag, Flag tag, His tag, biotin tag, and V5 tag. The label may be a fluorescent protein. In some embodiments, the covalently linked moiety is selected from the group consisting of an inflammatory agent, an anti-inflammatory agent, a cytokine, a toxin, a cytotoxic molecule, a radioactive isotope, or an antibody such as a single-chain Fv.

A binding protein may be conjugated to an agent used in imaging, research, therapeutics, theranostics, pharmaceuticals, chemotherapy, chelation therapy, targeted drug delivery, and radiotherapy. In some embodiments, a binding protein may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a binding protein. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent Non-limiting examples of fluorescent dyes that may be used as a conjugating molecule include DyLight-680, DyLight-750, VivoTag-750, DyLight-800, IRDye-800, VivoTag-680, Cy5.5, ZQ800, or indocyanine green (ICG). In some embodiments, near infrared dyes often include cyanine dyes (e.g., Cy7, Cy5.5, and Cy5). Additional, non-limiting examples of fluorescent dyes for use as a conjugating molecule in accordance with present invention include acradine orange or yellow, Alexa Fluors® (e.g., Alexa Fluor® 790, 750, 700, 680, 660, and 647) and any derivative thereof, 7-actinomycin D, 8-anilinonaphthalene-1-sulfonic acid, ATTO® dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight® Fluors® and any derivative thereof, epicocconone, ethidium bromide, FlAsH-EDT2®, Fluo dye and any derivative thereof, FluoProbe® and any derivative thereof, fluorescein and any derivative thereof, Fura® and any derivative thereof, GelGreen® and any derivative thereof, GelRed® and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green™ dyes (e.g., Oregon Green™ 488, 500, 514., etc.), Texas Red®, Texas Red®-X, SPECTRUM RED®, SPECTRUM GREEN®, cyanine dyes (e.g., CY-3, Cy-5, CY-3.5, CY-5.5, etc.), Alexa Fluor® dyes (e.g., Alexa Fluor® 350, 488, 532, 546, 568, 594, 633, 660, 680, etc.), BODIPY® dyes (e.g., BODIPY® FL, R6G, TMR, TR, 530/550, 558/568, 564/570, 576/589, 581/591, 630/650, 650/665, etc.), IRD dyes (e.g., IRD40™, IRD700™ IRD800™, etc.), and the like. Additional suitable detectable agents are well-known in the art (e.g., PCT Publ. No. PCT/US14/56177). Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Binding proteins may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the binding protein is fused with, or covalently or non-covalently linked to the agent, for example, directly or via a linker.

In some embodiments, the binding protein may be chemically modified. For example, a binding protein may be mutated to modify peptide properties such as detectability, stability, biodistribution, pharmacokinetics, half-life, surface charge, hydrophobicity, conjugation sites, pH, function, and the like. N-methylation is one example of methylation that can occur in a binding protein encompassed by the present invention. In some embodiments, a binding protein may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a binding protein with an Fc region may be a fusion Fc-protein.

A polyamino acid may include, for example, a poly amino acid sequence with repeated single amino acids (e.g., poly glycine), and a poly amino acid sequence with mixed poly amino acid sequences that may or may not follow a pattern, or any combination of the foregoing.

In some embodiments, the binding proteins encompassed by the present invention may be modified. In some embodiments, the modifications having substantial or significant sequence identity to a parent binding protein to generate a functional variant that maintains one or more biophysical and/or biological activities of the parent binding protein (e.g., maintain pMHC binding specificity). In some embodiments, the mutation is a conservative amino acid substitution.

In some embodiments, binding proteins encompassed by the present invention may comprise synthetic amino acids in place of one or more naturally-occurring amino acids. Such synthetic amino acids are well-known in the art, and include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, a-aminocyclopentane carboxylic acid, oc-aminocyclohexane carboxylic acid, a-aminocycloheptane carboxylic acid, a-(2-amino-2-norbomane)-carboxylic acid, α,γ-diaminobutyric acid, β-diaminopropionic acid, homophenylalanine, and oc-tert-butylglycine.

Binding proteins encompassed by the present invention may be glycosylated, amidated, carboxylated, phosphorylated, esterified, N-acylated, cyclized (e.g., via a disulfide bridge), or converted into an acid addition salt and/or optionally dimerized or polymerized, or conjugated.

In some embodiments, the attachment of a hydrophobic moiety, such as to the N-terminus, the C-terminus, or an internal amino acid, may be used to extend half-life of a peptide encompassed by the present invention. In other embodiments, a binding protein may include post-translational modifications (e.g., methylation and/or amidation), which can affect, for example, serum half-life. In some embodiments, simple carbon chains (e.g., by myristoylation and/or palmitylation) may be conjugated to the binding proteins. In some embodiments, the simple carbon chains may render the binding proteins easily separable from the unconjugated material. For example, methods that may be used to separate the binding proteins from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the binding proteins may be conjugated to myristic acid (tetradecanoic acid) or a derivative thereof. In other embodiments, a binding protein may be coupled (e.g., conjugated) to a half-life modifying agent Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a binding protein, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, binding proteins may be conjugated to other moieties that, for example, can modify or effect changes to the properties of the binding proteins.

A binding protein may be produced recombinantly or synthetically, such as by solid-phase peptide synthesis or solution-phase peptide synthesis. Polypeptide synthesis may be performed by known synthetic methods, such as using fluorenylmethyloxycarbonyl (Fmoc) chemistry or by butyloxycarbonyl (Boc) chemistry. Polypeptide fragments may be joined together enzymatically or synthetically.

In an aspect encompassed by the present invention, provided herein are methods of producing a binding protein described herein, comprising the steps of: (i) culturing a transformed host cell which has been transformed by a nucleic acid comprising a sequence encoding a binding protein described herein under conditions suitable to allow expression of said binding protein; and (ii) recovering the expressed binding protein.

Methods useful for isolating and purifying recombinantly produced binding protein, by way of example, may include obtaining supernatants from suitable host cell/vector systems that secrete the binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of binding proteins described herein include batch cell culture, which is monitored and controlled to maintain appropriate culture conditions. Purification of the binding protein may be performed according to methods described herein and known in the art.

In any of the herein disclosed embodiments, the encoded binding protein is capable of bind to a peptide-MHC (pMHC) complex comprising an HA-1 immunogenic peptide in the context of an MHC molecule (e.g., a MHC class I molecule). In some embodiments, the MHC molecule comprises an MHC alpha chain that is an HLA serotype HLA-A*02. In some embodiments, the HLA allele is selected from the group consisting of HLA-A*0201, HLA-A*0202, HLA-A*0203, HLA-A*0205, HLA-A*0206, and HLA-A*0207 allele.

A variety of assays are well-known for assessing binding affinity and/or determining whether a binding molecule binds (e.g., specifically and/or selectively) to a particular ligand (e.g., peptide antigen-MHC complex). It is within the level of a skilled artisan to determine the binding affinity of a binding protein for a target, such as a T cell peptide epitope of a target polypeptide, such as by using any of a number of binding assays that are well-known in the art. For example, in some embodiments, a Biacore™ machine may be used to determine the binding constant of a complex between two proteins. The dissociation constant (K_{D}) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoas says (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore™) analysis (see, e.g., Scatchard et al. (1949) Ann. N. Y. Acad. Sci. 51:660, Wilson (2002) Science 295:2103, Wolff et al. (1993) Cancer Res. 53:2560, and U.S. Pat. Nos. 5,283,173 and 5,468,614), flow cytometry, sequencing and other methods for detection of expressed nucleic acids. In one example, apparent affinity for a target is measured by assessing binding to various concentrations of tetramers, for example, by flow cytometry using labeled multimers, such as MHC-antigen tetramers. In one representative example, apparent K_{D} of a binding protein is measured using 2-fold dilutions of labeled tetramers at a range of concentrations, followed by determination of binding curves by non-linear regression, apparent K_{D} being determined as the concentration of ligand that yielded half-maximal binding.

### III. Nucleic Acids and Vectors

In an aspect encompassed by the present invention, provided herein are nucleic acid molecules that encode binding proteins (e.g., TCRs, antigen-binding fragments of the TCRs, CARs, and the like), peptides, and fragments thereof described herein.

In some embodiments, the nucleic acid molecule hybridizes, under stringent conditions, with the complement of a sequence with at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity, such as over the full length, to a nucleic acid encoding a polypeptide selected from the group consisting of the polypeptide sequences listed in Table 1.

In some embodiments, the nucleic acid molecule hybridizes, under stringent conditions, with the complement of a nucleic acid encoding a polypeptide selected from the group consisting of polypeptide sequences listed in Table 1.

In some embodiments, the nucleic acid molecule comprises (e.g., comprises, consists essentially of, or consists of) a nucleotide sequence encoding a polypeptide selected from the group consisting of polypeptide sequences listed in Table 1.

In some embodiments, the nucleic acids comprise (e.g., comprise, consist essentially of, or consist of) a nucleotide sequence encoding at least one (e.g., one, two, or three) TCR α-chain CDR set forth in Table 1. In some embodiments, the nucleic acids comprise (e.g., comprise, consist essentially of, or consist of) a nucleotide sequence encoding a TCR V_{α} domain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR V_{α} domain sequence set forth in Table 1. In some embodiments, the nucleic acids comprise (e.g., comprise, consist essentially of, or consist of) a nucleotide sequence encoding a TCR α-chain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR α-chain sequence set forth in Table 1.

In some embodiments, the nucleic acids comprise (e.g., comprise, consist essentially of, or consist of) a nucleotide sequence encoding at least one (e.g., one, two, or three) TCR β-chain CDR set forth in Table 1. In some embodiments, the nucleic acids comprise (e.g., comprise, consist essentially of, or consist of) a nucleotide sequence encoding a TCR V_{β} domain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR V_{β} domain sequence set forth in Table 1. In some embodiments, the nucleic acids comprise (e.g., comprise, consist essentially of, or consist of) a nucleotide sequence encoding a TCR β-chain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR β-chain sequence set forth in Table 1.

The term “nucleic acid” includes “polynucleotide,” “oligonucleotide,” and “nucleic acid molecule,” and generally means a polymer of DNA or RNA, which may be single-stranded or double-stranded, synthesized or obtained (e.g., isolated and/or purified) from natural sources, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. In an embodiment, the nucleic acid comprises complementary DNA (cDNA).

In some embodiments, the nucleic acids encompassed by the present invention are recombinant. As used herein, the term “recombinant” refers to (i) molecules that are constructed outside living cells by joining natural or synthetic nucleic acid segments to nucleic acid molecules that may replicate in a living cell, or (ii) molecules that result from the replication of those described in (i) above. For purposes herein, the replication may be in vitro/ex vivo replication or in vivo replication.

The nucleic acids can be constructed based on chemical synthesis and/or enzymatic ligation reactions using procedures known in the art. See, for example, Green and Sambrook et al. supra. For example, a nucleic acid may be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that may be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids encompassed by the present invention can be purchased from companies, such as Integrated DNA Technologies (Coralville, IA).

In one embodiment, the nucleic acid comprises a codon-optimized nucleotide sequence. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. In some embodiments, the nucleotide sequences described herein are codon-optimized for expression in a host cell (e.g., an immune cell, such as a T cell).

The present invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions may be rendered more stringent by the addition of increasing amounts of formamide.

The present invention also provides a nucleic acid comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, or more identical to any of the nucleic acids described herein.

Typically, said nucleic acid is a DNA or RNA molecule, which may be included in a suitable vector, such as a plasmid, cosmid, episome, artificial chromosome, phage or a viral vector.

The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. Thus, a further object encompassed by the present invention relates to a vector comprising a nucleic acid encompassed by the present invention.

Such vectors may comprise regulatory elements, such as a promoter, enhancer, terminator and the like, to cause or direct expression of said polypeptide upon administration to a subject Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell may be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, lentiviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses are well-known in the art and may be found, for instance, in PCT Publ. WO 95/14785, PCT. Publ. WO 96/22378, U.S. Pat. Nos. 5,882,877, 6,013,516, 4,861,719, 5,278,056, and PCT Publ. WO 94/19478.

In some embodiments, the composition comprises an expression vector comprising an open reading frame encoding a binding protein or a polypeptide described herein or a fragment thereof. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the binding protein, polypeptide or fragment thereof.

In some embodiments, the vector further comprises a nucleic acid sequence encoding CD8α and/or CD8β. In certain embodiments, the nucleic acid sequence encoding CD8α or CD8β is operably linked to a nucleic acid encoding a tag (e.g., a CD34 enrichment tag). In specific embodiments, the nucleic acid sequence encoding CD8α and/or CD8β are interconnected with an internal ribosome entry site or a nucleic acid sequence encoding a self-cleaving peptide, such as P2A, E2A, F2A or T2A, etc.

In some embodiments, the expression vector provided herein comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, or more identical to any of the nucleic acids set forth in SEQ ID NO. 27.

Examples of promoters include, but are not limited to, promoters from Simian Virus (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metalothionein. Examples of suitable polyadenylation signals include but are not limited to SV40 polyadenylation signals and LTR polyadenylation signals.

In addition to the regulatory elements required for expression, other elements may also be included in the nucleic acid molecule. Such additional elements include enhancers. Enhancers include the promoters described herein. In some embodiments, enhancers/promoters include, for example, human actin, human myosin, human hemoglobin, human muscle creatine and viral enhancers such as those from CMV, RSV and EBV.

In some embodiments, the nucleic acid may be operably incorporated in a carrier or delivery vector as described further below. Useful delivery vectors include but are not limited to biodegradable microcapsules, immuno-stimulating complexes (ISCOMs) or liposomes, and genetically engineered attenuated live carriers such as viruses or bacteria.

In some embodiments, the vector is a viral vector, such as lentiviruses, retroviruses, herpes viruses, adenoviruses, adeno-associated viruses, vaccinia viruses, baculoviruses, Fowl pox, AV-pox, modified vaccinia Ankara (MVA) and other recombinant viruses. For example, a lentivirus vector may be used to infect T cells.

In some embodiments, the recombinant expression vector is capable of delivering a polynucleotide to an appropriate host cell, for example, a T cell or an antigen-presenting cell, i.e., a cell that displays a peptide/MHC complex on its cell surface (e.g., a dendritic cell) and lacks CD8. In some embodiments, the host cell is a hematopoietic progenitor cell or a human immune system cell. For example, the immune system cell may be a CD4⁺ T cell, a CD8⁺ T cell, a CD4/CD8 double negative T cell, a gd T cell, a natural killer cell, a dendritic cell, or any combination thereof. In some embodiments, wherein a T cell is the host, the T cell may be naive, a central memory T cell, an effector memory T cell, or any combination thereof. The recombinant expression vectors may therefore also include, for example, lymphoid tissue-specific transcriptional regulatory elements (TREs), such as a B lymphocyte, T lymphocyte, or dendritic cell specific TREs. Lymphoid tissue specific TREs are known in the art (see, e.g., Thompson et al. (1992) Mol. Cell. Biol. 72:1043, Todd et al. (1993) J. Exp. Med. 777:1663, and Penix et al. (1993) J. Exp. Med. 775:1483).

In some embodiments, a recombinant expression vector comprises a nucleotide sequence encoding a TCR α chain, a TCR β chain, and/or a linker peptide. For example, in some embodiments, the recombinant expression vector comprises a nucleotide sequence encoding the full-length TCR alpha and TCR beta chains of the binding protein with a linker positioned between them, wherein the nucleotide sequence encoding the beta chain is positioned 5′ of the nucleotide sequence encoding the alpha chain. In some embodiments, the nucleotide sequence encodes the full-length TCR alpha and TCR beta chains with a linker positioned between them, wherein the nucleotide sequence encoding the TCR beta chain is positioned 3′ of the nucleotide sequence encoding the TCR alpha chain. In some embodiments, the full-length TCR alpha and/or TCR beta chains are replaced with fragments thereof.

As described further below, another aspect encompassed by the present invention relates to a cell which has been transfected, infected or transformed by a nucleic acid and/or a vector in accordance with the present invention. A host cell may include any individual cell or cell culture which may receive a vector or the incorporation of nucleic acids and/or proteins, as well as any progeny cells. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods (see, e.g., Sambrook et al., (1989) Molecular Cloning: A Laboratory Manual 2d ed. (Cold Spring Harbor Laboratory)). The term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids encompassed by the present invention may be used to produce a recombinant polypeptide encompassed by the present invention in a suitable expression system. The term “expression system” means a host cell and compatible vector under suitable conditions, e.g., for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al (1980), rat YB2/3HL.P2.G11.16Ag.20 cell (ATCC CRL 1662, hereinafter referred to as “YB2/0 cell”), and the like. In some embodiments, the YB2/0 cell is used since ADCC activity of chimeric or humanized binding proteins is enhanced when expressed in this cell.

The present invention also encompasses methods of producing a recombinant host cell expressing binding proteins, peptides and fragments thereof encompassed by the present invention, said method comprising the steps consisting of (i) introducing in vitro or ex vivo a recombinant nucleic acid or a vector as described above into a competent host cell, (ii) culturing in vitro or ex vivo the recombinant host cell obtained and (iii), optionally, selecting the cells which express said binding proteins, peptides and fragments thereof. Such recombinant host cells may be used for the diagnostic, prognostic, and/or therapeutic method encompassed by the present invention.

In another aspect, the present invention provides isolated nucleic acids that hybridize under selective hybridization conditions to a polynucleotide disclosed herein. Thus, the polynucleotides of this embodiment may be used for isolating, detecting, and/or quantifying nucleic acids comprising such polynucleotides. For example, polynucleotides encompassed by the present invention may be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. In some embodiments, the cDNA library comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, full-length sequences. The cDNA libraries may be normalized to increase the representation of rare sequences. Low or moderate stringency hybridization conditions are typically, but not exclusively, employed with sequences having a reduced sequence identity relative to complementary sequences. Moderate and high stringency conditions may optionally be employed for sequences of greater identity. Low stringency conditions allow selective hybridization of sequences having about 70% sequence identity and may be employed to identify orthologous or paralogous sequences. Optionally, polynucleotides encompassed by the present invention will encode at least a portion of a binding protein encoded by the polynucleotides described herein. The polynucleotides encompassed by the present invention embrace nucleic acid sequences that may be employed for selective hybridization to a polynucleotide encoding a binding protein encompassed by the present invention (see, e.g., Ausubel, supra and Colligan, supra).

### IV. Host Cells

In an aspect encompassed by the present invention, provided herein are host cells that express the binding proteins (e.g., TCRs, antigen-binding fragments of TCRs, CARs, or fusion proteins comprising a TCR and an effector domain) described herein. In some embodiments, the host cells comprise the nucleic acids or vectors described herein.

In some embodiments, a polynucleotide encoding a binding protein is used to transform, transfect, or transduce a host cell (e.g., a T cell) for use in adoptive transfer therapy. Advances in nucleic acid sequencing and particular TCR sequencing have been described (e.g., Robins et al. (2009) Blood 114:4099; Robins et al. (2010) Sci. Translat. Med. 2:47ra64, Robins et al. (2011) J. Imm. Meth., and Warren et al. (2011) Genome Res. 21:790) and may be employed in the course of practicing embodiments encompassed by the present invention. Similarly, methods for transfecting or transducing T cells with desired nucleic acids are well-known in the art (e.g., U.S. Pat. Publ. No. US 2004/0087025) as have adoptive transfer procedures using T cells of desired antigen-specificity (e.g., Schmitt et al. (2009) Hum. Gen. 20:1240, Dossett et al. (2009) Mol. Ther. 77:742, Till et al. (2008) Blood 772:2261, Wang et al. (2007) Hum. Gene Ther. 18:112, Kuball et al. (2007) Blood 709:2331, U.S. Pat. Publ. 2011/0243972, U.S. Pat. Publ. 2011/0189141, and Leen et al. (2007) Ann. Rev. Immunol. 25:243).

Any suitable immune cell may be modified to include a heterologous polynucleotide encompassed by the present invention, including, for example, a T cell, a NK cell, or a NK-T cell. In some embodiments, the cell may be a primary cell or a cell of a cell line. In some embodiments, a modified immune cell comprises a CD4⁺ T cell, a CD8⁺ T cell, or both. For purposes herein, the T cell may be any T cell, such as a cultured T cell, e.g., a primary T cell, or a T cell from a cultured T cell line, e.g., Jurkat, SupTl, etc., or a T cell obtained from a mammal. If obtained from a mammal, the T cell may be obtained from numerous sources, including but not limited to blood, bone marrow, lymph node, the thymus, or other tissues or fluids. T cells may also be enriched for or purified. In some embodiments, the T cell is a human T cell. In some embodiments, the T cell is a T cell isolated from a human. The T cell may be any type of T cell and may be of any developmental stage, including but not limited to, cytotoxic lymphocyte, cytotoxic lymphocyte precursor cell, cytotoxic lymphocyte progenitor cell, cytotoxic lymphocyte stem cell, CD4⁺/CD8⁺ double positive T cells, CD4⁺ helper T cells, e.g., Th1 and Th2 cells, CD4⁺ T cells, CD8⁺ T cells (e.g., cytotoxic T cells), tumor infiltrating lymphocytes (TILs), memory T cells (e.g., central memory T cells and effector memory T cells), naive T cells, and the like.

Any appropriate method may be used to transfect or transduce the cells, for example, T cells, or to administer the nucleotide sequences or compositions encompassed by methods described herein. Methods for delivering polynucleotides to host cells include, for example, use of cationic polymers, lipid-like molecules, and certain commercial products such as, for example, in vivo-jetPEI®. Other methods include ex vivo transduction, injection, electroporation, DEAE-dextran, sonication loading, liposome-mediated transfection, receptor-mediated transduction, microprojectile bombardment, transposon-mediated transfer, and the like. Still further methods of transfecting or transducing host cells employ vectors, described in further detail herein.

Modified immune cells as described herein may be functionally characterized using methodologies for assaying T cell activity, including determination of T cell binding, activation or induction and also including determination of T cell responses that are antigen-specific. Examples include determination of T cell proliferation, T cell cytokine release, antigen-specific T cell stimulation, MHC restricted T cell stimulation, CTL activity (e.g., by detecting ⁵¹Cr release from pre-loaded target cells), changes in T cell phenotypic marker expression, and other measures of T-cell functions.

Procedures for performing these and similar assays may be found, for example, in Lefkovits (Immunology Methods Manual: Hie Comprehensive Sourcebook of Techniques, 1998), as well as Current Protocols in Immunology, Weir, (1986) Handbook of Experimental Immunology, Blackwell Scientific, Boston, MA; Mishell and Shigii (eds.) (1979) Selected Methods in Cellular Immunology, Freeman Publishing, San Francisco, CA; Green and Reed (1998) Science 281:1309, and references cited therein.

In some embodiments, apparent affinity for a binding protein, such as a TCR or antigen-binding portion thereof, may be measured by assessing binding to various concentrations of MHC multimers. “MHC-peptide multimer staining” refers to an assay used to detect antigen-specific T cells, which, in some embodiments, features a tetramer of MHC molecules, each comprising an identical peptide having an amino acid sequence that is cognate (e.g., identical or related to) at least one antigen (e.g., an HA-1 immunogenic peptide), wherein the complex is capable of binding to a binding protein, such as a TCR or antigen-binding portion thereof, that recognizes the cognate antigen. Each of the MHC molecules may be tagged with a biotin molecule. Biotinylated MHC/peptides may be multimerized (e.g., tetramerized) by the addition of streptavidin, which may be fluorescently labeled.

The multimer may be detected by flow cytometry via the fluorescent label. In some embodiments, a pMHC multimer assay is used to detect or select enhanced affinity binding protein, such as a TCR or antigen-binding portion thereof, encompassed by the present invention. In some examples, apparent K_{D} of a binding protein, such as a TCR or antigen-binding portion thereof, is measured using 2-fold dilutions of labeled multimers at a range of concentrations, followed by determination of binding curves by non-linear regression, apparent K_{D} being determined as the concentration of ligand that yielded half-maximal binding.

Levels of cytokines may be determined using methods described herein, such as ELISA, ELISPOT, intracellular cytokine staining, and flow cytometry and combinations thereof (e.g., intracellular cytokine staining and flow cytometry).

Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-g, IL-12, IL-2, and TNF-b, and Type 2 cytokines, such as IL-4, IL-5, IL-9, IL-10, and IL-13.

A host cell encompassed by the present invention may comprise a single polynucleotide that encodes a binding protein as described herein, or the binding protein may be encoded by more than one polynucleotide. In other words, components or portions of a binding protein may be encoded by two or more polynucleotides, which may be contained on a single nucleic acid molecule or may be contained on two or more nucleic acid molecules.

In some embodiments, a polynucleotide encoding two or more components or portions of a binding protein encompassed by the present invention comprises the two or more coding sequences operatively associated in a single open reading frame. Such an arrangement can advantageously allow coordinated expression of desired gene products, such as, for example, contemporaneous expression of alpha- and beta-chains of a TCR, such that they are produced in about a 1:1 ratio. In some embodiments, two or more substituent gene products of a binding protein encompassed by the present invention, such as a TCR (e.g., alpha- and beta-chains) or CAR, are expressed as separate molecules and associate post-translationally. In further embodiments, two or more substituent gene products of a binding protein encompassed by the present invention are expressed as a single peptide with the parts separated by a cleavable or removable segment. For instance, self-cleaving peptides useful for expression of separable polypeptides encoded by a single polynucleotide or vector are known in the art and include, for example, a porcine teschovirus-1 2 A (P2A) peptide, a thoseaasigna virus 2A (T2A) peptide, an equine rhinitis A virus (ERAV) 2A (E2A) peptide, and a foot-and-mouth disease vims 2A (F2A) peptide.

In some embodiments, a binding protein encompassed by the present invention comprises one or more junction amino acids. “Junction amino acids” or “junction amino acid residues” refer to one or more (e.g., 2 to about 10) amino acid residues between two adjacent motifs, regions or domains of a polypeptide, such as between a binding domain and an adjacent constant domain or between a TCR chain and an adjacent self-cleaving peptide. Junction amino acids can result from the design of a construct that encodes a fusion protein (e.g., amino acid residues resulting from the use of a restriction enzyme site during the construction of a nucleic acid molecule encoding a fusion protein), or from cleavage of, for example, a self-cleaving peptide adjacent one or more domains of an encoded binding protein encompassed by the present invention (e.g., a P2A peptide disposed between a TCR α-chain and a TCR β-chain, the self-cleavage of which can leave one or more junction amino acids in the α-chain, the TCR β-chain, or both).

Engineered immune cells encompassed by the present invention may be administered as therapies for, e.g., a non-malignant disorder, a hyperproliferative disorder, or a relapse of a hyperproliferative disorder characterized by expression of an HA-1 antigen. In some circumstances, it may be desirable to reduce or stop the activity associated with a cellular immunotherapy. Thus, in some embodiments, an engineered immune cell encompassed by the present invention comprises a heterologous polynucleotide encoding a binding protein and an accessory protein, such as a safety switch protein, which can be targeted using a cognate drug or other compound to selectively modulate the activity (e.g., lessen or ablate) of such cells when desirable. Safety switch proteins used in this regard include, for example, a truncated EGF receptor polypeptide (huEGFRt) that is devoid of extracellular N-terminal ligand binding domains and intracellular receptor tyrosine kinase activity but retains the native amino acid sequence, type I transmembrane cell surface localization, and a conformationally intact binding epitope for pharmaceutical-grade anti-EGFR monoclonal antibody, cetuximab (Erbitux) tEGF receptor (tEGFr; Wang et al. (2011) Blood 118:1255-1263), a caspase polypeptide (e.g., iCasp9; Straathof et al. (2005) Blood 105:4247-4254, Di Stasi et al. (2011) N. Engl. J. Med. 365:1673-1683, Zhou and Brenner (2016) Hematol. pii: S0301-472X: 30513-30516), RQR8 (Philip et al. (2014) Blood 124:1277-1287), and a human c-myc protein tag (Kieback et al. (2008) Proc. Natl. Acad. Sci. USA 105:623-628) Other accessory components useful for therapeutic cells comprise a tag or selection marker (e.g., a CD34 enrichment tag) that allows the cells to be identified, sorted, isolated, enriched, or tracked. For example, marked immune cells having desired characteristics (e.g., an antigen-specific TCR and a safety switch protein) may be sorted away from unmarked cells in a sample and more efficiently activated and expanded for inclusion in a therapeutic product of desired purity.

As used herein, the term “selection marker” comprises a nucleic acid construct that confers an identifiable change to a cell permitting detection and positive selection of immune cells transduced with a polynucleotide comprising a selection marker. For example, RQR is a selection marker that comprises a major extracellular loop of CD20 and two minimal CD34 binding sites. In some embodiments, an RQR-encoding polynucleotide comprises a polynucleotide that encodes the 16 amino acid CD34 minimal epitope. In some embodiments, such as certain embodiments provided in the examples herein, the CD34 minimal epitope is incorporated at the amino terminal position of the CD8 stalk domain (Q8). In further embodiments, the CD34 minimal binding site sequence may be combined with a target epitope for CD20 to form a compact marker/suicide gene for T cells (RQR8) (Philip et al. 2014). This construct allows for the selection of immune cells expressing the construct, with for example, CD34-specific antibody bound to magnetic beads (Miltenyi) and that utilizes clinically accepted pharmaceutical antibody, rituximab, that allows for the selective deletion of a transgene expressing engineered T cell (e.g., Philip et al. (2014) Blood 124:1277-1287, U.S. Pat. Publ. 2015-0093401, and U.S. Pat. Publ. 2018-0051089).

Further exemplary selection markers include several truncated type I transmembrane proteins normally not expressed on T cells: the truncated low-affinity nerve growth factor, truncated CD19, and truncated CD34 (e.g., Di Stasi et al. (2011) N. Engl. J. Med. 365:1673-1683, Mavilio et al. (1994) Blood 83:1988-1997, and Fehse et al. (2000) Mol. Ther. 7:448-456). A particularly attractive feature of CD19 and CD34 is the availability of the off-the-shelf Miltenyi CliniMACs™ selection system that can target these markers for clinical-grade sorting. However, CD19 and CD34 are relatively large surface proteins that may tax the vector packaging capacity and transcriptional efficiency of an integrating vector. Surface markers containing the extracellular, non-signaling domains or various proteins (e.g., CD19, CD34, LNGFR, etc.) also may be employed. Any selection marker may be employed and should be acceptable for good manufacturing practices. In some embodiments, selection markers are expressed with a polynucleotide that encodes a gene product of interest (e.g., a binding protein encompassed by the present invention, such as a TCR or CAR, or antigen-binding fragment thereof). Further examples of selection markers include, for example, reporters such as GFP, EGFP, β-gal or chloramphenicol acetyltransferase (CAT). In some embodiments, a selection marker, such as, for example, CD34 is expressed by a cell and the CD34 may be used to select enrich for, or isolate (e.g., by immunomagnetic selection) the transduced cells of interest for use in the methods described herein. As used herein, a CD34 marker is distinguished from an anti-CD34 antibody, or, for example, a scFv, TCR, or other antigen recognition moiety that binds to CD34.

In some embodiments, a selection marker comprises an RQR polypeptide, a truncated low-affinity nerve growth factor (tNGFR), a truncated CD19 (tCD19), a truncated CD34 (tCD34), or any combination thereof.

By way of background, inclusion of CD4⁺ T cells in an immunotherapy cell product can provide antigen-induced IL-2 secretion and augment persistence and function of transferred cytotoxic CD8⁺ T cells (e.g., Kennedy et al. (2008) Immunol. Rev. 222:129 and Nakanishi et al. Nature (2009) 52:510). In some embodiments, a class I-restricted TCR in CD4⁺ T cells may require the transfer of a CD8 co-receptor to enhance sensitivity of the TCR to class I HLA peptide complexes. CD4 co-receptors differ in structure to CD8 and cannot effectively substitute for CD8 co-receptors (e.g., Stone & Kranz (2013) Front. Immunol. 4:244 and Cole et al. (2012) Immunology 737:139). Thus, another accessory protein for use in the compositions and methods encompassed by the present invention comprises a CD8 co-receptor or component thereof. Engineered immune cells comprising a heterologous polynucleotide encoding a binding protein encompassed by the present invention may, in some embodiments, further comprise a heterologous polynucleotide encoding a CD8 co-receptor protein, or a beta-chain or alpha-chain component thereof.

A host cell may be efficiently transduced to contain, and may efficiently express, a single polynucleotide that encodes the binding protein, safety switch protein, selection marker, and CD8 co-receptor protein.

In one embodiment, the host cell encompassed by the present invention further includes a nucleic acid encoding a co-stimulatory molecule, such that the modified T cell expresses the co-stimulatory molecule. In some embodiments, the co-stimulatory domain is selected from CD3, CD27, CD28, CD83, CD86, CD127, 4-1BB, 4-1BBL, PD1 and PD1L.

In any of the foregoing embodiments, a host cell that express the binding protein described herein may be a universal immune cell. A “universal immune cell” comprises an immune cell that has been modified to reduce or eliminate expression of one or more endogenous genes that encode a polypeptide product selected from PD-1, LAG-3, CTLA4, TIM3, TIGIT, an HLA molecule, a TCR molecule, or any combination thereof. Without wishing to be bound by theory, certain endogenously expressed immune cell proteins may downregulate the immune activity of the modified immune cells (e.g., PD-1, LAG-3, CTLA4, TIGIT), or may interfere with the binding activity of a heterologously expressed binding protein encompassed by the present invention (e.g., an endogenous TCR that binds a non-HA-1 antigen and interferes with the modified immune cell binding to a target cell that expresses an HA-1 antigen such as an HA-1 immunogenic peptide comprising the amino acid sequence VLHDDLLEA in the context of a MHC molecule. Further, endogenous proteins (e.g., immune cell proteins, such as an HLA allele) expressed on a donor immune cell may be recognized as foreign by an allogeneic host, which may result in elimination or suppression of the modified donor immune cell by the allogeneic host.

Accordingly, decreasing or eliminating expression or activity of such endogenous genes or proteins can improve the activity, tolerance, or persistence of the modified immune cells in an autologous or allogeneic host setting, and allows universal administration of the cells (e.g., to any recipient regardless of HLA type). In some embodiments, cells in accordance with the present invention are syngeneic, meaning that they are genetically identical or sufficiently identical and immunologically compatible as to allow for transplantation. In some embodiments, a universal immune cell is a donor cell (e.g., allogeneic) or an autologous cell. In some embodiments, a modified immune cell (e.g., a universal immune cell) encompassed by the present invention comprises a chromosomal gene knockout of one or more of a gene that encodes PD-1, LAG-3, CTLA4, TIM3, TIGIT, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai et al., (2016) Nature Sci. Rep. 6: 21757; Torikai et al. (2012) Blood 179: 5697; and Torikai et al. (2013) Blood 722: 1341, which also provide representative, exemplary gene editing techniques, compositions, and adoptive cell therapies useful according to the present invention).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product Alterations resulting in a chromosomal gene knockout may include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In some embodiments, a chromosomal gene knock-out or gene knock-in may be made by chromosomal editing of a host cell. Chromosomal editing may be performed using, for example, endonucleases. As used herein “endonuclease” refers to an enzyme capable of catalyzing cleavage of a phosphodiester bond within a polynucleotide chain. In some embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues may be changed to alter triplet sequence specificity (e.g., Desjarlais et al. (1993) Proc. Natl. Acad. Sci. 90:2256-2260 and Wolfe et al. (1999) J. Mol. Biol. 255:1917-1934). Multiple zinc finger motifs may be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In some embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a Fokl endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the repeat variable diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also well-known in the art (e.g., U.S. Pat. Publ. No. US 2011/0301073, which atypical RVDs are incorporated by reference herein in their entirety). TALENs may be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In some embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences may be introduced at the site of DSB via homology directed repair. The crRNA and tracrRNA may be engineered into a single guide RNA (sgRNA or gRNA) (e.g., Jinek et al. (2012) Science 337:816-821). Further, the region of the guide RNA complementary to the target site may be altered or programed to target a desired sequence (Xie et al. (2014) PLOS One 9: e100448, U.S. Pat Publ. No. US 2014/0068797, U.S. Pat Publ. No. US 2014/0186843, U.S. Pat. No. 8,697,359, and PCT Publ. No. WO 2015/071474). In some embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al. (2017) Clin. Cancer Res. 23:2255-2266, which provides representative, exemplary gRNAs, CAS9 DNAs, vectors, and gene knockout techniques.

As used herein, a “meganuclease,” also referred to as a “homing endonuclease,” refers to an endodeoxyribonuclease characterized by a large recognition site (double stranded DNA sequences of about 12 to about 40 base pairs). Meganucleases may be divided into five families based on sequence and structure motifs: LAGLIDADG, GlY-YIG, HNH, His-Cys box, and PD-(D/E)XK. Exemplary meganucleases include I-Scel, I-Ceu1, PI-PspI, RI-Sce, I-ScelV, I-Csml, I-Panl, I-Scell, I-Ppol, I-SceIII, I-Crel, I-Tevl, I-TevII and I-TevIII, whose recognition sequences are well-known (e.g., U.S. Pat. Nos. 5,420,032 and 6,833,252, Belfort et al. (1997) Nucl. Acids Res. 25:3379-3388, Dujon et al. (1989) Gene 52:115-118, Perler et al. (1994) Nucl. Acids Res. 22:1125-1127, Jasin (1996) Trends Genet. 72:224-228, Gimble et al. (1996) J. Mol. Biol. 263:163-180, and Argast et al. (1998) J Mol. Biol. 280: 345-353).

In some embodiments, naturally-occurring meganucleases may be used to promote site-specific genome modification of a target of interest, such as an immune checkpoint, an HLA-encoding gene, or a TCR component-encoding gene.

In other embodiments, an engineered meganuclease having a novel binding specificity for a target gene is used for site-specific genome modification (see, e.g., Porteus et al. (2005) Nat. Biotechnol. 23:967-73, Sussman et al. (2004) J. Mol. Biol. 342:31-41, Epinat et al. (2003) Nucl. Acids Res. 37:2952-2962, Chevalier et al. (2002) Mol. Cell 70:895-905, Ashworth et al. (2006) Nature 441:656-659, Paques et al. (2007) Curr. Gene Ther. 7:49-66, and U.S. Pat. Publ. Nos. US 2007/0117128, US 2006/0206949, US 2006/0153826, US 2006/0078552, and US 2004/0002092). In further embodiments, a chromosomal gene knockout is generated using a homing endonuclease that has been modified with modular DNA binding domains of TALENs to make a fusion protein known as a megaTAL. MegaTALs may be utilized to not only knock-out one or more target genes, but to also introduce (knock in) heterologous or exogenous polynucleotides when used in combination with an exogenous donor template encoding a polypeptide of interest.

In some embodiments, a chromosomal gene knockout comprises an inhibitory nucleic acid molecule that is introduced into a host cell (e.g., an immune cell) comprising a heterologous polynucleotide encoding an antigen-specific receptor that binds (e.g., specifically and/or selectively) to an HA-1 antigen, wherein the inhibitory nucleic acid molecule encodes a target-specific inhibitor and wherein the encoded target-specific inhibitor inhibits endogenous gene expression (i.e., of an immune checkpoint, an HLA component, or a TCR component, or any combination thereof) in the host immune cell.

A chromosomal gene knockout may be confirmed directly by DNA sequencing of the host immune cell following use of the knockout procedure or agent.

Chromosomal gene knockouts may also be inferred from the absence of gene expression (e.g., the absence of an mRNA or polypeptide product encoded by the gene) following the knockout.

In some embodiments, a host cell encompassed by the present invention is capable of specifically and/or selectively 50% or more of target cells that comprise a peptide-MHC (pMHC) complex comprising an HA-1 immunogenic peptide in the context of an MHC molecule.

In some embodiments, the modified immune cell is capable of producing a cytokine when contacted with target cells that comprise a peptide-MHC (pMHC) complex comprising an HA-1 immunogenic peptide in the context of an MHC molecule.

In some embodiments, the cytokine comprises IFN-γ or IL2. In some embodiments, the cytokine comprises TNF-α.

In some embodiments, the host cell is capable of producing a higher level of cytokine or a cytotoxic molecule when contacted with a target cell with expression of HA-1 at a level of less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 35 TPM). In some embodiments, the low HA-1 expression level is termed “heterozygous expression” meaning between about 1 TPM and about 35 TPM, or any range in between, inclusive, such as 1-32 TPM. For example, the host cell is capable of producing an at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, higher level of cytokine or a cytotoxic molecule.

In some embodiments, the host cell is capable of specifically and/or selectively killing a target cell expressing HA-1 (e.g., a hyperproliferative cell expressing HA-1). In certain embodiments, the target cell expresses: (i) a polypeptide comprising or consisting of an amino acid sequence VLHDDLLEA; and (ii) a matched MHC molecule.

In some embodiments, host cells do not express HA-1 antigen, are not recognized by a binding protein of any one of claims 1-30, are not of serotype HLA-A*02, and/or do not express an HLA-A*02 allele, such as HLA-A*02:01, HLA-A*02:02, HLA-A*02:03, HLA-A*02:05, HLA-A*02:06, or HLA-A*02:07 allele. For example, a patient may receive host cells from a healthy donor who is HA-1-negative or HLA-A*02:01-negative. Stem cells, such as hematopoietic stem cells, isolated from that donor (or engineered autologous cells) may be used as the source of transplant material. In parallel, T cells isolated from the same donor may be genetically engineered to recognize HA-1, such as by expressing an HA-1 binding protein described herein. Donor stem cells may be used to engraft cell populations, such as a reconstituted immune system, into the patient and host cells may be infused into the patient with the goal of eliciting a highly specific anti-tumor effect. The engineered donor T cells may be designed to recognize and eliminate HA-1-expressing cells, such as all of the patient's native blood cells, including, for example, cancer cells like residual leukemia cells, which are HA-1-positive, thereby preventing relapse and promoting complete cures. Because the patient's new healthy blood cells are derived from the donor and are therefore either HA-1-negative, HLA-A*02 serotype negative, and/or or HLA-A*02:01-negative, engineered cells described herein may have have minimal toxic side effects. Such patient-matched host cells and treatment methods may be used according to therapeutic methods described further below.

In some embodiments, the killing is determined by a killing assay. In some embodiment, the killing assay is carrier out by coculturing the host cell and the target cell at a ratio from 20:1 to 0.625:1, for example, from 15:1 to 1.25:1, from 10:1 to 1.5:1, from 8:1 to 3:1, from 6:1 to 5:1, 20:1 to 5:1, 10:1 to 2.5:1 etc. In some embodiments, the target cell is pulsed with 1 μg/mL to 50 μg/mL of HA-1 peptide, for example, from 1 ug/mL to 10 ng/mL, 500 ng/mL to 0.5 ng/mL, from 10 ng/mL to 10 pg/mL from 250 ng/mL to 1 ng/mL, from 50 ng/mL to 5 ng/mL, from 20 ng/mL to 10 ng/mL, etc.

In some embodiments, the host cell is capable of killing a higher number of target cells when contacted with target cells with a level of HA-1 less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 35 TPM). In some embodiments, the low HA-1 expression level is termed “heterozygous expression” meaning between about 1 TPM and about 35 TPM, or any range in between, inclusive, such as 1-32 TPM. For example, the host cell may be capable of killing an at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, higher number of target cells.

The present invention further provides a population of cells comprising at least one host cell described herein. The population of cells may be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells may be a substantially homogeneous population, in which the population comprises mainly of host cells (e.g., consisting essentially of) comprising the recombinant expression vector. The population also may be a clonal population of cells, in which all cells of the population are clones of a single host cell comprising a recombinant expression vector, such that all cells of the population comprise the recombinant expression vector. In one embodiment encompassed by the present invention, the population of cells is a clonal population comprising host cells comprising a recombinant expression vector as described herein.

In an embodiment encompassed by the present invention, the numbers of cells in the population may be rapidly expanded. Expansion of the numbers of T cells may be accomplished by any of a number of methods as are well-known in the art (e.g., U.S. Pat. Nos. 8,034,334 and 8,383,099, U.S. Pat. Publ. No. 2012/0244133, Dudley et al. (2003) J. Immunother. 26:332-242, and Riddell et al. (1990) J. Immunol. Methods 128:189-201). For example, expansion of the numbers of T cells may be carried out by culturing the T cells with OKT3 antibody, IL-2, and feeder PBMC (e.g., irradiated allogeneic PBMC).

### V. Pharmaceutical Compositions

In another aspect encompassed by the present invention, pharmaceutical compositions are provided herein comprising compositions described herein (e.g., binding proteins, nucleic acids, cells, and the like) and a pharmaceutically acceptable carrier, diluent, or excipient.

The term “pharmaceutically acceptable” refers to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Agents and other compositions encompassed by the present invention may be specially formulated for administration in solid or liquid form, including those adapted for various routes of administration, such as (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. Any appropriate form factor for an agent or composition described herein, such as, but not limited to, tablets, capsules, liquid syrups, soft gels, suppositories, and enemas, is contemplated.

Pharmaceutical compositions encompassed by the present invention may be presented as discrete dosage forms, such as capsules, sachets, or tablets, or liquids or aerosol sprays each containing a pre-determined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms may be prepared by any of the methods of pharmacy.

Suitable excipients include water, saline, dextrose, glycerol, or the like and combinations thereof. In some embodiments, compositions comprising host cells, binding proteins, or fusion proteins as disclosed herein further comprise a suitable infusion media. Suitable infusion media may be any isotonic medium formulation, typically normal saline, Normosol™-R (Abbott) or Plasma-Lyte™ A (Baxter), 5% dextrose in water, Ringer's lactate may be utilized. An infusion medium may be supplemented with human serum albumin or other human serum components. Unit doses comprising an effective amount of a host cell, or composition are also contemplated.

Also provided herein are unit doses that comprise an effective amount of a host cell or of a composition comprising the host cell. As described herein, host cells include immune cells, T cells (CD4⁺ T cells and/or CD8⁺ T cells), cytotoxic lymphocytes (e.g., cytotoxic T cells and/or natural killer (NK) cells), and the like. For example, in some embodiments, a unit dose comprises a composition comprising at least about 30%, at least about 40%, at least about 50%, at least about 60%), at least about 70%, at least about 80%, at least about 85%, at least about 90%, or at least about 95% engineered cells, either alone or in combination with other cells, such as comprising at least about 30%, at least about 40%, at least about 50%, at least about 60%), at least about 70%, at least about 80%, at least about 85%, at least about 90%, or at least about 95% other cells. In some embodiments, undesired cells are present at a reduced amount or substantially not present, such as less than about 50%, less than about 40%, less than about 30%, less than about 20%, less than about 10%, less than about 5%, or less then about 1% the population of cells in the composition.

The amount of cells in a composition or unit dose is at least one cell (for example, at least one engineered CD8⁺ T cell, engineered CD4⁺ T cell, and/or NK cell) or is more typically greater than 10² cells, for example, up to 10⁶, up to 10⁷, up to 10⁸ cells, up to 10⁹ cells, or more than 10¹⁰ cells. In some embodiments, the cells are administered in a range from about 10⁶ to about 10¹⁰ cells/m², such as in a range of about 10⁵ to about 10⁹ cells/m². The number of cells will depend upon the ultimate use for which the composition is intended as well the type of cells included therein. For example, cells modified to contain a binding protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 ml or less, 250 ml or less, or 100 ml or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells may be apportioned into multiple infusions that cumulatively equal or exceed 10⁶, 10⁷, 10⁸, 10⁹, 10¹⁰, or 10¹¹ cells. In some embodiments, a unit dose of the engineered immune cells may be co-administered with (e.g., simultaneously or contemporaneously) hematopoietic stem cells from an allogeneic donor.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutically effective amount” may be used in reference to treatment, whereas “prophylactically effective amount” may be used to describe administrating an effective amount to a subject that is susceptible or at risk of developing a disease or disease-state (e.g., recurrence) as a preventative course.

The pharmaceutical compositions described herein may be presented in unit-dose or multi-dose containers, such as sealed ampoules or vials. Such containers may be frozen to preserve the stability of the formulation until infusion into the patient. In some embodiments, a unit dose comprises a host cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of engineered immune cells or active compound calculated to produce the desired effect in association with an appropriate pharmaceutical carrier.

In some embodiments, the pharmaceutical composition described, when administered to a subject, can elicit an immune response against a cell of interest that expresses HA-1. Such pharmaceutical compositions may be useful as vaccines for prophylactic and/or therapeutic treatment of a non-malignant disorder, a hyperproliferative disorder, or a relapse of a hyperproliferative disorder characterized by expression of an HA-1 antigen.

In some embodiments, the pharmaceutical composition further comprises a physiologically acceptable adjuvant. In some embodiments, the adjuvant employed provides for increased immunogenicity of the pharmaceutical composition. Such a further immune response stimulating compound or adjuvant may be (i) admixed to the pharmaceutical composition in accordance with the present invention after reconstitution of the peptides and optional emulsification with an oil-based adjuvant as defined above, (ii) may be part of the reconstitution composition encompassed by the present invention defined above, (iii) may be physically linked to the peptide(s) to be reconstituted or (iv) may be administered separately to the subject, mammal or human, to be treated. The adjuvant may be one that provides for slow release of antigen (e.g., the adjuvant may be a liposome), or it may be an adjuvant that is immunogenic in its own right thereby functioning synergistically with antigens. For example, the adjuvant may be a known adjuvant or other substance that promotes antigen uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, bacto-adjuvant, synthetic polymers such as poly amino acids and co-polymers of amino acids, saponin, paraffin oil, and muramyl dipeptide. In some embodiments, the adjuvant is adjuvant 65, α-GalCer, aluminum phosphate, aluminum hydroxide, calcium phosphate, β-glucan peptide, CpG DNA, GM-CSF, GPI-0100, IFA, IFN-γ, IL-17, lipid A, lipopolysaccharide, Lipovant, Montanide™, N-acetyl-muramyl-L-alanyl-D-isoglutamine, pam3CSK4, quil A, trehalose dimycolate, or zymosan.

In some embodiments, the adjuvant is an immunomodulatory molecule. For example, the immunomodulatory molecule may be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12, IL-17 and IL-20), tumor necrosis factors (e.g., TNFα and TNFβ), erythropoietin (EPO), FLT-3 ligand, gIp10, TCA-3, MCP-1, MIF, MIP-L.alpha, MIP-10, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In some embodiments, an immunomodulatory chemokine that binds to a chemokine receptor, i.e., a CXC, CC, C, or CX3C chemokine receptor, also may be included in the compositions provided here. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, 1309, IL-8, Gcp-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

In some embodiments, the composition comprises a binding protein (e.g., a TCR, an antigen-binding fragment of a TCR, a CAR, or a fusion protein comprising a TCR and an effector domain), a TCRα and/or TCRβ polypeptide described herein. In some embodiments, the composition comprises a nucleic acid encoding a binding protein, a TCRα and/or TCRβ polypeptide described herein, such as a DNA molecule encoding a binding protein, a TCRα and/or TCRβ polypeptide. In some embodiments, the composition comprises an expression vector comprising an open reading frame encoding a binding protein, a TCRα and/or TCRβ polypeptide.

When taken up by a cell (e.g., T cells, NK cells, etc.), a DNA molecule may be present in the cell as an extrachromosomal molecule and/or may integrate into the chromosome. DNA may be introduced into cells in the form of a plasmid which may remain as separate genetic material. Alternatively, linear DNAs that may integrate into the chromosome may be introduced into the cell. Optionally, when introducing DNA into a cell, reagents which promote DNA integration into chromosomes may be added.

### VI. Uses and Methods

The compositions described herein may be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

In some uses and methods encompassed by the present invention, subjects or subject samples are utilized. In some embodiments, the subject is an animal. The animal may be of either sex and may be at any stage of development. In some embodiments, the animals is a vertebrate, such as a mammal. In some embodiments, the subject is a non-human mammal. In some embodiments, the subject is a domesticated animal, such as a dog, cat, cow, pig, horse, sheep, or goat. In some embodiments, the subject is a companion animal, such as a dog or cat. In some embodiments, the subject is a livestock animal, such as a cow, pig, horse, sheep, or goat. In some embodiments, the subject is a zoo animal. In some embodiments, the subject is a research animal, such as a rodent (e.g., mouse or rat), dog, pig, or non-human primate. In some embodiments, the animal is a genetically engineered animal. In some embodiments, the animal is a transgenic animal (e.g., transgenic mice and transgenic pigs). In some embodiments, the subject is a fish or reptile.

In some embodiments, the subject is a rodent, such as a mouse. In some such embodiments, the mouse is a transgenic mouse, such as a mouse expressing human MHC (i.e., HLA) molecules (e.g., Nicholson et al. (2012) Adv. Hematol. 2012: 404081). In some embodiments, the subject is a transgenic mouse expressing human TCRs or is an antigen-negative mouse (e.g., Li et al. (2010) Nat. Med. 16: 1029-1034 and Obenaus et al. (2015) Nat. Biotechnol. 33: 402-407). In some embodiments, the subject is a transgenic mouse expressing human HLA molecules and human TCRs. In some embodiments, such as where the subject is a transgenic HLA mouse, the identified TCRs are modified, e.g., to be chimeric or humanized. In some embodiments, the TCR scaffold is modified, such as analogous to known binding protein humanizing methods.

In some embodiments, the subject is a human. In some embodiments, the subject is an animal model of a disorder characterized by HA-1 expression (e.g., a non-malignant disorder, the hyperproliferative disorder, or the relapse of a hyperproliferative disorder characterized by expression of an HA-1 antigen). For example, the animal model may be an orthotopic xenograft animal model of a human-derived cancer.

In some embodiments, the subject is a human, such as a human with a disorder characterized by HA-1 expression.

The methods described herein may be used to treat a subject in need thereof. As used herein, a “subject in need thereof” includes any subject who has a disorder characterized by HA-1 expression, a relapse of a disorder characterized by HA-1 expression, and/or who is predisposed to a disorder characterized by HA-1 expression. As described herein, a disorder characterized by HA-1 expression may be a non-malignant disorder, a hyperproliferative disorder, or a relapse of a hyperproliferative disorder characterized by expression of an HA-1 antigen.

In some embodiments of the methods encompassed by the present invention, the subject has not undergone treatment for a disorder characterized by HA-1 expression, such as chemotherapy, radiation therapy, targeted therapy, and/or immunotherapies. In some embodiments, the subject has undergone treatment for a disorder characterized by HA-1 expression, such as chemotherapy, radiation therapy, targeted therapy, and/or immunotherapies.

In some embodiments, the subject has had surgery to remove cancerous or precancerous tissue. In some embodiments, the cancerous tissue has not been removed, e.g., the cancerous tissue may be located in an inoperable region of the body, such as in a tissue that is essential for life, or in a region where a surgical procedure would cause considerable risk of harm to the patient.

In some embodiments, the subject or cells thereof are resistant to a therapy of relevance, such as resistant to standard of care therapy, immune checkpoint inhibitor therapy, and the like. For example, modulating one or more biomarkers encompassed by the present invention may overcome resistance to immune checkpoint inhibitor therapy.

In some embodiments, the subjects are in need of modulation according to compositions and methods described herein, such as having been identified as having an unwanted absence, presence, or aberrant HA-1 expression.

a. Diagnostic Methods

In an aspect encompassed by the present invention, provided herein are diagnostic methods for detecting the presence or absence of an HA-1 antigen and/or a cell of interest expressing HA-1, comprising detecting the presence or absence of said HA-1 antigen in a sample by use of at least one binding protein, or at least one host cell described herein. In some embodiments, the method further comprising obtaining the sample (e.g., from a subject). In some embodiments, the at least one binding protein or the at least one host cell, forms a complex with an HA-1 peptide epitope in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay.

In an aspect encompassed by the present invention, provided herein are diagnostic methods for detecting the level of a non-malignant disorder, a hyperproliferative disorder, or a relapse of a hyperproliferative disorder characterized by expression of an HA-1 antigen in a subject, comprising: a) contacting a sample obtained from the subject with at least one binding protein, at least one host cell, or a population of host cells described herein; and b) detecting the level of reactivity, wherein a higher level of reactivity compared to a control level indicates that the level of a non-malignant disorder, a hyperproliferative disorder, or a relapse of a hyperproliferative disorder characterized by expression of an HA-1 antigen in the subject.

In some embodiments, the level of reactivity is indicated by T cell activation or effector function, such as, but not limited to, T cell proliferation, killing, or cytokine release. The control level may be a reference number or a level of a healthy subject who has no exposure to a non-alignant disorder, a hyperproliferative disorder, or a relapse of a hyperproliferative disorder characterized by expression of an HA-1 antigen.

A biological sample may be obtained from a subject for determining the presence and level of an immune response to a peptide antigen (e.g., an HA-1 antigen) as described herein. A “biological sample” as used herein may be a blood sample (from which serum or plasma may be prepared), biopsy specimen, body fluids (e.g., blood, isolated PBMCs, isolated T cells, lung lavage, ascites, mucosal washings, synovial fluid, etc.), bone marrow, lymph nodes, tissue explant, organ culture, or any other tissue or cell preparation from the subject or a biological source. Biological samples may also be obtained from the subject prior to receiving any pharmaceutical composition, which biological sample is useful as a control for establishing baseline data.

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

The level of an immune response, such as a cytotoxic T lymphocyte (CTL) immune response may be determined by any one of numerous immunological methods described herein and routinely practiced in the art. For example, the level of a CTL immune response may be determined prior to and following administration of any one of the herein described binding proteins expressed by, for example, a T cell. Cytotoxicity assays for determining CTL activity may be performed using any one of several techniques and methods routinely practiced in the art (e.g., Henkart et al., “Cytotoxic T-Lymphocytes” in Fundamental Immunology, Paul (ed.) (2003 Lippincott Williams & Wilkins, Philadelphia, PA), pages 1127-50, and references cited therein).

The present invention provides, in part, methods, systems, and code for accurately classifying whether a biological sample is associated with an output of interest, such as expression of a target of interest, such as HA-1. In some embodiments, the present invention is useful for classifying a sample (e.g., from a subject) as associated with or at risk for responding to or not responding to therapy for a disorder characterized by HA-1 expression using a statistical algorithm and/or empirical data.

An exemplary method for detecting the amount or activity of HA-1, and thus useful for classifying whether a sample is likely or unlikely to respond to a therapy for a disorder characterized by HA-1 expression involves contacting a biological sample with an agent, such as an HA-1 immunogenic peptide or binding agent described herein, capable of detecting the amount or activity of HA-1 in the biological sample. In some embodiments, the method further comprises obtaining a biological sample, such as from a test subject. In some embodiments, at least one agent is used, wherein two, three, four, five, six, seven, eight, nine, ten, or more such agents may be used in combination (e.g., in sandwich ELISAs) or in serial. In certain instances, the statistical algorithm is a single learning statistical classifier system. For example, a single learning statistical classifier system may be used to classify a sample as a based upon a prediction or probability value and the presence or level of the biomarker. The use of a single learning statistical classifier system typically classifies the sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method encompassed by the present invention further comprises sending the sample classification results to a clinician, e.g., an oncologist.

In some embodiments, the diagnosis of a subject is followed by administering to the individual a therapeutically effective amount of a defined treatment based upon the diagnosis.

In some embodiments, the methods further involve obtaining a control biological sample (e.g., biological sample from a subject who does not have a disorder characterized by HA-1 expression, a subject who is in remission, a subject whose disorder is susceptible to therapy, a subject whose disorder is progressing, or other subjects of interest).

In some embodiments of analytical methods described herein, HA-1 expression (e.g., in a sample from a subject) is compared to a pre-determined control (standard) sample. The sample from the subject is typically from a diseased tissue, such as cancer cells or tissues. The control sample may be from the same subject or from a different subject. The control sample is typically a normal, non-diseased sample. However, in some embodiments, such as for staging of disease or for evaluating the efficacy of treatment, the control sample may be from a diseased tissue. The control sample may be a combination of samples from several different subjects. In some embodiments, the HA-1 expression measurement(s) from a subject is compared to a pre-determined level. This pre-determined level is typically obtained from normal samples. As described herein, a “pre-determined” expression may be used to, by way of example only, evaluate a subject that may be selected for treatment, evaluate a response to cancer, and/or evaluate a response to a combination cancer therapy. A pre-determined biomarker amount and/or activity measurement(s) may be determined in populations of patients with or without a disorder characterized by HA-1 expression. The pre-determined biomarker amount and/or activity measurement(s) may be a single number, equally applicable to every patient, or the pre-determined biomarker amount and/or activity measurement(s) may vary according to specific sub-populations of patients. Age, weight, height, and other factors of a subject may affect the pre-determined biomarker amount and/or activity measurement(s) of the individual. Furthermore, the pre-determined biomarker amount and/or activity may be determined for each subject individually. In one embodiment, the amounts determined and/or compared in a method described herein are based on absolute measurements.

In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis may be based on the ratio of pre-treatment biomarker measurement as compared to post-treatment biomarker measurement. Pre-treatment biomarker measurement may be made at any time prior to initiation of a therapy. Post-treatment biomarker measurement may be made at any time after initiation of therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of therapy, and even longer toward indefinitely for continued monitoring. Treatment may comprise therapy to treat the disorder characterized by HA-1 expression, either alone or in combination with other agents, such as anti-cancer agents like chemotherapy or immune checkpoint inhibitors.

The pre-determined HA-1 expression may be any suitable standard. For example, the pre-determined HA-1 expression may be obtained from the same or a different subject for whom a subject selection is being assessed. In one embodiment, the pre-determined biomarker amount and/or activity measurement(s) may be obtained from a previous assessment of the same patient. In such a manner, the progress of the selection of the patient may be monitored over time. In addition, the control may be obtained from an assessment of another human or multiple humans, e.g., selected groups of humans, if the subject is a human. In such a manner, the extent of the selection of the human for whom selection is being assessed may be compared to suitable other humans, e.g., other humans who are in a similar situation to the human of interest, such as those suffering from similar or the same condition(s) and/or of the same ethnic group.

In some embodiments, the change of HA-1 expression from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cut-off values apply equally when the measurement is based on relative changes, such as based on the ratio of pre-treatment biomarker measurement as compared to post-treatment biomarker measurement.

In some embodiments, HA-1 expression may be detected and/or quantified by detecting or quantifying HA-1 polypeptide or antigen thereof, such as by using a composition described herein. The polypeptide may be detected and quantified by any of a number of means well-known to those of skill in the art, such as by immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991).

b. Therapeutic Methods

In an aspect encompassed by the present invention, provided herein are methods for preventing and/or treating a non-malignant disorder, a hyperproliferative disorder, or a relapse of a hyperproliferative disorder characterized by expression of an HA-1 antigen, and/or for inducing an immune response against a cell of interest, such as a hyperproliferative cell, expressing an HA-1 antigen. In some embodiments, the method comprises administering to a subject a therapeutically effective amount of a composition comprising cells expressing at least one binding protein described herein. The methods encompassed by the present invention also may be used to determine the responsiveness to therapy for many different disorders characterized by HA-1 expression in subjects, such as those described herein.

In some embodiments, provided herein are methods for preventing and/or treating certain hyperproliferative disorders (e.g., hematological malignancies). In certain embodiments, the hematological malignancy comprises a leukemia (e.g., an acute leukemia or a chronic leukemia). In specific embodiments, the leukemia comprises acute myeloid leukemia (AML), acute lymphocytic leukemia (ALL), mixed phenotype acute leukemia (MPAL), chronic myeloid leukemia (CML), B cell prolymphocytic leukemia, hairy cell leukemia, or chronic lymphocytic leukemia (CLL). In certain embodiments, the hematological malignancy comprises a lymphoma. In certain embodiments, the lymphoma comprises Hodgkin's lymphoma (HL), non-Hodgkin's lymphoma (NHL), a central nervous system lymphoma, small lymphocytic lymphoma (SLL), CD37⁺ dendritic cell lymphoma, lymphoplasmacytic lymphoma, splenic marginal zone lymphoma, plasma cell myeloma, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, precursor B-lymphoblastic lymphoma, immunoblastic large cell lymphoma, intravascular large B-cell lymphoma, primary effusion lymphoma, Burkitt's lymphoma/leukemia, B-cell proliferations of uncertain malignant potential, lymphomatoid granulomatosis, and post-transplant lymphoproliferative disorder. In certain embodiments, the hematological malignancy comprises a myelodysplastic disorder (MDS), such as, for example, refractory cytopenia with unilineage dysplasia (refractory anemia, refractory neutropenia, and refractory thrombocytopenia), refractory anemia with ring sideroblasts (RARS), refractory anemia with ring sideroblasts-thrombocytosis (RARS-t), refractory cytopenia with multinieage dysplasia (RCMD), refractory cytopenia with multinieage dysplasia and ring sideroblasts (RCMD-RS), refractory anemia with excess blasts (RAEB), myelodysplasia unclassifiable, refractory cytopenia of childhood, MDS with isolated del(5q), and other well-known MDS subtypes (see, for example, 2016 World Health Organization MDS classification available on the World Wide Web at nbci.nlm.nih.gov/pmc/articles/PMC655460/). In certain embodiments, the hematological malignancy comprises a myeloproliferative neoplasms (MPN), such as for example, nmyelofibrosis, polycythemia vera, essential thrombocythemia, chronic myelomonocytic leukemia, chronic myeloproliferative disease-unclassifiable, chronic neutrophilic leukemia, chronic eosinophilic leukemia and MPN—not otherwise specified. In further embodiments, the hematological malignancy comprises a myeloma.

In some embodiments, provided herein are methods for preventing and/or treating a non-malignant disorder. In certain embodiments, subjects afflicted with these non-malignant disorders have undergone or are undergoing hematopoietic cell transplantation.

In some embodiments, the non-malignant disorder is selected from the group consisting of non-malignant hematology (e.g., sickle cell anemia, thalassemia, aplastic anemia, hemophagocytic lymphohistiocytosis (HLH), severe aplastic anemia, marrow failure syndromes (including Fanconi anemia, Diamond-Blackfan anemia, Shwachman Diamond syndrome, and others); immune deficiency disorders (e.g., severe combined immunodeficiency (SCID), Wiskott-Aldrich syndrome, Omenn syndrome, X-linked lymphoproliferative syndrome, chronic granulomatous disease, leukocyte adhesion deficiency, DiGeorge syndrome and other well-known disorders, such as indications for hematopoietic stem cell transplantation (HCT) like those listed at bethematchclinical.org/workarea/downloadasset.aspx?id-3545); and autoimmune disorder (e.g., systemic sclerosis, glomerular nephritis, arthritis, dilated cardiomyopathy-like disease, ulceous colitis, Sjogren syndrome, Crohn disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis). In specific embodiments, the autoimmune disorder is systemic sclerosis or multiple sclerosis.

In addition, the compositions described herein may also be administered in combination therapy to further modulate a desired activity. Additional agents include, without limitations, chemotherapeutic agents, hormones, antiangiogens, radiolabelled, compounds, or with surgery, cryotherapy, and/or radiotherapy. The preceding treatment methods may be administered in conjunction with other forms of conventional therapy (e.g., standard-of-care treatments for cancer well-known to the skilled artisan), either consecutively with, pre- or post-conventional therapy. For example, these modulatory agents may be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and may be determined by the physician.

Therapy using one or more compositions described herein, either alone or in combination with other therapies, such as cancer therapies, may be used to contact HA-1-expressing cells and/or administered to a desired subject, such as a subject that is indicated as being a likely responder to therapy. In another embodiment, such therapy may be avoided once a subject is indicated as not being a likely responder to the therapy (e.g., as assessed according to a diagnostic or prognostic method described herein) and an alternative treatment regimen, such as targeted and/or untargeted cancer therapies, may be recommended and/or administered.

The term “targeted therapy” refers to administration of agents that selectively interact with a chosen biomolecule to thereby treat cancer. For example, targeted therapy regarding the inhibition of immune checkpoint inhibitor is useful in combination with the methods encompassed by the present invention.

The term “immunotherapy” or “immunotherapies” generally refers to any strategy for modulating an immune response in a beneficial manner and encompasses the treatment of a subject afflicted with, or at risk of contracting or suffering a recurrence of, a disease by a method comprising inducing, enhancing, suppressing or otherwise modifying an immune response, as well as any treatment that uses certain parts of a subject's immune system to fight diseases, such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” In some embodiments, an immunotherapy is specific for cells of interest, such as cancer cells. In some embodiments, immunotherapy may be “untargeted,” which refers to administration of agents that do not selectively interact with immune system cells, yet modulates immune system function. Representative examples of untargeted therapies include, without limitation, chemotherapy, gene therapy, and radiation therapy.

Some forms of immunotherapy are targeted therapies that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy may involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy may also focus on using the cytotoxic lymphocyte-recognized epitopes of cancer cell lines. Alternatively, antisense polynucleotides, ribozymes, RNA interference molecules, triple helix polynucleotides and the like, may be used to selectively modulate biomolecules that are linked to the initiation, progression, and/or pathology of a tumor or cancer. Similarly, immunotherapy may take the form of cell-based therapies. For example, adoptive cellular immunotherapy is a type of immunotherapy using immune cells, such as T cells, that have a natural or genetically engineered reactivity to a patient's cancer are generated and then transferred back into the cancer patient. The injection of a large number of activated tumor-specific T cells may induce complete and durable regression of cancers.

Immunotherapy may involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy may also focus on using the cytotoxic lymphocyte-recognized epitopes of cancer cell lines. Alternatively, antisense polynucleotides, ribozymes, RNA interference molecules, triple helix polynucleotides and the like, may be used to selectively modulate biomolecules that are linked to the initiation, progression, and/or pathology of a tumor or cancer.

In some embodiments, an immunotherapeutic agent is an agonist of an immune-stimulatory molecule; an antagonist of an immune-inhibitory molecule; an antagonist of a chemokine; an agonist of a cytokine that stimulates T cell activation; an agent that antagonizes or inhibits a cytokine that inhibits T cell activation; and/or an agent that binds to a membrane bound protein of the B7 family. In some embodiments, the immunotherapeutic agent is an antagonist of an immune-inhibitory molecule. In some embodiments, the immunotherapeutic agents may be agents for cytokines, chemokines and growth factors, for examples, neutralizing antibodies that neutralize the inhibitory effect of tumor associated cytokines, chemokines, growth factors and other soluble factors, including IL-10, TGF-β and VEGF.

In some embodiments, immunotherapy comprises inhibitors of one or more immune checkpoints. The term “immune checkpoint” refers to a group of molecules on the cell surface of CD4+ and/or CD8+ T cells that fine-tune immune responses by modulating anti-cancer immune responses, such as down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD200R, CD160, gp49B, PIR-B, KRLG-1, KIR family receptors, TIM-1, TIM-3, TIM-4, LAG-3 (CD223), IDO, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). The term further encompasses biologically active protein fragments, as well as nucleic acids encoding full-length immune checkpoint proteins.

Some immune checkpoints are “immune-inhibitory immune checkpoints” encompassing molecules (e.g., proteins) that inhibit, down-regulate, or suppress a function of the immune system (e.g., an immune response). For example, PD-L1 (programmed death-ligand 1), also known as CD274 or B7-H1, is a protein that transmits an inhibitory signal that reduces proliferation of T cells to suppress the immune system. CTLA-4 (cytotoxic T-lymphocyte-associated protein 4), also known as CD152, is a protein receptor on the surface of antigen-presenting cells that serves as an immune checkpoint (“off” switch) to downregulate immune responses. TIM-3 (T-cell immunoglobulin and mucin-domain containing-3), also known as HAVCR2, is a cell surface protein that serves as an immune checkpoint to regulate macrophage activation. VISTA (V-domain Ig suppressor of T cell activation) is a type I transmembrane protein that functions as an immune checkpoint to inhibit T cell effector function and maintain peripheral tolerance. LAG-3 (lymphocyte-activation gene 3) is an immune checkpoint receptor that negatively regulates proliferation, activation, and homeostasis of T cells. BTLA (B- and T-lymphocyte attenuator) is a protein that displays T cell inhibition via interactions with tumor necrosis family receptors (TNF-R). KIR (killer-cell immunoglobulin-like receptor) is a family of proteins expressed on NK cells, and a minority of T cells, that suppress the cytotoxic activity of NK cells. In some embodiments, immunotherapeutic agents may be agents specific to immunosuppressive enzymes such as inhibitors that may block the activities of arginase (ARG) and indoleamine 2,3-dioxygenase (IDO), an immune checkpoint protein that suppresses T cells and NK cells, which change the catabolism of the amino acids arginine and tryptophan in the immunosuppressive tumor microenvironment. The inhibitors may include, but are not limited to, N-hydroxy-L-Arg (NOHA) targeting to ARG-expressing M2 macrophages, nitroaspirin or sildenafil (Viagra®), which blocks ARG and nitric oxide synthase (NOS) simultaneously; and IDO inhibitors, such as 1-methyl-tryptophan. The term further encompasses biologically active protein fragment, as well as nucleic acids encoding full-length immune checkpoint proteins and biologically active protein fragments thereof. In some embodiment, the term further encompasses any fragment according to homology descriptions provided herein.

By contrast, other immune checkpoints are “immune-stimulatory” encompassing molecules (e.g., proteins) that activate, stimulate, or promote a function of the immune system (e.g., an immune response). In some embodiments, the immune-stimulatory molecule is CD28, CD80 (B7.1), CD86 (B7.2), 4-1BB (CD137), 4-1BBL (CD137L), CD27, CD70, CD40, CD40L, CD122, CD226, CD30, CD30L, OX40, OX40L, HVEM, BTLA, GITR and its ligand GITRL, LIGHT, LTβR, LTαβ, ICOS (CD278), ICOSL (B7-H2), and NKG2D. CD40 (cluster of differentiation 40) is a costimulatory protein found on antigen presenting cells that is required for their activation. OX40, also known as tumor necrosis factor receptor superfamily member 4 (TNFRSF4) or CD134, is involved in maintenance of an immune response after activation by preventing T-cell death and subsequently increasing cytokine production. CD137 is a member of the tumor necrosis factor receptor (TNF-R) family that co-stimulates activated T cells to enhance proliferation and T cell survival. CD122 is a subunit of the interleukin-2 receptor (IL-2) protein, which promotes differentiation of immature T cells into regulatory, effector, or memory T cells. CD27 is a member of the tumor necrosis factor receptor superfamily and serves as a co-stimulatory immune checkpoint molecule. CD28 (cluster of differentiation 28) is a protein expressed on T cells that provides co-stimulatory signals required for T cell activation and survival. GITR (glucocorticoid-induced TNFR-related protein), also known as TNFRSF18 and AITR, is a protein that plays a key role in dominant immunological self-tolerance maintained by regulatory T cells. ICOS (inducible T-cell co-stimulator), also known as CD278, is a CD28-superfamily costimulatory molecule that is expressed on activated T cells and play a role in T cell signaling and immune responses.

Immune checkpoints and their sequences are well-known in the art and representative embodiments are described further below. Immune checkpoints generally relate to pairs of inhibitory receptors and the natural binding partners (e.g., ligands). For example, PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells. The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell inhibitory signals, and the ability to modulate the immune response. The term “PD-1 ligand” refers to binding partners of the PD-1 receptor and includes both PD-L1 (Freeman et al. (2000) J. Exp. Med. 192:1027-1034) and PD-L2 (Latchman et al. (2001) Nat. Immunol. 2:261). The term “PD-1 ligand activity” includes the ability of a PD-1 ligand polypeptide to bind its natural receptor(s) (e.g., PD-1 or B7-1), the ability to modulate immune cell inhibitory signals, and the ability to modulate the immune response.

As used herein, the term “immune checkpoint therapy” refers to the use of agents that inhibit immune-inhibitory immune checkpoints, such as inhibiting their nucleic acids and/or proteins. Inhibition of one or more such immune checkpoints may block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that may either bind and/or inactivate or inhibit immune checkpoint proteins, or fragments thereof, as well as RNA interference, antisense, nucleic acid aptamers, etc. that may downregulate the expression and/or activity of immune checkpoint nucleic acids, or fragments thereof. Exemplary agents for upregulating an immune response include antibodies against one or more immune checkpoint proteins that block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g., the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents may directly block the interaction between the one or more immune checkpoints and its natural receptor(s) (e.g., antibodies) to prevent inhibitory signaling and upregulate an immune response. Alternatively, agents may indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain may binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. Therapeutic agents used for blocking the PD-1 pathway include antagonistic antibodies and soluble PD-L1 ligands. The antagonist agents against PD-1 and PD-L1/2 inhibitory pathway may include, but are not limited to, antagonistic antibodies to PD-1 or PD-L1/2 (e.g., 17D8, 2D3, 4H1, 5C4 (also known as nivolumab or BMS-936558), 4A11, 7D3 and 5F4 disclosed in U.S. Pat. No. 8,008,449; AMP-224, pidilizumab (CT-011), pembrolizumab, and antibodies disclosed in U.S. Pat. Nos. 8,779,105; 8,552,154; 8,217,149; 8,168,757; 8,008,449; 7,488,802; 7,943,743; 7,635,757; and 6,808,710. Similarly, additional representative checkpoint inhibitors may be, but are not limited to, antibodies against inhibitory regulator CTLA-4 (anti-cytotoxic T-lymphocyte antigen 4 anti-cytotoxic T-lymphocyte antigen 4), such as ipilimumab, tremelimumab (fully humanized), anti-CD28 antibodies, anti-CTLA-4 adnectins, anti-CTLA-4 domain antibodies, single chain anti-CTLA-4 antibody fragments, heavy chain anti-CTLA-4 fragments, light chain anti-CTLA-4 fragments, and other antibodies, such as those disclosed in U.S. Pat. Numbers 8,748, 815; 8,529,902; 8,318,916; 8,017,114; 7,744,875; 7,605,238; 7,465,446; 7,109,003; 7,132,281; 6,984,720; 6,682,736; 6,207,156; and 5,977,318, as well as EP Pat. No. 1212422, U.S. Pat Publ. Numbers 2002/0039581 and 2002/086014, and Hurwitz et al. (1998) Proc. Natd. Acad. Sci. U.S.A. 95:10067-10071.

The representative definitions of immune checkpoint activity, ligand, blockade, and the like exemplified for PD-1, PD-L1, PD-L2, and CTLA-4 apply generally to other immune checkpoints.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary agents include, but are not limited to, alkylating agents: nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Similarly, additional exemplary agents including platinum-ontaining compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AGO13736), bosutinib (SKI-606), cediranib (RECENTIN®, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE®)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. No. 36,397); and NU1025 (Bowman et al.). The mechanism of action is generally related to the ability of PARP inhibitors to bind PARP and decrease its activity. PARP catalyzes the conversion of beta-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard et. al. (2003) Exp. Hematol. 31:446-454); Herceg (2001)Mut. Res. 477:97-110). Poly(ADP-ribose) polymerase 1 (PARP1) is a key molecule in the repair of DNA single-strand breaks (SSBs) (de Murcia J. et al. (1997) Proc. Natl. Acad. Sci. U.S.A. 94:7303-7307; Schreiber et al. (2006) Nat. Rev. Mol. Cell Biol. 7:517-528; Wang et al. (1997) Genes Dev. 11:2347-2358). Knockout of SSB repair by inhibition of PARP1 function induces DNA double-strand breaks (DSBs) that may trigger synthetic lethality in cancer cells with defective homology-directed DSB repair (Bryant et al. (2005) Nature 434:913-917; Farmer et al. (2005) Nature 434:917-921). The foregoing examples of chemotherapeutic agents are illustrative and are not intended to be limiting.

In another embodiment, radiation therapy is used. The radiation used in radiation therapy may be ionizing radiation. Radiation therapy may also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy may be administered as external beam radiation or teletherapy wherein the radiation is directed from a remote source. The radiation treatment may also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments may comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In one aspect, provided herein is a method of eliciting in a subject an immune response to a cell that expresses an HA-1 antigen. In some embodiments, the method comprises administering to the subject a pharmaceutical composition described herein, wherein the pharmaceutical composition, when administered to the subject, elicits an immune response to the cell that expresses an HA-1 antigen.

In some embodiments, the immune response can include a cell-mediated immune response. A cellular immune response is a response that involves T cells and may be determined in vitro, ex vivo, or in vivo. For example, a general cellular immune response may be determined as the T cell proliferative activity in cells (e.g., peripheral blood leukocytes (PBLs)) sampled from the subject at a suitable time following the administering of a pharmaceutical composition. Following incubation of e.g., PBMCs with a stimulator for an appropriate period, [³H]thymidine incorporation may be determined. The subset of T cells that is proliferating may be determined using flow cytometry.

In another aspect encompassed by the present invention, the methods provided herein include administering to both human and non-human mammals as described above. Veterinary applications also are contemplated. In some embodiments, the subject may be any living organism in which an immune response may be elicited.

In some embodiments, the pharmaceutical composition may be administered at any time that is appropriate. For example, the administering may be conducted before or during treatment of a subject having a non-malignant disorder, a hyperproliferative disorder, or a relapse of a hyperproliferative disorder characterized by expression of an HA-1 antigen, and continued after the non-malignant disorder, hyperproliferative disorder, or the relapse of a hyperproliferative disorder characterized by expression of an HA-1 antigen becomes clinically undetectable. The administering also may be continued in a subject showing signs of recurrence.

In some embodiments, the pharmaceutical composition may be administered in a therapeutically or a prophylactically effective amount Administering the pharmaceutical composition to the subject may be carried out using known procedures, and at dosages and for periods of time sufficient to achieve a desired effect.

In some embodiments, the pharmaceutical composition may be administered to the subject at any suitable site. Administration may be accomplished using methods generally known in the art. Agents, including cells, may be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular administration. For example, subjects of interest may be engrafted with the transplanted cells by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. In certain embodiment, the cancer vaccine encompassed by the present invention is injected to the subject intratumorally or subcutaneously. Cells may be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427). In some embodiments, the dose may be administered in an amount and for a period of time effective in bringing about a desired response, be it eliciting the immune response or the prophylactic or therapeutic treatment of a non-malignant disorder, a hyperproliferative disorder, or a relapse of a hyperproliferative disorder characterized by expression of an HA-1 antigen and/or symptoms associated therewith.

The pharmaceutical composition may be given subsequent to, preceding, or contemporaneously with other therapies including therapies that also elicit an immune response in the subject. For example, the subject may previously or concurrently be treated by other forms of immunomodulatory agents, such other therapies may be provided in such a way so as not to interfere with the immunogenicity of the compositions described herein.

Administering may be properly timed by the care giver (e.g., physician, veterinarian), and may depend on the clinical condition of the subject, the objectives of administering, and/or other therapies also being contemplated or administered. In some embodiments, an initial dose may be administered, and the subject monitored for an immunological and/or clinical response. Suitable means of immunological monitoring include using patient's peripheral blood lymphocyte (PBL) as responders and immunogenic peptides or peptide-MHC complexes described herein as stimulators. An immunological reaction also may be determined by a delayed inflammatory response at the site of administering. One or more doses subsequent to the initial dose may be given as appropriate, typically on a monthly, semimonthly, or a weekly basis, until the desired effect is achieved. Thereafter, additional booster or maintenance doses may be given as required, particularly when the immunological or clinical benefit appears to subside.

In general, an appropriate dosage and treatment regimen provides the active molecules or cells in an amount sufficient to provide a benefit Such a response may be monitored by establishing an improved clinical outcome (e.g., more frequent remissions, complete or partial, or longer disease-free survival) in treated subjects as compared to non-treated subjects. Increases in preexisting immune responses to a viral protein generally correlate with an improved clinical outcome. Such immune responses may generally be evaluated using standard proliferation, cytotoxicity or cytokine assays, which are routine.

For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro, ex vivo, and in vivo animal studies) and clinical studies and analyzing data obtained therefrom by appropriate statistical, biological, and clinical methods and techniques, all of which can readily be practiced by an ordinarily skilled artisan.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., engineered immune cells with one or more cytokines; immunosuppressive therapy such as calcineurin inhibitors, corticosteroids, microtubule inhibitors, low dose of a mycophenolic acid prodrug, or any combination thereof).

In some embodiments, a plurality of doses of a host cell (e.g., an engineered immune cell) described herein is administered to the subject, which may be administered at intervals between administrations of about two to about four weeks.

Treatment or prevention methods encompassed by the present invention may be administered to a subject as part of a treatment course or regimen, which may comprise additional treatments prior to, or after, administration of the instantly disclosed unit doses, cells, or compositions. For example, in some embodiments, a subject receiving a unit dose of the host cell (e.g., an engineered immune cell) is receiving or had previously received a hematopoietic cell transplant (HCT; including myeloablative and non-myeloablative HCT).

In any of the foregoing embodiments, a hematopoietic cell used in an HCT may be a “universal donor” cell that is modified to reduce or eliminate expression of one or more endogenous genes that encode a polypeptide product selected from an MHC, antigen, and a binding protein (e.g., by a chromosomal gene knockout according to the methods described herein).

Techniques and regimens for performing cell transplantation are known in the art and may comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in some embodiments, a host cell (e.g., an engineered immune cell) encompassed by the present invention may be administered with or shortly after stem cell therapy.

Methods encompassed by the present invention may, in some embodiments, further include administering one or more additional agents to treat the disease or disorder (e.g., a non-malignant disorder, a hyperproliferative disorder, or a relapse of a hyperproliferative disorder characterized by expression of an HA-1 antigen) in a combination therapy. For example, in some embodiments, a combination therapy comprises administering host cell or binding protein encompassed by the present invention with (concurrently, simultaneously, or sequentially) an antiviral agent. In some embodiments, a combination therapy comprises administering a host cell or binding protein encompassed by the present invention with lopinavir/ritonavir, chloroquine, ribavirin, steroid drugs, hydroxychloroquine, and/or interferon α. In some embodiments, a combination therapy comprises administering a host cell, composition, or unit dose of the host cells encompassed by the present invention with a secondary therapy, such as a surgery, an antibody, a vaccine, or any combination thereof.

In some embodiments, the subject is a human, such as a human with a non-malignant disorder, a hyperproliferative disorder, or a relapse of a hyperproliferative disorder characterized by expression of an HA-1 antigen. In some embodiments, the subject is a rodent, such as a mouse. In some such embodiments, the mouse is a transgenic mouse, such as a mouse expressing human MHC (i.e., HLA) molecules, such as HLA-A2 (e.g., Nicholson et al. (2012) Adv. Hematol. 2012: 404081).

In some embodiments, the subject is a transgenic mouse expressing human TCRs or is an antigen-negative mouse (e.g., Li et al. (2010) Nat. Med. 16: 1029-1034 and Obenaus et al. (2015) Nat. Biotechnol. 33: 402-407). In some embodiments, the subject is a transgenic mouse expressing human HLA molecules and human TCRs.

In some embodiments, such as where the subject is a transgenic HLA mouse, the identified TCRs are modified, e.g., to be chimeric or humanized. In some embodiments, the TCR scaffold is modified, such as analogous to known binding protein humanizing methods.

c. Screening Methods

Another aspect encompassed by the present invention encompasses screening assays.

The present invention encompasses assays for screening agents, such as test proteins, that bind to, or modulate the activity of, HA-1 or an antigen thereof. Such agents include, without limitation, antibodies, proteins, fusion proteins, small molecules, and nucleic acids. In some embodiments, a method for identifying an agent which modulates an immune response entails determining the ability of the candidate agent to modulate HA-1 activity and further modulate an immune response of interest, such as modulated cytotoxic T cell activation and/or activity, sensitivity of cancer cells to immune checkpoint therapy, and the like.

In some embodiments, an assay is a cell-free or cell-based assay, comprising contacting a target, with a test agent, and determining the ability of the test agent to modulate (e.g., upregulate or downregulate) the amount and/or activity of the target, such as by measuring direct or indirect parameters as described below.

In some embodiments, an assay is a cell-based assay, such as one comprising contacting (a) a cell of interest with a test agent and determining the ability of the test agent to modulate the amount and/or activity of the target, such as binding characteristics. Determining the ability of the polypeptides to bind to, or interact with, each other may be accomplished, e.g., by measuring direct binding or by measuring a parameter of immune cell activation or function.

In another embodiment, an assay is a cell-based assay, comprising contacting a cell such as a cancer cell with immune cells (e.g., cytotoxic T cells) and a test agent, and determining the ability of the test agent to modulate the amount and/or activity of the target, and/or modulated immune responses, such as by measuring direct or indirect parameters as described below.

The methods described above and herein may also be adapted to test one or more agents that are already known to modulate the amount and/or activity of one or more biomarkers described herein to confirm modulation of the one or more biomarkers and/or to confirm the effects of the agents on readouts of a desired phenotype, such as modulated immune responses, sensitivity to immune checkpoint blockade, and the like.

In some embodiments, determining the ability of a test agent (e.g. antibodies, fusion proteins, peptides, or small molecules) to modulate the interaction between a given set of polypeptides may be accomplished by determining the activity of one or more members of the set of polypeptides. For example, the activity of a protein and/or one or more binding partners may be determined by detecting induction of a cellular second messenger (e.g., intracellular signaling), detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting a cellular response regulated by the protein and/or the one or more binding partners. Determining the ability of the test agent to bind to or interact with said polypeptide may be accomplished, for example, by measuring the ability of a compound to modulate immune cell costimulation or inhibition in a proliferation assay, or by interfering with the ability of said polypeptide to bind to antibodies that recognize a portion thereof.

Agents that modulate target amount and/or activity, such as interactions with one or more binding partners, may be identified by their ability to inhibit immune cell proliferation, and/or effector function, or to induce anergy, clonal deletion, and/or exhaustion when added to an in vitro assay. For example, cells may be cultured in the presence of an agent that stimulates signal transduction via an activating receptor. A number of recognized readouts of cell activation may be employed to measure, cell proliferation or effector function (e.g., antibody production, cytokine production, phagocytosis) in the presence of the agent. The ability of a test agent to block this activation may be readily determined by measuring the ability of the agent to effect a decrease in proliferation or effector function being measured, using techniques known in the art.

For example, agents encompassed by the present invention may be tested for the ability to inhibit or enhance costimulation in a T cell assay, as described in Freeman et al. (2000) J. Exp. Med. 192:1027 and Latchman et al. (2001) Nat. Immunol. 2:261. CD4⁺ T cells may be isolated from human PBMCs and stimulated with activating anti-CD3 antibody. Proliferation of T cells may be measured by ³H thymidine incorporation. An assay may be performed with or without CD28 costimulation in the assay. Similar assays may be performed with Jurkat T cells and PHA-blasts from PBMCs.

Alternatively, agents encompassed by the present invention may be tested for the ability to modulate cellular production of cytokines which are produced by or whose production is enhanced or inhibited in immune cells in response to modulation of the one or more biomarkers. Indicative cytokines released by immune cells of interest may be identified by ELISA or by the ability of an antibody which blocks the cytokine to inhibit immune cell proliferation or proliferation of other cell types that is induced by the cytokine, such as those described in the Examples section. An in vitro immune cell costimulation assay may also be used in a method for identifying cytokines which may be modulated by modulation of the one or more biomarkers. For example, if a particular activity induced upon costimulation, e.g., immune cell proliferation, cannot be inhibited by addition of blocking antibodies to known cytokines, the activity may result from the action of an unknown cytokine. Following costimulation, this cytokine may be purified from the media by conventional methods and its activity measured by its ability to induce immune cell proliferation. To identify cytokines which may play a role the induction of tolerance, an in vitro T cell costimulation assay as described above may be used. In this case, T cells would be given the primary activation signal and contacted with a selected cytokine, but would not be given the costimulatory signal. After washing and resting the immune cells, the cells would be rechallenged with both a primary activation signal and a costimulatory signal. If the immune cells do not respond (e.g., proliferate or produce cytokines) they have become tolerized and the cytokine has not prevented the induction of tolerance. However, if the immune cells respond, induction of tolerance has been prevented by the cytokine. Those cytokines which are capable of preventing the induction of tolerance may be targeted for blockage in vivo in conjunction with reagents which block B lymphocyte antigens as a more efficient means to induce tolerance in transplant recipients or subjects with autoimmune diseases.

In some embodiments, an assay encompassed by the present invention is a cell-free assay for screening for agents that modulate the interaction between a biomarker and/or one or more binding partners, comprising contacting a polypeptide and one or more natural binding partners, or biologically active portion thereof, with a test agent and determining the ability of the test compound to modulate the interaction between the polypeptide and one or more natural binding partners, or biologically active portion thereof. Binding of the test compound may be determined either directly or indirectly as described above. In one embodiment, the assay includes contacting the polypeptide, or biologically active portion thereof, with its binding partner to form an assay mixture, contacting the assay mixture with a test compound, and determining the ability of the test agent to interact with the polypeptide in the assay mixture, wherein determining the ability of the test agent to interact with the polypeptide comprises determining the ability of the test agent to preferentially bind to the polypeptide or biologically active portion thereof, as compared to the binding partner.

In some embodiments, whether for cell-based or cell-free assays, a test agent may further be assayed to determine whether it affects binding and/or activity of the interaction between the polypeptide and the one or more binding partners, with other binding partners. Other useful binding analysis methods include the use of real-time Biomolecular Interaction Analysis (BIA) (Sjolander and Urbaniczky (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., Biacore®). Changes in the optical phenomenon of surface plasmon resonance (SPR) may be used as an indication of real-time reactions between biological polypeptides. Polypeptides of interest may be immobilized on a Biacore® chip and multiple agents (blocking antibodies, fusion proteins, peptides, or small molecules) may be tested for binding to the polypeptide of interest. An example of using the BIA technology is described by Fitz et al. (1997) Oncogene 15:613.

The cell-free assays encompassed by the present invention are amenable to use of both soluble and/or membrane-bound forms of proteins. In the case of cell-free assays in which a membrane-bound form protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), orN-dodecyl=N,N-dimethyl-3-ammonio-1-propane sulfonate.

In one or more embodiments of the above described assay methods, it may be desirable to immobilize either polypeptides to facilitate separation of complexed from uncomplexed forms of one or both of the proteins, as well as to accommodate automation of the assay. Binding of a test compound to a polypeptide, may be accomplished in any vessel suitable for containing the reactants. Examples of such vessels include microtiter plates, test tubes, and micro-centrifuge tubes. In one embodiment, a fusion protein may be provided which adds a domain that allows one or both of the proteins to be bound to a matrix. For example, glutathione-S-transferase-based polypeptide fusion proteins, or glutathione-S-transferase/target fusion proteins, may be adsorbed onto glutathione sepharose beads (Sigma Chemical, St. Louis, MO) or glutathione derivatized microtiter plates, which are then combined with the test compound, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes may be dissociated from the matrix, and the level of polypeptide binding or activity determined using standard techniques.

The present invention further pertains to novel agents identified by the above-described screening assays. Accordingly, it is within the scope of the present invention to further use an agent identified as described herein in an appropriate model system. For example, an agent identified as described herein may be used in a model system to determine the efficacy, toxicity, or side effects of treatment with such an agent. Alternatively, an agent identified as described herein may be used in a model system to determine the mechanism of action of such an agent. Furthermore, the present invention pertains to uses of novel agents identified by the above-described screening assays for treatments as described herein.

d. Monitoring of Effects During Clinical Trials

Monitoring the influence of a non-malignant disorder, a hyperproliferative disorder, or a relapse of a hyperproliferative disorder characterized by expression of an HA-1 antigen therapy (e.g., compounds, drugs, vaccines, cell therapies, and the like) on immune responses, such as T cell reactivity (e.g., the presence of binding and/or T cell activation and/or effector function), may be applied not only in basic candidate HA-1 antigen binding molecule screening, but also in clinical trials. For example, the effectiveness of binding proteins and related compositions described herein, such as nucleic acids, host cells, pharmaceutical formulations, and the like, to increase immune response (e.g., T cell immune response) against cells of interest, such as hyperproliferative cells, expressing an HA-1 antigen, may be monitored in clinical trials of subjects afflicted with a non-malignant disorder, a hyperproliferative disorder, or a relapse of a hyperproliferative disorder characterized by expression of an HA-1 antigen. In such clinical trials, the presence of binding and/or T cell activation and/or effector function (e.g., T cell proliferation, killing, and/or cytokine release), may be used as a “read out” or marker of the phenotype of a particular cell, tissue, or system. Similarly, the effectiveness of an adaptive T cell therapy with T cells engineered to express a binding protein (e.g., a TCR, an antigen-binding fragment of a TCR, a CAR, or a fusion protein comprising a TCR and an effector domain) as described herein to increase immune response to cells of interest, such as hyperproliferative cells, that are expressing an HA-1 antigen, may be monitored in clinical trials of subjects having a non-malignant disorder, a hyperproliferative disorder, or a relapse of a hyperproliferative disorder characterized by expression of an HA-1 antigen. In such clinical trials, the presence of binding and/or T cell activation and/or effector function (e.g., T cell proliferation, killing, or cytokine release), may be used as a “read out” or marker of the phenotype of a particular cell, tissue, or system.

For example, increased administration of a non-malignant disorder, the hyperproliferative disorder, or the relapse of the hyperproliferative disorder characterized by expression of an HA-1 antigen therapy may be desirable to increase the presence or level of reactivity between a sample obtained from the subject and one or more binding proteins or related composition, such as to increase the effectiveness of the non-malignant disorder, the hyperproliferative disorder, or the relapse of the hyperproliferative disorder characterized by expression of an HA-1 antigen therapy. According to such an embodiment, the presence or level of reactivity between a sample obtained from the subject and one or more binding proteins or related composition may be used as an indicator of the effectiveness of a non-malignant disorder, a hyperproliferative disorder, or a relapse of a hyperproliferative disorder characterized by expression of an HA-1 antigen therapy, even in the absence of an observable phenotypic response. Similarly, analysis of the presence or level of reactivity between a sample obtained from the subject and one or more binding proteins or related composition, such as by a direct binding assay, fluorescence activated cell sorting (FACS), enzyme linked immunosorbent assay (ELISA), radioimmune assay (RIA), immunochemically, Western blot, or intracellular flow assay, may also be used to select patients who will receive the non-malignant disorder, the hyperproliferative disorder, or the relapse of the hyperproliferative disorder characterized by expression of an HA-1 antigen therapy.

For example, in a direct binding assay, immunogenic peptides or antigen peptide-MHC (pMHC) complexes may be coupled with a radioisotope or enzymatic label such that binding may be determined by detecting the labeled immunogenic peptides or pMHC complexes. For example, the immunogenic peptides or pMHC complexes may be labeled with ¹²⁵I, ³⁵S, ¹⁴C, or ³H, either directly or indirectly, and the radioisotope detected by direct counting of radioemission or by scintillation counting. Alternatively, the immunogenic peptides or pMHC complexes may be enzymatically labeled with, for example, horseradish peroxidase, alkaline phosphatase, or luciferase, and the enzymatic label detected by determination of conversion of an appropriate substrate to product Determining the interaction between immunogenic peptides or pMHC complexes and immune cells, such as T cells and/or NK cells, may also be accomplished using standard binding or enzymatic analysis assays. In one or more embodiments of the above described assay methods, it may be desirable to immobilize immunogenic peptides or pMHC complexes to accommodate automation of the assay.

It is also within the scope of the present invention to determine the ability of an agent to modulate a parameter of interest without the labeling of any of the interactants. For example, a microphysiometer may be used to detect interaction between polypeptides without the labeling of polypeptides to be monitored (McConnell et al. (1992) Science 257:1906-1912). As used herein, a “microphysiometer” (e.g., Cytosensor®) is an analytical instrument that measures the rate at which a cell acidifies its environment using a light-addressable potentiometric sensor (LAPS). Changes in this acidification rate may be used as an indicator of the interaction between compound and receptor.

Binding of immunogenic peptides or pMHC complexes to immune cells, such as T cells and/or NK cells, may be accomplished in any vessel suitable for containing the reactants. Non-limiting examples of such vessels include microtiter plates, test tubes, and micro-centrifuge tubes. Immobilized forms of the immunogenic peptides or pMHC complexes may also include immunogenic peptides or pMHC complexes bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In some embodiments, the reactivity of a sample obtained from the subject to one or more binding proteins or to one or more host cells described herein may be measured by detecting the presence of binding and/or T cell activation and/or effector function. The term “T cell activation” refers to T lymphocytes selected from proliferation, differentiation, cytokine secretion, release of cytotoxic effector molecules, cytotoxic activity, and expression of activation markers, particularly refers to one or more cellular responses of cytotoxic T lymphocytes.

Cytokine production and/or release may be measured by methods well-known in the art, for example, ELISA, enzyme-linked immune absorbent spot (ELISPOT), Luminex® assay, intracellular cytokine staining, and flow cytometry, and combinations thereof (e.g., intracellular cytokine staining and flow cytometry). It may be determined according to the method implemented.

The term “cytokine” as used herein refers to a molecule that mediates and/r regulates a biological or cellular function or process (e.g., immunity, inflammation, and hematopoiesis). The term “cytokine” as used herein includes “lymphokines”, “chemokines”, “monokines”, and “interleukins”. Examples of useful cytokines are GM-CSF, IL-1α, IL-10, IL-2, IL-3, IL-4, IL-5, IL-6, IL-7, IL-8, IL-10, IL-12, IL-15, IFN-α, IFN-0, IFN-γ, MIP-la, MIP-10, TGF-β, TNF-α, and TNF-β.

The proliferation and clonal expansion of T cells resulting from antigen-specific induction or stimulation of an immune response may be determined, for example, through incorporation of a non-radioactive assay such as a tritiated thymidine assay or MTT assay.

Cytotoxicity assays to determine CTL activity may be performed using any one of several techniques and methods routinely practiced in the art (e.g., Henkart et al. (2003) Fund. Immunol. 1127-1150). Additional description of methods for measuring antigen-specific T cell reactivity can be found in, for example, U.S. Pat. No. 10,208,086 and U.S. Pat. Publ. No. 2017/0209573.

e. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect encompassed by the present invention encompasses diagnostic assays for determining (e.g., detecting) the presence, absence, amount, and/or activity level of HA-1 or reactivity to HA-1 in the context of a biological sample (e.g., blood, serum, cells, or tissue) to thereby determine whether an individual afflicted with a disorder characterized by HA-1 expression is likely to respond to therapy, whether in an original state or as a recurrence. Such assays may be used for prognostic or predictive purpose to thereby prophylactically treat an individual prior to the onset or after recurrence of a disorder characterized by HA-1 expression.

The diagnostic methods described herein may furthermore be utilized to identify subjects having or at risk of developing a disorder associated with expression or lack thereof of HA-1. As used herein, the term “aberrant” includes an upregulation or downregulation of HA-1 from normal levels. Aberrant expression or activity includes increased or decreased expression or activity, as well as expression or activity which does not follow the normal developmental pattern of expression or the subcellular pattern of expression. For example, aberrant levels is intended to include the cases in which a mutation in the biomarker gene or regulatory sequence, or amplification of the chromosomal gene, thereof causes upregulation or downregulation of the biomarker of interest. As used herein, the term “unwanted” includes an unwanted phenomenon involved in a biological response, such as immune cell activity.

The assays described herein, such as the preceding diagnostic assays or the following assays, may be utilized to identify a subject having or at risk of developing a disorder associated with HA-1 misregulation. Thus, the present invention provides a method for identifying a disorder associated with aberrant or unwanted HA-1 regulation in which a test sample is obtained from a subject and HA-1 expression is detected, wherein the presence of HA-1 expression is diagnostic for a subject having or at risk of developing the disorder associated with aberrant or unwanted HA-1 expression. As used herein, a “test sample” refers to a biological sample obtained from a subject of interest. For example, a test sample may be a biological fluid (e.g., cerebrospinal fluid or serum), cell sample, or tissue, such as a histopathological slide of the tumor microenvironment, peritumoral area, and/or intratumoral area.

Furthermore, the prognostic assays described herein may be used to determine whether a subject may be administered an agent described herein to treat such a disorder associated with aberrant or unwanted HA-1 expression. For example, such methods may be used to determine whether a subject may be effectively treated with one or a combination of agents. Thus, the present invention provides methods for determining whether a subject may be effectively treated with one or more agents described herein for treating a disorder associated with aberrant or unwanted HA-1 expression.

The methods described herein may be performed, for example, by utilizing pre-packaged diagnostic kits comprising at least one antibody reagent described herein, which may be conveniently used, e.g., in clinical settings to diagnose patients exhibiting symptoms or family history of a disease or illness involving the biomarker of interest.

Furthermore, any cell type or tissue in which the biomarker of interest is expressed may be utilized in the prognostic assays described herein.

In addition, the prognostic methods described herein may be used to determine whether a subject may be administered a therapeutic agent to treat a disorder characterized by HA-1 expression.

f. Clinical Efficacy

Clinical efficacy may be measured by any method known in the art. For example, the response to a therapy relates to any response of the disorder characterized by HA-1 expression, e.g., a tumor, to the therapy, preferably to a change in the number of cancer cells, tumor mass, and/or tumor volume, such as after initiation of neoadjuvant or adjuvant chemotherapy. Tumor response may be assessed in a neoadjuvant or adjuvant situation where the size of a tumor after systemic intervention may be compared to the initial size and dimensions as measured by CT, PET, mammogram, ultrasound or palpation and the cellularity of a tumor may be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion such as percentage change in tumor volume or cellularity or by using a semi-quantitative scoring system such as residual cancer burden (Symmans et al. (2007) J. Clin. Oncol. 25:4414-4422) or Miller-Payne score (Ogston et al. (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy (e.g., after a few hours, days, weeks or preferably after a few months). A typical endpoint for response assessment is upon termination of neoadjuvant chemotherapy or upon surgical removal of residual tumor cells and/or the tumor bed. Additional guidance is provided in definitions of terms listed in Section I above.

### VII. Cell Therapy

In another aspect encompassed by the present invention, the methods include adoptive cell therapy, whereby genetically engineered cells expressing the provided molecules targeting an MHC-restricted epitope (e.g., cells expressing a binding protein (e.g., a TCR or CAR) or antigen-binding fragment thereof) are administered to subjects. Such administration may promote activation of immune cells (e.g., T cell activation) in an antigen-targeted manner, such that the cells of interest such as hyperproliferative cells, express an HA-1 antigen are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated (e.g., via adoptive cell therapy, such as by adoptive T cell therapy). In some embodiments, the cells or compositions are administered to the subject, such as a subject having or at risk for the disease or condition. In some embodiments, the methods thereby treat, e.g., ameliorate one or more symptom of the disease or condition.

Methods for administration of cells for adoptive cell therapy are known and may be used in connection with the provided methods and compositions (e.g., U.S. Pat. Publ. No. 2003/0170238, U.S. Pat. No. 4,690,915, Rosenberg (2011) Nat. Rev. Clin. Oncol. 8:577-585, Themeli et al. (2013) Nat. Biotechnol. 31:928-933, Tsukahara et al. (2013) Biochem. Biophys. Res. Commun. 438:84-89, and Davila et al. (2013) PLoS ONE 8: e61338).

In some embodiments, cell therapy (e.g., adoptive cell therapy, such as adoptive T cell therapy) may be carried out by autologous transfer, in which the cells are isolated and/or otherwise prepared from the subject who is to receive the cell therapy, or from a sample derived from such a subject. Thus, in some embodiments, the cells are derived from a subject, e.g., patient, in need of a treatment and the cells, following isolation and processing are administered to the same subject.

In some embodiments, the cell therapy (e.g., adoptive cell therapy, such as adoptive T cell therapy) may be carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical (syngeneic). In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject may be male or female and may be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The binding molecules, such as TCRs, antigen-binding fragments of TCRs (e.g., scTCRs) and chimeric receptors (e.g., CARs) containing the TCR, and cells expressing the same, may be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule or cell may depend on the type of disease to be treated, the type of binding molecule, the severity and course of the disease, whether the binding molecule is administered for preventive or therapeutic purposes, previous therapy, the patient's clinical history and response to the binding molecule, and the discretion of the attending physician. The compositions and molecules and cells are in some embodiments suitably administered to the patient at one time or over a series of treatments.

In some embodiments, cells may be administered at 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells transplanted may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiment, transplantation of at least about 0.1×10⁶, 0.5×10⁶, 1.0×10⁶, 2.0×10⁶, 3.0×10⁶, 4.0×10⁶, or 5.0×10⁶ total cells relative to an average size mouse is effective. For example, in some embodiments, cells, or individual populations of subtypes of cells, may be administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight Dosages may vary depending on attributes particular to the disease or disorder and/or patient and/or other treatments.

Engraftment of transplanted cells may be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3,4, 5, 6, 7, 8, 9, 10, 11, 12, 13, 14, 15, 16, 17, 18, 19, 20, 21, 22, 23, 24, 25, 26, 27, 28 days or may signal the time for tumor harvesting. Any such metrics are variables that may be adjusted according to well-known parameters in order to determine the effect of the variable on a response to anti-cancer immunotherapy. In addition, the transplanted cells may be co-transplanted with other agents, such as cytokines, extracellular matrices, cell culture supports, and the like.

Cells may also be administered before, concurrently with, or after, other anti-cancer agents.

Two or more cell types may be combined and administered, such as cell-based therapy and adoptive cell transfer of stem cells, cancer vaccines and cell-based therapy, and the like. For example, adoptive cell-based immunotherapies may be combined with the cell-based therapies encompassed by the present invention. In some embodiments, the cell-based agents may be used alone or in combination with additional cell-based agents, such as immunotherapies like adoptive T cell therapy (ACT). For example, T cells genetically engineered to recognize CD19 used to treat follicular B cell lymphoma. Immune cells for ACT may be dendritic cells, T cells such as CD8⁺ T cells and CD4⁺ T cells, natural killer (NK) cells, NK T cells, cytotoxic T lymphocytes (CTLs), tumor infiltrating lymphocytes (TILs), lymphokine activated killer (LAK) cells, memory T cells, regulatory T cells (Tregs), helper T cells, cytokine-induced killer (CIK) cells, and any combination thereof. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies may be further modified to express one or more gene products to further modulate immune responses, such as expressing cytokines like GM-CSF, and/or to express tumor-associated antigen (TAA) antigens, such as Mage-1, gp-100, and the like. The ratio of an agent encompassed by the present invention, such as cancer cells, to another agent encompassed by the present invention or other composition may be 1:1 relative to each other (e.g., equal amounts of 2 agents, 3 agents, 4 agents, etc.), but may modulated in any amount desired (e.g., 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

In some embodiments, for example, where the subject is a human, the dose includes fewer than about 1×10⁸ total binding protein (e.g., TCR or CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the cells or related compositions described herein, such as nucleic acids, host cells, pharmaceutical formulations, and the like, may be administered as part of a combination treatment, such as simultaneously with or sequentially with, in any order, another therapeutic intervention, such as another antibody or engineered cell or receptor or agent, such as a cytotoxic or therapeutic agent.

In some embodiments, the cells or related composition may be co-administered with one or more additional therapeutic agents or in connection with another therapeutic intervention, either simultaneously or sequentially in any order. In some contexts, the cells or related composition are co-administered with another therapy sufficiently close in time such that the cell populations enhance the effect of one or more additional therapeutic agents, or vice versa. In some embodiments, the cells or related composition are administered prior to the one or more additional therapeutic agents. In some embodiments, the cells or related composition are administered after to the one or more additional therapeutic agents.

In some embodiments, the biological activity of the cells or related composition is measured by any of a number of known methods once the cells or related composition are administered to a subject (e.g., a human). Parameters to assess include specific binding of an engineered or natural T cell or other immune cell to antigen, in vivo, e.g., by imaging, or in vitro/ex vivo, e.g., by ELISA or flow cytometry. In some embodiments, the ability of the cells to destroy target cells (cytotoxicity) may be measured using any suitable assay or method known in the art (e.g., Kochenderfer et al. (2009) J. Immunother. 32: 689-702 and Herman et al. (2004) J. Immunol. Meth. 285:25-40). In some embodiments, the biological activity of the cells also may be measured by assaying expression and/or secretion of certain cytokines, such as CD107a, IFNγ, IL-2, and TNF alpha. In some embodiments, the biological activity is measured by assessing clinical outcome, such as reduction in viral burden or load.

In some embodiments, cells are modified in any number of ways, such that their therapeutic or prophylactic efficacy is increased. For example, the binding protein (e.g., engineered TCR, CAR, or antigen-binding fragment thereof) expressed by the population may be conjugated either directly or indirectly through a linker to a targeting moiety. The practice of conjugating compounds to targeting moieties is well-known in the art (e.g., Wadwa et al. (1995) J. Drug Targeting 3:111 and U.S. Pat. No. 5,087,616).

Immune cells, such as cytotoxic lymphocytes, may be obtained from any suitable source such as peripheral blood, spleen, and lymph nodes. The immune cells may be used as crude preparations or as partially purified or substantially purified preparations, which may be obtained by standard techniques, including, but not limited to, methods involving immunomagnetic or flow cytometry techniques using antibodies.

In another aspect encompassed by the present invention, provided herein is a method for eliciting an immune response to a cell that expresses an HA-1 antigen, the method comprising administering to the subject cells described herein expressing a binding protein (e.g., engineered TCR, CAR, or antigen-binding fragment thereof) in effective amounts sufficient to elicit the immune response. In some embodiments, provided herein is a method for treatment or prophylaxis of a non-malignant disorder, a hyperproliferative disorder, or a relapse of a hyperproliferative disorder characterized by expression of an HA-1 antigen, the method comprising administering to the subject an effective amount of the cells described herein expressing a binding protein (e.g., engineered TCR, CAR, or antigen-binding fragment thereof). In one embodiment, the cells are administered systemically, such as by injection. Alternately, one may administer locally rather than systemically, for example, via injection directly into tissue, such as in a depot or sustained release formulation.

In some embodiments, the cells described herein expressing a binding protein (e.g., engineered TCR, CAR, or antigen-binding fragment thereof) may be used as active compounds in immunomodulating compositions for prophylactic or therapeutic treatment of a non-malignant disorder, a hyperproliferative disorder, or a relapse of a hyperproliferative disorder characterized by expression of an HA-1 antigen. In some embodiments, HA-1-primed antigen-presenting cells may be used for generating lymphocytes (e.g., CD8⁺ T lymphocytes, CD4⁺ T lymphocytes, and/or B lymphocytes), for further use in adoptive transfer to the subject with the cells described herein expressing a binding protein (e.g., engineered TCR, CAR, or antigen-binding fragment thereof).

In some embodiments, the cells described herein expressing a binding protein (e.g., engineered TCR, CAR, or antigen-binding fragment thereof), either alone or in combination with the lymphocytes, may be administered to a subject for eliciting an immune response, particularly for eliciting an immune response to cells are expressing an HA-1 antigen.

As described above, single or multiple administrations of the cells described herein expressing a binding protein (e.g., engineered TCR, CAR, or antigen-binding fragment thereof) cells, either alone or in combination with the lymphocytes, may be carried out with cell numbers and treatment being selected by the care provider (e.g., physician). Similarly, the cells, either alone or in combination with lymphocytes, may be administered in a pharmaceutically acceptable carrier. Suitable carriers may be growth medium in which the cells were grown, or any suitable buffering medium such as phosphate buffered saline. Cells may be administered alone or as an adjunct therapy in conjunction with other therapeutics.

### VIII. Kits and Devices

The present invention also encompasses kits and devices. For example, the kit or device may comprise binding proteins, nucleic acids or vectors comprising sequences encoding binding proteins, host cells comprising nucleic acids or vectors and/or expressing the binding proteins as described herein, stable MHC-peptide complexes, adjuvants, detection reagents, and combinations thereof, packaged in a suitable container and may further comprise instructions for using such reagents. The kit or devicemay also contain other components, such as administration tools packaged in a separate container. The kit or device may be promoted, distributed, or sold as a unit for performing the methods encompassed by the present invention.

The disclosure is further illustrated by the following examples which should not be construed as limiting.

### EXAMPLES

### Example 1: Materials and Methods for Example 2

A. Human Peripheral Blood Mononuclear Cell Collection

HLA-A*02:01-positive healthy donor leukopaks were collected by HemaCare (Los Angeles, CA) or StemExpress (Placerville, CA) using their IRB-approved protocols. Peripheral blood mononuclear cells (PBMCs) were isolated from fresh leukopaks from HemaCare by density gradient centrifugation using Lymphocyte Separation Medium (Corning, Corning, NY). PBMCs contained in the lymphocyte layer were collected following centrifugation, washed 3 times with DPBS (Cytiva, Marlborough, MA), and counted. PBMCs were isolated from StemExpress leukopaks either by density gradient centrifugation as above, or using a Custom Leukopak PBMC Isolation kit (Miltenyi Biotec, Auburn, CA) on the MultiMACS Cell24 Separator Plus instrument, version 3 (Miltenyi Biotec) per the manufacturer's instructions. Isolated PBMCs were frozen in CryoStor CS10 (StemCell Technologies, Cambridge, MA) and stored in liquid nitrogen.

b. HA-1 Genotyping

DNA was extracted from PBMCs of A*02:01-expressing healthy donors using the GeneJET Genomic DNA Purification Kit (Thermo Fisher Scientific, Waltham, MA) according to the manufacturer's instructions. PCR was performed using primers (5′ AGGACATCTCCCATCTGCTG 3′ and 5′ TTGAGCCAGTGTACGCTCAG 3′) to amplify the genomic region containing the HA-1 SNP (RS_1801284) using Phusion High-Fidelity PCR Master Mix (Thermo Fisher Scientific), with cycling conditions as follows: denaturation at 98° C. for 3 minutes, followed by 37 cycles at 98° C. for 5 seconds, 68° C. for seconds, and 72° C. for 30 seconds, and final extension at 72° C. for 5 minutes. PCR products were purified using the Monarch PCR and DNA Cleanup Kit (New England Biolabs, Ipswich, MA) according to the manufacturer's instructions. DNA concentration was determined using a Qubit 4 Fluorometer (Invitrogen, Carlsbad, CA), and sequencing of the purified products was performed by GENEWIZ, Inc. (Cambridge, MA).

c. TCR Discovery Screens

(i) DC Culture

Monocyte isolation was performed on day-4 using PBMCs isolated from HLA-A*02:01-positive healthy HA-1-negative (RS_1801284, G/G) donors with the EasySep Human CD14 Positive Selection Kit II (StemCell Technologies) according to the manufacturer's instructions. Purity and costimulatory molecule expression were assessed using fluorescently-labeled antibodies specific for CD14 (M5E2, BioLegend, Dedham, MA), HLA-A2 (BB7.2, BioLegend), CD80 (2D10, BioLegend), CD83 (HB15e, BioLegend), and CD86 (IT2.2, BioLegend); CD14 expression was >90%. CD14⁺ monocytes were resuspended in AIM-V media (Thermo Fisher Scientific) supplemented with recombinant human GM-CSF and IL-4 (R&D Systems, Minneapolis, MN) at final concentrations of 800 IU/mL and 1000 IU/mL, respectively. On day −2, recombinant human TNF-α (10 ng/mL), IL-6 (1000 IU/mL), and IL-1P (2 ng/mL) (R&D Systems) as well as PGE2 (1 μg/mL, StemCell Technologies) were added to cultured monocytes.

(ii) CD8 Naïve T Cell Isolation

On day −1, autologous CD8 naïve T cells were isolated from PBMCs from HLA-A*02:01-expressing healthy HA-1-negative (RS_1801284, G/G) donors using the EasySep Human Naïve CD8⁺ T Cell Isolation Kit II (StemCell Technologies) according to the manufacturer's instructions. Purity was assessed using fluorescently-labeled antibodies specific for CD8α (HIT8a, BioLegend), CD45RO (UCHL1, BioLegend), CD45RA (HI100, BioLegend), CD56 (5.1H11, BioLegend), CD57 (HCD57, BioLegend), and CCR7 (G043H7, BioLegend); purity of naive CD8α⁺ T cells was >90%. Cells were rested overnight at 37° C., 5% CO₂ in RPMI-1640 (ATCC, Manassas, VA) containing 10% human serum (Sigma Aldrich, St. Louis, MO) 1% penicillin-streptomycin (Thermo Fisher Scientific), and 50 μM β-mercaptoethanol (MP Biomedicals, Santa Ana, CA), supplemented with 10 ng/ml recombinant human IL-7 (R&D Systems).

(iii) Co-Culture

On day 0, CD8 T cell purity was reassessed using the identical antibody panel as on day 3 and DC maturation was confirmed by upregulation of HLA-A2, CD8β, CD83, and CD86, and downregulation of CD14. DCs were pulsed with 1 μM HA-1 peptide (VLHDDLLEA, GenScript [Piscataway, NJ]) for 3 hours at 37° C., 5% CO₂. Pulsed DCs were co-cultured with rested CD8 naïve T cells in T cell media supplemented with recombinant human IL-12 (10 ng/mL) and IL-21 (60 ng/mL) (R&D Systems). Co-cultures were supplemented with recombinant human IL-7 and IL-15 (R&D Systems) between days 3 and 10. Dextramer staining for HA-1-specific cells was performed on day 11, using A*02:01 HA-1 (VLHDDLLEA) dextramer (Immudex, Copenhagen, Denmark), CD8α and TCRα/β (IP26, BioLegend), and DAPI (Thermo Fisher Scientific) according to the manufacturer's instructions.

(iv) Rapid Expansion Protocol (REP)

Following identification of wells containing HA-1-specific cells, T cell clones were harvested and co-cultured with irradiated PBMC feeder cells on day 12 in the presence of 100 ng/mL anti-CD3 (OKT3, Invitrogen), and 50 IU/mL recombinant human IL-2 (Sigma Aldrich) in T cell media at 37° C., 5% CO₂ for 7 days.

(v) Antigen-Specific Cell Sorting

On day 19, cells were collected post-REP, stained with A*02:01 HA-1 (VLHDDLLEA) dextramer, with antibodies specific for CD8α and TCRα/β, and with DAPI as on day 11, and HA-1-positive cells (CD8α⁺, DAPI⁻, TCRα/β⁺, HA-1⁺) were sorted using a Sony SH800S cell sorter (Sony Biotechnology, San Jose, CA) or a BD FACSAria cell sorter (Becton Dickinson, Franklin Lakes, NJ). Sorted cells were subjected to single cell TCRα/β sequencing using the 10× Genomics platform (Pleasanton, CA).

(vi) Flow Cytometry

Cells were acquired using a CytoFLEX flow cytometer (Beckman Coulter, Indianapolis, IN) and analyzed using FlowJo software (version 10, TreeStar, Ashland, OR). Cell sorting was performed using a Sony SH800S cell sorter (Sony Biotechnology) or a BD FACSAria cell sorter (Becton Dickinson).

d. Single Cell TCRα/β Sequencing Using the 10× Genomics Platform Single-cell TCR-seq (scTCR-seq) libraries were prepared according to the 10× Genomics Single Cell V(D)J Reagent Kit (v1) protocol (10× Genomics). A target number of 10,000 cells per sample was captured in droplets (GEMs) within the 10× Genomics Chromium instrument before undergoing reverse transcription. Following reverse transcription, GEMs were broken, and barcoded cDNA was purified from the samples using Silane magnetic beads. cDNA was amplified as follows: 98° C. for 45 seconds; 13 cycles of 98° C. for 20 seconds, 67° C. for 30 seconds, 72° C. for 1 minute; 72° C. for 1 minute). Following sample purification with 0.6× SPRIselect beads (Beckman Coulter), 2 uL of each library was used for TCR sequence enrichment. TCR sequence enrichment consisted of 2 rounds of PCR to amplify both the TCRα and TCRβ chain transcripts. TCR-enriched libraries were subsequently fragmented, end-repaired, and amplified with indexing primers. The fully-assembled libraries were sequenced on an Illumina NextSeq instrument (Illumina, San Diego, CA) using a High Output v2.5 kit (150 cycles) with read lengths: 26 bp (read 1), 8 bp (i7 index), and 98 bp (read 2). Sequenced scTCRseq reads were processed using the cellranger 3.1.0 pipeline. Reads were aligned to the GRCh38 reference genome, and the cellranger vdj module was used to annotate TCR consensus sequences.

e. Lentiviral Packaging and Quantification of Lentiviral Titer

Lenti-X cells were plated at 75% confluency and transfected using jetPRIME transfection reagent (Polyplus, Illkirch, France). Briefly, HA-1 constructs were mixed with packaging plasmids (pREV/pTAT/pVSVG/pGAGPOL) and incubated with jetPRIME reagent according to the manufacturer's protocol, and Opti-Pro SFM medium was added at 24 hours post-transfection. Viral supernatants were harvested 48 hours after transfection and were concentrated using either Vivaspin 20 centrifugal concentrators or Vivaflow 50 cassettes (Sartorius, Bohemia, NY). Lentiviral titer was determined by either GFP or TCRα/β expression using a TCR^{-/-} Jurkat cell line. To quantify viral titers, TCR^{-/-} Jurkat cells were transduced with serial dilutions of HA-1-specific TCR-expressing viruses, and 48 to 72 hours post-transduction, GFP or TCR/P expression was assessed by flow cytometric analysis. Samples were analyzed using a CytoFLEX flow cytometer (Beckman Coulter). Titer was calculated as the percentage of cells expressing GFP or TCRα/P as TU/ml using the formula:

TU/ml=(% of GFP⁺or TCRα/β⁺)×(number of cells used in transduction)×(dilution factor)×1000

f. Evaluating HA-1-Specific TCR Function

(i) Engineering T Cells to Express HA-1-Specific TCRs

Pan T cells or CD4⁺ T cells were isolated from HLA-A*02:01-positive healthy HA-1-negative (RS_1801284, G/G) donor PBMCs using the EasySep Human T Cell isolation kit or the EasySep Human CD4⁺ T cell isolation kit (StemCell Technologies) as per the manufacturer's instructions. Isolated T cells were activated with ImmunoCult CD3/CD28/CD2 T cell activator cocktail (StemCell Technologies) and cultured overnight in complete T cell media (X-VIVO 15 [Lonza, Morristown, NJ] supplemented with 5% human serum [Sigma Aldrich], 1% penicillin-streptomycin [Thermo Fisher Scientific], 1× GlutaMax supplement [Thermo Fisher Scientific], 5 ng/mL IL-7 [R&D Systems] and 50 IU/mL IL-2 [Sigma Aldrich]). 24 hours post-activation, T cells were transduced with HA-1 TCR viral supernatants at an MOI (multiplicity of infection) of 5. 24 hours post-transduction, T cells were washed and transferred to G-Rex plates (Wilson Wolf, New Brighton, MN) or VECELL 96-well plates (Cosmo Bio, Carlsbad, CA), and expanded for a total of 7-8 days post-activation. T cell cultures were supplemented with fresh IL-2 (50 IU/mL) and IL-7 (5 ng/mL) every 2-3 days. Where indicated, enrichment of CD34⁺ transduced cells was performed using the Miltenyi CD34 Microbead Kit as per the manufacturer's instructions (Miltenyi Biotec). After enrichment, T cells were further expanded for 2-3 days in complete T cell media followed by flow cytometric analysis of purity and expression of the HA-1-specific TCR.

(ii) Flow Cytometry of Engineered HA-1 TCR-Transduced Pan T Cells

Engineered pan T cells were stained with HA-1 (VLHDDLLEA) dextramer and HA-1 (VLRDDLLEA) or MART-1 (ELAGIGILTV) (Immudex) dextramer, TCR α/β PE-Cy7 (IP26, BioLegend), CD8 PerCP-Cy5.5 (HIT8a, BioLegend) and CD4 APC-Cy7 (OKT4, Biolegend), CD34 APC (QBEND/10, R&D Systems) or CD34 FITC (QBEND/10, Invitrogen) and DAPI as per the manufacturers' instructions. Cells were then analyzed on the CytoFLEX flow cytometer (Beckman Coulter) and analyzed using FlowJo software (version 10, TreeStar) or CytExpert software (Beckman Coulter).

(iii) Cell Lines

The T lymphoblast cell line T2 (ATCC CRL-1992), AML cell lines TF-1 (ATCC CRL-2003) and THP-1 (ATCC TIB202), the lymphoma cell line SU-DHL1 (ATCC CRL 2955) and the acute T cell leukemia cell line Jurkat, (clone E6-1, ATCC TIB-152) were purchased from the American Type Culture Collection (ATCC). The lymphoma cell line SUP-M2 (ACC 509) was purchased from Deutsche Sammlung von Mikroorganismen und Zellkulturen (DSMZ). The Lenti-X cell line (632180) was purchased from Takara Bio (Mountain View, CA). T2 cells were cultured in IMDM+20% heat-inactivated FBS. TF-1 cells were cultured in RPMI 1640+10% heat-inactivated FBS+2 ng/mL recombinant human GM-CSF. THP-1 cells were maintained in RPMI 1640+10% heat-inactivated FBS +0.05 mM 2-mercaptoethanol. SU-DHL1 and SUP-M2 cells were maintained in RPMI 1640+10% and 20% heat-inactivated FBS, respectively. Jurkat cells were cultured in RPMI 1640+10% heat-inactivated FBS+1% penicillin-streptomycin. Lenti-X cells were cultured in DMEM medium (Thermo Fisher Scientific) supplemented with 10% FBS and 1% penicillin-streptomycin. HEK 293T cells were maintained in DMEM medium (Gibco), supplemented with 10% heat-inactivated fetal bovine serum (FBS), 100 IU/mL penicillin, and 100 μg/mL streptomycin.

(iv) Generation of Stable Cell Lines Expressing Incucyte® Nuclight Red

T2, TF-1, THP-1, SU-DHL1 and SUP-M2 cells were transduced with Incucyte® NucLight Red Lentivirus Reagent (EF-1α promoter, puromycin selection) (Sartorius) in serum-free media at an MOI between 3 and 5. 24 hours post transduction, cells were washed and resuspended in their respective cell line media and cultured at 37° C., 5% CO₂. 3 days post-transduction, puromycin (Gibco) was added to the cultures at a pre-determined concentration (ranging from 0.5 ug/mL to 1 ug/mL) to select for transduced cells. Cultures were expanded under puromycin selection until they were at least 98% Nuclight Red-positive as determined by flow cytometric analysis.

(v) Generation of a TCR Knockout Jurkat Cell Line

The TCR^{-/-} Jurkat cell line was generated by electroporating CRISPR RNPs targeting the TCRα chain (Guide sequence: AGAGTCTCTCAGCTGGTACA, S.p. Cas9 Nuclease V3, IDT, Coralville, IA) (Ren J. et. al. Clin Cancer Res. 2017). 5 days post-electroporation, cells were stained with anti-human TCRα/β (clone IP26, BioLegend), and the TCR^{-/-} population was sorted on a Sony SH800S cell sorter.

(vi) In Vitro Cytotoxicity Assay

In vitro cytotoxicity assays were performed in 384-well plates pre-coated with poly-L-lysine (Greiner, Monroe, NC) or in 96-well flat-bottom tissue culture plates coated with poly-L-ornithine (Sigma Aldrich) for 30 minutes at RT, after which the coating solution was removed and plates were allowed to dry for another 30 minutes at RT. Where indicated, T cells were co-cultured with Nuclight Red-expressing THP-1, SUP-M2, TF-1, SU-DHL1 or peptide-pulsed T2 cells (pulsed with concentrations ranging from 10 ng/ml to 10 μg/ml of HA-1 peptide [VLHDDLLEA, Genscript] concentrations) at E:T ratios ranging from 20:1 to 2.5:1. Data were acquired on an Incucyte S3 instrument (Sartorius) and cell growth was quantified on the Incucyte S3 as a readout of cytotoxicity of the T cells.

(vii) Cytokine Production Assay

T cells were co-cultured with Nuclight Red-expressing THP-1, SUP-M2, TF-1, SU-DHL1 and peptide-pulsed T2 cells (1 ng/mL HA-1 peptide [VLHDDLLEA, Genscript]) at an E:T of 1:1. Supernatants were harvested 24 hours later and frozen at −80° C. Supernatants were thawed and loaded on a multiAnalyte cartridge (ProteinSimple, San Jose, CA) to evaluate the levels of IFN-γ, TNF-α, IL-2 and granzyme B using the Ella instrument (ProteinSimple).

(viii) Proliferation Assay

T cells were labeled with 2.5 μM CellTrace™ Violet (Thermo Fisher Scientific) as per the manufacturer's instructions. T cells were co-cultured with Nuclight Red-expressing THP-1, SUP-M2, TF-1, SU-DHL1 and peptide-pulsed T2 cells (1 ng/mL HA-1 peptide [VLHDDLLEA, Genscript, Piscataway, NJ]) at an E:T of 1:1. After 96 hours, cells were harvested and stained with Fixable viability dye eFluor 660 (FisherScientific), CD4 PerCP-Cy5.5 (OKT4, BioLegend) and CD8 APC-Cy7 (HIT8a, BioLegend) antibodies. Cells were washed with EasySep™ buffer and fixed with BD Cytofix™ fixation buffer (BD Biosciences, Franklin Lakes, NJ). CountBright Absolute Counting Beads (Thermo Fisher Scientific) were diluted in EasySep™ buffer and added to samples prior to acquisition on the CytoFLEX flow cytometer to assess dilution of CellTrace™ Violet as an indicator of T cell proliferation. Data were analyzed using FlowJo software (version 10, TreeStar), and absolute counts of divided CD8 and CD4 T cells were determined using the formula:

#CD8 or CD4T cells in sample=(#CD8 or CD4T cell events in gate/#bead events in gate)×lot-specific assigned bead count

g. Alloreactivity Screens

(i) Generation of 96-Well-Based MHC-Expressing Arrays

Endogenous HLA-A/B/C were knocked out in HEK293T cells using CRISPR. Guide RNAs were designed against sequences conserved across the HLA-A, HLA-B and HLA-C loci using the multicrispr.net tool (Prykhozhij et al., Plos One, 2015). The following guides were selected: CRISPR-ALL-1: CGGCTACTACAACCAGAGCG, CRISPR-ALL-2: AGATCACACTGACCTGGCAG, CRISPR-ALL-3: AGGTCAGTGTGATCTCCGCA. gRNAs were cloned into the LentiCRISPR V2 vector using BsmBI sites. HEK 293T cells were transfected with plasmid guide constructs using Mirus TransIT (Mirus Bio, Madison, WI). After 7 days, MHC-negative cells were sorted using a pan-MHC antibody (BioLegend). Single cell clones were expanded, and the absence of MHC was verified by flow cytometry and western blot.

To express HA-1 in HEK239T cells, MHC-null HEK293T cells were transduced with a 90-mer construct expressing HA-1 (pHAGE-CMV-FHA-HAIH-IRES-Puro). Transduced wells were selected with puromycin for one week. To generate an MHC-expressing array, MHC-null HEK 293T cells with or without HA-1 expression were then transduced with the most common 110 most common MHCs (pHAGE-EFIa-MHC—UBC-NAT) in individual wells in 96-well plates. Transduced cells were selected with nourseothricin for one week. One hundred and four (104) MHCs have both the HA-1- and HA-1⁺ versions successfully established and were subject to the alloreactivity screen. Cells expressing the most common 108 MHCs with or without the HA-1 90-mer were passaged and stored in 96-well plates as an array.

(ii) Co-Culture for Alloreactivity Screens

Human CD8⁺ T cells expressing TSC-100 were generated as described in the Safety Screens section.

On day 0, target cells in the 96-well array were passaged and seeded. On day 1, human CD8 T cells expressing TSC-100 were added at an E:T of 3:1 and incubated with target cells overnight. On day 2, supernatant was harvested and stored at −80° C. The IFN-γ concentration in the samples was measured by an ELISA assay using the Ella machine and the IFN-γ 3rd Generation Kit (ProteinSimple).

h. Safety Screens

(i) Engineering T Cells to Express HA-1-Specific TCRs

CD8⁺ T cells from HA-1-negative or A*02:01-negative donors were isolated using the StraightFrom® Leukopak® CD8 Microbead Kit (Miltenyi Biotec) according to the manufacturer's instructions. Isolated cells were frozen and stored in liquid nitrogen. On day −1, CD8 T cells were thawed and rested overnight in T cell medium (RPMI 1640 supplemented with 10% heat-inactivated fetal bovine serum [FBS], 100 IU/mL penicillin, 100 μg/mL streptomycin, recombinant human IL-2 [50U/mL, PeproTech, Cranbury, NJ], recombinant human IL-15 [5 ng/mL, R&D Systems], and recombinant human IL-7 [5 ng/mL, (R&D Systems]). On day 0, the CD8 T cells were activated using ImmunoCult CD3/CD28/CD2 T cell activator cocktail (StemCell Technologies). On day 1, cells were washed and resuspended in T cell medium with IL-2, IL-7, and IL-15 and transduced with lentiviral particles to express HA-1-reactive TCRs. On day 2, cells were washed and resuspended in T cell medium with IL-2, IL-7, and IL-15 for expansion until day 5. On day 5, cells were harvested and resuspended in MACS buffer (Miltenyi Biotec) and incubated with FcR blocking reagent (100 uL/mL, StemCell Technologies) for 5 minutes at room temperature. Cell concentration was adjusted to 60e6 cells/mL and A*0201 HA-1 (VLHDDLLEA) dextramer PE (Immudex) was added at a 1:25 dilution for 10 minutes at room temperature. HA-1 dextramer PE-positive cells were isolated using Anti-PE Microbeads (Miltenyi Biotec) according to the manufacturer's instructions. Isolated cells were resuspended in T cell medium with IL-2, IL-7, and IL-15 and expanded until day 12, at which point cells were frozen.

(ii) Transduction of HEK 293T Cells with a Genome-Wide Peptide Library

MHC-null HEK 293T cells were engineered to express HLA-A*02:01, a fluorescent reporter and a cell-surface reporter, both activated by T cell killing. Reporter cells were transduced to express a library comprising >600,000 overlapping tiles of peptides comprised of 90 amino acids each, representative of the human proteome.

(iii) Sorting of Reporter Cells Following Co-Culture

CD8⁺ T cells engineered with the relevant TCRs were expanded by co-culturing with mitomycin C-treated (50 μg/mL, 30 min) allogeneic PBMCs in the presence of 0.1 μg/mL anti-CD3 (OKT3, eBioscience) and 50 U/mL recombinant IL-2 (Peprotech). After 7 days of expansion, the cells were collected and co-cultured with library-transduced reporter cells and incubated at 37° C. for 4 h. After incubation, cells were harvested by trypsinization and labeled with Annexin V magnetic microbeads (Miltenyi Biotec) according to the manufacturer's instructions. Annexin-labeled cells were isolated using an AutoMACS Pro (Miltenyi Biotec), and reporter-positive cells were sorted using a MoFlo Astrios EQ cell sorter (Beckman Coulter).

(iv) Genomic DNA Isolation and Sequencing Library Preparation

Genomic DNA (gDNA) was extracted from sorted cells using the GeneJET Genomic DNA Purification Kit (Thermo Fisher Scientific). The antigen cassette was amplified from the extracted gDNA by PCR then appended with sequencing adaptors and sample-specific index sequences in a second PCR reaction. Amplicons were sequenced on an Illumina NextSeq using the standard Illumina sequencing primer.

(v) Sequence Alignment and Epitope Identification

Nucleotide sequences were mapped to individual nucleotide tiles. The proportion of read counts for each tile was calculated for each screen replicate (n=8) and for the input for each pool of transduced reporter cells, and enrichments of each tile were calculated by dividing the proportion of the tile in the screen replicate by the proportion of the tile in the input library. A modified geometric mean of the enrichment of an identical tile across the 8 screen replicates was used to identify reproducible screen hits. Specific MHC-binding epitopes for each tile above the threshold of 2-fold enrichment were predicted using NetMHC4.0. Candidate epitopes for each tile were selected by identifying predicted strong-binding epitopes shared across overlapping adjacent and redundant tiles that were enriched in the screen.

i. In Vitro Cytotoxicity of Blood Cells Derived from Healthy Donors and Patients Afflicted with a Cancer, Such as AML or ALL

(i) Target Cells

Primary tumor samples were obtained from Tissue Solution. HLA-A*02:01-positive status was confirmed by flow cytometry and HA-1 genotype status was determined as described above. Healthy PBMCs were obtained from healthy donor leukopaks processed as described above.

The primary samples were thawed the day before the initiation of the co-culture and allowed to rest overnight in complete T cell media (X-VIVO 15 [Lonza, Morristown, NJ]) supplemented with 5% human serum [Sigma Aldrich], 1% penicillin-streptomycin [Thermo Fisher Scientific], 1× GlutaMax supplement [Thermo Fisher Scientific], 5 ng/mL IL-7 [R&D Systems] and 50 IU/mL IL-2 [Sigma Aldrich]). On D0 of co-culture, primary cancer cells and healthy PBMCs were washed with DPBS and labeled with 20 nM CellTrace™ FarRed (Thermo Fisher) following manufacturer's instructions. Labeled primary target cells were resuspended in cytokine-free T cell media.

(ii) Effector Cells

Memory T cells were isolated from HLA-A*02:01-negative healthy leukopaks from Discovery Life Sciences (Huntsville, AL) using a CliniMACS Prodigy instrument (Myltenyi biotec). CD45RA depletion, followed by the concurrent positive selection of CD4 and CD8, were performed following standard procedure to generate memory pan T cells. The isolated memory T cells obtained were frozen down until further processing. Isolated memory T cells were thawed and activated with ImmunoCult CD3/CD28/CD2 T cell activator cocktail (StemCell Technologies) and cultured overnight in complete T cell media (X-VIVO 15 [Lonza, Morristown, NJ] supplemented with 5% human serum [Sigma Aldrich], 1% penicillin-streptomycin [Thermo Fisher Scientific], 1× GlutaMax supplement [Thermo Fisher Scientific], 5 ng/mL IL-7 [R&D Systems] and 50 IU/mL IL-2 [Sigma Aldrich]). 24 hours post-activation, T cells were transduced with TSC-100 viral supernatants at an MOI (multiplicity of infection) of 5. 24 hours post-transduction, T cells were washed and transferred to G-Rex plates (Wilson Wolf, New Brighton, MN), and expanded for a total of 7 days post-activation. T cell cultures were supplemented with fresh IL-2 (50 IU/mL) and IL-7 (5 ng/mL) every 2-3 days. CD34⁺ transduced cells were enriched on D7 using the Miltenyi CD34 Microbead Kit as per the manufacturer's instructions (Miltenyi Biotec). After enrichment, T cells were further expanded for 7 days in complete T cell media with fresh IL-2 (50 IU/mL) and IL-7 (5 ng/mL) supplemented every 2-3 days followed by flow cytometric analysis of purity and expression of the HA-1-specific TCR. TSC-100 engineered T cells and non-transduced control T cells were frozen down on D14.

(iii) Co-Culture and Flow-Based Cytotoxicity Measurement

The effector cells were thawed the day before the initiation of the co-culture and allowed to rest overnight in complete T cell media. On D0 of coculture, effector cells were washed with DPBS and labeled with 2.5 μM CellTrace™ Violet following manufacturer's instructions. Labeled effector cells were resuspended in cytokine-free T cell media.

Labeled T cells (50,000 cells) and target cells (50,000) were co-cultured in 200 uL in a 96-well round bottom for 24h. Following incubation, cells were spun down, washed with EasySep-buffer, and stained with a fixable viability dye Live/Dead.Near IR (Thermo Fisher) following manufacturer's instructions. Samples were run on a Cytoflex S instrument and data were analyzed using FlowJo v.10 software. Effector cells and primary target cells were identified on the basis of CellTrace™ violet and CellTrace™ farRed status, respectively. The relative viability of the target cells was measured by gating on the CellTrace™ FarRed events and reporting the proportion of Live/Dead negative events. Data are expressed as viability of target cells relative to the non-transduced control T cell co-culture condition.

j. Materials

### Cell Lines

Lenti-X: Takara, 632180
Jurkat: ATCC, TIB-152
HEK 293T cells: ATCC, CRL-3216
T2: ATCC, CRL-1992
TF-1: ATCC, CRL-2003
THP-1: ATCC, TIB202
SU-DHL1: ATCC CRL, 2955
SUP-M2: ACC 509, DSMZ

### Media and Supplements

X-VIVO 15, serum-free hematopoietic cell medium, with L-Glutamine, gentamycin and phenol red: Lonza, 04-418Q
Human male AB serum (heat-inactivated): Sigma Aldrich, H3667-100ML
Penicillin streptomycin: Gibco, 15149-122
GlutaMAX supplement: Fisher Scientific, 35050061
RPMI-1640 medium: ATCC, 30-2001
Fetal bovine serum (FBS), heat-inactivated: Gibco, A3840102
IMDM: ATCC, 30-2005
RPMI medium 1640 (lx) [+] 4.5 g/L D-glucose, [+] 2.383 g/L HEPES buffer, [+] L-glutamine, [+] 1.5 g/L sodium bicarbonate, [+] 110 mg/L sodium pyruvate: Gibco, A10491-01
AIMV medium: [+] L-glutamine, [+] 50 μg/mL streptomycin sulfate, [+] 10 μg/mL gentamicin sulfate: Gibco, 12055-083
30% BSA in PBS: Sigma Aldrich, A9576-50ML
β-Mercaptoethanol: MP Biomedicals, 02190242
DMEM medium (1×) [+] 4.5 g/L D-glucose, [+] L-glutamine, [+] 3.7 g/L sodium bicarbonate: Thermo Fisher Scientific, 11965084
DMEM (1×) [+] 4.5 g/L D-glucose, [+] L-glutamine, [−] Sodium Pyruvate: Gibco, 11965
OptiPRO SFM medium: Thermo Fisher Scientific, 12309019

### Buffers

EasySep buffer: StemCell Technologies, 20144
DPBS (no Ca2+/Mg2+): Gibco, 14190-122
DPBS (no Ca2+/Mg2+): Cytiva, SH30028.03
AutoMACS running buffer: Miltenyi, 130-091-221

### Cytokines

Recombinant human IL-2: Sigma Aldrich, 11147528001
Recombinant human IL-2: PeproTech, 200-02
Recombinant human IL-12: R&D Systems, 219-IL-005
Recombinant human IL-15: R&D Systems, 247-ILB-005
Recombinant human GM-CSF: R&D Systems, 215-GM-050
Recombinant human IL-4: R&D Systems, 204-IL-050
Recombinant human TNF-α: R&D Systems, 210-TA-050
Recombinant human IL-10/IL-1F2: R&D Systems, 201-LB-025
Recombinant human IL-6: R&D Systems, 206-IL-050
PGE2: StemCell Technologies, 72194

### Kits

EasySep™ Human T Cell Isolation Kit: StemCell Technologies: 17951
EasySep™ Human CD4⁺ T Cell Isolation Kit: StemCell Technologies: 17952
EasySep™ Human Naïve CD8⁺ T Cell Isolation Kit II: STEMCELL Technologies, 17968
EasySep™ Human CD14 Positive Selection Kit II: STEMCELL Technologies, 17858
StraightFrom® Leukopak® CD8 Microbead Kit: Miltenyi Biotec, 130-117-019
CD34 Microbead Kit: Miltenyi Biotec, 130-046-702
Anti-PE microbeads: Miltenyi Biotec, 130-105-639
LS columns, Miltenyi Biotec, 130-042-401
Ella simple plex kit for 32 samples: ProteinSimple: SPCKC-PS-003027
Simple Plex Human IFN-gamma (3rd Gen) Cartridge: ProteinSimple, SPCKB-PS-002574

### Antibodies and Staining Reagents

Anti-human CD8α PerCβ-Cy5.5 (clone: HIT8a): BioLegend: 300924
Anti-human CD8α APC-Cy7 (clone: HIT8a): Biolegend: 300926
Anti-human CD4 APC-Cy7 (Clone: OKT4): BioLegend: 317418
Anti-human CD4 PerCP-Cy5.5 (clone OKT4): Biolegend: 317428
Anti-human CD56 PE (clone: 5.1H11): BioLegend: 362508
Anti-human CD57 PE (clone: HCD57): BioLegend: 322312
Anti-human TCRα/β PE-Cy7 (clone: IP26): BioLegend: 306720
Anti-human CD34 FITC (clone: QBEND/10): Invitrogen: MA1-10204
Anti-human CD34 APC: (Clone: QBEND/10): RD Systems, FAB7227A
Anti-human CD45RO FITC (clone: UCHL1): BioLegend, 304204
Anti-human CD45RA APC (clone: HI100): BioLegend, 304112
Anti-human CCR7 BV605 (clone: G043H7): BioLegend, 353224
Anti-Human CD14 Alexa Fluor 488® (clone: M5E2): BioLegend, 301811
Anti-human CD83 PE-Cy7 (clone: HB15e): BioLegend, 305326
Anti-human HLA-A2 PE (clone: BB7.2): BioLegend, 343306
Anti-human CD8β APC (clone: 2D10): BioLegend, 305220
Anti-human CD86 Brilliant Violet 605™ (clone IT2.2): BioLegend, 305430
Anti-human TCRα/β FITC (clone: IP26): BioLegend, 221058
Anti-human TCRα/β APC (clone IP26): BioLegend, 306718
Anti-human HLA-A,B,C APC Antibody: BioLegend, 311410
Anti-Human CD32 (Fc gamma RII) Blocker: STEMCELL Technologies, 18520
Cell Trace Violet (CTV): Fisher Scientific: C34571
Fixable viability Dye eFluor 660 (APC Channel) Thermo Fisher Scientific: 65-0864-14
LIVE/DEAD™ Fixable Near-IR Dead Cell Stain Kit: Thermo Fisher Scientific, L34976A
DAPI: Thermo Scientific: Thermo Scientific, 62248
CountBright™ Absolute Counting Beads: Fisher Scientific: C36950
Cytofix: BD Biosciences, 554655

### Peptides

MART1 (ELAGIGILTV): GenScript
HA-1 (VLHDDLLEA): GenScript

### Dextramers

HLA-A*02:01 VLHDDLLEAN HA-1 Dextramer: Immudex, WB2622-PE
HLA-A*02:01 VLRDDLLEAN HA-1 dextramer, WB5421-APC
HLA-A*02:01 ELAGIGILTV MART1 dextramer: Immudex, WB2162-APC

### CRISPR-Cas9 and Electroporation Reagents

Alt-R® S.p. Cas9 Nuclease V3 (Lot #0000417827): IDT, 1081059
Alt-R® CRISPR-Cas9 tracrRNA (Lot #0000415438): IDT, 1072534
4D-Nucleofector™ Core Unit: Lonza, AAF-1002B
4D-Nucleofector™ X Unit: Lonza, AAF-1002×
SE Cell Line 4D-Nucleofector™ X Kit L: Lonza V4XC-1012

### Tissue Culture Plates

G-Rex 24-well plate: Wilson Wolf, 80192M
G-Rex 6-well plate: Wilson Wolf, 80240M
VECELL 96-well plate: Cosmo Bio, VCL-V96WGPB-10-EX
TC-treated 6-well plate: Corning Costar, 3506
TC-treated 24-well plates: Corning Costar, 3524
Non-treated 6-well plate: Corning Costar, 3736
Non-treated 24-well plate: Corning Costar, 3738
96-well flat-bottom plates: Corning Costar, 3595
384-well, poly-L lysine-treated, flat-bottom microplate: Greiner Bio-One™, 781936.
VECELL 96-well plate: Cosmo Bio, VCL-V96WGPB-10-EX

### Leukopak Processing Reagents:

Multi-24 Column Block: Miltenyi Biotec, 130-095-692
Single-well Deep Well Plate: Miltenyi Biotec, 130-114-966
Custom Leukopak PBMC Isolation Kit, human: Miltenyi Biotec, 120-051-115
Lymphocyte Separation Medium: Corning, 25-072-CV
CryoStor CS10: StemCell Technologies, 07930

### Genotyping Reagents

GeneJet Genomic DNA Purification Kit: Thermo Fisher Scientific, K0722
Phusion High-Fidelity PCR Master Mix with HF Buffer: Thermo Fisher Scientific, F531L
Monarch PCR & DNA Cleanup Kit (5 μg): New England Biolabs, T1030L
Nuclease-Free Water: Ambion, AM9937

### 10× Genomics Reagents

Chromium Single Cell 5′ Library & Gel Bead kit v1: 10× Genomics, PN1000006
Chromium Single Cell V(D)J Enrichment Kit, Human T Cell: 10× Genomics, PN1000005
Chromium Chip A Single Cell Kit: 10× Genomics, PN1000152
Single Index Kit T Set A: 10× Genomics, PN1000213
SPRIselect reagent kit: Beckman Coulter, B23318
High Sensitivity D5000 ScreenTape: Agilent Technologies, 5067-5592
High Sensitivity D5000 Reagents: Agilent Technologies, 5067-5593
Qubit™ dsDNA HS Assay Kit: Invitrogen, Q32854

### Packaging and Lentivirus Production Reagents

jetPRIME transfection reagent: Polyplus-transfection, 114-75
Vivaspin 20: Sartorius, VS2041
Vivaflow 50 cassettes: Sartorius, VF05P4

### CliniMACS Prodigy Reagents

CliniMACS CD4 MicroBeads: Miltenyi bioteck, 170-076-702
CliniMACS CD8 MicroBeads: Miltenyi bioteck, 170-076-703
CliniMACS CD45RA Microbeads: Miltenyi bioteck, 170-098-025
CliniMACS Prodigy TS 520: Miltenyi bioteck, 170-076-600
CliniMACS PBS/EDTA Buffer (2×3 L): Miltenyi bioteck, 130-021-201

### Other Reagents

ImmunoCult™ Human CD3/CD28/CD2 T cell activator: StemCell Technologies, 10970
Poly-L-ornithine solution 0.01%: Millipore-Sigma, P4957-50ML
ViaStain AO/PI staining solution in PBS: Nexcelom Bioscience, CS2-0106-5 mL
Puromycin: Gibco, A11138-03
Nourseothricin: GoldBio, N-500-1

### Example 2: Discovery of TSC-100: A Natural HA-1-Specific TCR to Treat Leukemia Following Hematopoietic Stem Cell Transplant Therapy

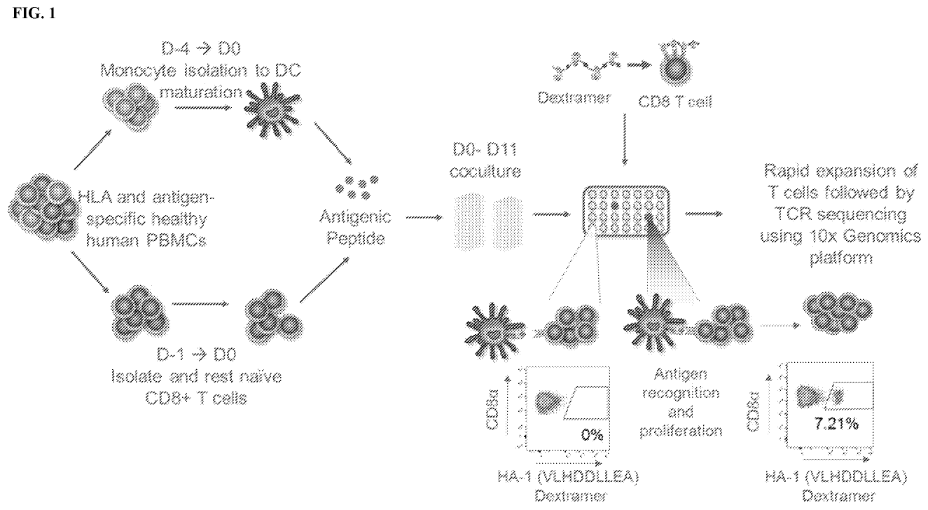

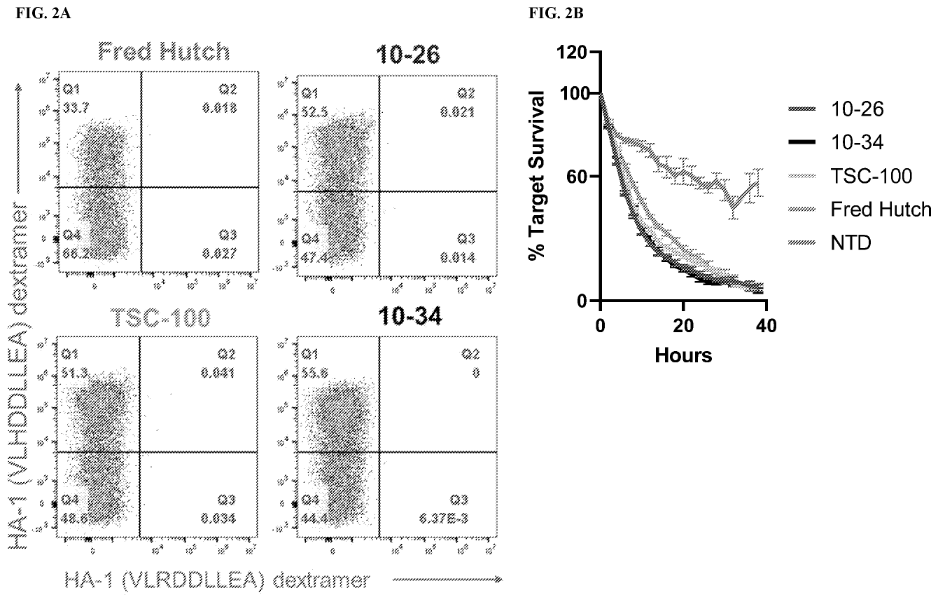

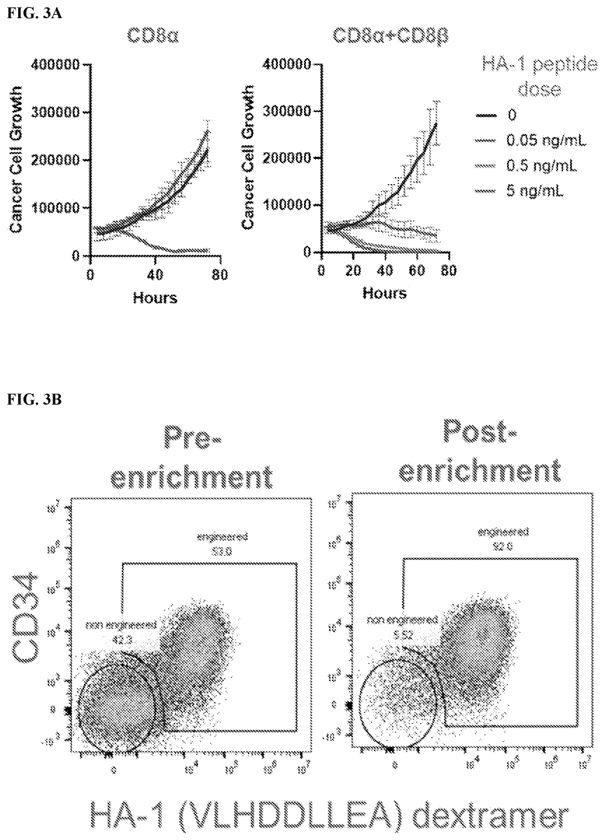

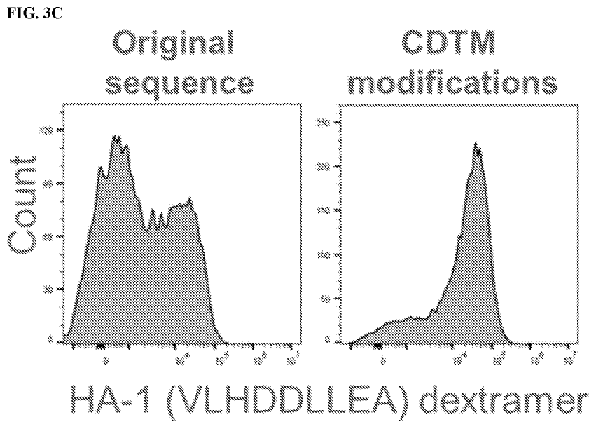

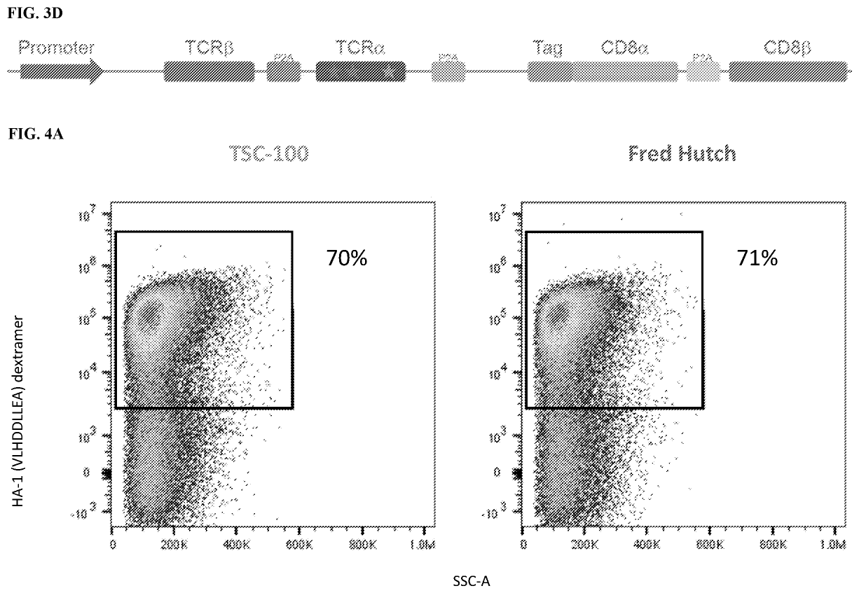

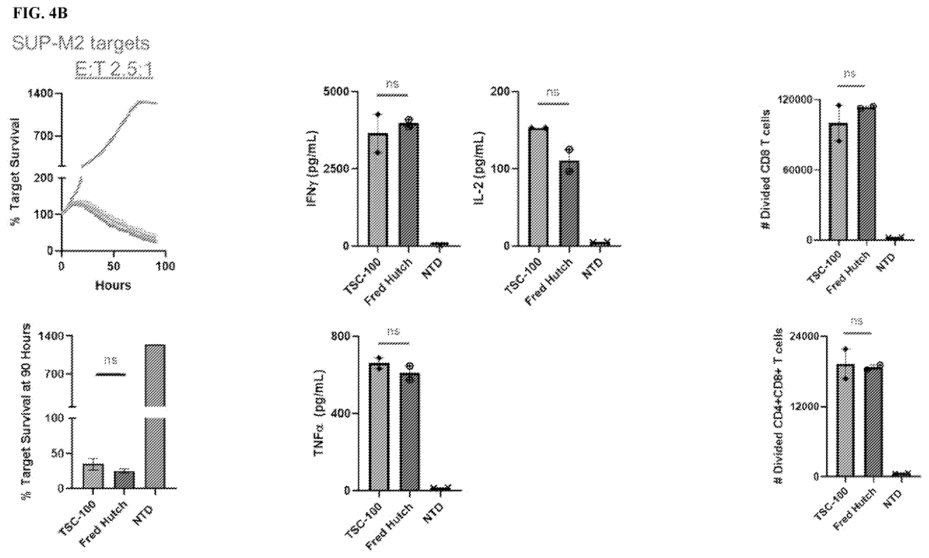

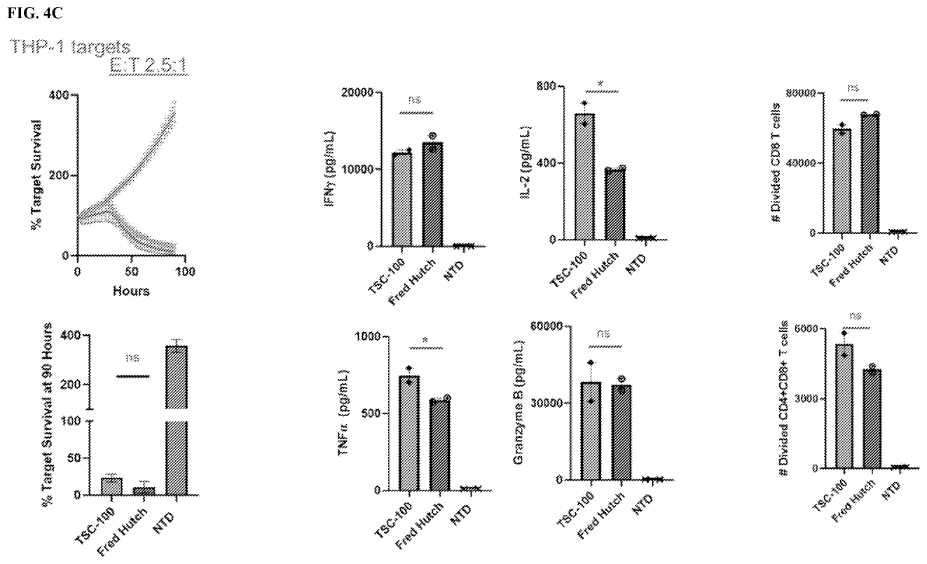

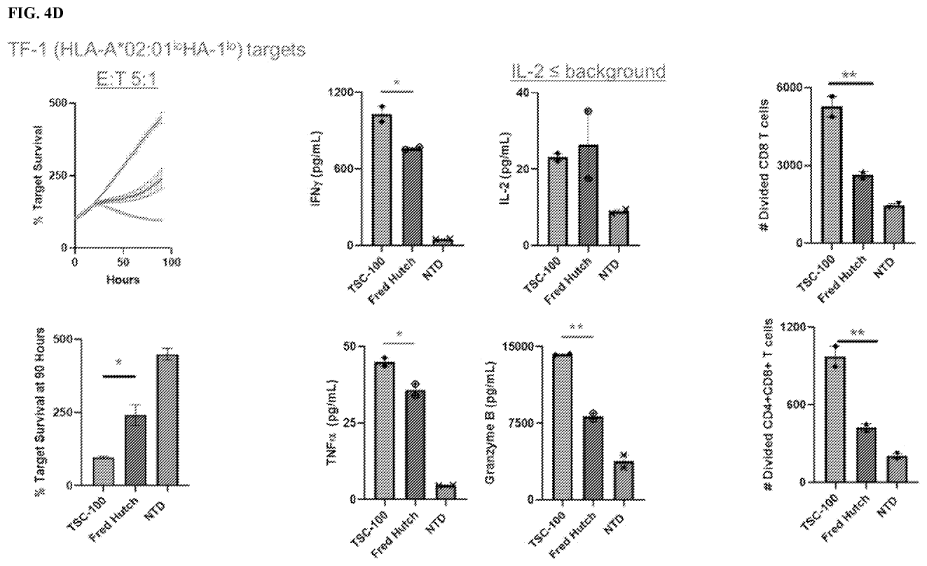

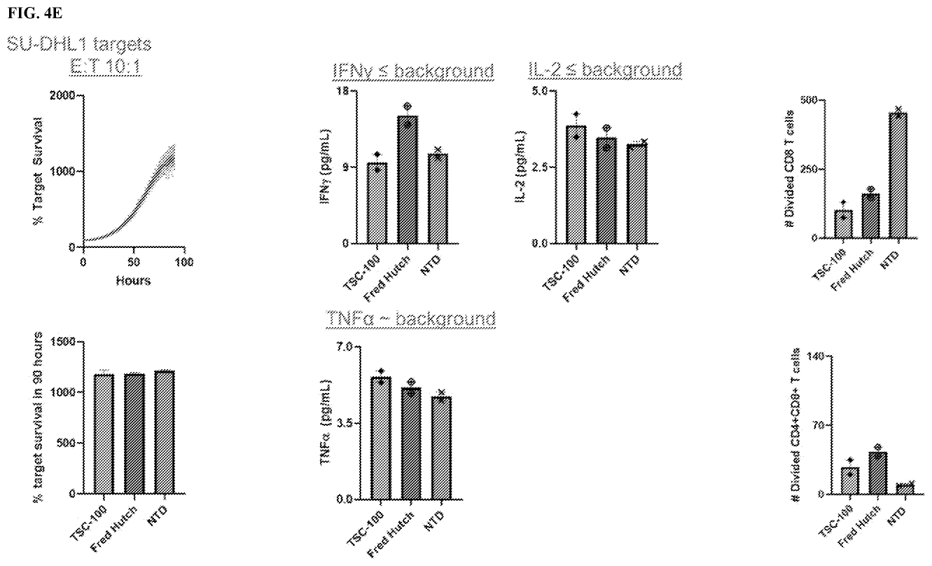

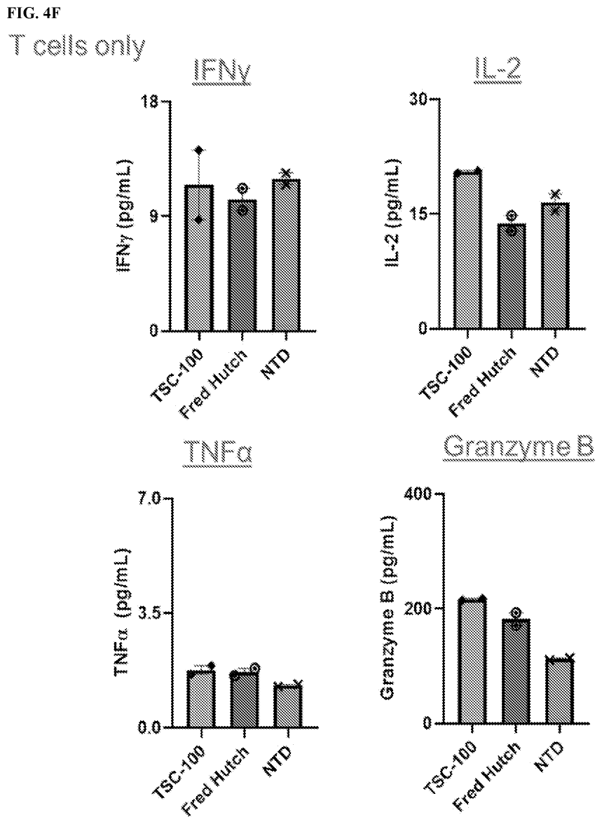

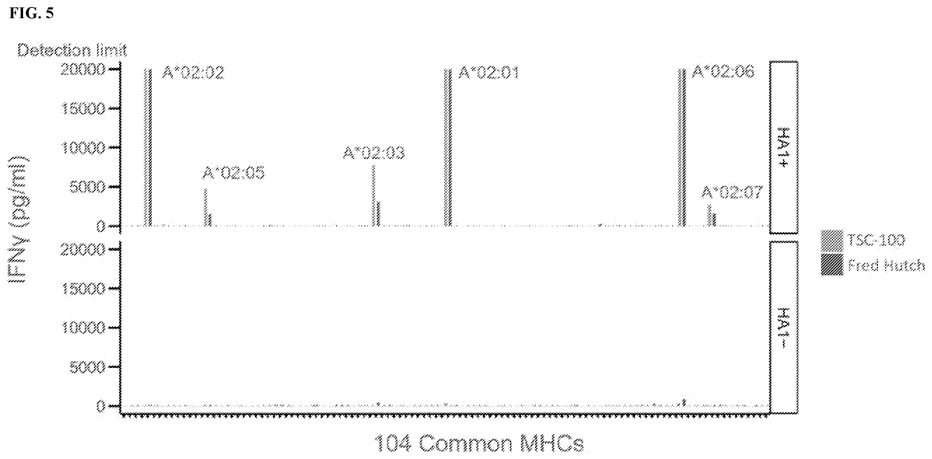

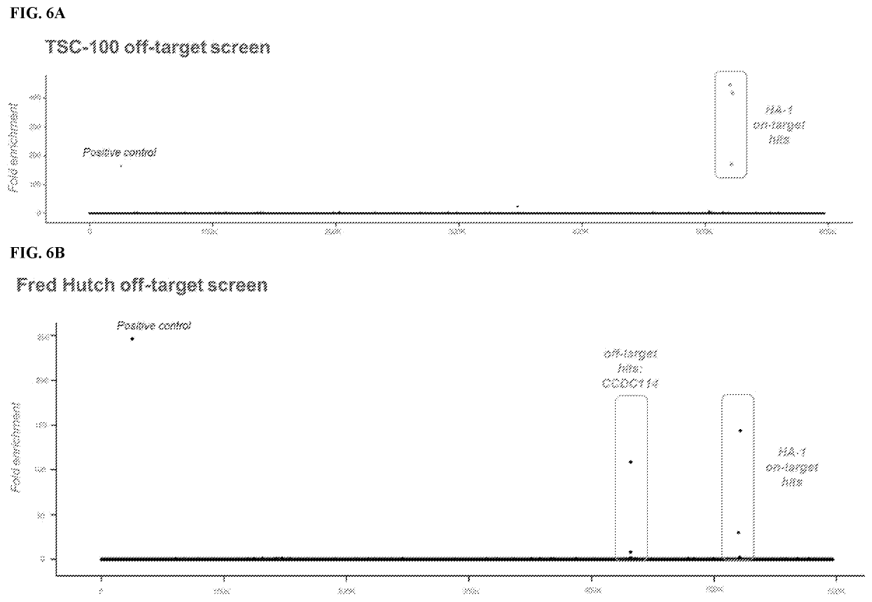

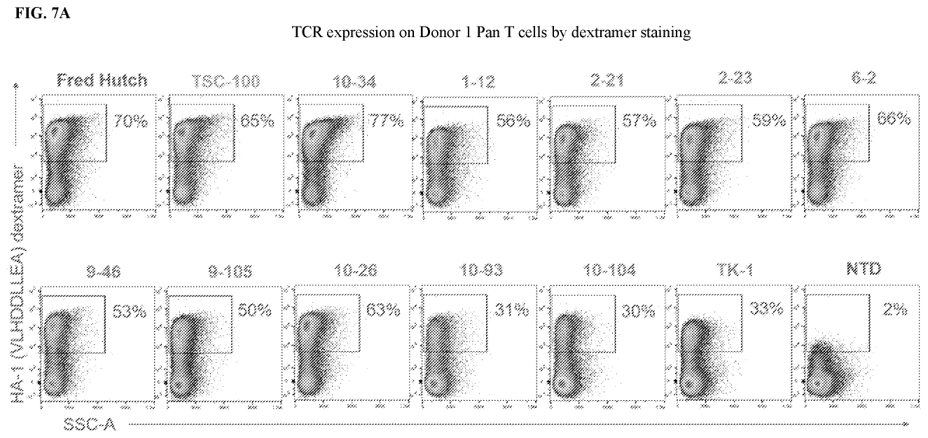

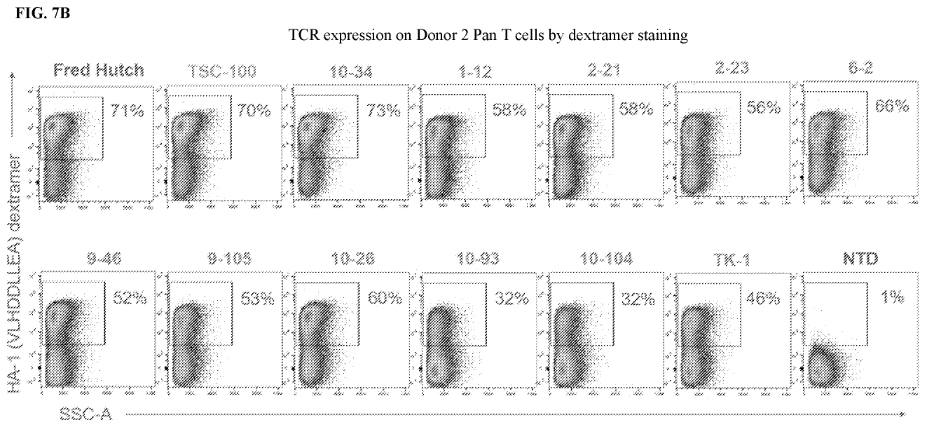

A high-throughput TCR discovery platform that enables rapid cloning of antigen-specific TCRs from healthy donors was developed. This platform was then used to screen 178.3 million naïve CD8⁺ T cells from six unique HA-1⁻ (VLRDDLLEA, genotype RS_1801284 G/G) donors, and 329 HA-1-specific TCRs were identified (FIG. 1). Peptide concentrations used in the TCR discovery platform is 1 μg/mL. Each TCR was tested for expression and the ability to kill HA-1 peptide (VLHDDLLEA) (10 ng/mL-10 pg/mL) pulsed T2 cells, using a previously published, clinical-stage HA-1-specific TCR as a benchmark for these studies (FIG. 2) (Dossa et al. (2018) Blood 131:108-120). In parallel, TCR constant region modifications were tested to promote expression and proper pairing of exogenous TCR alpha and beta chains and a lentiviral vector was designed to co-deliver CD8 coreceptors as well as a CD34 enrichment tag to enable purification of engineered T cells (FIGS. 3A-3D). The top 11 candidates were cloned into TScan's optimized backbone and evaluated for cytotoxicity, cytokine production, and T cell proliferation using a panel of HLA-A*02:01⁺HA-1⁺ cell lines (FIGS. 4A-4F). The E:T ratio from 20:1 to 2.5:1 were used. Finally, the top two TCRs were evaluated for allo-reactivity (FIG. 5, including TSC-100 and clone HA1-10-34 described herein) and off-target cross-reactivity (FIGS. 6A and 6B) using the proprietary genome-wide T-Scan platform.

The TCR discovery and evaluation platform described here identified 329 HA-1-specific TCRs from a total of 178.3 million naïve T cells (FIG. 1), and TSC-100 as the most active TCR. Defined mutations in the constant region of TSC-100 enhanced its surface expression while decreasing expression of endogenous TCRs (FIG. 3C), and co-introduction of CD8 enabled efficient engagement and function of engineered CD4 cells (FIG. 3A). Overall, TSC-100 exhibited comparable activity to a clinical-stage benchmark TCR when challenged with cell lines expressing moderate to high levels of HA-1 (FIG. 4B), and superior activity when incubated with cell lines expressing low levels of both HA-1 and MHC-I (FIGS. 4C-4D) (Dossa et al. (2018) Blood 131:108-120). RNA expression for HA-1 and HLA A0201 cell lines from CCLE are shown in Table 5 below.

TABLE 5
Reference for cell line expression

| | HA1 | HA1 | HLA A |
|---|---|---|---|
| Cell line | genotype | expression* | expression* |
| SUP-M2 | +/+ | 84 | 448 |
| THP-1 | +/− | 120 | 275 |
| TF-1 | +/− | 32 | 55 |
| SU-DHL1 | −/− | 69 | 288 |

*Expression from CCLE in transcripts per million (TPM)

In addition, TSC-100 exhibited no detectable allo-reactivity to 108 different HLA types tested (FIG. 5), and minimal off-target effects when challenged with a genome-wide library expressing peptides derived from human proteins (FIG. 6A).

TSC-100 exhibits comparable or superior activity to a clinical-stage therapeutic TCR, with minimal allo-reactivity or off-target effects.

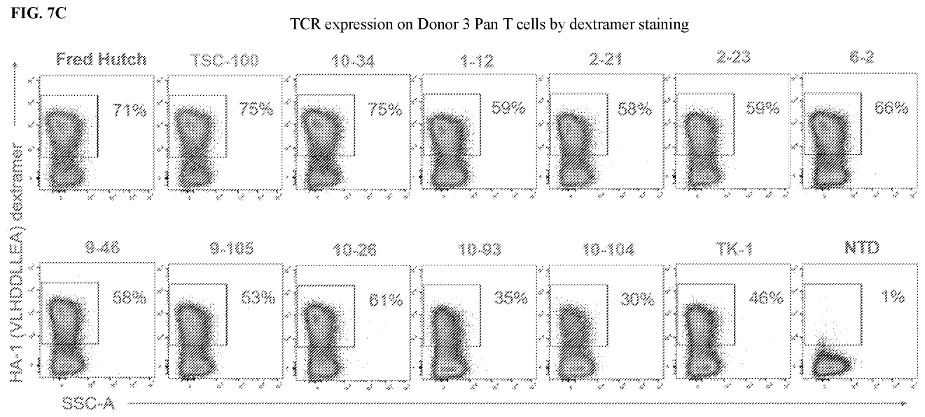

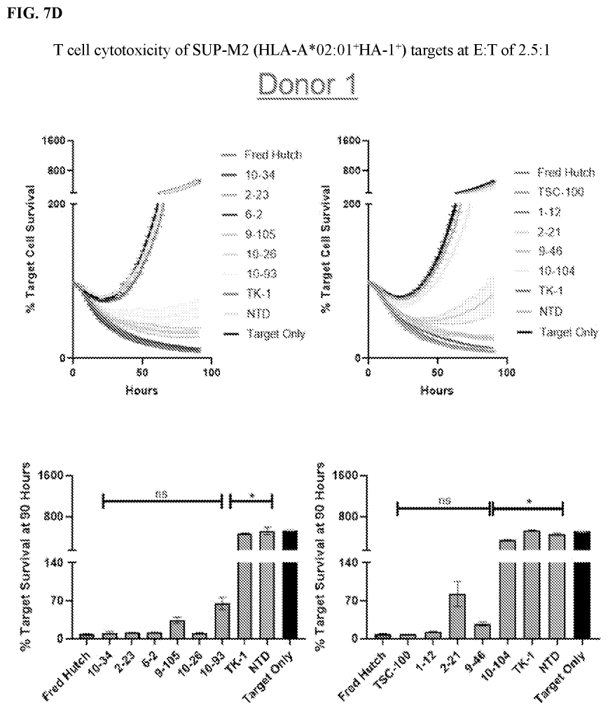

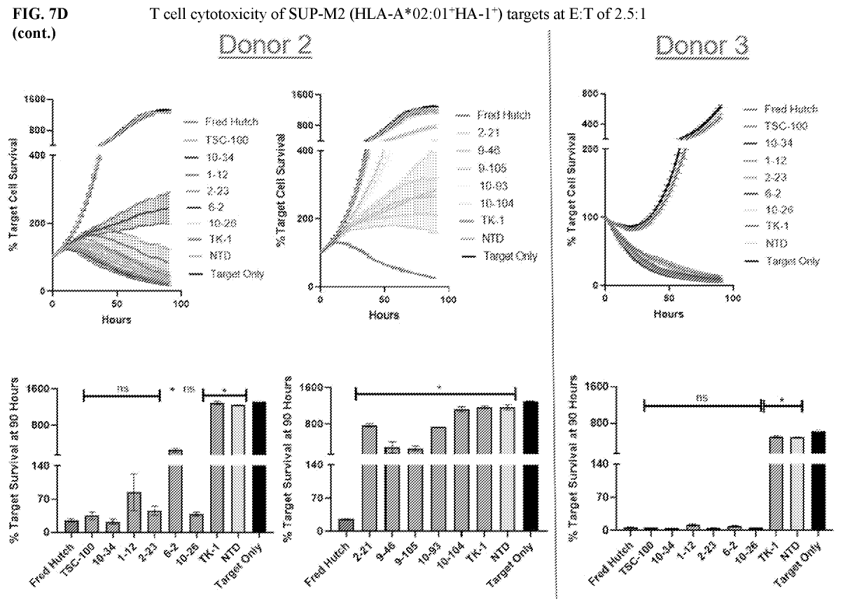

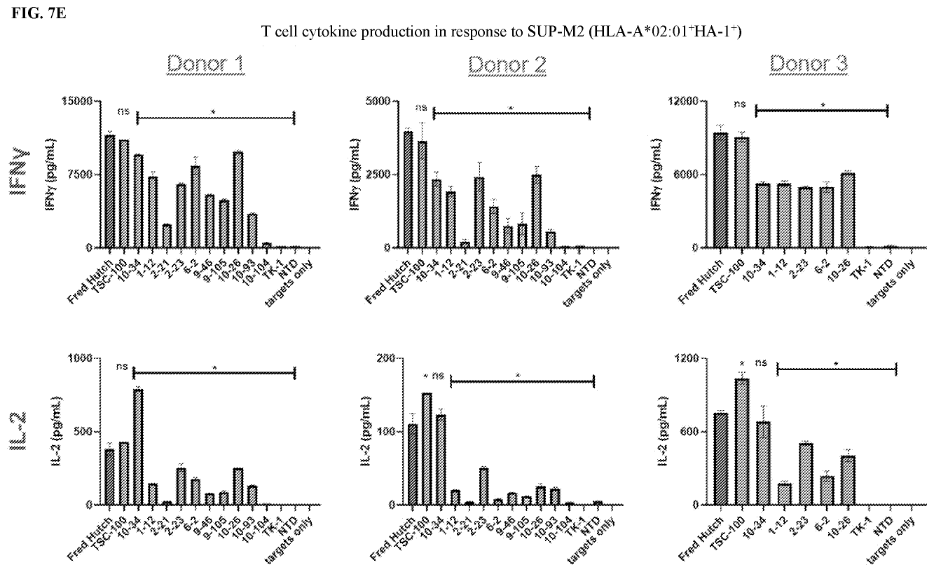

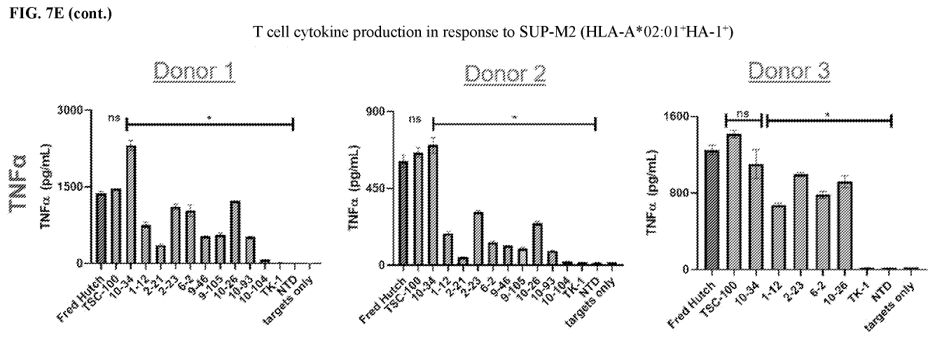

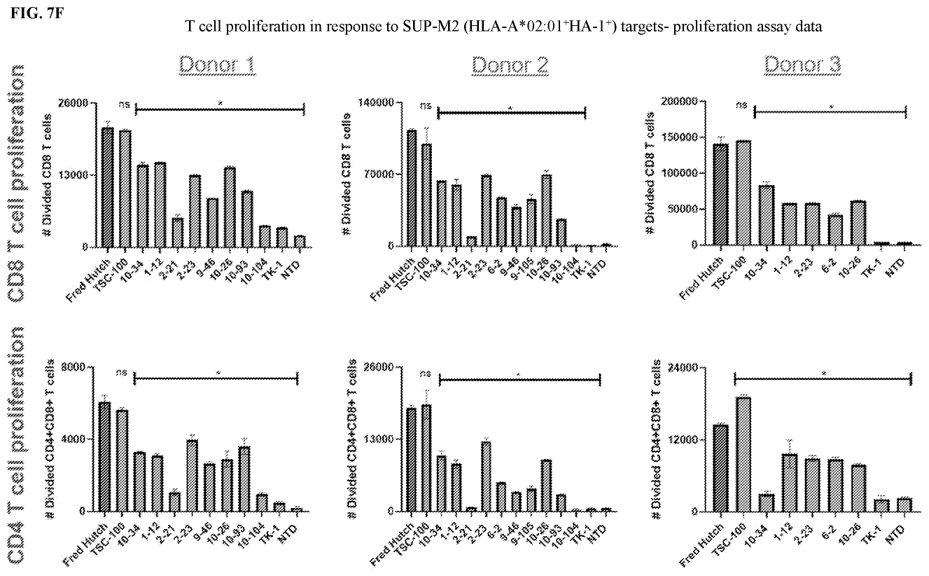

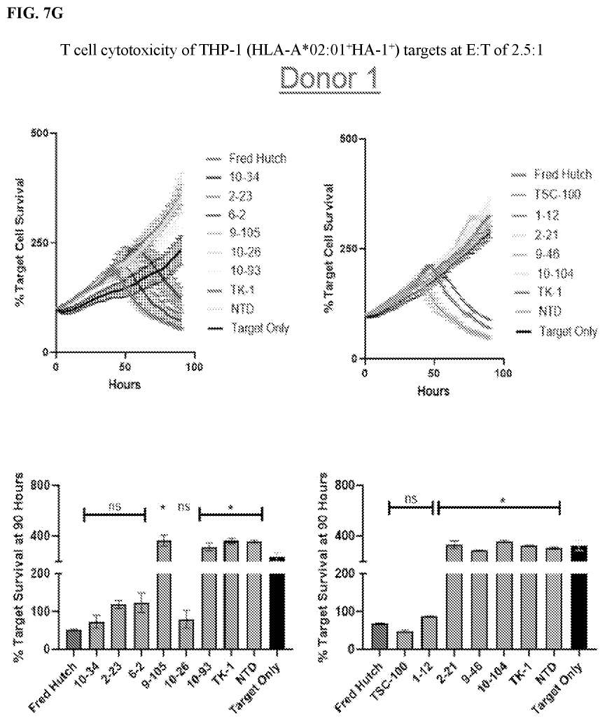

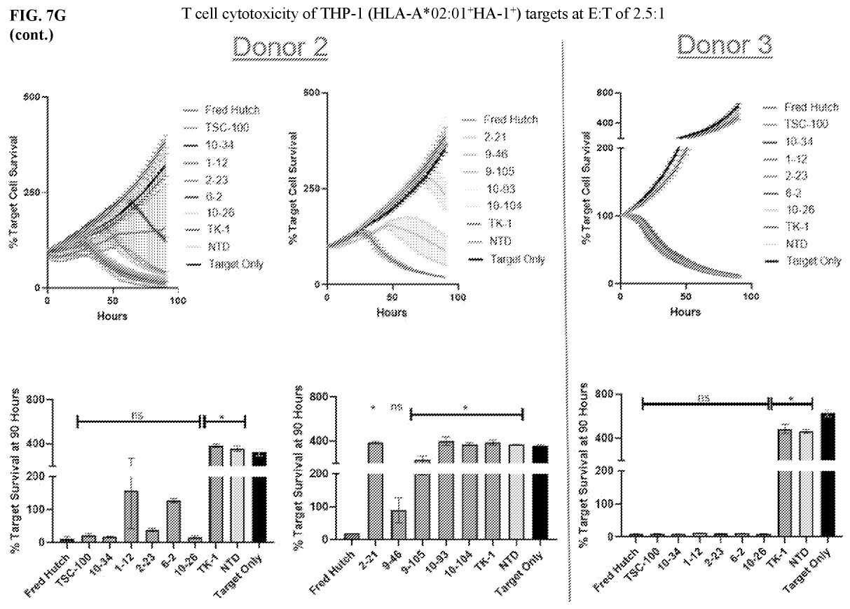

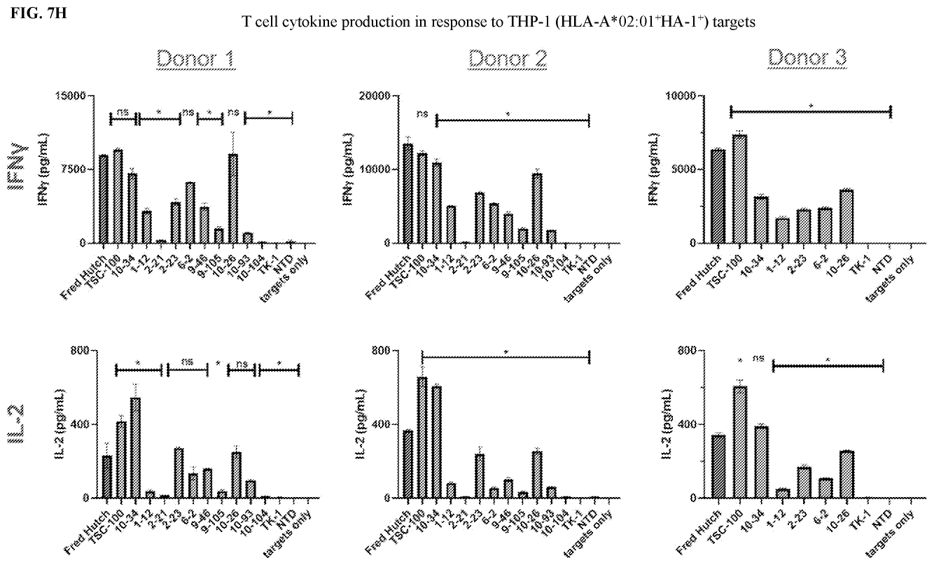

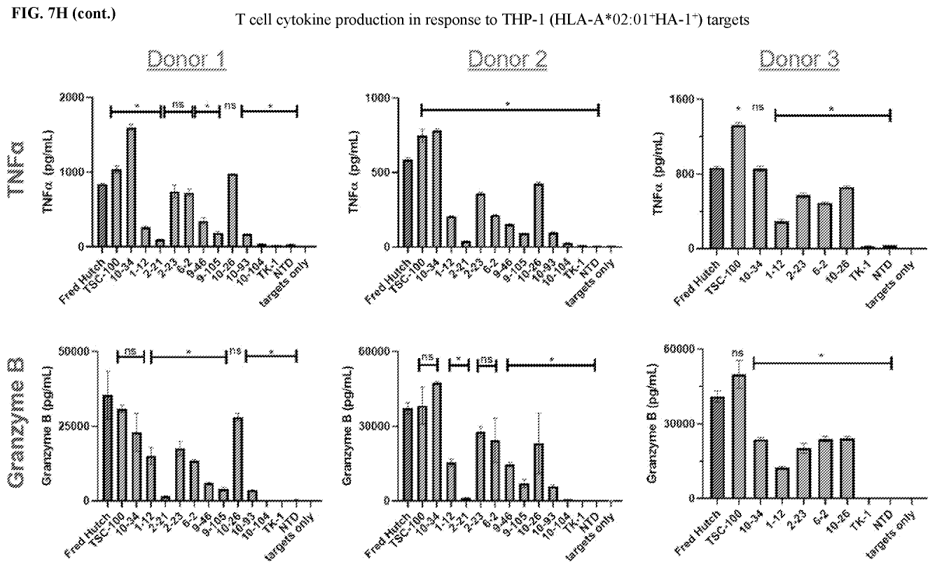

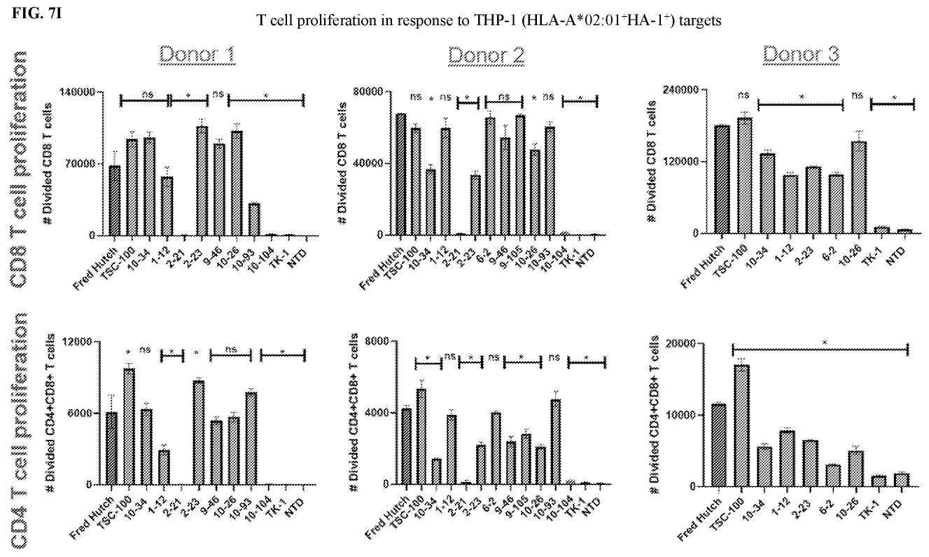

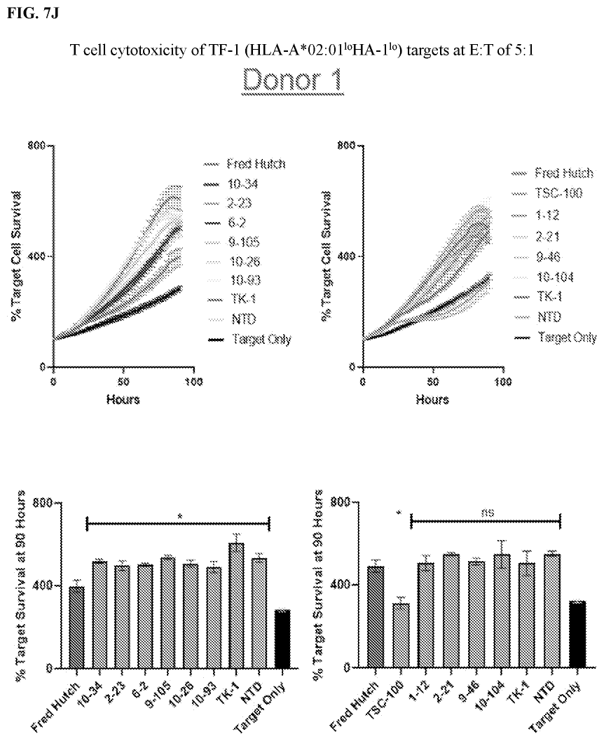

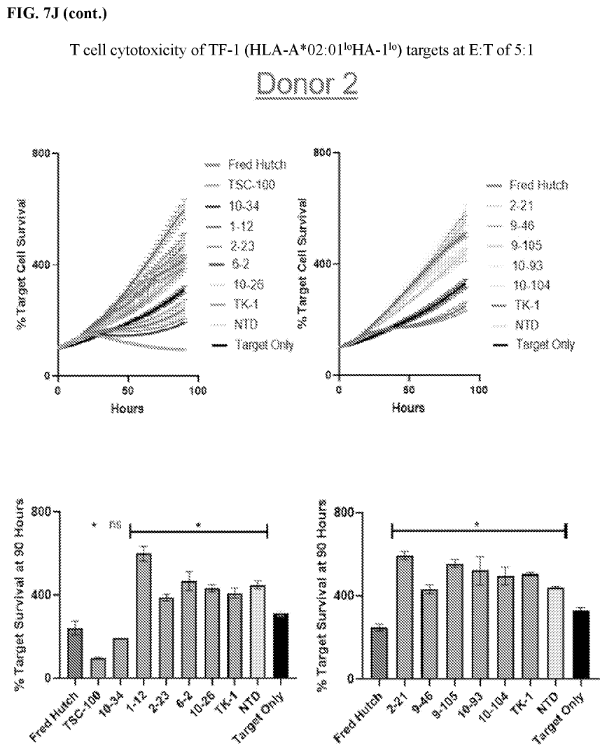

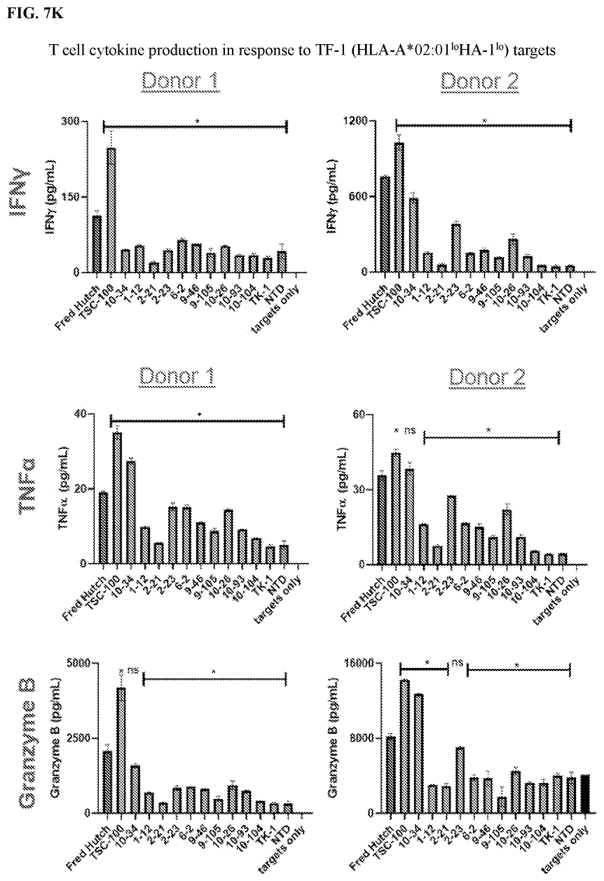

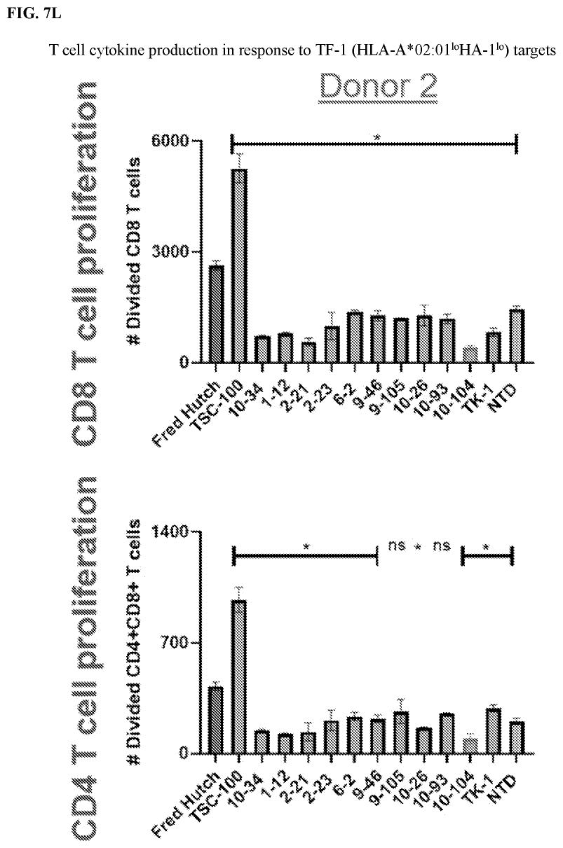

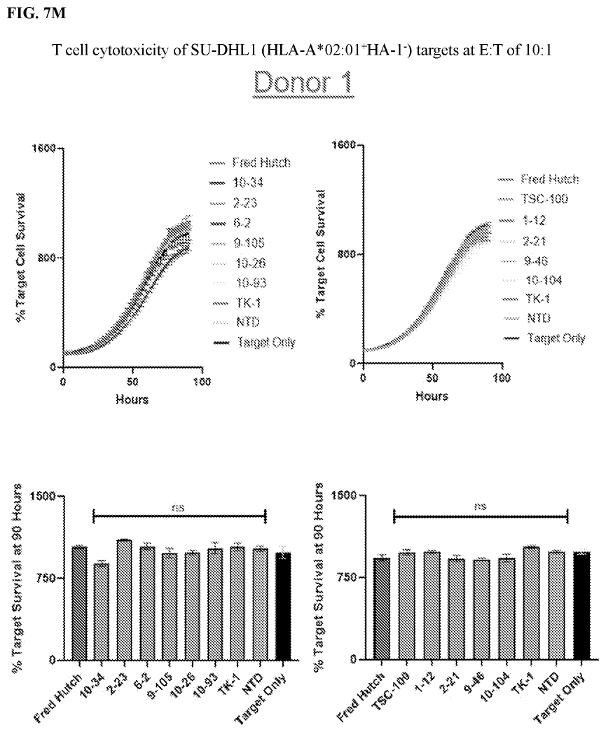

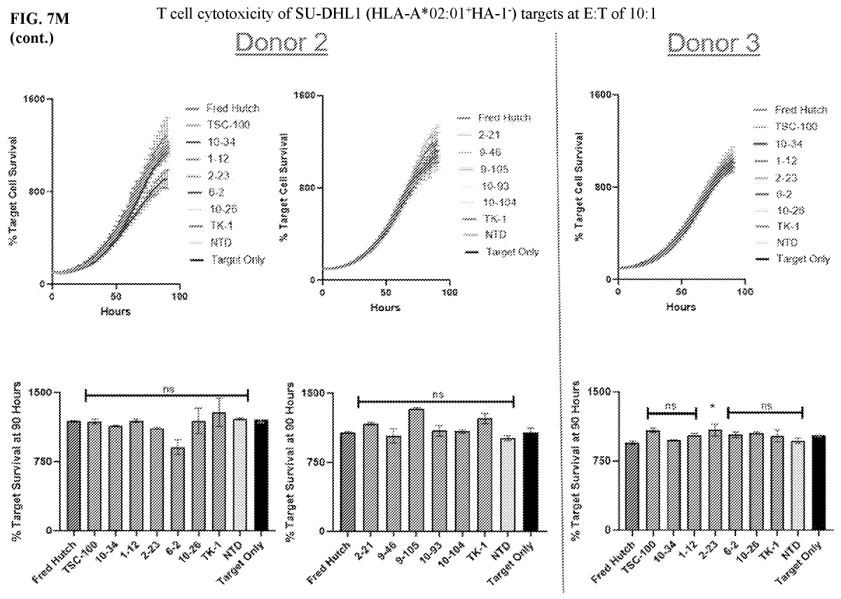

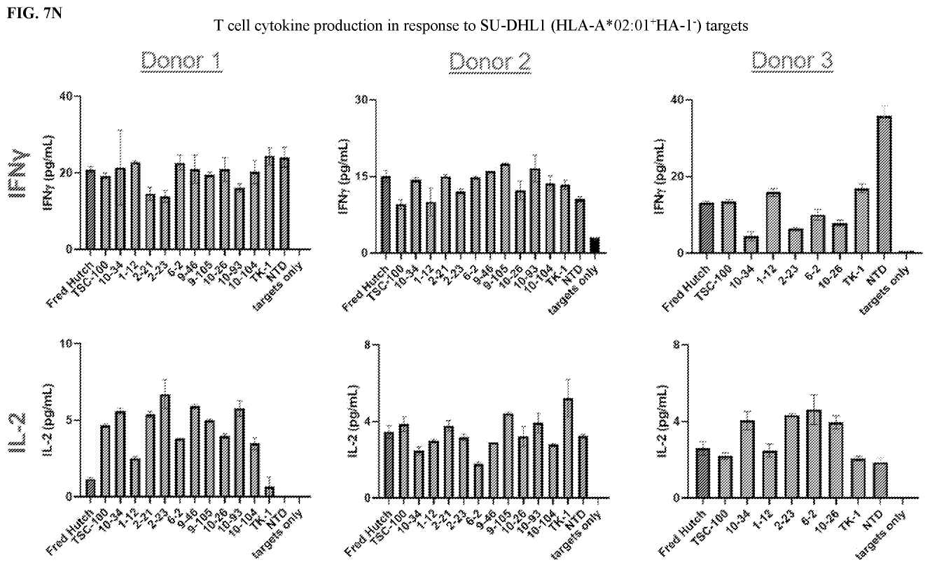

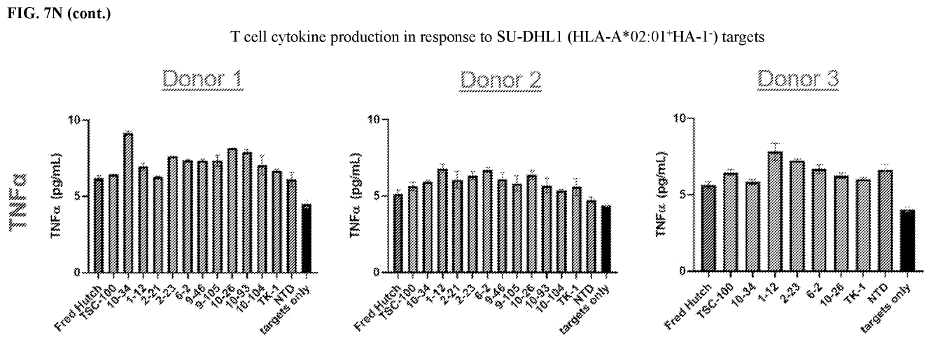

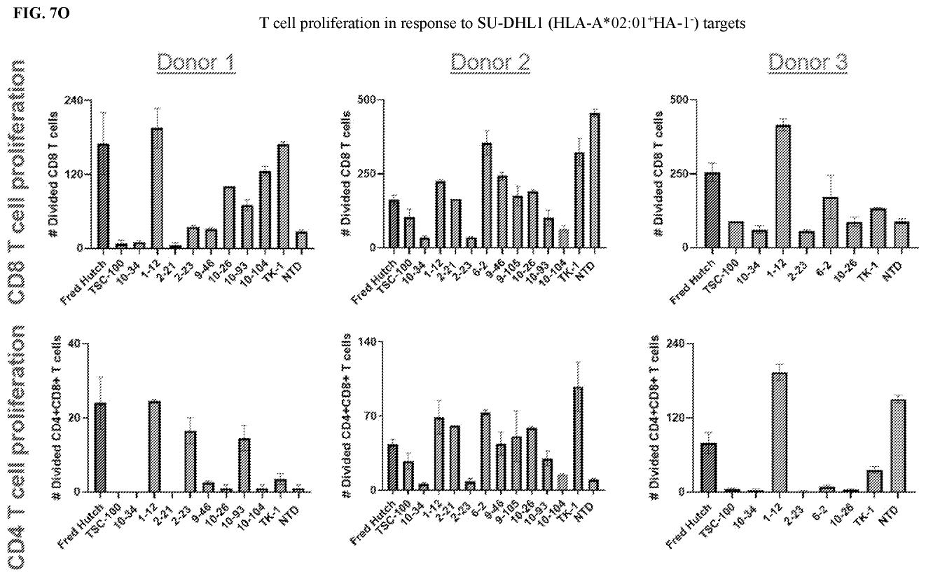

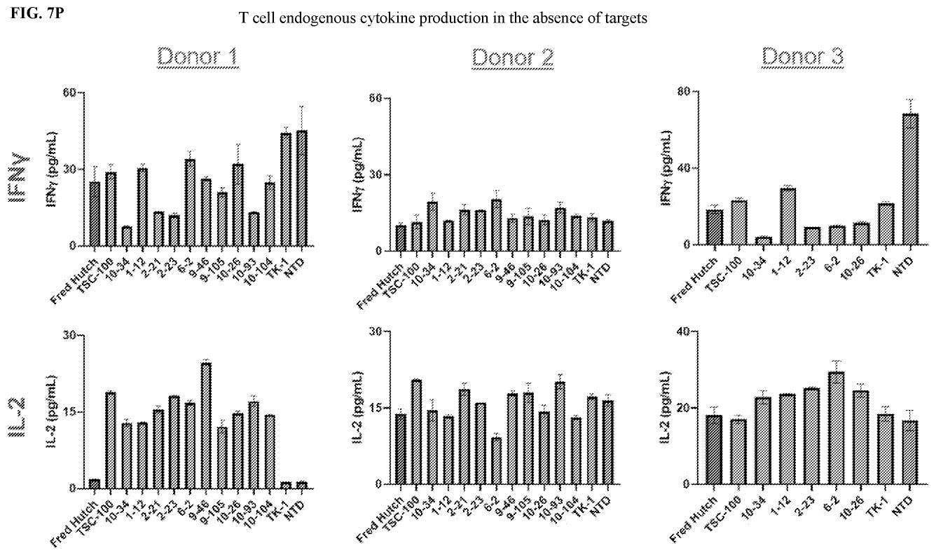

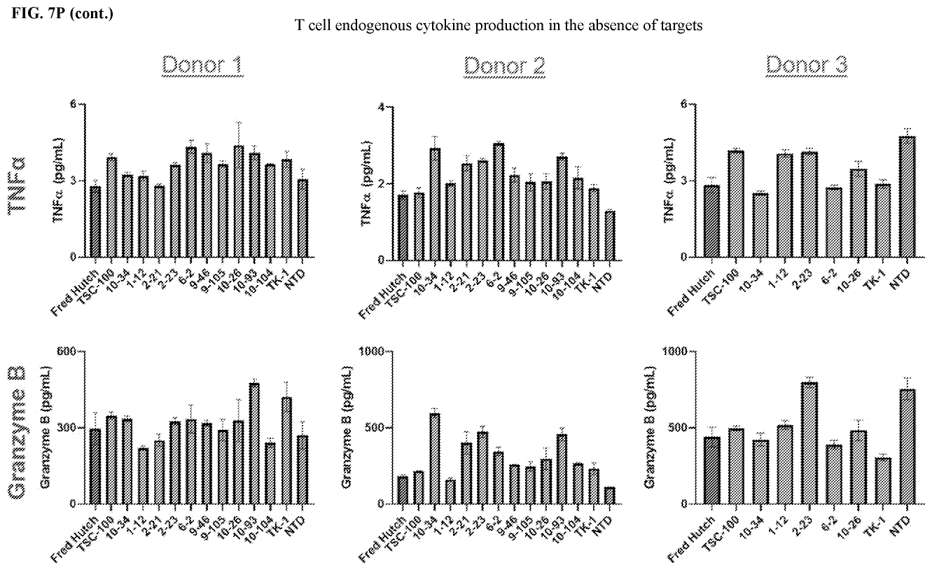

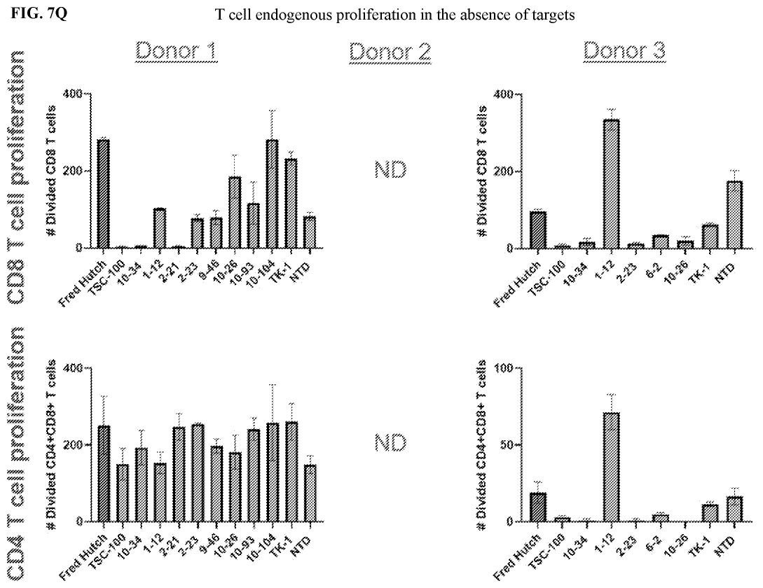

The full data set, which includes the data from TSC-100 described above in conjunction with data from 10 other top candidates cloned into Tscan's optimized backbone from the same experiments and using the same methods (e.g., evaluations of cytotoxicity, cytokine production, T cell proliferation effects, etc.) are shown in FIGS. 7A-7Q, which confirm functionality of the 11 top candidates.

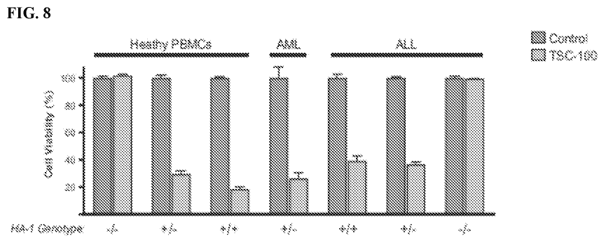

In addition, TSC-100 has been demonstrated to kill HA-1-expressing cells from a variety of healthy donors and cancer cell types. Because people inherit two copies of every chromosome, one from their mother and one from their father, everyone has two copies of the ARHGAP45 gene. HA-1-positive patients can therefore be either homozygous for HA-1 (+/+), with both genes encoding the HA-1-positive peptide (VLHDDLLEA), or heterozygous for HA-1 (+/−), with one gene encoding the HA-1-positive peptide and the other encoding the HA-1-negative peptide (VLRDDLLEA). To ensure that TSC-100 is able to effectively eliminate healthy blood cells and cancer cells, such as leukemia cells, that are either homozygous HA-1-positive (+/+) or heterozygous HA-1-positive (+/−), the activity of TSC-100 against blood cells derived from a variety of healthy donors and patients with cancer (e.g., AML and ALL) was determined. As shown in FIG. 8, TSC-100 eliminates both homozygous and heterozygous healthy blood and cancer cells.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS AND SCOPE

The details of one or more embodiments encompassed by the present invention are set forth in the description above. Although representative, exemplary materials and methods have been described above, any materials and methods similar or equivalent to those described herein may be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which the present invention belongs. In the case of conflict, the present description provided above will control.

Those skilled in the art will recognize or be able to ascertain using no more than routine experimentation, many equivalents to the specific embodiments encompassed by the present invention described herein. The scope encompassed by the present invention is not intended to be limited to the description provided herein and such equivalents are intended to be encompassed by the appended claims.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges in different embodiments encompassed by the present invention, to the tenth of the unit of the lower limit of the range, unless the context clearly dictates otherwise.

In addition, it is to be understood that any particular embodiment encompassed by the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions encompassed by the present invention (e.g., any antibiotic, therapeutic or active ingredient; any method of production; any method of use; etc.) may be excluded from any one or more claims, for any reason, whether or not related to the existence of prior art.

It is to be understood that the words which have been used are words of description rather than limitation, and that changes may be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope encompassed by the present invention.

What is claimed is:

1. A binding protein comprising:
a) a T cell receptor (TCR) alpha chain CDR sequence with at least about 80% identity to a TCR alpha chain CDR sequence selected from the group consisting of TCR alpha chain CDR sequences listed in Table 1; and/or
b) a TCR beta chain CDR sequence with at least about 80% identity to a TCR beta chain CDR sequence selected from the group consisting of TCR beta chain CDR sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M.

2. A binding protein comprising:
a) a TCR alpha chain variable (V_{α}) domain sequence with at least about 80% identity to a TCR V_{α} domain sequence selected from the group consisting of TCR V_{α} domain sequences listed in Table 1; and/or
b) a TCR beta chain variable (V_{β}) domain sequence with at least about 80% identity to a TCR V_{β} domain sequence selected from the group consisting of TCR V_{β} domain sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M.

3. A binding protein comprising:
a) a TCR alpha chain sequence with at least about 80% identity to a TCR alpha chain sequence selected from the group consisting of TCR alpha chain sequences listed in Table 1; and/or
b) a TCR beta chain sequence with at least about 80% identity to a TCR beta chain sequence selected from the group consisting of TCR beta chain sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M.

4. A binding protein comprising:
a) a TCR alpha chain CDR sequence selected from the group consisting of TCR alpha chain CDR sequences listed in Table 1; and/or
b) a TCR beta chain CDR sequence selected from the group consisting of TCR beta chain CDR sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M.

5. A binding protein comprising:
a) a TCR alpha chain variable (V_{α}) domain sequence selected from the group consisting of TCR V_{α} domain sequences listed in Table 1; and/or
b) a TCR beta chain variable (V_{β}) domain sequence selected from the group consisting of TCR V_{β} domain sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M.

6. A binding protein comprising:
a) a TCR alpha chain sequence selected from the group consisting of TCR alpha chain sequences listed in Table 1; and/or
b) a TCR beta chain sequence selected from the group consisting of TCR beta chain sequences listed in Table 1, wherein the binding protein is capable of binding to an HA-1 immunogenic peptide-MHC (pMHC) complex, optionally wherein the binding affinity has a K_{d} less than or equal to about 5×10⁻⁴ M.

7. The binding protein of any one of claims 1-6, wherein 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 1, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Table 1, and/or 3) each CDR of the binding protein has up to five amino acid substitutions, insertions, deletions, or a combination thereof as compared to the cognate reference CDR sequence listed in Table 1.

8. The binding protein of any one of claims 1-7, wherein the HA-1 immunogenic peptide comprises the amino acid sequence VLHDDLLEA.

9. The binding protein of any one of claims 1-8, wherein the binding protein is chimeric, humanized, or human.

10. The binding protein of any one of claims 1-9, wherein the binding protein is a TCR, an antigen-binding fragment of a TCR, a single chain TCR (scTCR), a chimeric antigen receptor (CAR), or a fusion protein comprising a TCR and an effector domain, optionally wherein the binding domain comprises a transmembrane domain and an effector domain that is intracellular.

11. The binding protein of any one of claims 1-10, wherein the TCR alpha chain and the TCR beta chain are covalently linked, optionally wherein the TCR alpha chain and the TCR beta chain are covalently linked through a linker peptide.

12. The binding protein of any one of claims 1-11, wherein the TCR alpha chain and/or the TCR beta chain are covalently linked to a moiety, optionally wherein the covalently linked moiety comprises an affinity tag or a label.

13. The binding protein of claim 12, wherein the affinity tag is selected from the group consisting of CD34 enrichment tag, Glutathione-S-Transferase (GST), calmodulin binding protein (CBP), protein C tag, Myc tag, HaloTag, HA tag, Flag tag, His tag, biotin tag, and V5 tag, and/or wherein the label is a fluorescent protein.

14. The binding protein of any one of claims 1-13, wherein the covalently linked moiety is selected from the group consisting of an inflammatory agent, cytokine, toxin, cytotoxic molecule, radioactive isotope, or antibody or antigen-binding fragment thereof.

15. The binding protein of any one of claims 1-14, wherein the binding protein binds to the pMHC complex on a cell surface.

16. The binding protein of any one of claims 1-15, wherein the MHC is a MHC multimer, optionally wherein the MHC multimer is a tetramer.

17. The binding protein of any one of claims 1-16, wherein the MHC is a MHC class I molecule.

18. The binding protein of any one of claims 1-17, wherein the MHC comprises an MHC alpha chain that is an HLA serotype HLA-A*02.

19. The binding protein of any one of claims 1-18, wherein the HLA allele is selected from the group consisting of HLA-A*0201, HLA-A*0202, HLA-A*0203, HLA-A*0205, HLA-A*0206, and HLA-A*0207 allele.

20. The binding protein of any one of claims 1-19, wherein binding of the binding protein to the HA-1 peptide-MHC (pMHC) complex elicits an immune response, optionally wherein the immune response is a T cell response.

21. The binding protein of any one of claims 1-20, wherein the T cell response is selected from the group consisting of T cell expansion, cytokine release, and/or cytotoxic killing.

22. The binding protein of any one of claims 1-21, the binding protein is capable of specifically and/or selectively binding to the HA-1 immunogenic peptide-MHC (pMHC) complex with a K_{d} less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, or less than or equal to about 1×10⁻¹² M.

23. The binding protein of any one of claims 1-22, wherein the binding protein has a higher binding affinity to the peptide-MHC (pMHC) than does a known T-cell receptor.

24. The binding protein of any one of claims 1-23, wherein the binding protein has at least 1.05 fold higher binding affinity to the peptide-MHC (pMHC) than does a known T-cell receptor.

25. The binding protein of any one of claims 1-24, wherein the binding protein induces higher T cell expansion, cytokine release, and/or cytotoxic killing than does a known T-cell receptor when contacted with target cells with a heterozygous expression of HA-1.

26. The binding protein of any one of claims 1-25, wherein the binding protein induces at least 1.05-fold increase in T cell expansion, cytokine release, and/or cytotoxic killing than does a known T-cell receptor when contacted with target cells with a heterozygous expression of HA-1.

27. The binding protein of claim 25 or 26, wherein the target cell is THP-1 or TF1 cell line.

28. The binding protein of claim 25 or 26, wherein the target cell is a cancer cell, optionally wherein the cancer cell is a hematopoietic cancer cell, a leukemia cell, a lymphoma cell, a myelodysplastic disorder cell, a myeloproliferative neoplasm cell, or a myeloma cell.

29. The binding protein of any one of claims 22-28, wherein the binding protein does not bind to HA-1^{R} peptide-MHC (pMHC) complex, wherein the HA-1^{R} peptide comprises the amino acid sequence VLRDDLLEA.

30. The binding protein of any one of claims 22-29, wherein the binding protein does not bind to a CCDC114 peptide-MHC (pMHC) complex, optionally wherein the CCDC114 peptide is SMIDDLLGV.

31. A TCR alpha chain and/or beta chain selected from the group consisting of TCR alpha chain and beta chain sequences listed in Table 1.

32. An isolated nucleic acid molecule that hybridizes, under stringent conditions, with the complement of a nucleic acid encoding a polypeptide selected from the group consisting of polypeptide sequences listed in Table 1, or a sequence with at least about 80% homology to a nucleic acid encoding a polypeptide selected from the group consisting of the polypeptide sequences listed in Table 1, optionally wherein the isolated nucleic acid molecule comprises 1) a TRAV, TRAJ, and/or TRAC gene or fragment thereof selected from the group of TRAV, TRAJ, and TRAC genes listed in Table 1 and/or 2) a TRBV, TRBJ, and/or TRBC gene or fragment thereof selected from the group of TRBV, TRBJ, and TRBC genes listed in Table 1.

33. The isolated nucleic acid of claim 32, wherein the nucleic acid is codon optimized for expression in a host cell.

34. A vector comprising the isolated nucleic acid of claim 32 or 33.

35. The vector of claim 34, wherein the vector is a cloning vector, expression vector, or viral vector.

36. The vector of claim 34 or 35, wherein the vector further comprises a nucleic acid sequence encoding CD8α and/or CD8β.

37. The vector of any one of claims 34-36, wherein the nucleic acid sequence encoding CD8α or CD8β is operably linked to a nucleic acid encoding a tag.

38. The vector of any one of claims 34-37, wherein the nucleic acid encoding a tag is at the 5′ upstream of the nucleic acid sequence encoding CD8α or CD8β such that the tag is fused to the N-terminal of CD8α or CD8β.

39. The vector of any one of claims 34-38, wherein the tag is a CD34 enrichment tag.

40. The vector of any one of claims 34-39, wherein the isolated nucleic acid of claim 23 or 24, and the nucleic acid sequence encoding CD8α and/or CD8β are interconnected with an internal ribosome entry site or a nucleic acid sequence encoding a self-cleaving peptide.

41. The vector of any one of claims 34-40, wherein the self-cleaving peptide is P2A, E2A, F2A or T2A.

42. A host cell which comprises the isolated nucleic acid of claim 32 or 33, comprises the vector of any one of claims 34-41, and/or expresses the binding protein of any one of claims 1-30, optionally wherein the cell is genetically engineered.

43. The host cell of claim 42, wherein the host cell comprises a chromosomal gene knockout of a TCR gene, an HLA gene, or both.

44. The host cell of claim 42 or 43, wherein the host cell comprises a knockout of an HLA gene selected from an α1 macroglobulin gene, α2 macroglobulin gene, α3 macroglobulin gene, R31 microglobulin gene, β2 microglobulin gene, and combinations thereof.

45. The host cell of any one of claims 42-44, wherein the host cell comprises a knockout of a TCR gene selected from a TCR a variable region gene, TCR β variable region gene, TCR constant region gene, and combinations thereof.

46. The host cell of any one claims 42-45, wherein the host cell expresses CD8α and/or CD8β.

47. The host cell of claim 46, wherein the CD8α and/or CD8β is fused to a CD34 enrichment tag.

48. The host cell of claim 47, wherein host cells are enriched using the CD34 enrichment tag.

49. The host cell of any one of claims 42-48, wherein the host cell is a hematopoietic progenitor cell, peripheral blood mononuclear cell (PBMC), cord blood cell, or immune cell.

50. The host cell of any one of claims 42-49, wherein the immune cell is a cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or combination thereof.

51. The host cell of any one of claims 42-50, wherein the T cell is a naive T cell, central memory T cell, effector memory T cell, or a combination thereof.

52. The host cell of any one of claims 42-51, wherein the T cell is a primary T cell or a cell of a T cell line.

53. The host cell of any one of claims 42-52, wherein the T cell does not express or has a lower surface expression of an endogenous TCR.

54. The host cell of any one of claims 42-53, wherein the host cell is capable of producing a cytokine or a cytotoxic molecule when contacted with a target cell that comprises a peptide-MHC (pMHC) complex comprising the HA-1 peptide epitope in the context of an MHC molecule.

55. The host cell of claim 54, wherein the host cell is contacted with the target cell in vitro, ex vivo, or in vivo.

56. The host cell of claim 54 or 55, wherein the cytokine is TNF-α, IL-2, and/or IFN-γ.

57. The host cell of any one of claims 54-56, wherein the cytotoxic molecule is perforins and/or granzymes, optionally wherein the cytotoxic molecule is granzyme B.

58. The host cell of any one of claims 54-57, wherein the host cell is capable of producing a higher level of cytokine or a cytotoxic molecule when contacted with a target cell with a heterozygous expression of HA-1.

59. The host cell of claim 58, wherein the host cell is capable of producing an at least 1.05-fold higher level of cytokine or a cytotoxic molecule.

60. The host cell of any one of claims 54-59, wherein the host cell is capable of killing a target cell that comprises a peptide-MHC (pMHC) complex comprising the HA-1 peptide epitope in the context of an MHC molecule.

61. The host cell of claim 61, wherein the killing is determined by a killing assay.

62. The host cell of claim 60 or 61, wherein the ratio of the host cell and the target cell in the killing assay is from 20:1 to 0.625:1.

63. The host cell of any one of claims 60-62, wherein a target cell is a T2 cell pulsed with 1 μg/mL to 50 μg/mL of HA-1 peptide.

64. The host cell of any one of claims 60-62, wherein the host cell is capable of killing a higher number of target cells when contacted with target cells with a heterozygous expression of HA-1.

65. The host cell of claim 64, wherein the host cell is capable of killing an at least 1.05-fold higher number of target cells.

66. The host cell of any one of claims 60, 61, 64 and 65, wherein the target cell is THP-1 or TF1 cell line.

67. The host cell of any one of claims 54-66, wherein the HA-1 immunogenic peptide comprises the amino acid sequence VLHDDLLEA.

68. The host cell of any one of claims 54-67, wherein the MHC molecule is a MHC class I molecule.

69. The host cell of any one of claims 54-68, wherein the MHC molecule comprises an MHC alpha chain that is an HLA serotype HLA-A*02.

70. The host cell of any one of claims 54-69, wherein the HLA allele is selected from the group consisting of HLA-A*02:01, HLA-A*02:02, HLA-A*02:03, HLA-A*02:05, HLA-A*02:06, and HLA-A*02:07 allele.

71. The host cell of any one of claims 54-70, wherein the target cell is a cell line selected from the group consisting of SupM2, THP-1, and TF1 cell line, is a hematopoietic cancer cell expressing HA-1, or is not SU-DHL1.

72. The host cell of claim 71, wherein the cancer cell is of a hematological malignancy selected from the group consisting of a leukemia, a lymphoma, a myelodysplastic disorder, a myeloproliferative neoplasm, or myeloma.

73. The host cell of any one of claims 54-72, wherein a) the host cell does not induce T cell expansion, cytokine release, or cytotoxic killing when contacted with a target cell that comprises a peptide-MHC (pMHC) complex comprising a CCDC114 peptide epitope in the context of an MHC molecule and/or b) the host cell does not express HA-1 antigen, is not recognized by a binding protein of any one of claims 1-30, is not of serotype HLA-A*02, and/or does not express an HLA-A*02 allele, optionally wherein the HLA-A*02 allele is HLA-A*02:01 and/or HLA-A*02:06.

74. A population of host cells of any one of claims 42-73.

75. A composition comprising: a) a binding protein according to any one of claims 1-30, b) an isolated nucleic acid according to claim 32 or 33, c) a vector according to any one of claims 34-41, d) a host cell according to any one of claims 42-73, and/or e) a population of host cells according to claim 74, and a carrier.

76. A device or kit comprising: a) a binding protein according to any one of claims 1-30, b) an isolated nucleic acid according to claim 32 or 33, c) a vector according to any one of claims 34-41, d) a host cell according to any one of claims 42-73, and/or e) a population of host cells according to claim 74, said device or kit optionally comprising a reagent to detect binding of a), d) and/or e) to a pMHC complex.

77. A method of producing a binding protein according to any one of claims 1-30, wherein the method comprises the steps of: (i) culturing a transformed host cell which has been transformed by a nucleic acid comprising a sequence encoding a binding protein according to any one of claims 1-30 under conditions suitable to allow expression of said binding protein; and (ii) recovering the expressed binding protein.

78. A method of producing a host cell expressing a binding protein according to any one of claims 1-30, wherein the method comprises the steps of: (i) introducing a nucleic acid comprising a sequence encoding a binding protein according to any one of claims 1-30 into the host cell; (ii) culturing the transformed host cell under conditions suitable to allow expression of said binding protein.

79. A method of detecting the presence or absence of an HA-1 antigen and/or a cell expressing HA-1, optionally wherein the cell is a hyperproliferative cell, comprising detecting the presence or absence of said HA-1 antigen in a sample by use of at least one binding protein according to any one of claims 1-30, or at least one host cell according to any one of claims 42-73, wherein detection of the HA-1 antigen is indicative of the presence of an HA-1 antigen and/or cell expressing HA-1.

80. The method of claim 79, wherein the at least one binding protein, or the at least one host cell, forms a complex with the HA-1 peptide in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay.

81. The method of claim 79 or 80, further comprising obtaining the sample from a subject.

82. The method of any one of claims 79-81, further comprising confirming cells expressing HA-1 by bone marrow biopsy.

83. A method of detecting the level of a non-malignant disorder, a hyperproliferative disorder, or a relapse of a hyperproliferative disorder characterized by expression of an HA-1 antigen in a subject, comprising:
a) contacting a sample obtained from the subject with at least one binding protein according to any one of claims 1-30, at least one host cell according to any one of claims 42-73, or a population of host cells according to claim 74; and
b) detecting the level of reactivity,
wherein a higher level of reactivity compared to a control level indicates the level of a non-malignant disorder, a hyperproliferative disorder, or a relapse of a hyperproliferative disorder characterized by expression of an HA-1 antigen in the subject.

84. The method of claim 83, wherein the control level is a reference number.

85. The method of claim 83, wherein the control level is a level of a subject without the non-malignant disorder, a hyperproliferative disorder, or a relapse of a hyperproliferative disorder characterized by expression of an HA-1 antigen.

86. A method for monitoring the progression of a non-malignant disorder, a hyperproliferative disorder, or a relapse of a hyperproliferative disorder characterized by expression of an HA-1 antigen in a subject, the method comprising:
a) detecting in a subject sample at a first point in time the level of the HA-1 antigen or the cell of interest expressing HA-1, according to any one of claims 79-85;
b) repeating step a) at a subsequent point in time; and
c) comparing the level of HA-1 antigen or the cell of interest expressing HA-1 detected in steps a) and b) to monitor the progression of a non-malignant disorder, a hyperproliferative disorder, or a relapse of a hyperproliferative disorder characterized by expression of an HA-1 antigen in the subject, wherein an absent or reduced level of the HA-1 antigen or the cell of interest expressing HA-1 detected in step b) compared to step a) indicates an inhibited progression of the non-malignant disorder, the hyperproliferative disorder, or the relapse of a hyperproliferative disorder characterized by expression of an HA-1 antigen in the subject.

87. The method of claim 86, wherein between the first point in time and the subsequent point in time, the subject has undergone treatment to treat the non-malignant disorder, the hyperproliferative disorder, or the relapse of a hyperproliferative disorder characterized by expression of an HA-1 antigen.

88. A method of assessing the efficacy of a therapy for a non-malignant disorder, a hyperproliferative disorder, or a relapse of a hyperproliferative disorder characterized by expression of an HA-1 antigen comprising:
a) determining the presence or level of reactivity between a sample obtained from the subject and at least one binding protein according to any one of claims 1-30, at least one host cell according to any one of claims 42-73, or a population of host cells according to claim 74, in a first sample obtained from a subject prior to providing at least a portion of the therapy for the non-malignant disorder, the hyperproliferative disorder, or the relapse of a hyperproliferative disorder characterized by expression of an HA-1 antigen to the subject, and
b) determining the presence or level of reactivity between a sample obtained from the subject and at least one binding protein according to any one of claims 1-30, at least one host cell according to any one of claims 42-73, or a population of host cells according to claim 74, in a second sample obtained from the subject following provision of the portion of the therapy for the non-malignant disorder, the hyperproliferative disorder, or the relapse of a hyperproliferative disorder characterized by expression of an HA-1 antigen,
wherein the absence or a reduced level of reactivity in the second sample, relative to the first sample, is an indication that the therapy is efficacious for treating the non-malignant disorder, the hyperproliferative disorder, or the relapse of a hyperproliferative disorder characterized by expression of an HA-1 antigen in the subject.

89. The method of any one of claims 83-89, wherein the level of reactivity is indicated by a) the presence of binding and/or b) T cell activation and/or effector function.

90. The method of claim 89, wherein the T cell activation or effector function is T cell proliferation, killing, or cytokine release.

91. The method of any one of claims 83-90, wherein the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

92. A method of preventing and/or treating a non-malignant disorder, a hyperproliferative disorder or a relapse of a hyperproliferative disorder characterized by expression of an HA-1 antigen in a subject comprising administering to the subject a therapeutically effective amount of a composition comprising cells expressing at least one binding protein of any one of claims 1-30.

93. The method of claim 92, wherein the cell is an allogeneic cell, syngeneic cell, or autologous cell.

94. The method of claim 92 or 93, wherein the cell is genetically modified.

95. The method of any one of claims 92-94, wherein the cell comprises a chromosomal gene knockout of a TCR gene, an HLA gene, or both a TCR gene and an HLA gene.

96. The method of any one of claims 92-95, wherein the cell comprises a knockout of an HLA gene selected from an α1 macroglobulin gene, α2 macroglobulin gene, α3 macroglobulin gene, β1 microglobulin gene, β2 microglobulin gene, and a combination thereof.

97. The method of any one of claims 92-96, wherein the cell comprises a knockout of a TCR gene selected from a TCR α variable region gene, TCR β variable region gene, TCR constant region gene, and combinations thereof.

98. The method of any one claims 92-97, wherein the cell expresses CD8α and/or CD8β,
optionally wherein the CD8α and/or CD8β is fused to a CD34 enrichment tag.

99. The method of claim 98, wherein cells are enriched using the CD34 enrichment tag.

100. The method of any one of claims 92-99, wherein the cell is a hematopoietic progenitor cell, peripheral blood mononuclear cell (PBMC), cord blood cell, or immune cell.

101. The method of any one of claims 92-100, wherein the immune cell is a cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or combination thereof.

102. The method of any one of claims 92-101, wherein the T cell is a naive T cell, central memory T cell, effector memory T cell, or combination thereof.

103. The method of any one of claims 92-102, wherein the T cell is a primary T cell or a cell of a T cell line.

104. The method of any one of claims 92-103, wherein the T cell does not express or has a lower surface expression of an endogenous TCR.

105. The method of any one of claims 92-104, wherein the cell is capable of producing a cytokine or a cytotoxic molecule when contacted with a target cell that comprises a peptide-MHC (pMHC) complex comprising the HA-1 peptide eiptope in the context of an MHC molecule.

106. The method of any one of claims 92-105, wherein the cytokine is TNF-α, IL-2, and/or IFN-γ.

107. The method of any one of claims 92-106, wherein the cytotoxic molecule is perforins and/or granzymes, optionally wherein the cytotoxic molecule is granzyme B.

108. The method of any one of claims 92-107, wherein the cell is capable of producing a higher level of cytokine or a cytotoxic molecule when contacted with a target cell with a heterozygous expression of HA-1.

109. The method of claim 108, wherein the cell is capable of producing an at least 1.05-fold higher level of cytokine or a cytotoxic molecule.

110. The method of any one of claims 92-109, wherein the host cell is capable of killing a target cell that comprises a peptide-MHC (pMHC) complex comprising an HA-1 peptide eiptope in the context of an MHC molecule.

111. The method of any one of claims 92-110, wherein the host cell is capable of killing a higher number of target cells when contacted with target cells with a heterozygous expression of HA-1.

112. The method of claim 111, wherein the host cell is capable of killing an at least 1.05-fold higher number of target cells.

113. The method of any one of claims 92-112, wherein the HA-1 immunogenic peptide comprises the amino acid sequence VLHDDLLEA.

114. The method of any one of claims 92-113, wherein the MHC molecule is an MHC class I molecule.

115. The method of any one of claims 92-114, wherein the MHC molecule comprises an MHC alpha chain that is an HLA serotype HLA-A*02.

116. The method of any one of claims 92-115, wherein the HLA allele is selected from the group consisting of HLA-A*02:01, HLA-A*02:02, HLA-A*02:03, HLA-A*02:05, HLA-A*02:06, and HLA-A*02:07 allele.

117. The method of any one of claims 92-116, wherein the target cell is a non-malignant cell or a hyperproliferating cell expressing the HA-1 antigen in the subject.

118. The method of any one of claims 92-117, wherein the composition further comprises a pharmaceutically acceptable carrier.

119. The method of any one of claims 92-118, wherein the composition induces an immune response against the non-malignant cells or the hyperproliferating cells expressing the HA-1 antigen in the subject.

120. The method of any one of claims 92-119, wherein the composition induces an antigen-specific T cell immune response against the non-malignant cells or the hyperproliferating cells expressing the HA-1 antigen in the subject.

121. The method of any one of claims 92-120, wherein the antigen-specific T cell immune response comprises at least one of a CD4⁺ helper T lymphocyte (Th) response and a CD8+ cytotoxic T lymphocyte (CTL) response.

122. The method of any one of claims 83-121, wherein the hyperproliferative disorder comprises a hematological malignancy.

123. The method of any one of claims 83-122, wherein the hematological malignancy comprises a leukemia, a lymphoma, a myelodysplastic disorder, a myeloproliferative neoplasm, or a myeloma.

124. The method of any one of claims 83-123, wherein the hematological malignancy comprises a leukemia.

125. The method of any one of claims 83-124, wherein the leukemia is selected from acute myeloid leukemia (AML), acute lymphocytic leukemia (ALL), mixed phenotype acute leukemia (MPAL), chronic myeloid leukemia (CML), B cell prolymphocytic leukemia, hairy cell leukemia, or chronic lymphocytic leukemia (CLL).

126. The method of any one of claims 83-125, wherein the hematological disorder comprises a lymphoma.

127. The method of any one of claims 83-125, wherein the lymphoma is selected from Hodgkin's lymphoma (HL), non-Hodgkin's lymphoma (NHL), a central nervous system lymphoma, small lymphocytic lymphoma (SLL), CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, precursor B-lymphoblastic lymphoma, immunoblastic large cell lymphoma, intravascular large B-cell lymphoma, primary effusion lymphoma, or Burkitt's lymphoma.

128. The method of any one of claims 83-123, wherein the hematological malignancy comprises a myelodysplastic disorder (MDS), optionally wherein the MDS is selected from refractory cytopenia with unilineage dysplasia (refractory anemia, refractory neutropenia, and refractory thrombocytopenia), refractory anemia with ring sideroblasts (RARS), refractory anemia with ring sideroblasts-thrombocytosis (RARS-t), refractory cytopenia with multinieage dysplasia (RCMD), refractory cytopenia with multinieage dysplasia and ring sideroblasts (RCMD-RS), refractory anemia with excess blasts (RAEB), myelodysplasia unclassifiable, refractory cytopenia of childhood, or MDS with isolated del(5q).

129. The method of any one of claims 83-123, wherein the non-malignant disorder is an immune deficiency disorder, optionally wherein the immune deficiency disorder is selected from the group consisting of severe combined immunodeficiency (SCID), Wiskott-Aldrich syndrome, Omenn syndrome, X-linked lymphoproliferative syndrome, chronic granulomatous disease, leukocyte adhesion deficiency, DiGeorge syndrome, and indications for hematopoietic stem cell transplantation (HCT).

130. The method of any one of claims 83-121, wherein the non-malignant disorder is a non-malignant hematology, optionally wherein the non-malignant hematology disorder is selected from the group consisting of as sickle cell anemia, thalassemia, aplastic anemia, hemophagocytic lymphohistiocytosis (HLH), severe aplastic anemia, marrow failure syndromes, Fanconi anemia, Diamond-Blackfan anemia, and Shwachman Diamond syndrome.

131. The method of any one of claims 83-121, wherein the non-malignant disorder is an autoimmune disorder, optionally wherein the autoimmune disorder is systemic sclerosis or multiple sclerosis.

132. The method of any one of claims 92-131, wherein the subject is receiving or previously received a hematopoietic cell transplant (HCT), optionally wherein the HCT comprises cells that do not express HA-1 antigen, are not recognized by a binding protein of any one of claims 1-30, are not of serotype HLA-A*02, and/or do not express an HLA-A*02:01 allele.

133. The method of claim 132, wherein the HCT comprises a donor hematopoeitic cell comprising a chromosomal knockout of a gene that encodes an HLA component, a chromosomal knockout of a gene that encodes a TCR component, or both.

134. The method of any one of claims 92-133, wherein the subject had previously received lymphodepleting chemotherapy.

135. The method of claim 134, wherein the lymphodepleting chemotherapy comprised cyclophosphamide, fludarabine, anti-thymocyte globulin, or a combination thereof.

136. The method of any one of claims 92-135, further comprising administering at least one additional treatment for the non-malignant disorder, the hyperproliferative disorder or the relapse of a hyperproliferative disorder to the subject.

137. The method of any one of claims 92-136, wherein the at least one additional treatment for the non-malignant disorder, the hyperproliferative disorder or the relapse of a hyperproliferative disorder is administered concurrently or sequentially with the composition.

138. The method of any one of claims 81-137, wherein the subject is an animal model of a disorder characterized by HA-1 expression and/or the mammal, optionally wherein the mammal is a human, a primate, or a rodent.

139. An expression vector comprising a promoter operably linked to a nucleic acid sequence encoding CD8α and/or CD8β.

140. The vector of claim 139, wherein the nucleic acid sequence encoding CD8α or CD8β is operably linked to a nucleic acid encoding a tag such that the tag is fused to the CD8α or CD8β.

141. The vector of claim 139 or 140, wherein the nucleic acid encoding a tag is at the 5′ upstream of the nucleic acid sequence encoding CD8α or CD8β such that the tag is fused to the N-terminal of CD8α or CD8β.

142. The vector of any one of claims 139-141, wherein the tag is a CD34 enrichment tag.

143. The vector of any one of claims 139-142, wherein the vector further comprises a nucleic sequence encoding a TCRα and/or TCRβ.

144. The vector of any one of claims 139-143, wherein the TCRα and/or TCRβ comprises a mutated transmembrane domain and/or a mutated constant domain.

145. The vector of any one of claims 139-144, wherein the mutated transmembrane domain and/or mutated constant domain enhance cellular surface expression of TCRα and/or TCRβ while decreasing expression of endogenous TCRα and/or TCRβ.

146. The vector of any one of claims 139-145, wherein the nucleic acid sequence encoding CD8α and/or CD8β and the nucleic sequence encoding TCRα and/TCRβ, are interconnected with an internal ribosome entry site or a nucleic acid sequence encoding a self-cleaving peptide.

147. The vector of any one of claims 139-146, wherein the self-cleaving peptide is P2A, E2A, F2A or T2A.

148. The vector of any one of claims 139-147, wherein the vector further comprises a nucleic sequence encoding a polypeptide selected from the group consisting of polypeptide sequences listed in Table 1, or a sequence with at least about 80% homology to a nucleic acid encoding a polypeptide selected from the group consisting of the polypeptide sequences listed in Table 1, optionally wherein the isolated nucleic acid molecule comprises 1) a TRAV, TRAJ, and/or TRAC gene or fragment thereof selected from the group of TRAV, TRAJ, and TRAC genes listed in Table 1 and/or 2) a TRBV, TRBJ, and/or TRBC gene or fragment thereof selected from the group of TRBV, TRBJ, and TRBC genes listed in Table 1.

149. The vector of any one of claims 139-148, wherein the vector has a nucleic sequence set forth in Table 3.


SEQUENCE LISTING (53 sequences)

SEQ ID NO: 1 - protein, 273 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRGGTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWS

SEQ ID NO: 2 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSRTAGDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 3 - protein, 273 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRGGTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 4 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSRTAGDTQYFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 5 - protein, 275 aa, Artificial Sequence
  Description of Artificial Sequence Synthetic polypeptide
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVHLNAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 6 - protein, 309 aa, Artificial Sequence
  Description of Artificial Sequence Synthetic polypeptide
MGPGLLCWVLLCLLGAGPVDAGVTQSPTHLIKTRGQHVTLRCSPISGHKSVSWYQQVLGQGPQFIFQYYEKEERGRGNFPDRFSARQFPNYSSELNVNALLLGDSALYLCASSFDRGTNGYTFGSGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 7 - protein, 266 aa, Artificial Sequence
  Description of Artificial Sequence Synthetic polypeptide
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASIMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 8 - protein, 309 aa, Artificial Sequence
  Description of Artificial Sequence Synthetic polypeptide
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLSVVDEQFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 9 - protein, 267 aa, Artificial Sequence
  Description of Artificial Sequence Synthetic polypeptide
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAVGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 10 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPTTINEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 11 - protein, 275 aa, Artificial Sequence
  Description of Artificial Sequence Synthetic polypeptide
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVANAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 12 - protein, 306 aa, Artificial Sequence
  Description of Artificial Sequence Synthetic polypeptide
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASGRSGTKFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 13 - protein, 266 aa, Artificial Sequence
  Description of Artificial Sequence Synthetic polypeptide
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCPIRQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 14 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVVVDTQYFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 15 - protein, 273 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 16 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASILVGGTEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 17 - protein, 271 aa, Artificial Sequence
  Description of Artificial Sequence Synthetic polypeptide
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAARGPGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWS

SEQ ID NO: 18 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLSDRFSAERPKGSFSTLEIQRTEQGDSAMYLCASVAGTVYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 19 - protein, 271 aa, Artificial Sequence
  Description of Artificial Sequence Synthetic polypeptide
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAARGPGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 20 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLSDRFSAERPKGSFSTLEIQRTEQGDSAMYLCASVAGTVYNEQFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 21 - protein, 275 aa, Artificial Sequence
  Description of Artificial Sequence Synthetic polypeptide
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASGLGSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWS

SEQ ID NO: 22 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLLAGEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 23 - protein, 275 aa, Artificial Sequence
  Description of Artificial Sequence Synthetic polypeptide
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASGLGSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 24 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLLAGEKLFFGSGTQLSVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 25 - protein, 268 aa, Artificial Sequence
  Description of Artificial Sequence Synthetic polypeptide
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGPSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 26 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLLTTNEQFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 27 - protein, 275 aa, Artificial Sequence
  Description of Artificial Sequence Synthetic polypeptide
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCALYSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 28 - protein, 310 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASFELAGGYEQFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 29 - protein, 272 aa, Artificial Sequence
  Description of Artificial Sequence Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVIGLGGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWS

SEQ ID NO: 30 - protein, 309 aa, Artificial Sequence
  Description of Artificial Sequence Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVKGEKLFFGSGTQLSVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 31 - DNA, 9683 bp, Artificial Sequence
  Description of Artificial Sequence Synthetic polynucleotide
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacaggcgcgccagagatccagtttggacctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatgggcaccagcctcctctgctggatggccctgtgtctcctgggggcagatcacgcagatactggagtctcccaggaccccagacacaagatcacaaagaggggacagaatgttactttcaggtgtgatccaatttctgaacacaaccgcctttattggtaccgccagaccctggggcagggcccagagtttctgacttacttccagaatgaagctcaacttgaaaaatcaaggctgctcagtgatcggttctctgcagagaggcctaagggatctttctccaccttggagatccagcgcacagagcagggggactctgccatgtatctctgtgccagcagccgcactgctggagatactcagtattttggcccaggcacccggctgacagtgctcgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaccctcttgggcctgcttatcctttggctgcagctgcaatgggtgagcagcaaacaggaggtgactcagattcctgcagctctgagtgtcccagaaggagaaaacttggttctcaactgcagtttcactgatagcgctatttacaacctccagtggtttaggcaggaccctgggaaaggcctcacatctctgttgcttattcagtcaagtcagagagagcaaacaagtggacgccttaatgcctctctggataaatcatcaggacgcagtactctttacattgcagcttctcagcctggtgattcagccacctacctgtgcgctgtgaggggtggtacctcaggaacctacaaatacatctttggaacaggcaccaggctgaaggttcttgcaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatggcgctgcccgtcaccgcgctgctgctgcccctggcgctgctgttacacgccgctcggccagagcttcccacccagggcacattctccaacgtgtccaccaatgtgtcgggaggcggcggatcgtcccagttcagagtgtcccctctggaccgcacctggaacctgggcgagaccgtggagctgaaatgtcaggtcctgctgagcaacccgacctccgggtgcagttggctgttccagccgcgtggtgctgccgcaagccctacgttcctgctttacctgagccagaacaagcccaaggcggccgagggcctggacacccagagattctccggcaagcgcctgggggacacattcgtgcttactttgagcgatttccgcagagagaacgagggctactatttctgttcggcgctgagcaattccatcatgtatttcagccactttgtgccagtgttcctgcctgccaagcctaccacaacaccagctccccgtcccccgactccggcgcctaccatcgcgagtcaaccgttgagcctgaggcctgaggcttgtcggcccgctgcggggggtgccgtccacaccaggggcctcgactttgcgtgcgacatctatatttgggcgcctctggcgggtacctgcggggtgctgctgctgtcattggtgattaccctgtactgcaatcaccgcaaccgccggcgggtctgtaagtgcccacggcctgtggtcaagtccggtgacaaaccgtcgctctcggctcgctacgtgcgcgctaagcgcagcggttccggggccaccaacttttcattgctgaagcaggccggtgatgtggaggagaatccagggcccatgcgccccaggctttggctccttcttgctgctcagctcactgtcttgcatggcaactccgttctgcagcagactcccgcctacatcaaggtgcagacgaacaagatggtgatgctgtcatgcgaggccaagatctctctttcaaatatgagaatttattggctacgacagcgccaggccccctccagcgacagccaccacgagttcctggcgctttgggattctgctaaaggcaccatccatggagaggaggtggaacaggagaagatagctgtcttccgcgacgcatcccgcttcatcctgaacctgaccagcgtgaagccggaggacagcggcatctacttctgtatgatcgttggctcccccgagctgaccttcggcaaaggcacccagctgtccgtggtggacttcctgcccaccacagcccagccaaccaagaaatccaccctcaagaagcgcgtgtgccgactgccccgccctgaaacccagaagggccctctgtgctcccccatcacccttggactgctggtggcgggagtcctggtgctgctcgtatctctgggtgtcgccatccacctgtgctgccgccgccgccgcgcccgcctgaggtttatgaaacagttttacaagtgataaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 32 - DNA, 5709 bp, Artificial Sequence
  Description of Artificial Sequence Synthetic polynucleotide
aattaattgcttgcaattaaccctttaacggttataaggatctagatgagatagaaagatttggttttcggatttgtgttacataagatgcctaaaataaaaattgagattcaattttttttaaacttttttttaattggtggtaagaatattccctctacctgtttgagagtaatgaaattgtagtatgatttttcaacaaactaaaaaaacaacataaatctcacataataactttatttcaatcacacaattgaataccaatattaattaatggttgacagtacttaccagcctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatgggcaccagcctcctctgctggatggccctgtgtctcctgggggcagatcacgcagatactggagtctcccaggaccccagacacaagatcacaaagaggggacagaatgttactttcaggtgtgatccaatttctgaacacaaccgcctttattggtaccgccagaccctggggcagggcccagagtttctgacttacttccagaatgaagctcaacttgaaaaatcaaggctgctcagtgatcggttctctgcagagaggcctaagggatctttctccaccttggagatccagcgcacagagcagggggactctgccatgtatctctgtgccagcagccgcactgctggagatactcagtattttggcccaggcacccggctgacagtgctcgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaccctcttgggcctgcttatcctttggctgcagctgcaatgggtgagcagcaaacaggaggtgactcagattcctgcagctctgagtgtcccagaaggagaaaacttggttctcaactgcagtttcactgatagcgctatttacaacctccagtggtttaggcaggaccctgggaaaggcctcacatctctgttgcttattcagtcaagtcagagagagcaaacaagtggacgccttaatgcctctctggataaatcatcaggacgcagtactctttacattgcagcttctcagcctggtgattcagccacctacctgtgcgctgtgaggggtggtacctcaggaacctacaaatacatctttggaacaggcaccaggctgaaggttcttgcaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatggcgctgcccgtcaccgcgctgctgctgcccctggcgctgctgttacacgccgctcggccagagcttcccacccagggcacattctccaacgtgtccaccaatgtgtcgggaggcggcggatcgtcccagttcagagtgtcccctctggaccgcacctggaacctgggcgagaccgtggagctgaaatgtcaggtcctgctgagcaacccgacctccgggtgcagttggctgttccagccgcgtggtgctgccgcaagccctacgttcctgctttacctgagccagaacaagcccaaggcggccgagggcctggacacccagagattctccggcaagcgcctgggggacacattcgtgcttactttgagcgatttccgcagagagaacgagggctactatttctgttcggcgctgagcaattccatcatgtatttcagccactttgtgccagtgttcctgcctgccaagcctaccacaacaccagctccccgtcccccgactccggcgcctaccatcgcgagtcaaccgttgagcctgaggcctgaggcttgtcggcccgctgcggggggtgccgtccacaccaggggcctcgactttgcgtgcgacatctatatttgggcgcctctggcgggtacctgcggggtgctgctgctgtcattggtgattaccctgtactgcaatcaccgcaaccgccggcgggtctgtaagtgcccacggcctgtggtcaagtccggtgacaaaccgtcgctctcggctcgctacgtgcgcgctaagcgcagcggttccggggccaccaacttttcattgctgaagcaggccggtgatgtggaggagaatccagggcccatgcgccccaggctttggctccttcttgctgctcagctcactgtcttgcatggcaactccgttctgcagcagactcccgcctacatcaaggtgcagacgaacaagatggtgatgctgtcatgcgaggccaagatctctctttcaaatatgagaatttattggctacgacagcgccaggccccctccagcgacagccaccacgagttcctggcgctttgggattctgctaaaggcaccatccatggagaggaggtggaacaggagaagatagctgtcttccgcgacgcatcccgcttcatcctgaacctgaccagcgtgaagccggaggacagcggcatctacttctgtatgatcgttggctcccccgagctgaccttcggcaaaggcacccagctgtccgtggtggacttcctgcccaccacagcccagccaaccaagaaatccaccctcaagaagcgcgtgtgccgactgccccgccctgaaacccagaagggccctctgtgctcccccatcacccttggactgctggtggcgggagtcctggtgctgctcgtatctctgggtgtcgccatccacctgtgctgccgccgccgccgcgcccgcctgaggtttatgaaacagttttacaagtgataaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtatacaggttacctcagtctcctaggtacgtcttatatctatgaaaaaacattcaaaagcacaacatctagaagaacttaccttttttcaccactctattgcaaagatatgtaccgatttctctcgaagtacaaaaaaccgctagttttcaaattcacctcaagactttgaaaaaaaattgaatctgtcaatgtcaaataaaatcagaaacaaatgtcataatgttacgttaatgttgtcaggtcgaaaaataaaattgcaaatagaaattttgttccttttttattggtttttattggtgggaaaaatattccctctaactgcaaaagggttaattatgttagaggtagagtcgacgctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtacc

SEQ ID NO: 33 - DNA, 2995 bp, Homo sapiens
ccgccctccagcgcgcggacgtggtaggcggccaagcgtttttggctgcttcccgggaagccgttggtcgcgcttgcgtagtgaaccgctgaggctgtgttgagcgctccttctccgcgcaactcccggcttcgacaccgccttccttcctatgaatccttaatttaacgtggtcacagtcagcttaagaccggcacctctcgggctccgtcttgtaggacctcggcctggcttcgagggcttccatacaggtcaccgcaggctctttcaactcccgccgcttccaggcccgcgtaaccagaccacaacactgaacttggagcaccaagaacaggagccccaccctgtttggccaggatgcctttgggacgcttggcagggagcgcccgctccgaggagggaagcgaggcatttctggagggaatggtggattgggagctgagtaggctgcagcgacaatgcaaagtgatggaaggggagaggcgggcctacagcaaggaagtccaccagcgcatcaacaagcaacttgaggagatccggcgcttggaggaggtacggggcgatctccaggtgcagatcagcgcagcccagaaccaggtcaagcggcttcgggacagtcagcggctggagaacatggaccgcctgctgaagggccgggcccaggtgcaggcggagatcgaggagctgcaggagcagaccagggccctggacaagcagatccaggagtgggagacgcggatctttacccacagtaagaatgtcaggtccccgggattcatcctggatcagaaggtcaagatcaggcgaaggatcaggatcctagaaaaccagttggacagggtcacctgtcactttgacaaccagctggtacggaatgcggccctgcgggaggagctggatctgctgcggatcgacaggaaccgctatctgaacgtggaccgcaagctgaagaaggagatccaccacctgcatcacctggtcagcacccttatcctctcctccacctctgcctacgccgtcagggaggaggcgaaggccaagatgggcttgctgcgggagcgcgcggagaaagaggaggcccagagcgagatggaggcgcaggtcctgcagcggcagatcttgcacctggagcagctgcaccacttcctcaagctcaagaacaacgaccggcagccggatcccgatgtcctggagaagcgtgaaaagcaggccggggaggtggccgagggcgtctggaagacctcccaggagaggctggtgctttgctacgaggacgccctgaataaactgtcccagctgatgggggagagtgaccctgacctgttggtgcagaagtatctggagatcgaggagcgcaactttgctgagttcaacttcatcaacgagcagaacttggagctggagcatgtgcaggaagagatcaaggagatgcaggaggctttggtgagcgcacgtgccagcaaggatgaccagcatttgctgcaggagcagcagcagaaggtgttgcagcagcgcatggacaaggtgcactcggaggctgagcgccttgaggcccgcttccaggatgtgcggggacagctggagaagctcaaggctgatatccagctcctcttcaccaaggcccattgcgacagcagcatgatcgatgacctccttggggtcaagaccagcatgggagaccgggacatgggcctcttcctgagcctcattgagaagcggctggtggagctcctgacagtgcaggccttcctacatgcccagagcttcacctccctggccgacgctgccctcctagtgctgggccagagcctggaggaccttccgaagaagatggccccacttcagccccctgacactctagaagaccccccgggttttgaggccagcgatgactaccccatgagcagggaggagctgctgagccaagtggagaagctggtggagctccaggagcaggcggaggcgcagcgccagaaggacctggccgccgccgccgcgaagctggacggcaccctgagcgtggacctggccagcacccagagggccggctccagtaccgtcctggtgcccaccaggcacccccatgccatccccgggtccattttgagccacaagactagcagagaccgtggctctcttggccacgtcacttttggcggcctcagctccagcactgggcatttgcccagccacatcacgcacggtgaccccaacactggccacgtgaccttcggctccaccagtgcctcgagtgggggccacgtgaccttcagacccgtcagcgccagcagctacctgggctccactggatacgtggggtccagcaggggcggagaaaacacagagggtggtgtggagagcggaggcacagcgtctgattcgagcggaggcctcgggtccagcagagaccacgtctccagcaccggccctgcctccagcactggcccgggctcctccaccagcaaagactcccggggctaacacgaggggcacgcagcccccaccctgccctggctctctgtggggtctttttttgtgtctctacttcccctgtttgccttgtgggtctctgtctttttctcccagtggcaggcctctgtctttttgccccttcaactgttttcatctgcccttcatcaccgcctcacctgtgtctcccggttcctttctctctgctcctgacactgcttttccctctgactcctgatctcctgttctctgggcttcctggtcagtgtctgtccgtcttgtctccatatctgccccttccctgtctgctgagccctggccccctgtggatcccccacctcctgtttccggctctctctcctcatctccatctcccaggctctccacgcatcctgtgtccctgtctctgtagctccttactccccaagagccccccagtgctgacaggtgtgggaaaagtcagggcgaagacgccaggttcagcatgggaaaatgggactgtggaaataaagtatatgtgtggaga

SEQ ID NO: 34 - DNA, 2658 bp, Homo sapiens
aatcacccgctgattgaaaactactgggcctttggagatggggagcatacgggcaagactggagagtggattgggagctgagtaggctgcagcgacaatgcaaagtgatggaaggggagaggcgggcctacagcaaggaagtccaccagcgcatcaacaagcaacttgaggagatccggcgcttggaggaggtacggggcgatctccaggtgcagatcagcgcagcccagaaccaggtcaagcggcttcgggacagtcagcggctggagaacatggaccgcctgctgaagggccgggcccaggtgcaggcggagatcgaggagctgcaggagcagaccagggccctggacaagcagatccaggagtgggagacgcggatctttacccacagtaagaatgtcaggtccccgggattcatcctggatcagaaggtcaagatcaggcgaaggatcaggatcctagaaaaccagttggacagggtcacctgtcactttgacaaccagctggtacggaatgcggccctgcgggaggagctggatctgctgcggatcgacaggaaccgctatctgaacgtggaccgcaagctgaagaaggagatccaccacctgcatcacctggtcagcacccttatcctctcctccacctctgcctacgccgtcagggaggaggcgaaggccaagatgggcttgctgcgggagcgcgcggagaaagaggaggcccagagcgagatggaggcgcaggtcctgcagcggcagatcttgcacctggagcagctgcaccacttcctcaagctcaagaacaacgaccggcagccggatcccgatgtcctggagaagcgtgaaaagcaggccggggaggtggccgagggcgtctggaagacctcccaggagaggctggtgctttgctacgaggacgccctgaataaactgtcccagctgatgggggagagtgaccctgacctgttggtgcagaagtatctggagatcgaggagcgcaactttgctgagttcaacttcatcaacgagcagaacttggagctggagcatgtgcaggaagagatcaaggagatgcaggaggctttggtgagcgcacgtgccagcaaggatgaccagcatttgctgcaggagcagcagcagaaggtgttgcagcagcgcatggacaaggtgcactcggaggctgagcgccttgaggcccgcttccaggatgtgcggggacagctggagaagctcaaggctgatatccagctcctcttcaccaaggcccattgcgacagcagcatgatcgatgacctccttggggtcaagaccagcatgggagaccgggacatgggcctcttcctgagcctcattgagaagcggctggtggagctcctgacagtgcaggccttcctacatgcccagagcttcacctccctggccgacgctgccctcctagtgctgggccagagcctggaggaccttccgaagaagatggccccacttcagccccctgacactctagaagaccccccgggttttgaggccagcgatgactaccccatgagcagggaggagctgctgagccaagtggagaagctggtggagctccaggagcaggcggaggcgcagcgccagaaggacctggccgccgccgccgcgaagctggacggcaccctgagcgtggacctggccagcacccagagggccggctccagtaccgtcctggtgcccaccaggcacccccatgccatccccgggtccattttgagccacaagactagcagagaccgtggctctcttggccacgtcacttttggcggcctcagctccagcactgggcatttgcccagccacatcacgcacggtgaccccaacactggccacgtgaccttcggctccaccagtgcctcgagtgggggccacgtgaccttcagacccgtcagcgccagcagctacctgggctccactggatacgtggggtccagcaggggcggagaaaacacagagggtggtgtggagagcggaggcacagcgtctgattcgagcggaggcctcgggtccagcagagaccacgtctccagcaccggccctgcctccagcactggcccgggctcctccaccagcaaagactcccggggctaacacgaggggcacgcagcccccaccctgccctggctctctgtggggtctttttttgtgtctctacttcccctgtttgccttgtgggtctctgtctttttctcccagtggcaggcctctgtctttttgccccttcaactgttttcatctgcccttcatcaccgcctcacctgtgtctcccggttcctttctctctgctcctgacactgcttttccctctgactcctgatctcctgttctctgggcttcctggtcagtgtctgtccgtcttgtctccatatctgccccttccctgtctgctgagccctggccccctgtggatcccccacctcctgtttccggctctctctcctcatctccatctcccaggctctccacgcatcctgtgtccctgtctctgtagctccttactccccaagagccccccagtgctgacaggtgtgggaaaagtcagggcgaagacgccaggttcagcatgggaaaatgggactgtggaaataaagtatatgtgtggagagctggcttcaaaaaaaaaaaaaaa

SEQ ID NO: 35 - protein, 707 aa, Homo sapiens
MPLGRLAGSARSEEGSEAFLEGMVDWELSRLQRQCKVMEGERRAYSKEVHQRINKQLEEIRRLEEVRGDLQVQISAAQNQVKRLRDSQRLENMDRLLKGRAQVQAEIEELQEQTRALDKQIQEWETRIFTHSKNVRSPGFILDQKVKIRRRIRILENQLDRVTCHFDNQLVRNAALREELDLLRIDRNRYLNVDRKLKKEIHHLHHLVSTLILSSTSAYAVREEAKAKMGLLRERAEKEEAQSEMEAQVLQRQILHLEQLHHFLKLKNNDRQPDPDVLEKREKQAGEVAEGVWKTSQERLVLCYEDALNKLSQLMGESDPDLLVQKYLEIEERNFAEFNFINEQNLELEHVQEEIKEMQEALVSARASKDDQHLLQEQQQKVLQQRMDKVHSEAERLEARFQDVRGQLEKLKADIQLLFTKAHCDSSMIDDLLGVKTSMGDRDMGLFLSLIEKRLVELLTVQAFLHAQSFTSLADAALLVLGQSLEDLPKKMAPLQPPDTLEDPPGFEASDDYPMSREELLSQVEKLVELQEQAEAQRQKDLAAAAAKLDGTLSVDLASTQRAGSSTVLVPTRHPHAIPGSILSHKTSRDRGSLGHVTFGGLSSSTGHLPSHITHGDPNTGHVTFGSTSASSGGHVTFRPVSASSYLGSTGYVGSSRGGENTEGGVESGGTASDSSGGLGSSRDHVSSTGPASSTGPGSSTSKDSRG

SEQ ID NO: 36 - protein, 670 aa, Homo sapiens
MEGERRAYSKEVHQRINKQLEEIRRLEEVRGDLQVQISAAQNQVKRLRDSQRLENMDRLLKGRAQVQAEIEELQEQTRALDKQIQEWETRIFTHSKNVRSPGFILDQKVKIRRRIRILENQLDRVTCHFDNQLVRNAALREELDLLRIDRNRYLNVDRKLKKEIHHLHHLVSTLILSSTSAYAVREEAKAKMGLLRERAEKEEAQSEMEAQVLQRQILHLEQLHHFLKLKNNDRQPDPDVLEKREKQAGEVAEGVWKTSQERLVLCYEDALNKLSQLMGESDPDLLVQKYLEIEERNFAEFNFINEQNLELEHVQEEIKEMQEALVSARASKDDQHLLQEQQQKVLQQRMDKVHSEAERLEARFQDVRGQLEKLKADIQLLFTKAHCDSSMIDDLLGVKTSMGDRDMGLFLSLIEKRLVELLTVQAFLHAQSFTSLADAALLVLGQSLEDLPKKMAPLQPPDTLEDPPGFEASDDYPMSREELLSQVEKLVELQEQAEAQRQKDLAAAAAKLDGTLSVDLASTQRAGSSTVLVPTRHPHAIPGSILSHKTSRDRGSLGHVTFGGLSSSTGHLPSHITHGDPNTGHVTFGSTSASSGGHVTFRPVSASSYLGSTGYVGSSRGGENTEGGVESGGTASDSSGGLGSSRDHVSSTGPASSTGPGSSTSKDSRG

SEQ ID NO: 37 - protein, 9 aa, Unknown
  Description of Unknown HA-1 sequence
VLHDDLLEA

SEQ ID NO: 38 - protein, 9 aa, Unknown
  Description of Unknown HA-1 sequence
VLRDDLLEA

SEQ ID NO: 39 - protein, 9 aa, Unknown
  Description of Unknown CCDC114 sequence
SMIDDLLGV

SEQ ID NO: 40 - protein, 20 aa, Artificial Sequence
  Description of Artificial Sequence Synthetic peptide
  SITE (1)..(10): This region may encompass 0-10 residues
  SITE (11)..(20): This region may encompass 0-10 residues
  See specification as filed for detailed description of substitutions and preferred embodiments
GGGGGGGGGGSSSSSSSSSS

SEQ ID NO: 41 - protein, 10 aa, Artificial Sequence
  Description of Artificial Sequence Synthetic peptide
GGGGSGGGGS

SEQ ID NO: 42 - protein, 8 aa, Artificial Sequence
  Description of Artificial Sequence Synthetic peptide
GGGSGGGS

SEQ ID NO: 43 - protein, 11 aa, Artificial Sequence
  Description of Artificial Sequence Synthetic peptide
GGGSGGGSGGS

SEQ ID NO: 44 - protein, 9 aa, Unknown
  Description of Unknown “LAGLIDADG” family peptide motif sequence
LAGLIDADG

SEQ ID NO: 45 - DNA, 20 bp, Artificial Sequence
  Description of Artificial Sequence Synthetic primer
aggacatctcccatctgctg

SEQ ID NO: 46 - DNA, 20 bp, Artificial Sequence
  Description of Artificial Sequence Synthetic primer
ttgagccagtgtacgctcag

SEQ ID NO: 47 - protein, 10 aa, Unknown
  Description of Unknown MART-1 sequence
ELAGIGILTV

SEQ ID NO: 48 - DNA, 20 bp, Artificial Sequence
  Description of Artificial Sequence Synthetic oligonucleotide
agagtctctcagctggtaca

SEQ ID NO: 49 - DNA, 20 bp, Artificial Sequence
  Description of Artificial Sequence Synthetic oligonucleotide
cggctactacaaccagagcg

SEQ ID NO: 50 - DNA, 20 bp, Artificial Sequence
  Description of Artificial Sequence Synthetic oligonucleotide
agatcacactgacctggcag

SEQ ID NO: 51 - DNA, 20 bp, Artificial Sequence
  Description of Artificial Sequence Synthetic oligonucleotide
aggtcagtgtgatctccgca

SEQ ID NO: 52 - protein, 10 aa, Unknown
  Description of Unknown HA-1 sequence
VLHDDLLEAN

SEQ ID NO: 53 - protein, 10 aa, Unknown
  Description of Unknown HA-1 sequence
VLRDDLLEAN